(19) United States
(12) Patent Application Publication
(10) Pub. No.: US 2026/0162799 A1
(43) Pub. Date: Jun. 11, 2026

(54) CANCER BIOMARKERS FOR IMMUNE CHECKPOINT INHIBITORS

(71) Applicant: Strata Oncology, Inc., Ann Arbor, MI (US)

(72) Inventors: Daniel Reed Rhodes, Chelsea, MI (US); Scott Arthur Tomlins, Ann Arbor, MI (US); David Bryan Johnson, Chelsea, MI (US); Nikolay Khazanov, Ann Arbor, MI (US)

(21) Appl. No.: 18/708,534

(22) PCT Filed: Nov. 8, 2022
(86) PCT No.: PCT/US2022/049332

§ 371 Date: May 8, 2024

Related U.S. Application Data
(60) Provisional application No. 63/277,158, filed on Nov. 8, 2021.
(60) Provisional application No. 63/407,606, filed on Sep. 16, 2022.

(51) Int. Cl. G16H 20/17; C12Q 1/686; C12Q 1/6869; C12Q 1/6886; G16B 20/20; G16B 25/10

(52) U.S. Cl. CPC G16H 20/17; C12Q 1/686; C12Q 1/6869; C12Q 1/6886; G16B 20/20; G16B 25/10; C12Q 2600/106

(57) ABSTRACT

Disclosed herein are methods for identifying and treating cancer patients that benefit from immune checkpoint inhibitor therapies.

### RELATED APPLICATION

This application claims priority to, and the benefit of, co-pending U.S. Provisional Application No. 63/277,158, filed Nov. 8, 2021, and co-pending U.S. Provisional Application No. 63/407,606, filed Sep. 16, 2022. The disclosures of both provisional applications are hereby incorporated by reference in their entireties.

### BACKGROUND OF THE INVENTION

Anti-PD-1 and anti-PD-L1 (PD-[L]1) monoclonal antibodies, known as checkpoint inhibitors (CPIs), have transformed cancer care, and are approved for use in multiple tumor types and pan tumor indications (microsatellite instability high/mismatch repair deficient [MSI-H/dMMR] and tumor mutation burden [TMB]≥10 mutations/megabase [Muts/Mb])¹⁻³. Improved biomarkers capable of predicting anti-PD-(L)1 benefit have the potential to expand CPIs to additional patient populations outside of currently approved indications, and to focus their application more effectively on likely responsive patients when alternative therapies exist. Additionally, this focused application reduce unnecessary activation of the immune system via checkpoint inhibitors in subjects that are unlikely to respond to the therapy, thus reducing the number of adverse events in these subjects, which may include colitis, hepatitis, adrenocorticotropic hormone insufficiency, hypothyroidism, type 1 diabetes, acute kidney injury and myocarditis. PD-L1 immunohistochemistry (IHC) is required for treatment in many tumor types and serves as a companion diagnostic biomarker; although antibodies, staining platforms, PD-L1 expressing cells included in scoring algorithms, and cutoffs vary across tumor types⁴⁻¹⁴. In addition, high TMB predicts CPI response across multiple tumor types, although TMB determination approaches vary across studies and tests, only a fraction of TMB high (TMB-H) patients benefit, and a single TMB cutoff may not be optimum across tumor types or CPIs¹⁵⁻²⁴. For example, in the KEYNOTE-158 study of 9 tumor types leading to pan-solid tumor approval of second-line pembrolizumab (anti-PD-1) in patients with TMB≥10 Muts/Mb by the FoundationOne companion diagnostic (CDx) comprehensive genomic profiling (CGP) device, objective responses were observed in 37%, 13%, and 6% of patients with TMB≥13 Muts/Mb, ≥10 and <13 Muts/Mb, and <10 Muts/Mb, respectively^{25,26}.

Additionally, although only pembrolizumab is approved for patients with high TMB, numerous retrospective and prospective analyses support the clinical utility of high TMB by comprehensive genomic profiling (CGP) for predicting durable responses to other anti-PD-(L)1 monotherapies, including both other PD-1 (e.g. nivolumab) and PD-L1 (e.g. atezolizumab) monoclonal antibodies²⁷⁻³². Notably, in prospective basket studies of patients with ≥2^{nd} line solid tumors having high TMB by FoundationOne CDx treated with nivolumab or atezolizumab, ORRs of 28% (n=10/36) and 19% (n=17/90), respectively, were observed in patients with TMB≥10 Mut/Mb, with increased ORRs of 47% (n=8/17) and 38% (n=16/42), respectively, in patients with TMB≥16 Mut/Mb^{31,32}. In addition to potentially identifying patients outside of current indications who may benefit from PD-(L)1 monotherapy, given the increasing number of approved PD-(L)1 combination therapy regimens and the thousands of ongoing combination trials, biomarkers enabling the identification of PD-(L)1 monotherapy benefit is of particular importance in tumor types where only combination therapy regimens are approved (or monotherapy is only approved in later lines) as combination regimens have increased clinical and financial toxicity and a recent meta-analysis demonstrating essentially no evidence for additive or synergistic benefit between PD-(L)1 therapies and other agents in approved combination regimens³³.

Numerous translational research studies have demonstrated that PD-L1 expression, TMB (with clonal TMB showing increased predictive ability vs. TMB methods including all somatic mutations), and other immune related gene expression markers focusing on the tumor microenvironment (TME) are independent predictors of response^{15,34-47}. For example, in bladder cancer, multiple studies have demonstrated the potential for PD-L1 by IHC, TMB, and T-cell-inflamed gene expression to predict PD-(L)1 therapy benefit, whether alone or in combination with chemotherapy, with an only increasing need to maximize PD-(L)1 benefit given the number of other approved agents in different therapy classes (chemotherapy, antibody drug conjugates and small molecule inhibitors) that must sequenced⁴⁸⁻⁵³. Importantly, however, a single, integrative, clinically applicable and validated test for treatment selection across solid tumors is lacking.

### SUMMARY OF THE INVENTION

By leveraging PD-(L)1 therapy treatment data and CGP plus quantitative transcriptomic profiling (CGP+qTP) data from the Strata Trial (NCT03061305)—an observational clinical trial evaluating the impact of molecular profiling on patients with advanced solid tumors—the inventors have developed and validated an integrated Immunotherapy Response Score (IRS) that predicts pan-solid tumor PD-(L)1 benefit by both real-world progression free survival (rwPFS) and overall survival (OS) by an analytically and clinically validated CGP+qTP laboratory developed test (LDT) applicable to minute formalin-fixed paraffin-embedded (FFPE) tissue specimens.

Some aspects of the present invention are directed to a method of treatment, comprising: (a)(i) measuring expression levels of RNA transcripts for at least two of PD-1, TOP2A, PD-L1 and ADAM12 obtained from a tumor specimen from a subject; (b) measuring tumor mutation burden (TMB) in the biological sample and log 2 transforming the TMB measurement to provide a transformed TMB measurement; (c) calculating an Immunotherapy Response Score (IRS) from the expression levels of the RNA transcripts of the at least two of PD-1, TOP2A, PD-L1 and ADAM12 obtained in step (a), and the transformed TMB measurement, wherein the IRS is positively correlated with the likelihood that the patient has a beneficial response to checkpoint inhibitor therapy, and obtaining an IRS having a value indicating a beneficial response to checkpoint inhibitor therapy; and (d) administering the checkpoint inhibitor therapy to the subject.

In some embodiments, the step of measuring expression levels of RNA transcripts comprises measuring expression levels of RNA transcripts for at least three of PD-1, TOP2A, PD-L1 and ADAM12, and the step of calculating an IRS comprises calculating the IRS from the expression levels of the RNA transcripts of the at least three of PD-1, TOP2A, PD-L1 and ADAM12, and the transformed TMB measurement. In some embodiments, the step of measuring expression levels of RNA transcripts comprises measuring expression levels of RNA transcripts for all of PD-1, TOP2A, PD-L1 and ADAM12, and the step of calculating an IRS comprises calculating the IRS from the expression levels of the RNA transcripts of all of PD-1, TOP2A, PD-L1 and ADAM12, and the transformed TMB measurement. In some embodiments, the step of measuring expression levels of RNA transcripts comprises measuring expression levels of RNA transcripts of at least PD-1 and PD-L1, and the step of calculating an IRS comprises calculating the IRS from the expression levels of the RNA transcripts of at least PD-1 and PD-L1, and the transformed TMB measurement.

In some embodiments, the step of measuring expression levels of RNA transcripts comprises measuring expression levels of RNA transcripts of PD-1, PD-L1, and ADAM12 and wherein the step of calculating an IRS comprises calculating the IRS from the expression levels of the RNA transcripts of PD-1, PD-L1, and ADAM12, and the transformed TMB measurement. In some embodiments, step (a) further comprises ii) measuring expression levels of RNA transcripts for at least one reference gene in the biological sample, and iii) normalizing the measured expression levels of the measured RNA transcripts of the at least two, the at least three, or all of PD-1, TOP2A, PD-L1 and ADAM12 against the level of the RNA transcripts of the at least one reference gene to provide normalized expression levels of the PD-1, TOP2A, PD-L1 and ADAM12 RNA transcripts. In some embodiments, the expression levels of RNA transcripts used to calculate the IRS comprises normalized expression levels of RNA transcripts.

In some embodiments, step (a) further comprises iv) median centering the measured expression levels of RNA transcripts of the at least two, the at least three, or all of PD-1, TOP2A, PD-L1 and ADAM12, prior or after to normalizing the expression levels of the measured RNA transcripts.

In some embodiments, step (a) further comprises v) log 2 transforming the measured expression levels, the median centered expression levels, the normalized expression levels or the median centered normalized expression levels of RNA transcripts of the at least two, the at least three, or all of PD-1, TOP2A, PD-L1 and ADAM12, and wherein the expression levels utilized to calculate the IRS in step c are transformed expression levels, transformed median centered expression levels, transformed normalized expression levels or transformed median centered normalized expression levels.

In some embodiments, the IRS is calculated as follows: IRS=approximately 0.27*[transformed TMB measurement]+approximately 0.11*[transformed PD-1 level]+approximately 0.06*[transformed PD-L1 level]−approximately 0.06 [transformed ADAM12 level]−approximately 0.077*[transformed TOP2A level],

wherein the transformed PD-1 level, the transformed PD-L1 level, the transformed ADAM12 level, the transformed TOP2A level, are each one of a transformed expression level, a transformed median centered expression level, a transformed normalized expression level, or a transformed median centered normalized expression level.

In some embodiments, the IRS is calculated as follows: IRS=0.27*[transformed TMB measurement]+0.11*[transformed PD-1 level]+0.06*[transformed PD-L1 level]−0.06 [transformed ADAM12 level]−0.077*[transformed TOP2A level],

wherein the transformed PD-1 level, the transformed PD-L1 level, the transformed ADAM12 level, the transformed TOP2A level, are each one of a transformed expression level, a transformed median centered expression level, a transformed normalized expression level, or a transformed median centered normalized expression level.

In some embodiments, the IRS is calculated as follows: IRS=0.273758*[transformed TMB measurement]+0.112641*[transformed PD-1 level]+0.061904*[transformed PD-L1 level]−0.057991 [transformed ADAM12 level]−0.077011*[transformed TOP2A level],

wherein the transformed PD-1 level, the transformed PD-L1 level, the transformed ADAM12 level, the transformed TOP2A level, are each one of a transformed expression level, a transformed median centered expression level, a transformed normalized expression level, or a transformed median centered normalized expression level.

In some embodiments, the IRS value indicating beneficial response to checkpoint inhibitor therapy is approximately 0.87 or more. In some embodiments, the IRS value indicating beneficial response to checkpoint inhibitor therapy is 0.873569 or higher.

In some embodiments, the one or more reference genes comprise three genes selected from CIAO1, EIF2B1, HMBS, CTCF, GGNBP2, ITGB7, MYC and SLC4A1AP. In some embodiments, the one or more reference genes comprise the combination of CIAO1, EIF2B1 and HMBS.

In some embodiments, the tumor specimen is a formalin-fixed paraffin-embedded (FFPE) tumor specimen. In some embodiments, the tumor specimen contains at least 20% tumor content.

In some embodiments, the tumor specimen is breast cancer, central or peripheral nervous system cancer, cancer of unknown primary, colorectal cancer, endometrial cancer, a gastrointestinal stromal tumor, glioma, hepatobiliary cancer, neuroendocrine cancer, ovarian cancer, pancreatic cancer, prostate cancer salivary gland cancer, sarcoma, or thyroid cancer.

In some embodiments, the tumor specimen is assessed as having microsatellite instability-low or microsatellite stability. In some embodiments, the tumor specimen is assessed as having a low tumor mutational burden, wherein low tumor mutational burden is classified as having less than 10 mutations per megabase (muts/Mb). In some embodiments, the expression levels of RNA transcripts are measured using PCR and next-generation sequencing.

In some embodiments, the checkpoint inhibitor is an anti-PD-1 antibody, an anti-CTLA-4 antibody, an anti-PD-L1 antibody, or an anti-PD-L2. In some embodiments, the checkpoint inhibitor is nivolumab, pembrolizumab, atezolizumab, cemiplimab, durvalumab, pidilizumab, atezolimumab, PDR001, BMS-936559, avelumab, ipilimumab, or SHR-1210. In some embodiments, the checkpoint inhibitor therapy is administered as a monotherapy. In some embodiments, the checkpoint inhibitor therapy is administered in combination with one or more other chemotherapeutic agents.

In some embodiments, the tumor specimen shows a TPS score of 1-49%. In some embodiments, the checkpoint inhibitor is administered as part of a 1^{st} line treatment regimen. In some embodiments, the checkpoint inhibitor is administered as part of a 2^{nd} line treatment regimen or higher.

Some aspects of the present disclosure are directed to a method of identifying a subject that would benefit from checkpoint inhibitor therapy, comprising: (a) measuring expression levels of RNA transcripts for PD-1, TOP2A, PD-L1 and ADAM12, and one or more reference genes in a biological sample obtained from a tumor specimen from the subject, wherein the one or more reference gene comprises three genes selected from CIAO1, EIF2B1, HMBS, CTCF, GGNBP2, ITGB7, MYC and SLC4A1AP; (b) log 2 transforming, median centering, and normalizing the measured expression levels of the RNA transcripts against the level of the RNA transcripts of at the least one reference gene to provide normalized levels of the RNA transcripts; (c) measuring tumor mutation burden (TMB) in the biological sample and log 2 transforming the TMB measurement to provide a transformed TMB measurement; (d) calculating a Immunotherapy Response Score (IRS) from the transformed normalized levels of the RNA transcripts of PD-1, TOP2A, PD-L1 and ADAM12, and the transformed TMB measurement, wherein the IRS is positively correlated with the likelihood that the patient has a beneficial response to checkpoint inhibitor therapy, and obtaining an IRS having a value indicating beneficial response to checkpoint inhibitor therapy; and (e) identifying the subject as benefiting from the checkpoint inhibitor therapy.

In some embodiments, the tumor specimen is from a cancer not approved for labeled use of the checkpoint inhibitor therapy. In some embodiments, the tumor specimen is assessed as having microsatellite instability-low or microsatellite stability. In some embodiments, the tumor specimen is assessed as having a low tumor mutational burden, wherein low tumor mutational burden is classified as less than 10 mutations per megabase (muts/Mb). In some embodiments, the tumor specimen shows a TPS score of 1-49%.

Some aspects of the present disclosure are directed to a method of identifying a subject that would benefit from checkpoint inhibitor therapy, comprising: (a) receiving, by a processor, measured expression levels of RNA transcripts for at least two of PD-1, TOP2A, PD-L1 and ADAM12, and at least one reference gene in a biological sample obtained from a tumor specimen from the subject; (b) log 2 transforming, median centering, and normalizing, by a processor, the measured expression levels of the RNA transcripts against the level of the RNA transcripts of at the least one reference gene to provide transformed normalized levels of the RNA transcripts; (c) receiving, by a processor, measured tumor mutation burden (TMB) in the biological sample; (d) log 2 transforming, by a processor, the TMB measurement to provide a transformed TMB measurement; (e) calculating, by a processor, an Immunotherapy Response Score (IRS) from the transformed normalized levels of the RNA transcripts of the at least two of PD-1, TOP2A, PD-L1 and ADAM12, and the transformed TMB measurement, that is positively correlated with the likelihood that the patient has a beneficial response to checkpoint inhibitor therapy; and (f) providing a determination if the subject has a checkpoint inhibitor responsive cancer.

In some embodiments, the IRS is calculated as follows: IRS=0.27*[transformed TMB measurement]+0.11*[transformed PD-1 normalized level]+0.06*[transformed PD-L1 normalized level]−0.06 [transformed ADAM12 normalized level]−0.077*[transformed TOP2A normalized level].

In some embodiments, the IRS is calculated as follows: IRS=0.273758*[transformed TMB measurement]+0.112641*[transformed PD-1 normalized level]+0.061904*[transformed PD-L1 normalized level]−0.057991 [transformed ADAM12 normalized level]−0.077011*[transformed TOP2A normalized level].

In some embodiments, the IRS value indicating beneficial response to checkpoint inhibitor therapy is approximately 0.87 or more. In some embodiments, the IRS value indicating beneficial response to checkpoint inhibitor therapy is 0.873569 or higher. In some embodiments, the one or more reference genes comprise three genes selected from CIAO1, EIF2B1, HMBS, CTCF, GGNBP2, ITGB7, MYC and SLC4A1AP. In some embodiments, the one or more reference genes comprise the combination of CIAO1, EIF2B1 and HMBS.

Some aspects of the present disclosure are directed to a method of treatment of a subject in need thereof with a checkpoint inhibitor therapy, comprising administering to said subject the checkpoint inhibitor therapy, wherein said subject in need thereof was previously identified by a method comprising: (a) measuring expression levels of RNA transcripts for at least two of PD-1, TOP2A, PD-L1 and ADAM12, and one or more reference genes in a biological sample obtained from a tumor specimen from the subject, wherein the one or more reference gene comprises three genes selected from CIAO1, EIF2B1, HMBS, CTCF, GGNBP2, ITGB7, MYC and SLC4A1AP; (b) log 2 transforming, median centering, and normalizing the measured expression levels of the RNA transcripts against the level of the RNA transcripts of at the least one reference gene to provide transformed normalized levels of the RNA transcripts; (c) measuring tumor mutation burden (TMB) in the biological sample and log 2 transforming the TMB measurement to provide a transformed TMB measurement; (d) calculating a Immunotherapy Response Score (IRS) from the transformed normalized levels of the RNA transcripts of the at least two of PD-1, TOP2A, PD-L1 and ADAM12, and the transformed TMB measurement, wherein the IRS is positively correlated with the likelihood that the patient has a beneficial response to checkpoint inhibitor therapy, and obtaining an IRS having a value indicating beneficial response to checkpoint inhibitor therapy.

In some embodiments, the IRS is calculated as follows: IRS=0.27*[transformed TMB measurement]+0.11*[transformed PD-1 normalized level]+0.06*[transformed PD-L1 normalized level]−0.06 [transformed ADAM12 normalized level]−0.077*[transformed TOP2A normalized level].

In some embodiments, the IRS is calculated as follows: IRS=0.273758*[transformed TMB measurement]+0.112641*[transformed PD-1 normalized level]+0.061904*[transformed PD-L1 normalized level]−0.057991 [transformed ADAM12 normalized level]−0.077011*[transformed TOP2A normalized level].

In some embodiments, the IRS value indicating beneficial response to checkpoint inhibitor therapy is approximately 0.87 or more. In some embodiments, the IRS value indicating beneficial response to checkpoint inhibitor therapy is 0.873569 or higher.

In some embodiments, the one or more reference genes comprise the combination of HMBS, CIAO1 and EIF2B1. In some embodiments, the tumor specimen is from a cancer not approved for labeled use of the checkpoint inhibitor therapy. In some embodiments, the tumor specimen is assessed as having microsatellite instability-low or microsatellite stability. In some embodiments, the tumor specimen is assessed as having a low tumor mutational burden, wherein low tumor mutational burden is classified as less than 10 mutations per megabase (muts/Mb).

In some embodiments, the checkpoint inhibitor therapy is administered as a monotherapy. In some embodiments, the checkpoint inhibitor therapy is administered in combination with one or more other chemotherapeutic agents. In some embodiments, the tumor specimen shows a TPS score of 1-49%.

In some embodiments, the tumor specimen is a formalin-fixed paraffin-embedded (FFPE) tumor specimen. In some embodiments, the tumor specimen is breast cancer, central or peripheral nervous system cancer, cancer of unknown primary, colorectal cancer, endometrial cancer, a gastrointestinal stromal tumor, glioma, hepatobiliary cancer, neuroendocrine cancer, ovarian cancer, pancreatic cancer, prostate cancer salivary gland cancer, sarcoma, or thyroid cancer.

In some embodiments, the expression levels of RNA transcripts are measured using PCR and next-generation sequencing.

In some embodiments, the checkpoint inhibitor is an anti-PD-1 antibody, an anti-CTLA-4 antibody, an anti-PD-L1 antibody, or an anti-PD-L2. In some embodiments, the checkpoint inhibitor is nivolumab, pembrolizumab, atezolizumab, cemiplimab, durvalumab, pidilizumab, atezolimumab, PDR001, BMS-936559, avelumab, ipilimumab, or SHR-1210.

Some aspects of the present invention are directed to a method of treatment of a subject in need thereof with a checkpoint inhibitor therapy, comprising administering to said subject the checkpoint inhibitor therapy, wherein said subject in need thereof was previously identified by a method comprising: a. measuring expression levels of RNA transcripts for PD-1 and PD-L2 obtained from a tumor specimen from a subject, b. log 2 transforming, median centering, and normalizing the measured expression levels of the RNA transcripts against the level of the RNA transcripts of the one or more reference genes to provide transformed normalized levels of the RNA transcripts; c. measuring tumor mutation burden (TMB) in the biological sample and log 2 transforming the TMB measurement to provide a transformed TMB measurement; and d. calculating a Immunotherapy Response Score (IRS) from the expression levels or normalized levels of the RNA transcripts of PD-1 and PD-L2, and the transformed TMB measurement, wherein the IRS is positively correlated with the likelihood that the patient has a beneficial response to checkpoint inhibitor therapy, and obtaining an IRS having a value indicating a beneficial response to checkpoint inhibitor therapy.

In some embodiments, the IRS is calculated as follows: IRS=3.97*exp(0.301*[transformed TMB measurement]+0.110*[PD-1 normalized level]+0.078*[PD-L2 normalized level]).

In some embodiments, step a. further comprises measuring the expression level of RNA transcripts for ADAM12. In some embodiments, the IRS is calculated as follows: IRS=3.90*exp(0.307*[transformed TMB measurement]+0.115*[PD-1 normalized level]+0.106*[PD-L2 normalized level]+−0.070*[ADAM12 normalized level]).

In some embodiments, step a. further comprises measuring the expression level of RNA transcripts for PD-L1. In some embodiments, the IRS is calculated as follows: IRS=3.91*exp(0.296*[transformed TMB measurement]+0.097*[PD-1 normalized level]+0.056*[PD-L2 normalized level]+0.0043*[PD-L1 normalized level]).

In some embodiments, step a. further comprises measuring the expression level of RNA transcripts for CD4. In some embodiments, the IRS is calculated as follows: IRS=4.10*exp(0.281*[transformed TMB measurement]+0.139*[PD-1 normalized level]+0.112*[PD-L2 normalized level]+−0.128*[CD4 normalized level]).

In some embodiments, the expression levels of RNA transcripts for PD-1, PD-L2, CD4, ADAM12, and PD-L1 are measured, and wherein the IRS is calculated as follows: IRS=3.95*exp(0.280*[transformed TMB measurement]+0.134*[PD-1 normalized level]+0.122*[PD-L2 normalized level]+−0.070*[ADAM12 normalized level]+−0.154*[CD4 normalized level]+0.052*[PD-L1 normalized level]).

In some embodiments, the expression levels of RNA transcripts for PD-1, PD-L2, CD4, and ADAM12 are measured, and wherein the IRS is calculated as follows: IRS=4.03*exp(0.287*[transformed TMB measurement]+0.147*[PD-1 normalized level]+0.143*[PD-L2 normalized level]−0.138*[CD4 normalized level]−0.073*[ADAM12 normalized level]).

In some embodiments, step a. further comprises measuring the expression level of RNA transcripts for VTCN1. In some embodiments, the IRS is calculated as follows: IRS=3.90*exp(0.309*[transformed TMB measurement]+0.104*[PD-1 normalized level]+0.087*[PD-L2 normalized level]+0.021*[VTCN1 normalized level]).

In some embodiments, the expression levels of RNA transcripts for PD-1, PD-L2, CD4, ADAM12, and VTCN1 are measured, and wherein the IRS is calculated as follows: IRS=3.95*exp(0.295*[transformed TMB measurement]+0.142*[PD-1 normalized level]+0.150*[PD-L2 normalized level]+0.020*[VTCN1 normalized level]+−0.070*[ADAM12 normalized level]+−0.139*[CD4 normalized level]).

In some embodiments of the methods disclosed herein, step a. further comprises measuring expression levels of RNA transcripts for at least one reference gene in the biological sample, step b. comprises normalizing the measured expression levels of the other measured RNA transcripts against the level of the RNA transcripts of at the least one reference gene to provide normalized levels of the other RNA transcripts, and step c. comprises calculating the IRS from the normalized levels.

In some embodiments, the IRS value indicating beneficial response to checkpoint inhibitor therapy is 10 or more. In some embodiments, the one or more reference genes comprise three genes selected from LRP1, MRPL13, TBP, HMBS, ITGB7, MYC, CIAO1, CTCF, EIF2B1, GGNBP2, SLC4A1AP.

In some embodiments, the tumor specimen is a formalin-fixed paraffin-embedded (FFPE) tumor specimen. In some embodiments, the tumor specimen is adrenal cancer, biliary cancer, bladder cancer, brain cancer, breast cancer, cervical cancer, colon cancer, rectum cancer, endometrial cancer, esophageal cancer, head or neck cancer, kidney cancer, liver cancer, non-small cell lung cancer, lung cancer, lymphoma, melanoma, meninges cancer, non-melanoma skin cancer, ovarian cancer, pancreatic cancer, prostate cancer, sarcoma, small intestine cancer, or stomach cancer.

In some embodiments, the expression levels of RNA transcripts are measured using PCR and next-generation sequencing.

In some embodiments, the checkpoint inhibitor is an anti-PD-1 antibody, an anti-CTLA-4 antibody, an anti-PD-L1 antibody, or an anti-PD-L2. In some embodiments, the checkpoint inhibitor is nivolumab, pembrolizumab, atezolizumab, durvalumab, pidilizumab, PDR001, BMS-936559, avelumab, or SHR-1210.

Some aspects of the present disclosure are directed to a method of identifying a subject that would benefit from checkpoint inhibitor therapy, comprising: a. measuring expression levels of RNA transcripts for PD-1, PD-L2 and, optionally, expression levels of RNA transcripts for one or more of CD4, ADAM12, PD-L1, and VTCN1, and at least one reference gene in a biological sample obtained from a tumor specimen from the subject; b. log 2 transforming, median centering, and normalizing the measured expression levels of the RNA transcripts against the level of the RNA transcripts of at the least one reference gene to provide normalized levels of the RNA transcripts; c. measuring tumor mutation burden (TMB) in the biological sample and log 2 transforming the TMB measurement to provide a transformed TMB measurement; d. calculating a Immunotherapy Response Score (IRS) from the normalized levels of the RNA transcripts of PD-1, PD-L2 and, optionally, one or more of CD4, ADAM12, PD-L1, and VTCN1, and the transformed TMB measurement, wherein the IRS is positively correlated with the likelihood that the patient has a beneficial response to checkpoint inhibitor therapy, and obtaining an IRS having a value indicating beneficial response to checkpoint inhibitor therapy; and e. identifying the subject as benefiting from the checkpoint inhibitor therapy.

In some embodiments, the tumor specimen is from a cancer not approved for labeled use of the checkpoint inhibitor therapy. In some embodiments, the calculated IRS value indicates that the median time-to-next-treatment (TNTT) is 24 months or greater.

Some aspects of the present disclosure are directed to a method of identifying a subject that would benefit from checkpoint inhibitor therapy, comprising: a. receiving, by a processor, measured expression levels of RNA transcripts for PD-1, PD-L2 and, optionally, expression levels of RNA transcripts for one or more of CD4, ADAM12, PD-L1, and VTCN1, and at least one reference gene in a biological sample obtained from a tumor specimen from the subject; b. log 2 transforming, median centering, and normalizing, by a processor, the measured expression levels of the RNA transcripts against the level of the RNA transcripts of at the least one reference gene to provide normalized levels of the RNA transcripts; c. receiving, by a processor, measured tumor mutation burden (TMB) in the biological sample; d. log 2 transforming, by a processor, the TMB measurement to provide a transformed TMB measurement; e. calculating, by a processor, a Immunotherapy Response Score (IRS) from the normalized levels of the RNA transcripts of PD-1, PD-L2 and, optionally, one or more of CD4, ADAM12, PD-L1, and VTCN1, and the transformed TMB measurement, that is positively correlated with the likelihood that the patient has a beneficial response to checkpoint inhibitor therapy; and f. providing a determination if the subject has a checkpoint inhibitor responsive cancer.

In some embodiments, the IRS is calculated as follows: IRS=3.97*exp(0.301*[transformed TMB measurement]+0.110*[PD-1 normalized level]+0.078*[PD-L2 normalized level]).

In some embodiments, the IRS is calculated as follows: IRS=4.03*exp(0.287*[transformed TMB measurement]+0.147*[PD-1 normalized level]+0.143*[PD-L2 normalized level]−0.138*[CD4 normalized level]−0.073*[ADAM12 normalized level]).

In some embodiments, the IRS is calculated as follows: IRS=3.91*exp(0.296*[transformed TMB measurement]+0.097*[PD-1 normalized level]+0.056*[PD-L2 normalized level]+0.0043*[PD-L1 normalized level]).

In some embodiments, the IRS is calculated as follows: IRS=4.10*exp(0.281*[transformed TMB measurement]+0.139*[PD-1 normalized level]+0.112*[PD-L2 normalized level]+−0.128*[CD4 normalized level]).

In some embodiments, the IRS is calculated as follows: IRS=3.90*exp(0.309*[transformed TMB measurement]+0.104*[PD-1 normalized level]+0.087*[PD-L2 normalized level]+0.021*[VTCN1 normalized level]).

In some embodiments, the IRS is calculated as follows: IRS=3.95*exp(0.295*[transformed TMB measurement]+0.142*[PD-1 normalized level]+0.150*[PD-L2 normalized level]+0.020*[VTCN1 normalized level]+−0.070*[ADAM12 normalized level]+−0.139*[CD4 normalized level]).

In some embodiments, the IRS is calculated as follows: IRS=3.95*exp(0.280*[transformed TMB measurement]+0.134*[PD-1 normalized level]+0.122*[PD-L2 normalized level]+−0.070*[ADAM12 normalized level]+−0.154*[CD4 normalized level]+0.052*[PD-L1 normalized level]).

In some embodiments, the IRS is calculated as follows: IRS=4.03*exp(0.287*[transformed TMB measurement]+0.147*[PD-1 normalized level]+0.143*[PD-L2 normalized level]−0.138*[CD4 normalized level]−0.073*[ADAM12 normalized level]).

Some aspects of the present disclosure are directed to a method of treatment of a subject in need thereof with a checkpoint inhibitor therapy, comprising administering to said subject the checkpoint inhibitor therapy, wherein said subject in need thereof was previously identified by a method comprising: a. measuring expression levels of RNA transcripts for PD-1, PD-L2 and, optionally, expression levels of RNA transcripts for one or more of CD4, ADAM12, PD-L1, and VTCN1, and one or more reference genes in a biological sample obtained from a tumor specimen from a subject, wherein the one or more reference genes comprise three genes selected from LRP1, MRPL13, TBP, HMBS, ITGB7, MYC, CIAO1, CTCF, EIF2B1, GGNBP2, SLC4A1AP; b. log 2 transforming, median centering, and normalizing the measured expression levels of the RNA transcripts against the level of the RNA transcripts of at the least one reference gene to provide normalized levels of the RNA transcripts; c. measuring tumor mutation burden (TMB) in the biological sample and log 2 transforming the TMB measurement to provide a transformed TMB measurement; d. calculating a Immunotherapy Response Score (IRS) from the normalized levels of the RNA transcripts of PD-1, PD-L2 and, optionally, one or more of CD4, ADAM12, PD-L1, and VTCN1, and the transformed TMB measurement, wherein the IRS is positively correlated with the likelihood that the patient has a beneficial response to checkpoint inhibitor therapy, and obtaining an IRS having a value indicating beneficial response to checkpoint inhibitor therapy.

In some embodiments, the IRS is calculated using the Cox model as 10 times the inverse of the patient hazard ratio as compared to the median hazard rate.

In some embodiments, the IRS is calculated as follows: IRS=4.03*exp(0.287*[transformed TMB measurement]+0.147*[PD-1 normalized level]+0.143*[PD-L2 normalized level]−0.138*[CD4 normalized level]−0.073*[ADAM12 normalized level]).

In some embodiments, the IRS value indicating beneficial response to checkpoint inhibitor therapy is 10 or more.

Some aspects of the present disclosure are directed to a method of treatment, comprising: a. measuring expression levels of RNA transcripts for PD-1, PD-L2, CD4, ADAM12, and at least one reference gene in a biological sample obtained from a tumor specimen from a subject; b. log 2 transforming, median centering, and normalizing the measured expression levels of the RNA transcripts of PD-1, PD-L2, CD4, and ADAM12 against the level of the RNA transcripts of at the least one reference gene to provide normalized levels of the RNA transcripts of PD-1, PD-L2, CD4, and ADAM12; c. measuring tumor mutation burden (TMB) in the biological sample and log 2 transforming the TMB measurement to provide a transformed TMB measurement; d. calculating an Immunotherapy Response Score (IRS) from the normalized levels of the RNA transcripts of PD-1, PD-L2, CD4, and ADAM12 and the transformed TMB measurement, wherein the IRS is positively correlated with the likelihood that the patient has a beneficial response to checkpoint inhibitor therapy, and obtaining an IRS having a value indicating a beneficial response to checkpoint inhibitor therapy; and e. administering the checkpoint inhibitor therapy to the subject.

In some embodiments, the IRS is calculated as follows: IRS=4.03*exp(0.287*[transformed TMB measurement]+0.147*[PD-1 normalized level]+0.143*[PD-L2 normalized level]−0.138*[CD4 normalized level]−0.073*[ADAM12 normalized level]). In some embodiments, the IRS value indicating beneficial response to checkpoint inhibitor therapy is 10 or more. In some embodiments, the tumor specimen is a formalin-fixed paraffin-embedded (FFPE) tumor specimen. In some embodiments, the tumor specimen is adrenal cancer, biliary cancer, bladder cancer, brain cancer, breast cancer, cervical cancer, colon cancer, rectum cancer, endometrial cancer, esophageal cancer, head or neck cancer, kidney cancer, liver cancer, non-small cell lung cancer, lung cancer, lymphoma, melanoma, meninges cancer, non-melanoma skin cancer, ovarian cancer, pancreatic cancer, prostate cancer, sarcoma, small intestine cancer, or stomach cancer. In some embodiments, the expression levels of RNA transcripts are measured using PCR and next-generation sequencing. In some embodiments, the checkpoint inhibitor is an anti-PD-1 antibody, an anti-CTLA-4 antibody, an anti-PD-L1 antibody, or an anti-PD-L2. In some embodiments, the checkpoint inhibitor is nivolumab, pembrolizumab, atezolizumab, durvalumab, pidilizumab, PDR001, BMS-936559, avelumab, or SHR-1210.

All patents, patent applications, and other publications (e.g., scientific articles, books, websites, and databases) mentioned herein are incorporated by reference in their entirety. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.

The above discussed, and many other features and attendant advantages of the present inventions will become better understood by reference to the following detailed description of the invention.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 provides a flowchart of the process used to obtain the present methods

FIG. 2 shows expression by 3′ and 5′ amplicons for PD-L1 or PD-1 highly correlate and therefore were averaged together.

FIG. 3 shows the partial-AIC/BIC for each tested model when trained on all 708 samples in dataset (i.e., the full dataset).

FIG. 4 shows the median partial-AIC and log-likelihood for each model when trained on a random ⅔ of dataset×100 iterations. Also shown is the median log-likelihood score of the test sets (the ⅓ left-out).

FIG. 5 shows the process by which the IRS scores were divided into 3 groups, a low, medium, and high group, wherein the high group has the greatest benefit from ICI treatment.

FIG. 6 shows pembrolizumab TTNT is correlated to overall survival (OS).

FIG. 7 shows covariant correlations for the top 10 biomarkers.

FIG. 8 shows survival for pembrolizumab (pembro) and chemotherapy (chemo) high/medium/low IRS groups, showing that the IRS score determined by the methods disclosed herein are predictive of response to ICI instead of predictive of overall effectiveness of any cancer treatment.

FIG. 9 shows real world progression-free survival per low/medium/high IRS groups with pembro or chemo treatment.

FIG. 10 shows real world progression-free survival of NSCLC patients and other cancer patients per low/medium/high IRS groups with pembro.

FIG. 11 shows IRG rates (i.e., IRS groups) for on-label (Melanoma, Lung—NSCLC, Lung—Other, Head and Neck, Lymphoma, Bladder, Esophagus, Biliary, Stomach, Cervical, Liver, Kidney, Melanoma) and off-label cancers.

FIG. 12 shows IRG rates across Strata Trial cohort by cancer type.

FIG. 13 shows Pembro monotherapy vs pembro in combination with chemotherapy for IRS groups.

FIG. 14 shows real world progression-free survival for IRS groups divided into TMB-High (TMB-H) and TMB-Low (TMB-L) groups. TMB-High is defined as 10 or more mutations per megabase. TMB low is less than 10 mutations per megabase.

FIG. 15 shows rates of Immunotherapy Response Groups vs. TMB-H/L.

FIG. 16 shows real world progression-free survival of Pembro treated patients by IRG and sample tumor content.

FIG. 17 shows real world progression-free survival of Chemo treated patients by IRG and sample tumor content.

FIG. 18 shows the ⅔-dataset brute force search for the best 2 covariates to add to the bare-bones model (PD-1, PD-L2, and TMB). The model with the lowest AIC was selected to derive train/test statistics for that cut.

FIG. 19 is the results of the small model search showing the claimed method with ADAM12+CD4+PD-1+PD-L2+TMB.

FIG. 20 shows the results of backward selection starting with 21 markers and TMB. A multivariant fit was performed and the least significant markers dropped and removed. Partial AIC was compared before and after drop.

FIG. 21 shows the best model via brute-force training on a full dataset.

FIG. 22 shows the best model with PD-L1 added via brute-force training on a full dataset.

FIG. 23 shows the bare-bones model with just PD-1, PD-L2 and TMB trained on the full data set.

FIG. 24 shows a large model derived from backwards selection

FIG. 25 shows coefficients from iterations of ⅔ cross-validation. “Brute-force” model without and with PD-L1, Yellow lines are final model coefficients from the model w/o PD-L1.

FIG. 26 shows coefficients from iterations of ⅔ cross-validation. Small (“Bare-bones”) and Large (“Backward Selection”) models. Yellow lines are brute-force (“bru”) model coefficients from the model w/o PD-L1.

FIG. 27 shows model cross validation wherein the specimen is collected after pembro start date.

FIG. 28 shows an alternate biomarker equation with TMB+PD-L2+PD-1+ADAM12+CD4+PD-L1.

FIG. 29 shows an alternate biomarker equation with TMB+PD-L2+PD-1.

FIG. 30 shows an alternate biomarker equation with TMB+PD-L2+PD-1+ADAM12.

FIG. 31 shows an alternate biomarker equation with TMB+PD-L2+PD-1+PD-L1.

FIG. 32 shows an alternate biomarker equation with TMB+PD-L2+PD-1+CD4.

FIG. 33 shows an alternate biomarker equation with TMB+PD-L2+PD-1+VTCN1.

FIG. 34 shows an alternate biomarker equation with TMB+PD-L2+PD-1+CD4+ADAM12+VTCN1.

FIGS. 35a-c depict development of an integrative immunotherapy response score (IRS) model to stratify PD-(L)1 therapy benefit in patients with advanced solid tumors. FIG. 35a) depicts real-world treatment and molecular profiling data from formalin fixed paraffin embedded (FFPE) tumor tissue from patients enrolled in the StrataTrial (NCT03061305) are collected in the Strata Clinical Molecular Database (SCMD). Molecular data from both DNA (yellow) and RNA (blue) include both comprehensive genomic profiling (CGP) with both DNA and RNA components, and in-parallel quantitative transcriptional profiling (qTP) comprised of RNA from analytically and clinically validated tests. To develop an integrative predictor of PD-(L)1 therapy benefit, a cohort of 648 patients (from 26 tumor types) was identified with available molecular information who were treated with a pembrolizumab (pembro; PD-1) containing systemic therapy line of treatment. Lasso-penalized Cox proportional hazards modeling with five-cross validation was used to develop the IRS model for predicting real world progression free survival (rwPFS; by time to next therapy), which includes tumor mutation burden (TMB; from CGP) and expression of PD-1, PD-L1, ADAM12 and TOP2A (from qTP). The locked IRS model and threshold to assign patients to IRS-Low [L] or IRS-High [H; increased benefit] was then applied to an independent validation cohort of 248 patients (from 24 tumor types) treated with non-pembrolizumab PD-[L]1systemic monotherapy. Pie charts for the development and validation cohorts show tumor type distributions for the 11 most common tumor types and other tumor types. FIG. 35b) depicts that IRS stratifies pembrolizumab rwPFS in the development cohort. Pembrolizumab rwPFS in the development cohort stratified by IRS groups is shown by Kaplan Meier analysis with the adjusted hazard ratio (HR) and p value (adjusted by variables shown in FIG. 35c) for IRS-H vs. IRS-L. The number (n) of patients, events, and median rwPFS (with 95% confidence intervals [CI]) for each group are shown. FIG. 35c depicts that IRS is robust to potential confounders in the development cohort. Forest plot of variables included in the adjusted Cox proportional hazards model used to evaluate the ability of IRS to stratify pembrolizumab rwPFS. Adjusted hazard ratios with 95% confidence intervals (CIs) are shown for each variable with statistically significant variables bolded.

FIGS. 36a-f depict PD-[L]1 monotherapy real-world progression-free survival (rwPFS) and overall survival (OS) by Immunotherapy Response Score (IRS) Status. FIG. 36a depicts rwPFS for monotherapy pembrolizumab (pembro; PD-1 therapy) treated patients in the discovery cohort. Pembrolizumab monotherapy rwPFS in the development cohort stratified by IRS groups is shown by Kaplan-Meier analysis with the adjusted hazard ratio (HR) and p-value for IRS-High [H] vs. IRS-Low [L] groups. The number (n) of patients, events, and median rwPFS (with 95% confidence intervals [CI]) for each group are shown. FIG. 36b depicts Kaplan-Meier analysis as in 36a, except for OS. FIGS. 36c-d depict Kaplan-Meier analysis as in FIGS. 36a-b, except assessing rwPFS (36c) and OS (36d) in the independent validation cohort of patients treated with non-pembrolizumab PD-(L)1 monotherapy. FIG. 36e depicts Forest plots of adjusted HRs with 95% CI for IRS and tumor mutation burden (TMB; TMB-High [H]≥10 mutations/megabase) in otherwise equivalent models separately adjusted for IRS and TMB (H vs. L for each) in both cohorts for rwPFS and OS. The Venn diagrams show the number (n) and overlap of the IRS-H (blue) and TMB-H (red) populations in both cohorts. FIG. 36f depicts overlap of IRS-H and TMB-H populations in the 24,463 patients with informative IRS and TMB status (regardless of treatment status) in the Strata Clinical Molecular Database (SCMD).

FIG. 37a-c depicts confirmation of the predictive nature of the Immunotherapy Response Score (IRS) biomarker. To establish the predictive nature of the IRS model, we assessed an internal comparator in the pembrolizumab monotherapy cohort, consisting of the 146 patients who had received a prior line of systemic therapy prior to pembrolizumab monotherapy. FIG. 37a depicts for each patient, rwPFS was determined for the line of systemic therapy immediately prior to pembrolizumab (yellow) and the pembrolizumab monotherapy line (purple), with rwPFS for each group then stratified by IRS status. FIG. 37b depicts Kaplan-Meier analysis of pembrolizumab monotherapy rwPFS (purple) vs. prior systemic therapy rwPFS (yellow) in the IRS-Low [L] subset of patients (log-rank p-value shown). The number (n) of patients, events, and median rwPFS (with 95% confidence intervals [CI]) for each group are shown. FIG. 37c depicts Kaplan-Meier analysis of pembrolizumab monotherapy rwPFS (purple) vs. prior systemic therapy rwPFS (yellow) in the IRS-H subset of patients (log-rank p-value shown). The likelihood ratio test (LRT) p-value for interaction between pembrolizumab vs. immediately prior treatment line and IRS status (IRS-L vs. IRS-High [H]) is also shown.

FIGS. 38a-c depicts Immunotherapy Response Score (IRS) for predicting pembrolizumab monotherapy vs. combination chemotherapy benefit in first line NSCLC. Propensity score matching (see Methods) was used to identify matched cohorts of patients with NSCLC treated with 1st line systemic pembrolizumab (pembro) monotherapy (n=77) or pembrolizumab+chemotherapy (chemo) combination therapy (n=77) that did not significantly differ in age, gender, TMB status, PD-L1 expression by quantitative transcriptomic profiling (qTP; the expression biomarker component of IRS), or IRS status; PD-L1 immunohistochemistry (IHC) was only available for 24/154 samples in the matched cohort (see FIG. 13 for validation of PD-L1 by qTP vs. PD-L1 IHC). FIG. 38a depicts a Kaplan-Meier analysis of pembrolizumab monotherapy rwPFS (orange) vs. pembrolizumab+chemotherapy combination therapy (yellow) in the IRS-Low [L] subset of patients (log-rank p-value shown). The number (n) of patients, events, and median rwPFS (with 95% confidence intervals [CI]) for each group are shown. FIG. 38b depicts a Kaplan-Meier analysis of pembrolizumab monotherapy rwPFS (orange) vs. pembrolizumab+chemotherapy combination therapy (yellow) in the IRS-High [H] subset of patients (log-rank p-value shown). The number (n) of patients, events, and median rwPFS (with 95% confidence intervals [CI]) for each group are shown. FIG. 38c depicts the distribution of IRS status in a separate cohort of NSCLC tumor samples with PD-L1 IHC (Fig S8) stratified by clinically relevant tumor proportion score (TPS) bins.

FIGS. 39a-d depict pan-solid tumor distribution of immunotherapy response score (IRS) groups. FIG. 39a depicts that IRS groups were determined for all 24,463 patients in the Strata Clinical Molecular Database (SCMD) with informative tumor mutation burden (TMB) and gene expression data needed to generate IRS. IRS group (Low [L; light blue] vs. High [H; dark blue]) distribution is shown by box plot (numbers indicated percentages); FIG. 39b depicts stratification of the 24,463 patients by approved and non-approved PD-(L)1 monotherapy tumor types; FIG. 39c depicts a breakdown of FIG. 39b by individual tumor types; FIG. 39d depicts Breakdown of FIG. 39b by IRS and TMB (High [H] vs. Low [L]; TMB-H as ≥10 mutations/megabase). Results may not add up to 100% or be equivalent in sub-analyses due to rounding. Tumor type abbreviations: NSCLC (non-small cell lung cancer), RCC (renal cell carcinoma), NMSC (non-melanoma skin cancer), SCLC (small cell lung cancer), CNS and PNS (central nervous system and peripheral nervous system), CUP (cancer of unknown primary), CRC (colorectal cancer), GIST (gastrointestinal stromal tumor).

FIG. 40 depicts Overall study diagram from the Strata Clinical Molecular Database (SCMD) used to develop and validate the Immunotherapy Response Score (IRS). Disposition of patients from the Strata Trial (NCT03061305) used to develop and validate IRS are shown. Included populations are indicated by gray boxes. As patients could contribute to multiple analyses (e.g., a subject treated with first line angiogenesis inhibitor and second line pembrolizumab could be eligible for both the “Non-IO 1st line analysis” and the “Discovery cohort” as long as they met both inclusion/exclusion criteria [including the sample was collected before both lines of therapy]), the number of shared patients is indicated by green arrows at the highest branch point. The overall SCMD population is shown in bolded yellow. No patients were shared between the discovery and validation cohorts (bolded blue). Analyses on groups are indicated by Figure numbers. *For the “Clonal samples with IRS” group, shared subjects are not shown due to diagrammatic complexity (greatest shared subjects n=26 with “Non-IO 1st line”).

FIG. 41 depicts Assignment of therapy lines from real world treatment data. For all Strata Trial (NCT03061305) subjects with treatment data (treatment start and stop dates), standardized assignment of adjuvant/systemic therapy lines was performed accounting for adjuvant/systemic therapy, monotherapy/combination therapy, potential overlap of treatment start/stop dates and repeating lines of therapy (whether monotherapy in combination). Assigned treatment lines and an example of real world-progression free survival measurement by time to next therapy (TTNT; start date of therapy to start date of subsequent therapy) are shown for a patient with metastatic renal cell carcinoma.

FIG. 42 depicts Time to next therapy (TTNT) of patients in the Strata Clinical Molecular Database (SCMD) by line of therapy. Real-world progression-free survival by TTNT per first (Line 1, blue line), second (Line 2, green line) or third or more (Line 3+, orange line) line of therapy for the 9,899 patients in the SCMD having treatment data from at least one systemic antineoplastic agent. The number (n) of patients, events, and median rwPFS (with 95% confidence intervals [CI]) for each group are shown, along with the overall log rank p value.

FIGS. 43a-c depict Strata Clinical Molecular Database (SCMD) non-small cell lung cancer (NSCLC) analysis. FIG. 43a Real world progression free survival (rwPFS, by time to next therapy) in SCMD of patients with first line NSCLC when treated with first generation (gen) targeted EGFR, ALK, ROS1 or MET tyrosine kinase inhibitors ([TKI]; erlotinib, gefitinib, or crizotinib, n=37) versus later-generation inhibitors (n=120, green) is shown by Kaplan-Meier analysis with the adjusted hazard ratio (HR) and p-value shown for later vs. first generation inhibitor. The number (n) of patients, events, and median rwPFS (with 95% confidence intervals [CI]) for each group are shown. FIG. 43b rwPFS in SCMD patients with first line NSCLC when treated with a first line oncogene NCCN preferred targeted monotherapy TKI based on whether the treatment occurred before (n=57, blue line; treatment decision made from orthogonal testing) or after (n=72, green line; treatment decision made using StrataNGS) receiving StrataNGS CGP test results is shown by Kaplan-Meier analysis with the adjusted hazard ratio (HR) and p-value shown for treatment before vs. after receiving CGP results. FIG. 43c rwPFS in SCMD of patients with first line NSCLC treated with a biomarker matched, first line oncogene NCCN preferred targeted monotherapy TKI after receiving StrataNGS results based on whether the sample 1) passed both StrataNGS sample input criteria and relevant sequencing QC metrics (n=48) or 2) did not meet sample input or failed sequencing QC metrics but reported a therapy matched biomarker (n=17) is shown by Kaplan-Meier analysis with the adjusted hazard ratio (HR) and p-value shown for failed (2) vs. passed (1) QC metrics.

FIG. 44 depicts Correlation of real-world pembrolizumab progression-free survival (rwPFS) and overall survival (OS). Correlation for pembrolizumab rwPFS by time to next therapy (TTNT) and OS for patients in the discovery cohort with more than one line of systemic therapy. Colored boxes indicate patients discussed in the Supplementary Results.

FIGS. 45a-d depict PD-(L)1 monotherapy real world progression free survival (rwPFS) and overall survival (OS) by tumor mutation burden (TMB) status. FIG. 45a depicts Pembrolizumab monotherapy (PD-1) rwPFS (by time to next therapy) in the discovery cohort stratified by TMB groups (TMB-High [H]≥10 mutations/megabase by StrataNGS testing vs. TMB-Low [L]) is shown by Kaplan Meier analysis with the adjusted hazard ratio (HR) and p value for TMB-H vs. -L groups. The number (n) of patients, events, and median rwPFS (with 95% confidence intervals [CI]) for each group are shown. FIG. 45b as in FIG. 45a except OS. FIGS. 45c-d, as in FIGS. 45a-b, except assessing the independent validation cohort of patients treated with non-pembrolizumab PD-(L)1 monotherapy.

FIGS. 46a-f depict Housekeeping gene selection and validation, accuracy vs qRT-PCR, and replicate amplicon correlation for the quantitative expression component of the integrated comprehensive genomic profiling and quantitative transcriptional profiling (CGP+qTP) laboratory developed test used to report the Immunotherapy Response Score (IRS). IRS is reported from an integrated CGP+qTP test that combines comprehensive genomic profiling (CGP) from the analytically and clinically validated StrataNGS test with in-parallel quantitative transcriptional profiling (qTP) by multiplex RT-PCR based next generation sequencing. FIG. 46a depicts initial pre-clinical versions of the qTP panel contained 6 “positive control” genes across two RNA primer pools previously used in the RNA fusion component of the Oncomine Focus/Precision Assay (OPA positive). To evaluate the suitability of these markers as pan-cancer housekeeping genes for quantitative expression profiling, we performed a multi-part evaluation of transcriptome profiles of pan-cancer, pan-normal tissue stability. Average expression levels (in transcripts per million [TPM]), and coefficient of variation (CV) are shown from ≥20,000 tumor, normal and cancer cell line samples for OPA positive genes, additional candidate housekeeping genes from an assessment of TCGA (TCGA stable) and the commonly used housekeeping gene GAPDH. The eight bolded genes were included in gene expression panels used to develop the CGP+qTP test and IRS. FIG. 46b depicts letter-value plots of normalized expression for the three final housekeeping genes used in the qTP panel (CIAO1, EIF2B1 and HMBS) and the remaining five candidates are shown from a consecutive 4-month period of CGP+qTP clinical testing of samples with reportable quantitative expression using the current test version (n=3,417; regardless of tumor content). FIG. 46c depicts the clinical accuracy of the qTP component was first determined by determining target gene expression concordance with hydrolysis probe based qRT-PCR through representational validation on 24 FFPE tumor samples. Expression of included individual target gene amplicons (n=32) are shown by color. The concordance correlation coefficient for the panel-wide validation as well as only the four IRS expression biomarkers (PD-L1, PD-1, ADAM12 and TOP2A) are shown. The line of equality is shown. FIGS. 46d-f depicts two separate PD-L1, PD-1 and ADAM12 amplicons are present in the current qTP panel (only one of two ADAM12 amplicons was also present on all previous panels used to develop and validate IRS). As multiplex PCR based qTP enables unambiguous read assignment to each target gene amplicon, we determined the correlation coefficients of the replicate amplicons across the 24,463 Strata Trial samples used to assess IRS distribution (n=7,911 samples on panels with both ADAM12 amplicons). Scatter plots are shown overlying a density heatmap. The line of equality is shown.

FIGS. 47a-d depict Accuracy vs. clinical immunohistochemistry and reproducibility for the quantitative expression component of the integrated comprehensive genomic profiling and quantitative transcriptional profiling (CGP+qTP) laboratory developed test used to report the Immunotherapy Response Score (IRS). IRS is reported from an integrated CGP+qTP test that combines comprehensive genomic profiling (CGP) from the analytically and clinically validated StrataNGS test with in-parallel quantitative transcriptional profiling (qTP) by multiplex RT-PCR based next generation sequencing. FIG. 47a depicts the accuracy of the PD-L1 qTP component of IRS was validated against clinical IHC using a cohort of 276 non-small cell lung cancer (NSCLC) formalin fixed paraffin embedded (FFPE) tumor samples with reportable qTP (including tumor content [TC]≥20%) and PD-L1 IHC expression by the 22C3 clone (using tumor proportion score [TPS]) in accompanying pathology reports. Box plots of qTP PD-L1 expression stratified by TPS bin (0%, 1-49%, and ≥50%) are shown, along with results from the Kruskal Wallis [K.W.] test (with Jonckheere-Terpstra [J.T.] trend test [increasing median from 0%, 1-49%, and ≥50%]). Only 24 of these samples came from the 154 patients in the propensity matched first line NSCLC treatment analysis (see FIG. 4), precluding direct assessment of IRS vs. PD-L1 IHC for predicting pembrolizumab benefit. However, IRS status could be generated for all 276 NSCLC samples with PD-L1 IHC and the percentage of IRS-H samples by TPS bin is shown in dark blue. FIG. 47b depicts accuracy of the TOP2A qTP component of IRS was validated against clinical IHC using a cohort of 956 FFPE tumor tissue samples (36 tumor types) with reportable qTP (including TC≥20%) with proliferation index (percentage of Ki67 positive tumor cells) in accompanying pathology reports. The Pearson correlation coefficient of qTP TOP2A expression vs. clinical proliferation index from the scatter plot is shown with 95% confidence interval [CI] and p-value, with points overlying a density heatmap and the line of best fit indicated by the dashed line. FIG. 47c depicts the panel wide qTP reproducibility between operators, lots, and instrumentation was established using separate replicate nucleic acid aliquots isolated from FFPE tumor samples. Twenty-seven unique samples were assessed by two operators on different days using different library preparation instrumentation, different library preparation reagent lots, and different templating and sequencing lots and instruments. For each sample, the maximum and minimum nRPM for each target gene across all replicates was plotted (individual target gene amplicons are shown by color) and the concordance correlation coefficient was determined. FIG. 47d, as in FIG. 47c, except reproducibility of IRS was determined by plotting the maximum and minimum IRS across all replicates for each sample and the concordance correlation coefficient was determined. Qualitative agreement of IRS status (High vs. Low) from the maximum and minimum IRS score across all replicates was also determined.

FIG. 48 depict Lasso-penalized Cox proportional hazards regression for Immunotherapy Response Score (IRS) development. To develop an integrative PD-1/PD-L1 blockade benefit predictive model, we performed Lasso-penalized Cox proportional hazards regression with five-fold cross-validation in the 648 patient pembrolizumab (PD-1 therapy) discovery cohort to perform feature selection from tumor mutation burden (TMB; log 2) and 23 candidate immune and proliferation expression biomarkers associated with pembrolizumab TTNT. The Lasso penalty term was chosen as the value which maximized the concordance index (top panel; gray line) via 5-fold cross validation, with the coefficients shown for TMB and the 23 candidate expression biomarkers vs. alpha (α) (bottom panel), resulting in a five-term model including TMB, PD-1, PD-L1, ADAM12, and TOP2A.

FIGS. 49a-b depict Pembrolizumab overall survival (OS) by Immunotherapy Response Score (IRS) status. FIG. 49a depicts the Pembrolizumab OS in the discovery cohort stratified by IRS groups is shown by Kaplan Meier analysis with the adjusted hazard ratio (HR) and p value (adjusted by variables shown in b) for IRS-H vs. -L. The number (n) of patients, events, and median rwPFS (with 95% confidence intervals [CI]) for each group are shown. FIG. 49b depicts the Forest plot of variables included in the adjusted Cox proportional hazards model used to evaluate the ability of IRS to stratify pembrolizumab OS. Adjusted hazard ratios with 95% confidence intervals (CI) are shown for each variable. Statistically significant variables are bolded.

FIGS. 50a-d depict Real world progression free survival (rwPFS) and overall survival (OS) by Immunotherapy Response Score (IRS) status in the validation cohort stratified by PD-1 vs. PD-L1 therapy. FIG. 50a depicts the rwPFS (by time to next therapy) for the monotherapy PD-L1 treated subset of the validation cohort stratified by IRS groups is shown by Kaplan Meier analysis with the adjusted hazard ratio (HR) and p value for IRS-H vs. -L groups. The number (n) of patients, events, and median rwPFS (with 95% confidence intervals [CI]) for each group are shown. FIG. 50b, as in FIG. 50a, except assessing OS. FIGS. 50c&d, as in FIGS. 50a&b, except assessing the TTNT (FIG. 50c) and the OS (FIG. 50d) for the monotherapy PD-1 treated subset of the validation cohort. In addition to the adjusted HRs and p-value, the log-rank p-value is also shown.

FIGS. 51a-d depict PD-(L)1 monotherapy real world progression free survival (rwPFS) and overall survival (OS) by Immunotherapy Response Score (IRS) status and Tumor Mutation Burden (TMB). FIG. 51a depicts the Pembrolizumab monotherapy rwPFS in the discovery cohort stratified by IRS (IRS-High [-H] vs. -Low [L]) and TMB (TMB-H [≥10 mutations/megabase] vs. TMB-L is shown by Kaplan Meier analysis. Benjamini Hochberg (BH) adjusted p-value for pairwise log-rank test between the IRS-H/TMB-H and IRS-H/TMB-L groups is shown. The number (n) of patients, events, and median rwPFS (with 95% confidence intervals [CI]) for the analyzed groups are shown. FIG. 51b, as in FIG. 51a, expect OS. FIGS. 51c&d, as in FIGS. 51a&b, except assessing rwPFS (FIG. 51c) and OS (FIG. 51d) in the independent validation cohort of patients treated with non-pembrolizumab PD-(L)1 monotherapy.

FIGS. 52a-d depict CDKN2A deep deletion status does not add to Immunotherapy Response Score (IRS) for predicting PD-(L)1 monotherapy real world progression free survival (rwPFS) or overall survival (OS). FIG. 52a depicts the Pembrolizumab monotherapy rwPFS in the subset (n=310) of the discovery cohort evaluable for CDKN2A deep deletion (equivalent to homozygous/two copy deletion if diploid) status (≥40% tumor content and evaluable copy number alterations) stratified by IRS groups is shown by Kaplan Meier analysis with the adjusted hazard ratio (HR) and p value for IRS-High vs. -L groups. The number (n) of patients, events, and median rwPFS (with 95% confidence intervals [CI]) for each group are shown. The forest plot shows adjusted hazard ratios with 95% confidence intervals (CI) for IRS (IRS-H vs. IRS-L) and CDKN2A deep deletion status (CDKN2A deep deletion present vs. CDKN2A deep deletion not present) in the same adjusted model. FIG. 52b, as in FIG. 52a, except assessing OS. FIGS. 52c&d, as in FIGS. 52a&b, except assessing TTNT (FIG. 52c) and OS (FIG. 52d) in the subset (n=199) of the independent validation cohort evaluable for CDKN2A deep deletion treated with non-pembrolizumab PD-(L)1 monotherapy.

FIGS. 53a-b depict Immunotherapy Response Score (IRS) is robust to pre-PD-(L)i sample collection timing. FIG. 53a depicts the Pearson correlation of IRS from clonal tumor specimens from the same patient with different collection dates and no checkpoint-inhibitor therapy in between tested sample collection dates (n=104 patients). FIG. 53b depicts the PD-(L)1 rwPFS stratified by IRS group in 181 patients who otherwise would have been included in the discovery or validation cohorts but had their samples collected after starting PD-(L)1 therapy is shown by Kaplan Meier analysis with the adjusted hazard ratio (HR) and p value for IRS-H vs. IRS-L. The number (n) of patients, events, and median rwPFS (with 95% confidence intervals [CI]) for each group are shown.

FIGS. 54a-e depict Immunotherapy Response Score (IRS) is robust to variable tumor content. FIG. 54a depicts a continuous tumor content term was included in the adjusted Cox proportional hazards (CPH) model for pembrolizumab real world progression free survival (rwPFS; by time to next therapy) in the overall discovery cohort (including age, gender, most common tumor type [NSCLC] vs. others, therapy type [monotherapy/combination], and line of therapy). Adjusted hazard ratios with 95% confidence intervals (CIs) are shown for each variable with statistically significant variables bolded. FIGS. 54b-d depicts the Pembrolizumab rwPFS binned by tumor content (20-35%, 40-70%, and ≥70%) and stratified by IRS groups is shown by Kaplan Meier analysis. The number (n) of patients, events, and median rwPFS (with 95% confidence intervals [CI]) for each group are shown. FIG. 54e depicts the PD-(L)1 rwPFS-stratified by IRS group in 64 patients who otherwise would have been included in the discovery or validation cohorts except the tested sample tumor content was <20% is shown by Kaplan Meier analysis with the adjusted hazard ratio (HR) and p value for IRS-H vs. -L groups. The number (n) of patients, events, and median rwPFS (with 95% confidence intervals [CI]) for each group are shown

FIGS. 55a-d depict Additional analyses supporting the predictive nature of the Immunotherapy Response Score (IRS) biomarker. FIGS. 55a&b: to establish the predictive nature of the IRS model, we assessed an internal comparator cohort for the pembrolizumab monotherapy cohort, consisting of the 146 patients who had received a previous line of systemic therapy prior to monotherapy pembrolizumab therapy. For each patient, real-world progression free survival (rwPFS) was determined for the line of systemic therapy immediately prior to pembrolizumab and the pembrolizumab monotherapy line, with rwPFS stratified by IRS status (see FIGS. 37a-c). Here, Kaplan Meier analysis of pembrolizumab monotherapy rwPFS (purple) vs. prior systemic therapy rwPFS (yellow) in the subset of non-microsatellite instability high (MSI-H) tumors in non-PD-(L)1 monotherapy approved tumor types is shown, along with the log-rank p-value between pembrolizumab and the prior therapy in the (FIG. 55b) IRS-high [H] and (FIG. 55a) IRS-low [L] populations. The number (n) of patients, events, and median rwPFS (with 95% confidence intervals [CI]) for each group are shown. FIG. 55c, To confirm the predictive nature of the IRS model we determined rwPFS in 3,184 patients in the SCMD treated with systemic first-line non-immunotherapy (IO), who otherwise met criteria for the discovery and validation cohorts. Kaplan-Meier analysis of non-IO systemic first line rwPFS stratified by IRS status is shown with the adjusted hazard ratio (HR) and p-value for IRS-H vs. IRS-L groups. FIG. 55d depicts the Ipilimumab+nivolumab (ipi+nivo) rwPFS in 70 patients who otherwise would have been eligible for the validation cohort but received combination ipilimumab (CTLA4)+nivolumab (PD-1) therapy stratified by IRS status is shown by Kaplan Meier analysis with the adjusted hazard ratio (HR) and p value for IRS-H vs. -L groups.

FIGS. 56a-g depict Clinical utility of integrated comprehensive genomic profiling and quantitative transcriptional profiling (CGP+qTP) outside of immunotherapy treatment decision making. IRS is reported from an integrated CGP+qTP test that combines comprehensive genomic profiling (CGP) from the analytically and clinically validated StrataNGS test with in-parallel quantitative transcriptional profiling (qTP) by multiplex RT-PCR based next generation sequencing. FIG. 56a depicts the accuracy of ESRI (estrogen receptor; ER) by qTP validated against clinical IHC using a cohort of 300 breast cancer formalin fixed paraffin embedded (FFPE) tumor samples with reportable qTP (including tumor content [TC]≥20%) and ER IHC expression (by % tumor cells positive) in accompanying pathology reports. The entire cohort was used for accuracy, however the cohort was randomly split into equivalent training (n=150) and validation (n=150) cohorts to establish clinical validity (see FIG. 56d) prior to performing the accuracy assessment. The correlation coefficient of qTP ER expression vs. clinical ER % tumor cells positive (log 2) from the scatter plot is shown with 95% confidence interval [CI] and p-value, with points overlying a density heatmap and the line of best fit indicated by the dashed line. FIG. 56b depicts the accuracy of PGR (progesterone receptor; PR) by qTP validated against clinical IHC using a cohort of 291 breast cancer formalin fixed paraffin embedded (FFPE) tumor samples with reportable qTP (including tumor content [TC]≥20%) and PR IHC expression (by % tumor cells positive) in accompanying pathology reports. The entire cohort was used for accuracy, however the cohort was randomly split into equivalent training (n=145) and validation (n=146) cohorts to establish clinical validity (see FIG. 56e) prior to performing the accuracy assessment. The correlation coefficient of qTP PR expression vs. clinical PR % tumor cells positive (log 2) from the scatter plot is shown with 95% confidence interval [CI] and p-value, with points overlying a density heatmap and the line of best fit indicated by the dashed line. FIG. 56c depicts the accuracy of the HER2 (ERBB2) by qTP was validated against clinical IHC using a cohort of 545 breast cancer formalin fixed paraffin embedded (FFPE) tumor samples with reportable qTP (including tumor content [TC]≥20%) and HER2 IHC expression (0, 1+, 2+ or 3+) in accompanying pathology reports. The entire cohort was used for accuracy, however the cohort was randomly split into equivalent training (n=273) and validation (n=272) cohorts to establish clinical validity (see FIG. 56f) prior to performing the accuracy assessment. Box plots of qTP HER2 expression stratified by clinical IHC category are shown in the accuracy cohort, along with the Kruskal Wallis (K.W.) test p-value and Jonckheere-Terpstra [J.T.] trend test p-value (increasing median from 0 to 1+ to 2+ to 3+). FIG. 56d, depicts that the clinical validity for ER status by qTP was established by setting thresholds for qTP ER Negative (<12.75; green dashed line) and Positive (>14.5; red dashed line) in the training cohort (n=150) of breast cancer FFPE tissue samples with clinical ER status (by % tumor cells positive) in accompanying pathology reports (see FIG. 56a) based on the clinical IHC defined categories of ER Negative (Neg.; 0%), Low (1-10%) and Positive (Pos; >10%). Expression between the Negative and Positive thresholds were defined as qTP ER inconclusive (light gray). Desired sensitivity (sens; positive percent agreement [PPA]) and specificity (spec; negative percent agreement [NPA]) for qTP ER Negative/Positive status (vs. IHC Negative and Positive) was pre-specified as >95% each. Locked thresholds were then applied to the validation cohort (n=150), with box plots of qTP ER expression by clinical IHC categories shown, along with PPA and NPA values and 95% confidence intervals (CI). In this validation cohort, the qTP ER inconclusive category (n=8 of 150 validation samples) correctly identified 6/7 clinical IHC ER-low samples, supporting the clinical utility of this category. FIG. 56e, clinical validity for PR status by qTP was established by setting a threshold for qTP PR Negative (<12.3; red dashed line) in the training cohort (n=145) of breast cancer FFPE tissue samples with clinical PR status (by % tumor cells positive) in accompanying pathology reports (see b). Although PR does not have a “Low” clinical IHC reporting group, three clinical IHC defined categories of PR Negative (Neg.; 0%), Low (1-10%) and Positive (Pos; >10%) were used in the training cohort to facilitate appropriate balancing of PPA and NPA in the threshold setting. As the potential clinical implications of false positive PR status, namely inappropriately considering an ER negative/HER2 negative breast cancer as hormone receptor positive (vs. triple negative) are more impactful than false negative PR status (it is unclear if ER negative/PR positive breast cancer are biologically plausible), the threshold was set to favoring NPA and pre-specified acceptable NPA (versus PR 0% IHC) of greater than 95% was set. The locked threshold was then applied to the validation cohort (n=146), with box plots of qTP PR expression by IHC categories shown, along with PPA and NPA values and 95% confidence intervals (CI). f) Clinical validity for HER2 status by qTP was established by setting thresholds in the training cohort (n=273) of breast cancer FFPE tissue samples with clinical HER2 status (by the clinically recognized 0, 1+, 2+ or 3+ categories) in accompanying pathology reports (see c). As with ER, given that the clinical utility of HER2 IHC 2+ is to reflex to FISH/ISH (and StrataNGS provides ERBB2 copy status), and the unclear validity of 0 vs. 1+ expression in retrospective samples clinically scored before the FDA approval of trastuzumab deruxtecan in HER2 1+ and 2+(FISH/ISH negative) breast cancer, we set thresholds for qTP HER2 Low (<18.0; green dashed line) and High (>19.2; red dashed line), with expression in between those thresholds reported as qTP HER2 Inconclusive (light gray); the threshold was set by balancing desired maximum sensitivity vs. IHC 3+ with the observation that the majority of IHC 3+ tumors with the lowest qTP HER2 expression also lacked ERBB2 amplifications in the training cohort. Hence, desired NPA and PPA for qTP HER2 Low/High status (vs. IHC 0-1+ and 3+) was pre-specified as NPA>95% and PPA>70%; no performance metrics for IHC 2+ samples were prespecified. Locked thresholds were then applied to the validation cohort (n=272 [including 51 IHC 2+ not formally evaluated]), with box plots of qTP HER2 expression by clinical IHC categories shown, stratified by ERBB2 copy number status (red=amplified, green=not amplified [wildtype; wt], gray=copy number status not evaluable), along with PPA and NPA values and 95% confidence intervals (CI). Notably, of the three false negative samples in the validation cohort (IHC 3+but qTP HER2 Low), two lacked ERBB2 amplifications by StrataNGS testing. Additionally, more than 50% (n=7) of the of the qTP HER2 inconclusive category (n=12 of 272 total validation samples) was IHC 2+, supporting the clinical utility of this category and deferral to amplification status. FIG. 56g, Although the above analyses support clinical utility of ER and PR (collectively hormone receptor [HR]) and HER2 status by qTP as the clinical utility of these biomarkers is already established, as an additional demonstration of the clinical utility of integrating qTP results with CGP results, we determined the impact of qTP HR status on PIK3CA mutation treatment association in patients with breast cancer (standard of care [SOC] PIK3CA mutations are associated with FDA-approved alpelisib+fulvestrant therapy only in patients with hormone receptor positive/HER2 negative breast cancer [green box]) from a 4-month period of consecutively tested pan-solid FFPE tumor samples (n=3,904) submitted for clinical CGP testing. As shown in the sample disposition diagram, of the 3,904 samples, 288 samples were breast cancer and met qTP and CGP QC metrics (including the final ≥20% tumor content requirement) needed to evaluate HR status, PIK3CA mutations and ERBB2 copy number status, of which 31% (n=90) harbored SOC PIK3CA mutations associated with alpelisib therapy. Of the 90 PIK3CA mutant samples, 2 (2%) would be correctly identified as not associated with alpelisib therapy by CGP testing alone (based on the presence of an ERBB2 amplification; pink boxes), while 11 (12%) could only be correctly identified as not associated with alpelisib therapy by integration of qTP findings (based on HR negative status; dark red box).

FIGS. 57a-c depict Exploratory analysis defining an Immunotherapy Response Score (IRS) ultra-low subset. In a post-hoc, exploratory analysis in the combined discovery (n=648; pembrolizumab [pembro] treated) and validation (n=248; non-pembrolizumab PD-[L]1 treated) cohorts, we identified a threshold (<0.41) that subdivided the IRS-Low (-L) group into intermediate (IRS-L [I]) and ultra-low (IRS-L [U]) subsets. FIG. 57a depicts the PD-(L)1 real-world progression free survival (rwPFS) in the combined cohorts stratified by IRS-High [H], IRS-L (I), and IRS-L (U) groups is shown by Kaplan Meier analysis with the Benjamini Hochberg (BH) adjusted p-value for pairwise log-rank test between the IRS-L (I) vs. IRS-L (U) groups and the adjusted hazard ratio (HR) and p value for IRS-L (I) vs. IRS-L (U) groups shown. The Cox proportional hazard model was adjusted for age, gender, most common tumor type (NSCLC vs. other), line of therapy, type of therapy (monotherapy vs. combination therapy) and IRS (-H, -L [I], and -L [U]). The number (n) of patients, events, and median rwPFS (with 95% confidence intervals [CI]) for the analyzed groups are shown. FIG. 57b, as in FIG. 57a, except overall survival (OS). FIG. 57c, this three group IRS classification was applied to all 24,463 patients in the Strata Clinical Molecular Database (SCMD) with valid tumor mutation burden (TMB) and gene expression data. IRS group distribution is shown by box plot (numbers indicated percentages). Stratification and breakdown of PD-(L)1 monotherapy approved tumor types is shown. Tumor type abbreviations: NSCLC (non-small cell lung cancer), RCC (renal cell carcinoma), NMSC (non-melanoma skin cancer), SCLC (small cell lung cancer).

FIGS. 58a-d depict the confirmation of the predictive nature of the Immunotherapy Response Score (IRS) Biomarker when an ultra-low subset is defined. To establish the predictive nature of the IRS model, we assessed an internal comparator in the pembrolizumab (pembro) monotherapy cohort, consisting of the 146 patients who had received a prior line of systemic therapy prior to pembrolizumab monotherapy. Here, we subdivided the IRS-Low (-L) group into intermediate (IRS-L [I]) and ultra-low (IRS-L [U]) subsets as defined in FIGS. 57a-c. FIG. 58a depicts that for each patient, rwPFS was determined for the line of systemic therapy immediately prior to pembrolizumab and the pembrolizumab monotherapy line, with rwPFS stratified by IRS status. FIG. 58a depicts Kaplan-Meier analysis of the immediately prior systemic therapy rwPFS in the IRS-High [H], IRS-L (I), and IRS-L (U) groups (overall log-rank p-value is shown). The number (n) of patients, events, and median rwPFS (with 95% confidence intervals [CI]) for each group are shown. FIGS. 58b-d depict Kaplan-Meier analysis of pembrolizumab monotherapy rwPFS (purple) vs. prior systemic therapy rwPFS (yellow) in the (FIG. 58b) IRS-L (U) subset, (FIG. 58c) the IRS-L (I) subset, and (FIG. 58d) the IRS-H group of patients (log-rank p-value shown).

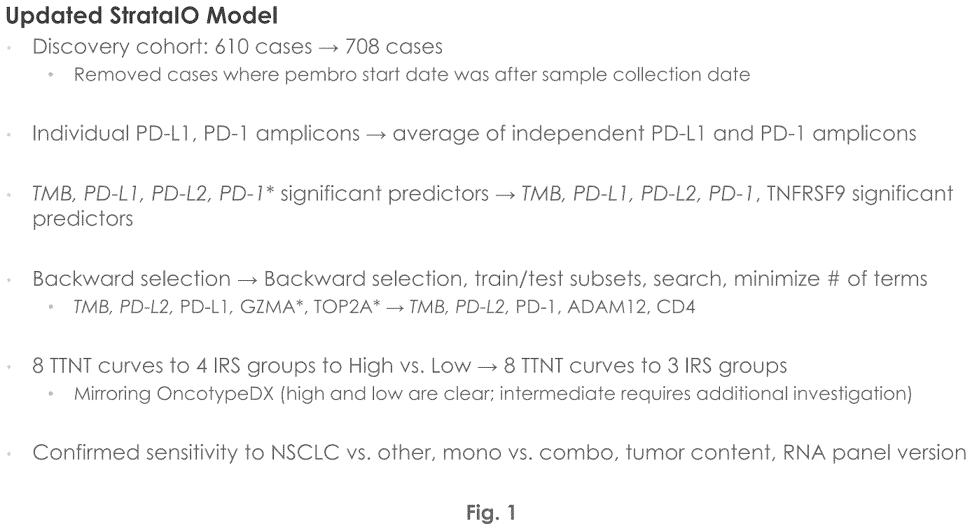

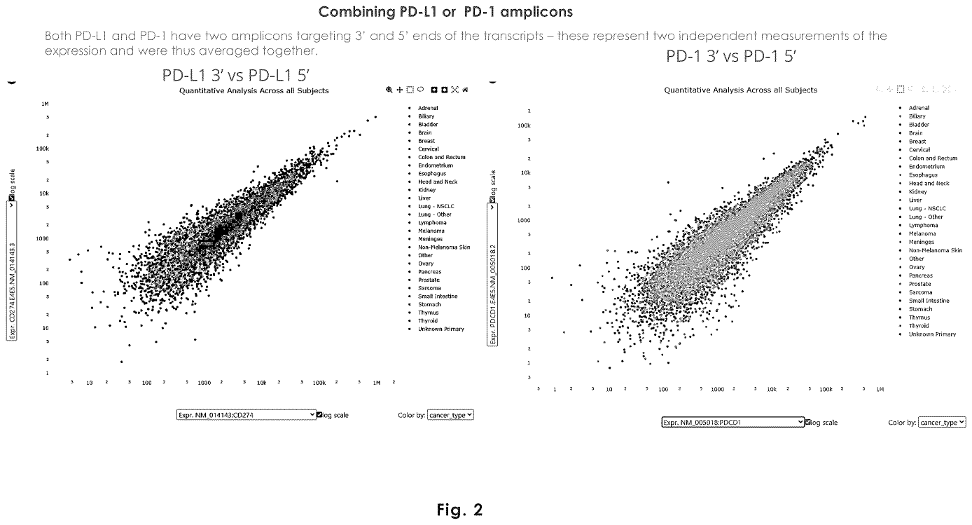

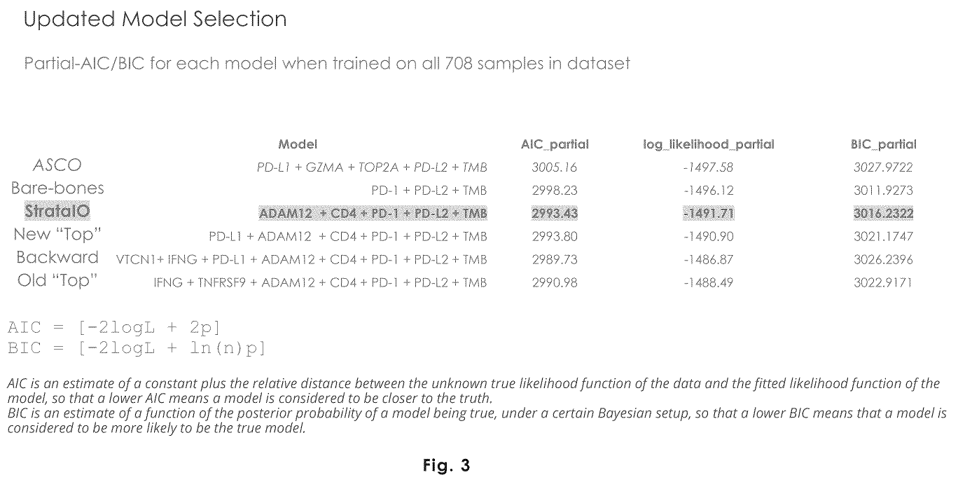

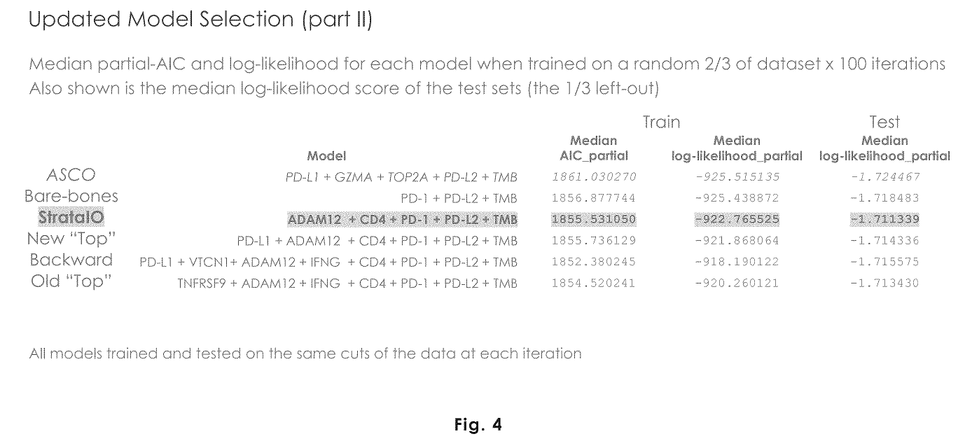

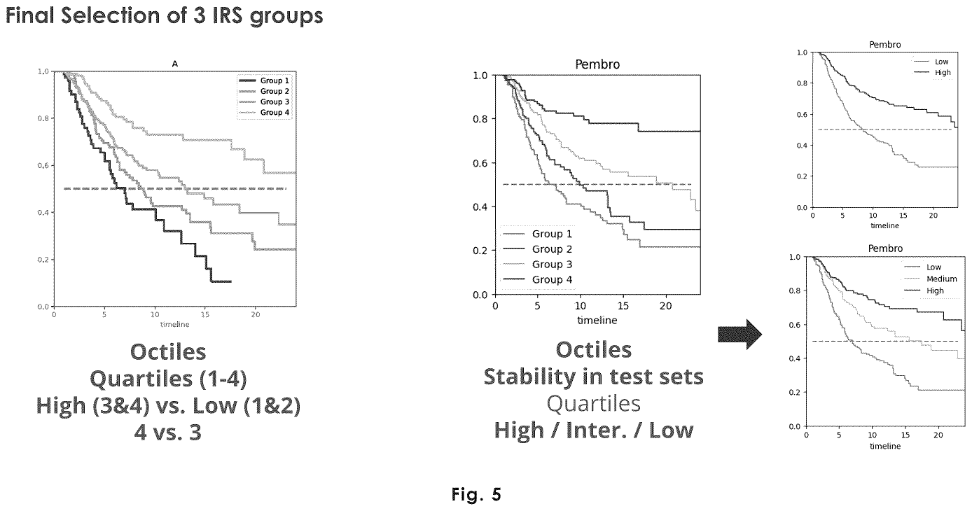

### DETAILED DESCRIPTION OF THE INVENTION

Immune checkpoint inhibitors are FDA-approved and provide clinical benefit across a wide range of tumor types. However, in most indicated tumor types, only a minority of patients benefit, and additional patients benefit outside of indicated tumor types. Thus, improved diagnostic tools are required to select patients for immunotherapy treatment. Leveraging real-world pembrolizumab outcome data combined with DNA mutation and RNA expression data from a clinical NGS test for 610 diverse solid tumor patients, the inventors demonstrated that TMB, PD-L1 and PD-L2 were independent predictors of treatment benefit and that a multivariate Immunotherapy Response Score (IRS) predicted pembrolizumab benefit relative to chemotherapy across solid tumors. IRS scores are characterized across nearly 20,000 advanced solid tumors and showed that the proportion of patients in high IRS groups predicted the observed pembrolizumab tumor type response rates. In another aspect, leveraging PD-(L)1 therapy treatment data and CGP plus quantitative transcriptomic profiling (CGP+qTP) data from the Strata Trial (NCT03061305), enabled the development and cross-validation of an integrated Immunotherapy Response Score (IRS) that predicts pan-solid tumor PD-(L)1 benefit by both real-world progression free survival (rwPFS) and overall survival (OS) by an analytically and clinically validated CGP+qTP laboratory developed test (LDT) applicable to minute formalin-fixed paraffin-embedded (FFPE) tissue specimens. The IRS diagnostic algorithms disclosed herein markedly improve the selection of patients for immunotherapy.

Some aspects of the present invention are directed to a method of treatment, comprising: (a)(i) measuring expression levels of RNA transcripts for at least two of PD-1, TOP2A, PD-L1 and ADAM12 obtained from a tumor specimen from a subject; (b) measuring tumor mutation burden (TMB) in the biological sample and log 2 transforming the TMB measurement to provide a transformed TMB measurement; (c) calculating an Immunotherapy Response Score (IRS) from the expression levels of the RNA transcripts of the at least two of PD-1, TOP2A, PD-L1 and ADAM12 obtained in step (a), and the transformed TMB measurement, wherein the IRS is positively correlated with the likelihood that the patient has a beneficial response to checkpoint inhibitor therapy, and obtaining an IRS having a value indicating a beneficial response to checkpoint inhibitor therapy; and (d) administering the checkpoint inhibitor therapy to the subject.

In some embodiments, the step of measuring expression levels of RNA transcripts comprises measuring expression levels of RNA transcripts for at least three of PD-1, TOP2A, PD-L1 and ADAM12, and the step of calculating an IRS comprises calculating the IRS from the expression levels of the RNA transcripts of the at least three of PD-1, TOP2A, PD-L1 and ADAM12, and the transformed TMB measurement. In some embodiments, the step of measuring expression levels of RNA transcripts comprises measuring expression levels of RNA transcripts for all of PD-1, TOP2A, PD-L1 and ADAM12, and the step of calculating an IRS comprises calculating the IRS from the expression levels of the RNA transcripts of all of PD-1, TOP2A, PD-L1 and ADAM12, and the transformed TMB measurement. In some embodiments, the step of measuring expression levels of RNA transcripts comprises measuring expression levels of RNA transcripts of at least PD-1 and PD-L1, and the step of calculating an IRS comprises calculating the IRS from the expression levels of the RNA transcripts of at least PD-1 and PD-L1, and the transformed TMB measurement.

In some embodiments, the step of measuring expression levels of RNA transcripts comprises measuring expression levels of RNA transcripts of PD-1, PD-L1, and ADAM12 and wherein the step of calculating an IRS comprises calculating the IRS from the expression levels of the RNA transcripts of PD-1, PD-L1, and ADAM12, and the transformed TMB measurement. In some embodiments, expression levels of both PD-1 and PD-L1 are positively correlated with the likelihood that the patient has a beneficial response to checkpoint inhibitor therapy. In some embodiments, a high transformed level of TMB is positively correlated with the likelihood that the patient has a beneficial response to checkpoint inhibitor therapy. In some embodiments, expression levels of both PD-1 and PD-L1 are as well as a high transformed level of TMB are positively correlated with the likelihood that the patient has a beneficial response to checkpoint inhibitor therapy. In some embodiments, expression levels of ADAM12 are negatively correlated with the likelihood that the patient has a beneficial response to checkpoint inhibit therapy. In some embodiments, both of the expression levels of PD-L and PD-L1 as well as a high transformed level of TMB are positively correlated with the likelihood that the patient has a beneficial response to checkpoint inhibitor therapy, while expression levels of ADAM12 are negatively correlated with the likelihood that the patient has a beneficial response to checkpoint inhibitor therapy.

In some embodiments, step (a) further comprises ii) measuring expression levels of RNA transcripts for at least one reference gene in the biological sample, and iii) normalizing the measured expression levels of the measured RNA transcripts of the at least two, the at least three, or all of PD-1, TOP2A, PD-L1 and ADAM12 against the level of the RNA transcripts of the at least one reference gene to provide normalized expression levels of the PD-1, TOP2A, PD-L1 and ADAM12 RNA transcripts. In some embodiments, the one or more reference genes comprise one, two, three, four, five, six, seven, eight, nine, ten, eleven, twelve, thirteen, fourteen, fifteen or more housekeeping genes. In some embodiments, the reference genes are selected from CIAO1, EIF2B1, HMBS, CTCF, GGNBP2, ITGB7, MYC and SLC4A1AP. In some embodiments, the one or more reference genes comprise three or more of CIAO1, EIF2B1, HMBS, CTCF, GGNBP2, ITGB7, MYC and SLC4A1AP. In some embodiments, the one or more reference genes comprise the combination of CIAO1, EIF2B1 and HMBS. Thus, in some embodiments, the expression levels of RNA transcripts used to calculate the IRS comprises normalized expression levels of RNA transcripts.

In some embodiments, normalized expression levels of both PD-1 and PD-L1 as well as a high transformed level of TMB are positively correlated with the likelihood that the patient has a beneficial response to checkpoint inhibitor therapy. In some embodiments, normalized expression levels of both ADAM12 and TOP2A are negatively correlated with the likelihood that the patient has a beneficial response to checkpoint inhibitor therapy. In some embodiments, both of the normalized expression levels of PD-L and PD-L1 as well as a high transformed level of TMB are positively correlated with the likelihood that the patient has a beneficial response to checkpoint inhibitor therapy, while expression levels of ADAM12 and TOP2A are negatively correlated with the likelihood that the patient has a beneficial response to checkpoint inhibitor therapy.

In some embodiments, step (a) further comprises iv) median centering the measured expression levels of RNA transcripts of the at least two, the at least three, or all of PD-1, TOP2A, PD-L1 and ADAM12, prior or after to normalizing the expression levels of the measured RNA transcripts.

In some embodiments, step (a) further comprises v) log 2 transforming the measured expression levels, the median centered expression levels, the normalized expression levels or the median centered normalized expression levels of RNA transcripts of the at least two, the at least three, or all of PD-1, TOP2A, PD-L1 and ADAM12, and wherein the expression levels utilized to calculate the IRS in step (c) are transformed expression levels, transformed median centered expression levels, transformed normalized expression levels or transformed median centered normalized expression levels. In some embodiments, transformed expression levels, transformed median centered expression levels, transformed normalized expression levels or transformed median centered normalized expression levels of both PD-1 and PD-L1 as well as a high transformed level of TMB are positively correlated with the likelihood that the patient has a beneficial response to checkpoint inhibitor therapy. In some embodiments, transformed expression levels, transformed median centered expression levels, transformed normalized expression levels or transformed median centered normalized expression levels of both ADAM12 and TOP2A are negatively correlated with the likelihood that the patient has a beneficial response to checkpoint inhibitor therapy. In some embodiments, transformed expression levels, transformed median centered expression levels, transformed normalized expression levels or transformed median centered normalized expression levels of PD-L and PD-L1 as well as a high transformed level of TMB are positively correlated with the likelihood that the patient has a beneficial response to checkpoint inhibitor therapy, while transformed expression levels, transformed median centered expression levels, transformed normalized expression levels or transformed median centered normalized expression levels of ADAM12 and TOP2A are negatively correlated with the likelihood that the patient has a beneficial response to checkpoint inhibitor therapy.

In some embodiments, a determination that the IRS is positively correlated with the likelihood that the patient has a beneficial response to checkpoint inhibitor therapy is based upon the IRS exceeding a preset threshold. The IRS itself is derived from the Cox Proportional Hazards Model, and the IRS may represent a hazard function H(t), wherein H(t)=H₀(t)*exp(b₁x₂+b₂x₂+ . . . bₙxₙ), wherein H(t) is the baseline hazard, (x₁, x₂, . . . , xₙ) are covariates that determine the hazard at time (t), (b₁, b₂, . . . , bₙ) are coefficients which signal the influence of the different covariates in determining the hazard at time (t). In some embodiments, the IRS is derived from the Cox Proportional Hazards Model and may represent the relative risk H(t)/H₀(t), wherein the IRS is H(t)/H₀(t)=exp(b₁x₂+b₂x₂+ . . . bₙxₙ). In some further embodiments, the IRS is derived from the Cox Proportional Hazards Model and may represent the natural logarithm of the relative risk H(t)/H₀(t), such that IRS is ln[H(t)/H₀(t)]=(b₁x₂+b₂x₂+ . . . bₙxₙ). Thus, it will be appreciated by one of ordinary skill in the art that the threshold utilized to determine that the IRS is positively correlated with the likelihood that the patient has a beneficial response to checkpoint inhibitor therapy, will vary based upon how the IRS is derived from the Cox Proportional Hazard Model.

In some embodiments, the IRS is calculated as follows: IRS=approximately 0.27*[transformed TMB measurement]+approximately 0.11*[transformed PD-1 level]+approximately 0.06*[transformed PD-L1 level]−approximately 0.06 [transformed ADAM12 level]−approximately 0.077*[transformed TOP2A level], wherein the transformed PD-1 level, the transformed PD-L1 level, the transformed ADAM12 level, the transformed TOP2A level, are each one of a transformed expression level, a transformed median centered expression level, a transformed normalized expression level, or a transformed median centered normalized expression level.

In some embodiments, the IRS is calculated as follows: IRS=0.27*[transformed TMB measurement]+0.11*[transformed PD-1 level]+0.06*[transformed PD-L1 level]−0.06 [transformed ADAM12 level]−0.077*[transformed TOP2A level], wherein the transformed PD-1 level, the transformed PD-L1 level, the transformed ADAM12 level, the transformed TOP2A level, are each one of a transformed expression level, a transformed median centered expression level, a transformed normalized expression level, or a transformed median centered normalized expression level.

In some embodiments, the IRS is calculated as follows: IRS=0.273758*[transformed TMB measurement]+0.112641*[transformed PD-1 level]+0.061904*[transformed PD-L1 level]−0.057991 [transformed ADAM12 level]−0.077011*[transformed TOP2A level], wherein the transformed PD-1 level, the transformed PD-L1 level, the transformed ADAM12 level, the transformed TOP2A level, are each one of a transformed expression level, a transformed median centered expression level, a transformed normalized expression level, or a transformed median centered normalized expression level.

In some embodiments, the IRS value indicating beneficial response to checkpoint inhibitor therapy is approximately 0.70, 0.75, 0.80, 0.85, 0.90, 0.95, 1.00, 1.05, 1.10, 1.15, 1.20, 1.25, 1.30 or more. In some embodiments, the IRS value indicating beneficial response to checkpoint inhibitor is 0.80, 0.81, 0.82, 0.83, 0.84, 0.85, 0.86, 0.87, 0.88, 0.89, 0.90. 0.91 or more. In some embodiments, the IRS value indicating beneficial response to checkpoint inhibitor therapy is 0.87 or higher. In some embodiments, the IRS value indicating beneficial response to checkpoint inhibitor therapy is 0.873569 or higher.

Some aspects of the present invention are directed to a method of treatment, comprising: a. measuring expression levels of RNA transcripts for PD-1 and PD-L2 obtained from a tumor specimen from a subject, b. measuring tumor mutation burden (TMB) in the biological sample and log 2 transforming the TMB measurement to provide a transformed TMB measurement; c. calculating a Immunotherapy Response Score (IRS) from the expression levels or normalized levels of the RNA transcripts of PD-1 and PD-L2, and the transformed TMB measurement, wherein the IRS is positively correlated with the likelihood that the patient has a beneficial response to checkpoint inhibitor therapy, and obtaining an IRS having a value indicating a beneficial response to checkpoint inhibitor therapy; and d. administering the checkpoint inhibitor therapy to the subject.

In some embodiments, each of the transformed TMB expression, PD-1 expression and PD-L2 expression are positively correlated with the likelihood that the patient has a beneficial response to checkpoint inhibitor therapy. In some embodiments, each of the transformed TMB expression, normalized PD-1 expression and normalized PD-L2 expression are positively correlated with the likelihood that the patient has a beneficial response to checkpoint inhibitor therapy. In some embodiments, the IRS is calculated as follows: IRS=3.97*exp(0.301*[transformed TMB measurement]+0.110*[PD-1 normalized level]+0.078*[PD-L2 normalized level]).

In some embodiments, step a. further comprises measuring the expression level of RNA transcripts for ADAM12. In some embodiments, each of the transformed TMB expression, PD-1 expression, and PD-L2 expression are positively correlated with the likelihood that the patient has a beneficial response to checkpoint inhibitor therapy and ADAM12 expression is negatively correlated with the likelihood that the patient has a beneficial response to checkpoint inhibitor therapy. In some embodiments, each of the transformed TMB expression, normalized PD-1 expression, and normalized PD-L2 expression are positively correlated with the likelihood that the patient has a beneficial response to checkpoint inhibitor therapy and normalized ADAM12 expression is negatively correlated with the likelihood that the patient has a beneficial response to checkpoint inhibitor therapy. In some embodiments, the IRS is calculated as follows: IRS=3.90*exp(0.307*[transformed TMB measurement]+0.115*[PD-1 normalized level]+0.106*[PD-L2 normalized level]+−0.070*[ADAM12 normalized level]).

In some embodiments, step a. further comprises measuring the expression level of RNA transcripts for PD-L1. In some embodiments, each of the transformed TMB expression, PD-1 expression, PD-L1 expression, and PD-L2 expression are positively correlated with the likelihood that the patient has a beneficial response to checkpoint inhibitor therapy. In some embodiments, each of the transformed TMB expression, normalized PD-1 expression, normalized PD-L1 expression and normalized PD-L2 expression are positively correlated with the likelihood that the patient has a beneficial response to checkpoint inhibitor therapy. In some embodiments, the IRS is calculated as follows: IRS=3.91*exp(0.296*[transformed TMB measurement]+0.097*[PD-1 normalized level]+0.056*[PD-L2 normalized level]+0.0043*[PD-L1 normalized level]).

In some embodiments, step a. further comprises measuring the expression level of RNA transcripts for CD4. In some embodiments, each of the transformed TMB expression, PD-1 expression, and PD-L2 expression are positively correlated with the likelihood that the patient has a beneficial response to checkpoint inhibitor therapy and CD4 expression is negatively correlated with the likelihood that the patient has a beneficial response to checkpoint inhibitor therapy. In some embodiments, each of the transformed TMB expression, normalized PD-1 expression, and normalized PD-L2 expression are positively correlated with the likelihood that the patient has a beneficial response to checkpoint inhibitor therapy and normalized CD4 expression is negatively correlated with the likelihood that the patient has a beneficial response to checkpoint inhibitor therapy. In some embodiments, the IRS is calculated as follows: IRS=4.10*exp(0.281*[transformed TMB measurement]+0.139*[PD-1 normalized level]+0.112*[PD-L2 normalized level]+−0.128*[CD4 normalized level]).

In some embodiments, the expression levels of RNA transcripts for PD-1, PD-L2, CD4, ADAM12, and PD-L1 are measured. In some embodiments, each of the transformed TMB expression, PD-1 expression, PD-L1, and PD-L2 expression are positively correlated with the likelihood that the patient has a beneficial response to checkpoint inhibitor therapy and CD4 and ADAM12 expression are negatively correlated with the likelihood that the patient has a beneficial response to checkpoint inhibitor therapy. In some embodiments, each of the transformed TMB expression, normalized PD-1 expression, normalized PD-L1, and normalized PD-L2 expression are positively correlated with the likelihood that the patient has a beneficial response to checkpoint inhibitor therapy and normalized CD4 expression and normalized ADAM12 expression are negatively correlated with the likelihood that the patient has a beneficial response to checkpoint inhibitor therapy. In some embodiments, the expression levels of RNA transcripts for PD-1, PD-L2, CD4, ADAM12, and PD-L1 are measured, and wherein the IRS is calculated as follows: IRS=3.95*exp(0.280*[transformed TMB measurement]+0.134*[PD-1 normalized level]+0.122*[PD-L2 normalized level]+−0.070*[ADAM12 normalized level]+−0.154*[CD4 normalized level]+0.052*[PD-L1 normalized level]).

In some embodiments, the expression levels of RNA transcripts for PD-1, PD-L2, CD4, and ADAM12 are measured. In some embodiments, each of the transformed TMB expression, PD-1 expression, and PD-L2 expression are positively correlated with the likelihood that the patient has a beneficial response to checkpoint inhibitor therapy and CD4 and ADAM12 expression are negatively correlated with the likelihood that the patient has a beneficial response to checkpoint inhibitor therapy. In some embodiments, each of the transformed TMB expression, normalized PD-1 expression, and normalized PD-L2 expression are positively correlated with the likelihood that the patient has a beneficial response to checkpoint inhibitor therapy and normalized CD4 expression and normalized ADAM12 expression are negatively correlated with the likelihood that the patient has a beneficial response to checkpoint inhibitor therapy. In some embodiments, the expression levels of RNA transcripts for PD-1, PD-L2, CD4, and ADAM12 are measured, and wherein the IRS is calculated as follows: IRS=4.03*exp(0.287*[transformed TMB measurement]+0.147*[PD-1 normalized level]+0.143*[PD-L2 normalized level]−0.138*[CD4 normalized level]−0.073*[ADAM12 normalized level]).

In some embodiments, step a. further comprises measuring the expression level of RNA transcripts for VTCN1. In some embodiments, each of the transformed TMB expression, PD-1 expression, VTCN1 expression, and PD-L2 expression are positively correlated with the likelihood that the patient has a beneficial response to checkpoint inhibitor therapy. In some embodiments, each of the transformed TMB expression, normalized PD-1 expression, normalized VTCN1 expression and normalized PD-L2 expression are positively correlated with the likelihood that the patient has a beneficial response to checkpoint inhibitor therapy. In some embodiments, the IRS is calculated as follows: IRS=3.90*exp(0.309*[transformed TMB measurement]+0.104*[PD-1 normalized level]+0.087*[PD-L2 normalized level]+0.021*[VTCN1 normalized level]).

In some embodiments, the expression levels of RNA transcripts for PD-1, PD-L2, CD4, ADAM12, and VTCN1 are measured In some embodiments, each of the transformed TMB expression, PD-1 expression, and PD-L2 expression are positively correlated with the likelihood that the patient has a beneficial response to checkpoint inhibitor therapy and CD4 expression is negatively correlated with the likelihood that the patient has a beneficial response to checkpoint inhibitor therapy. In some embodiments, each of the transformed TMB expression, normalized PD-1 expression, and normalized PD-L2 expression are positively correlated with the likelihood that the patient has a beneficial response to checkpoint inhibitor therapy and normalized CD4 expression is negatively correlated with the likelihood that the patient has a beneficial response to checkpoint inhibitor therapy. In some embodiments, the expression levels of RNA transcripts for PD-1, PD-L2, CD4, ADAM12, and VTCN1 are measured, and wherein the IRS is calculated as follows: IRS=3.95*exp(0.295*[transformed TMB measurement]+0.142*[PD-1 normalized level]+0.150*[PD-L2 normalized level]+0.020*[VTCN1 normalized level]+−0.070*[ADAM12 normalized level]+−0.139*[CD4 normalized level]).

In some embodiments of each of the methods disclosed herein, the IRS value indicating beneficial response to checkpoint inhibitor therapy is 6 or more, 7 or more, 8 or more, 9 or more, 9.5 or more, 10 or more, 10.5 or more, 11 or more, 12 or more, 13 or more, 14 or more, 15 or more, or 20 or more. In some embodiments of the methods disclosed herein, the IRS value indicating beneficial response to checkpoint inhibitor therapy is 8 or more. In some embodiments of the methods disclosed herein, the IRS value indicating beneficial response to checkpoint inhibitor therapy is 10 or more. In some embodiments of the methods disclosed herein, the IRS value indicating beneficial response to checkpoint inhibitor therapy is 12 or more. In some embodiments of the methods disclosed herein, the IRS is calculated using the Cox model as 8, 10 or 12 times the inverse of the patient hazard ratio as compared to the median hazard rate. In some embodiments of the methods disclosed herein, the IRS is calculated using the Cox model as 10 times the inverse of the patient hazard ratio as compared to the median hazard rate. In some embodiments of the methods disclosed herein, the IRS is calculated using the Cox model as between 8 and 12 times the inverse of the patient hazard ratio as compared to the median hazard rate.

In some embodiments, the tumor specimen is a formalin-fixed paraffin-embedded (FFPE) tumor specimen. In some embodiments, the tumor specimen contains at least 20% tumor content. In some embodiments, the tumor specimen is breast cancer, central or peripheral nervous system cancer, cancer of unknown primary, colorectal cancer, endometrial cancer, a gastrointestinal stromal tumor, glioma, hepatobiliary cancer, neuroendocrine cancer, ovarian cancer, pancreatic cancer, prostate cancer salivary gland cancer, sarcoma, or thyroid cancer. In some embodiments, the tumor specimen is adrenal cancer, biliary cancer, bladder cancer, brain cancer, breast cancer, cervical cancer, colon cancer, rectum cancer, endometrial cancer, esophageal cancer, head or neck cancer, kidney cancer, liver cancer, non-small cell lung cancer, lung cancer, lymphoma, melanoma, meniges cancer, non-melanoma skin cancer, ovarian cancer, pancreatic cancer, prostate cancer, sarcoma, small intestine cancer, or stomach cancer.

In some embodiments, the tumor specimen is assessed as having microsatellite instability-low or microsatellite stability. In some embodiments, the tumor specimen is assessed as having a low tumor mutational burden, wherein low tumor mutational burden is classified as having less than 10 mutations per megabase (muts/Mb). In some embodiments, the expression levels of RNA transcripts are measured using PCR and next-generation sequencing.

In some embodiments, the checkpoint inhibitor is an anti-PD-1 antibody, an anti-CTLA-4 antibody, an anti-PD-L1 antibody, or an anti-PD-L2. In some embodiments, the checkpoint inhibitor is nivolumab, pembrolizumab, atezolizumab, cemiplimab, durvalumab, pidilizumab, atezolimumab, PDR001, BMS-936559, avelumab, ipilimumab, or SHR-1210.

In some embodiments, the checkpoint inhibitor therapy is administered as a monotherapy. In some embodiments, the checkpoint inhibitor therapy is administered in combination with one or more other chemotherapeutic agents.

In some embodiments, the tumor specimen shows a TPS score of 1-49%. In some embodiments, the checkpoint inhibitor is administered as part of a 1^{st} line treatment regimen. In some embodiments, the checkpoint inhibitor is administered as part of a 2^{nd} line treatment regimen or higher.

In some embodiments of the methods disclosed herein, step a. further comprises measuring expression levels of RNA transcripts for at least one reference gene in the biological sample, step b. comprises normalizing the measured expression levels of the other measured RNA transcripts against the level of the RNA transcripts of at the least one reference gene to provide normalized levels of the other RNA transcripts, and step c. comprises calculating the IRS from the normalized levels. In some embodiments, the one or more reference genes comprise three genes selected from LRP1, MRPL13, TBP, HMBS, ITGB7, MYC, CIAO1, CTCF, EIF2B1, GGNBP2, SLC4A1AP. In some embodiments, the one or more reference genes comprise three genes selected from CIAO1, EIF2B1, HMBS, CTCF, GGNBP2, ITGB7, MYC and SLC4A1AP. In some embodiments, the one or more reference genes comprise the combination of CIAO1, EIF2B1 and HMBS.

Some aspects of the present disclosure are directed to a method of identifying a subject that would benefit from checkpoint inhibitor therapy, comprising: (a) measuring expression levels of RNA transcripts for PD-1, TOP2A, PD-L1 and ADAM12, and at least one reference gene in a biological sample obtained from a tumor specimen from the subject; (b) log 2 transforming, median centering, and normalizing the measured expression levels of the RNA transcripts against the level of the RNA transcripts of at the least one reference gene to provide normalized levels of the RNA transcripts; (c) measuring tumor mutation burden (TMB) in the biological sample and log 2 transforming the TMB measurement to provide a transformed TMB measurement; (d) calculating a Immunotherapy Response Score (IRS) from the transformed normalized levels of the RNA transcripts of PD-1, TOP2A, PD-L1 and ADAM12, and the transformed TMB measurement, wherein the IRS is positively correlated with the likelihood that the patient has a beneficial response to checkpoint inhibitor therapy, and obtaining an IRS having a value indicating beneficial response to checkpoint inhibitor therapy; and (e)identifying the subject as benefiting from the checkpoint inhibitor therapy.

In some embodiments, the tumor specimen is from a cancer not approved for labeled use of the checkpoint inhibitor therapy. In some embodiments, the tumor specimen is assessed as having microsatellite instability-low or microsatellite stability. In some embodiments, the tumor specimen is assessed as having a low tumor mutational burden, wherein low tumor mutational burden is classified as less than 10 mutations per megabase (muts/Mb). In some embodiments, the tumor specimen shows a TPS score of 1-49%.

Some aspects of the present disclosure are directed to a method of identifying a subject that would benefit from checkpoint inhibitor therapy, comprising: (a) receiving, by a processor, measured expression levels of RNA transcripts for at least two of PD-1, TOP2A, PD-L1 and ADAM12, and at least one reference gene in a biological sample obtained from a tumor specimen from the subject; (b) log 2 transforming, median centering, and normalizing, by a processor, the measured expression levels of the RNA transcripts against the level of the RNA transcripts of at the least one reference gene to provide transformed normalized levels of the RNA transcripts; (c) receiving, by a processor, measured tumor mutation burden (TMB) in the biological sample; (d) log 2 transforming, by a processor, the TMB measurement to provide a transformed TMB measurement; (e) calculating, by a processor, an Immunotherapy Response Score (IRS) from the transformed normalized levels of the RNA transcripts of the at least two of PD-1, TOP2A, PD-L1 and ADAM12, and the transformed TMB measurement, that is positively correlated with the likelihood that the patient has a beneficial response to checkpoint inhibitor therapy; and (f) providing a determination if the subject has a checkpoint inhibitor responsive cancer.

In some embodiments, the IRS is calculated as follows: IRS=0.27*[transformed TMB measurement]+0.11*[transformed PD-1 normalized level]+0.06*[transformed PD-L1 normalized level]−0.06 [transformed ADAM12 normalized level]−0.077*[transformed TOP2A normalized level].

In some embodiments, the IRS is calculated as follows: IRS=0.273758*[transformed TMB measurement]+0.112641*[transformed PD-1 normalized level]+0.061904*[transformed PD-L1 normalized level]−0.057991 [transformed ADAM12 normalized level]−0.077011*[transformed TOP2A normalized level].

In some embodiments, the IRS value indicating beneficial response to checkpoint inhibitor therapy is approximately 0.87 or more. In some embodiments, the IRS value indicating beneficial response to checkpoint inhibitor therapy is 0.873569 or higher. In some embodiments, the one or more reference genes comprise three genes selected from CIAO1, EIF2B1, HMBS, CTCF, GGNBP2, ITGB7, MYC and SLC4A1AP. In some embodiments, the one or more reference genes comprise the combination of CIAO1, EIF2B1 and HMBS.

Some aspects of the present disclosure are directed to a method of treatment of a subject in need thereof with a checkpoint inhibitor therapy, comprising administering to said subject the checkpoint inhibitor therapy, wherein said subject in need thereof was previously identified by a method comprising: (a) measuring expression levels of RNA transcripts for at least two of PD-1, TOP2A, PD-L1 and ADAM12, and at least one reference gene in a biological sample obtained from a tumor specimen from the subject; (b) log 2 transforming, median centering, and normalizing the measured expression levels of the RNA transcripts against the level of the RNA transcripts of at the least one reference gene to provide transformed normalized levels of the RNA transcripts; (c) measuring tumor mutation burden (TMB) in the biological sample and log 2 transforming the TMB measurement to provide a transformed TMB measurement; (d) calculating a Immunotherapy Response Score (IRS) from the transformed normalized levels of the RNA transcripts of the at least two of PD-1, TOP2A, PD-L1 and ADAM12, and the transformed TMB measurement, wherein the IRS is positively correlated with the likelihood that the patient has a beneficial response to checkpoint inhibitor therapy, and obtaining an IRS having a value indicating beneficial response to checkpoint inhibitor therapy.

In some embodiments, the IRS is calculated as follows: IRS=0.27*[transformed TMB measurement]+0.11*[transformed PD-1 normalized level]+0.06*[transformed PD-L1 normalized level]−0.06 [transformed ADAM12 normalized level]−0.077*[transformed TOP2A normalized level].

In some embodiments, the IRS is calculated as follows: IRS=0.273758*[transformed TMB measurement]+0.112641*[transformed PD-1 normalized level]+0.061904*[transformed PD-L1 normalized level]−0.057991 [transformed ADAM12 normalized level]−0.077011*[transformed TOP2A normalized level].

In some embodiments, the IRS value indicating beneficial response to checkpoint inhibitor therapy is approximately 0.87 or more. In some embodiments, the IRS value indicating beneficial response to checkpoint inhibitor therapy is 0.873569 or higher.

In some embodiments, the one or more reference genes comprise three genes selected from CIAO1, EIF2B1, HMBS, CTCF, GGNBP2, ITGB7, MYC and SLC4A1AP. In some embodiments, the one or more reference genes comprise the combination of HMBS, CIAO1 and EIF2B1.

In some embodiments, the tumor specimen is from a cancer not approved for labeled use of the checkpoint inhibitor therapy. In some embodiments, the tumor specimen is assessed as having microsatellite instability-low or microsatellite stability. In some embodiments, the tumor specimen is assessed as having a low tumor mutational burden, wherein low tumor mutational burden is classified as less than 10 mutations per megabase (muts/Mb).

In some embodiments, the checkpoint inhibitor therapy is administered as a monotherapy. In some embodiments, the checkpoint inhibitor therapy is administered in combination with one or more other chemotherapeutic agents. In some embodiments, the tumor specimen shows a TPS score of 1-49%.

Some aspects of the present disclosure are directed to a method of treatment, comprising: (a) measuring expression levels of RNA transcripts PD-1, TOP2A, PD-L1 and ADAM12, and at least one reference gene in a biological sample obtained from a tumor specimen from a subject; (b) log 2 transforming, median centering, and normalizing the measured expression levels of the RNA transcripts of PD-1, TOP2A, PD-L1 and ADAM12 against the level of the RNA transcripts of at the least one reference gene to provide transformed normalized levels of the RNA transcripts of PD-1, TOP2A, PD-L1 and ADAM12; (c) measuring tumor mutation burden (TMB) in the biological sample and log 2 transforming the TMB measurement to provide a transformed TMB measurement; (d) calculating a Immunotherapy Response Score (IRS) from the transformed normalized levels of the RNA transcripts of PD-1, TOP2A, PD-L1 and ADAM12 and the transformed TMB measurement, wherein the IRS is positively correlated with the likelihood that the patient has a beneficial response to checkpoint inhibitor therapy, and obtaining an IRS having a value indicating a beneficial response to checkpoint inhibitor therapy; and (e) administering the checkpoint inhibitor therapy to the subject.

In some embodiments, the IRS is calculated as follows: IRS=0.27*[transformed TMB measurement]+0.11*[transformed PD-1 normalized level]+0.06*[transformed PD-L1 normalized level]−0.06 [transformed ADAM12 normalized level]−0.077*[transformed TOP2A normalized level].

In some embodiments, the IRS is calculated as follows: IRS=0.273758*[transformed TMB measurement]+0.112641*[transformed PD-1 normalized level]+0.061904*[transformed PD-L1 normalized level]−0.057991 [transformed ADAM12 normalized level]−0.077011*[transformed TOP2A normalized level].

In some embodiments, the IRS value indicating beneficial response to checkpoint inhibitor therapy is approximately 0.87 or more. In some embodiments, the IRS value indicating beneficial response to checkpoint inhibitor therapy is 0.873569 or higher.

In some embodiments, the one or more reference genes comprise three genes selected from CIAO1, EIF2B1, HMBS, CTCF, GGNBP2, ITGB7, MYC and SLC4A1AP. In some embodiments, the one or more reference genes comprise the combination of HMBS, CIAO1 and EIF2B1.

In some embodiments, the tumor specimen is a formalin-fixed paraffin-embedded (FFPE) tumor specimen. In some embodiments, the tumor specimen is breast cancer, central or peripheral nervous system cancer, cancer of unknown primary, colorectal cancer, endometrial cancer, a gastrointestinal stromal tumor, glioma, hepatobiliary cancer, neuroendocrine cancer, ovarian cancer, pancreatic cancer, prostate cancer salivary gland cancer, sarcoma, or thyroid cancer.

In some embodiments, the expression levels of RNA transcripts are measured using PCR and next-generation sequencing.

In some embodiments, the checkpoint inhibitor is an anti-PD-1 antibody, an anti-CTLA-4 antibody, an anti-PD-L1 antibody, or an anti-PD-L2. In some embodiments, the checkpoint inhibitor is nivolumab, pembrolizumab, atezolizumab, cemiplimab, durvalumab, pidilizumab, atezolimumab, PDR001, BMS-936559, avelumab, ipilimumab, or SHR-1210.

Some aspects of the present disclosure are directed to a method of identifying a subject that would benefit from checkpoint inhibitor therapy, comprising: (a) measuring expression levels of RNA transcripts for PD-1, TOP2A, PD-L1 and ADAM12, and at least one reference gene in a biological sample obtained from a tumor specimen from the subject; (b) log 2 transforming, median centering, and normalizing the measured expression levels of the RNA transcripts of PD-1, TOP2A, PD-L1 and ADAM12 against the level of the RNA transcripts of at the least one reference gene to provide transformed normalized levels of the RNA transcripts of PD-1, TOP2A, PD-L1 and ADAM12; (c) measuring tumor mutation burden (TMB) in the biological sample and log 2 transforming the TMB measurement to provide a transformed TMB measurement; (d) calculating a Immunotherapy Response Score (IRS) from the transformed normalized levels of the RNA transcripts of PD-1, TOP2A, PD-L1 and ADAM12 and the transformed TMB measurement, wherein the IRS is positively correlated with the likelihood that the patient has a beneficial response to checkpoint inhibitor therapy, and obtaining an IRS having a value indicating beneficial response to checkpoint inhibitor therapy; and (e) identifying the subject as benefiting from the checkpoint inhibitor therapy.

In some embodiments, the tumor specimen is from a cancer not approved for labeled use of the checkpoint inhibitor therapy. In some embodiments, the tumor specimen is assessed as having microsatellite instability-low or microsatellite stability. In some embodiments, the tumor specimen is assessed as having a low tumor mutational burden, wherein low tumor mutational burden is classified as less than 10 mutations per megabase (muts/Mb). In some embodiments, the tumor specimen contains at least 20% tumor content.

In some embodiments, the checkpoint inhibitor therapy is administered as a monotherapy. In some embodiments, the checkpoint inhibitor therapy is administered in combination with one or more other chemotherapeutic agents. In some embodiments, the tumor specimen shows a TPS score of 1-49%.

In some embodiments of each of the methods disclosed herein a subject is identified as benefiting from checkpoint inhibitor therapy (e.g., pembrolizumab) or treated with checkpoint inhibitor therapy (e.g., pembrolizumab) when the tumor type is not approved for use with the checkpoint inhibitor (off-label) and the IRS value is 0.90 or greater. In some embodiments, a subject is identified as benefiting from checkpoint inhibitor therapy (e.g., pembrolizumab) or treated with checkpoint inhibitor therapy (e.g., pembrolizumab) when the tumor type is not approved for use with the checkpoint inhibitor (off-label) and the IRS value is 0.88 or greater. In some embodiments, a subject is identified as benefiting from checkpoint inhibitor therapy (e.g., pembrolizumab) or treated with checkpoint inhibitor therapy (e.g., pembrolizumab) when the tumor type is not approved for use with the checkpoint inhibitor (off-label), the TMB is 10 mutations per megabase (MPM) or more and the IRS value is 0.87 or greater. In some embodiments, a subject is identified as benefiting from checkpoint inhibitor therapy (e.g., pembrolizumab) or treated with checkpoint inhibitor therapy (e.g., pembrolizumab) when the tumor type is not approved for use with the checkpoint inhibitor (off-label) , the TMB is 10 MPM or more and the IRS value is 0.873 or greater. In some embodiments, a subject is identified as benefiting from checkpoint inhibitor therapy (e.g., pembrolizumab) or treated with checkpoint inhibitor therapy (e.g., pembrolizumab) when the TMB is 10 MPM or more and the IRS value is 0.8736 or greater. In some embodiments, a subject is identified as benefiting from checkpoint inhibitor therapy (e.g., pembrolizumab) or treated with checkpoint inhibitor therapy (e.g., pembrolizumab) when the TMB is 10 MPM or more and the IRS value is 0.873569 or greater.

Some aspects of the present disclosure are directed to a method of identifying a subject that would benefit from checkpoint inhibitor therapy, comprising: a. measuring expression levels of RNA transcripts for PD-1, PD-L2 and, optionally, expression levels of RNA transcripts for one or more of CD4, ADAM12, PD-L1, and VTCN1, and at least one reference gene in a biological sample obtained from a tumor specimen from the subject; b. log 2 transforming, median centering, and normalizing the measured expression levels of the RNA transcripts against the level of the RNA transcripts of at the least one reference gene to provide normalized levels of the RNA transcripts; c. measuring tumor mutation burden (TMB) in the biological sample and log 2 transforming the TMB measurement to provide a transformed TMB measurement; d. calculating a Immunotherapy Response Score (IRS) from the normalized levels of the RNA transcripts of PD-1, PD-L2 and, optionally, one or more of CD4, ADAM12, PD-L1, and VTCN1, and the transformed TMB measurement, wherein the IRS is positively correlated with the likelihood that the patient has a beneficial response to checkpoint inhibitor therapy, and obtaining an IRS having a value indicating beneficial response to checkpoint inhibitor therapy; and e. identifying the subject as benefiting from the checkpoint inhibitor therapy.

In some embodiments, the tumor specimen is from a cancer not approved for labeled use of the checkpoint inhibitor therapy. In some embodiments, the calculated IRS value indicates that the median time-to-next-treatment (TNTT) is 24 months or greater.

In some embodiments, each of the transformed TMB expression, PD-1 expression and PD-L2 expression are positively correlated with the likelihood that the patient has a beneficial response to checkpoint inhibitor therapy. In some embodiments, each of the transformed TMB expression, normalized PD-1 expression and normalized PD-L2 expression are positively correlated with the likelihood that the patient has a beneficial response to checkpoint inhibitor therapy. In some embodiments, the IRS is calculated as follows: IRS=3.97*exp(0.301*[transformed TMB measurement]+0.110*[PD-1 normalized level]+0.078*[PD-L2 normalized level]).

In some embodiments, step a. further comprises measuring the expression level of RNA transcripts for ADAM12. In some embodiments, each of the transformed TMB expression, PD-1 expression, and PD-L2 expression are positively correlated with the likelihood that the patient has a beneficial response to checkpoint inhibitor therapy and ADAM12 expression is negatively correlated with the likelihood that the patient has a beneficial response to checkpoint inhibitor therapy. In some embodiments, each of the transformed TMB expression, normalized PD-1 expression, and normalized PD-L2 expression are positively correlated with the likelihood that the patient has a beneficial response to checkpoint inhibitor therapy and normalized ADAM12 expression is negatively correlated with the likelihood that the patient has a beneficial response to checkpoint inhibitor therapy. In some embodiments, the IRS is calculated as follows: IRS=3.90*exp(0.307*[transformed TMB measurement]+0.115*[PD-1 normalized level]+0.106*[PD-L2 normalized level]+−0.070*[ADAM12 normalized level]).

In some embodiments, step a. further comprises measuring the expression level of RNA transcripts for PD-L1. In some embodiments, each of the transformed TMB expression, PD-1 expression, PD-L1 expression, and PD-L2 expression are positively correlated with the likelihood that the patient has a beneficial response to checkpoint inhibitor therapy. In some embodiments, each of the transformed TMB expression, normalized PD-1 expression, normalized PD-L1 expression and normalized PD-L2 expression are positively correlated with the likelihood that the patient has a beneficial response to checkpoint inhibitor therapy. In some embodiments, the IRS is calculated as follows: IRS=3.91*exp(0.296*[transformed TMB measurement]+0.097*[PD-1 normalized level]+0.056*[PD-L2 normalized level]+0.0043*[PD-L1 normalized level]).

In some embodiments, step a. further comprises measuring the expression level of RNA transcripts for CD4. In some embodiments, each of the transformed TMB expression, PD-1 expression, and PD-L2 expression are positively correlated with the likelihood that the patient has a beneficial response to checkpoint inhibitor therapy and CD4 expression is negatively correlated with the likelihood that the patient has a beneficial response to checkpoint inhibitor therapy. In some embodiments, each of the transformed TMB expression, normalized PD-1 expression, and normalized PD-L2 expression are positively correlated with the likelihood that the patient has a beneficial response to checkpoint inhibitor therapy and normalized CD4 expression is negatively correlated with the likelihood that the patient has a beneficial response to checkpoint inhibitor therapy. In some embodiments, the IRS is calculated as follows: IRS=4.10*exp(0.281*[transformed TMB measurement]+0.139*[PD-1 normalized level]+0.112*[PD-L2 normalized level]+−0.128*[CD4 normalized level]).

In some embodiments, the expression levels of RNA transcripts for PD-1, PD-L2, CD4, ADAM12, and PD-L1 are measured. In some embodiments, each of the transformed TMB expression, PD-1 expression, PD-L1, and PD-L2 expression are positively correlated with the likelihood that the patient has a beneficial response to checkpoint inhibitor therapy and CD4 and ADAM12 expression are negatively correlated with the likelihood that the patient has a beneficial response to checkpoint inhibitor therapy. In some embodiments, each of the transformed TMB expression, normalized PD-1 expression, normalized PD-L1, and normalized PD-L2 expression are positively correlated with the likelihood that the patient has a beneficial response to checkpoint inhibitor therapy and normalized CD4 expression and normalized ADAM12 expression are negatively correlated with the likelihood that the patient has a beneficial response to checkpoint inhibitor therapy. In some embodiments, the expression levels of RNA transcripts for PD-1, PD-L2, CD4, ADAM12, and PD-L1 are measured, and wherein the IRS is calculated as follows: IRS=3.95*exp(0.280*[transformed TMB measurement]+0.134*[PD-1 normalized level]+0.122*[PD-L2 normalized level]+−0.070*[ADAM12 normalized level]+−0.154*[CD4 normalized level]+0.052*[PD-L1 normalized level]).

In some embodiments, the expression levels of RNA transcripts for PD-1, PD-L2, CD4, and ADAM12 are measured. In some embodiments, each of the transformed TMB expression, PD-1 expression, and PD-L2 expression are positively correlated with the likelihood that the patient has a beneficial response to checkpoint inhibitor therapy and CD4 and ADAM12 expression are negatively correlated with the likelihood that the patient has a beneficial response to checkpoint inhibitor therapy. In some embodiments, each of the transformed TMB expression, normalized PD-1 expression, and normalized PD-L2 expression are positively correlated with the likelihood that the patient has a beneficial response to checkpoint inhibitor therapy and normalized CD4 expression and normalized ADAM12 expression are negatively correlated with the likelihood that the patient has a beneficial response to checkpoint inhibitor therapy. In some embodiments, the expression levels of RNA transcripts for PD-1, PD-L2, CD4, and ADAM12 are measured, and wherein the IRS is calculated as follows: IRS=4.03*exp(0.287*[transformed TMB measurement]+0.147*[PD-1 normalized level]+0.143*[PD-L2 normalized level]−0.138*[CD4 normalized level]−0.073*[ADAM12 normalized level]).

In some embodiments, step a. further comprises measuring the expression level of RNA transcripts for VTCN1. In some embodiments, each of the transformed TMB expression, PD-1 expression, VTCN1 expression, and PD-L2 expression are positively correlated with the likelihood that the patient has a beneficial response to checkpoint inhibitor therapy. In some embodiments, each of the transformed TMB expression, normalized PD-1 expression, normalized VTCN1 expression and normalized PD-L2 expression are positively correlated with the likelihood that the patient has a beneficial response to checkpoint inhibitor therapy. In some embodiments, the IRS is calculated as follows: IRS=3.90*exp(0.309*[transformed TMB measurement]+0.104*[PD-1 normalized level]+0.087*[PD-L2 normalized level]+0.021*[VTCN1 normalized level]).

In some embodiments, the expression levels of RNA transcripts for PD-1, PD-L2, CD4, ADAM12, and VTCN1 are measured In some embodiments, each of the transformed TMB expression, PD-1 expression, and PD-L2 expression are positively correlated with the likelihood that the patient has a beneficial response to checkpoint inhibitor therapy and CD4 expression is negatively correlated with the likelihood that the patient has a beneficial response to checkpoint inhibitor therapy. In some embodiments, each of the transformed TMB expression, normalized PD-1 expression, and normalized PD-L2 expression are positively correlated with the likelihood that the patient has a beneficial response to checkpoint inhibitor therapy and normalized CD4 expression is negatively correlated with the likelihood that the patient has a beneficial response to checkpoint inhibitor therapy. In some embodiments, the expression levels of RNA transcripts for PD-1, PD-L2, CD4, ADAM12, and VTCN1 are measured, and wherein the IRS is calculated as follows: IRS=3.95*exp(0.295*[transformed TMB measurement]+0.142*[PD-1 normalized level]+0.150*[PD-L2 normalized level]+0.020*[VTCN1 normalized level]+−0.070*[ADAM12 normalized level]+−0.139*[CD4 normalized level]).

Some aspects of the present disclosure are directed to a method of identifying a subject that would benefit from checkpoint inhibitor therapy, comprising: a. receiving, by a processor, measured expression levels of RNA transcripts for PD-1, PD-L2 and, optionally, expression levels of RNA transcripts for one or more of CD4, ADAM12, PD-L1, and VTCN1, and at least one reference gene in a biological sample obtained from a tumor specimen from the subject; b. log 2 transforming, median centering, and normalizing, by a processor, the measured expression levels of the RNA transcripts against the level of the RNA transcripts of at the least one reference gene to provide normalized levels of the RNA transcripts; c. receiving, by a processor, measured tumor mutation burden (TMB) in the biological sample; d. log 2 transforming, by a processor, the TMB measurement to provide a transformed TMB measurement; e. calculating, by a processor, a Immunotherapy Response Score (IRS) from the normalized levels of the RNA transcripts of PD-1, PD-L2 and, optionally, one or more of CD4, ADAM12, PD-L1, and VTCN1, and the transformed TMB measurement, that is positively correlated with the likelihood that the patient has a beneficial response to checkpoint inhibitor therapy; and f. providing a determination if the subject has a checkpoint inhibitor responsive cancer.

In some embodiments, each of the transformed TMB expression, PD-1 expression and PD-L2 expression are positively correlated with the likelihood that the patient has a beneficial response to checkpoint inhibitor therapy. In some embodiments, each of the transformed TMB expression, normalized PD-1 expression and normalized PD-L2 expression are positively correlated with the likelihood that the patient has a beneficial response to checkpoint inhibitor therapy. In some embodiments, the IRS is calculated as follows: IRS=3.97*exp(0.301*[transformed TMB measurement]+0.110*[PD-1 normalized level]+0.078*[PD-L2 normalized level]).

In some embodiments, step a. further comprises measuring the expression level of RNA transcripts for ADAM12. In some embodiments, each of the transformed TMB expression, PD-1 expression, and PD-L2 expression are positively correlated with the likelihood that the patient has a beneficial response to checkpoint inhibitor therapy and ADAM12 expression is negatively correlated with the likelihood that the patient has a beneficial response to checkpoint inhibitor therapy. In some embodiments, each of the transformed TMB expression, normalized PD-1 expression, and normalized PD-L2 expression are positively correlated with the likelihood that the patient has a beneficial response to checkpoint inhibitor therapy and normalized ADAM12 expression is negatively correlated with the likelihood that the patient has a beneficial response to checkpoint inhibitor therapy. In some embodiments, the IRS is calculated as follows: IRS=3.90*exp(0.307*[transformed TMB measurement]+0.115*[PD-1 normalized level]+0.106*[PD-L2 normalized level]+−0.070*[ADAM12 normalized level]).

In some embodiments, step a. further comprises measuring the expression level of RNA transcripts for PD-L1. In some embodiments, each of the transformed TMB expression, PD-1 expression, PD-L1 expression, and PD-L2 expression are positively correlated with the likelihood that the patient has a beneficial response to checkpoint inhibitor therapy. In some embodiments, each of the transformed TMB expression, normalized PD-1 expression, normalized PD-L1 expression and normalized PD-L2 expression are positively correlated with the likelihood that the patient has a beneficial response to checkpoint inhibitor therapy. In some embodiments, the IRS is calculated as follows: IRS=3.91*exp(0.296*[transformed TMB measurement]+0.097*[PD-1 normalized level]+0.056*[PD-L2 normalized level]+0.0043*[PD-L1 normalized level]).

In some embodiments, step a. further comprises measuring the expression level of RNA transcripts for CD4. In some embodiments, each of the transformed TMB expression, PD-1 expression, and PD-L2 expression are positively correlated with the likelihood that the patient has a beneficial response to checkpoint inhibitor therapy and CD4 expression is negatively correlated with the likelihood that the patient has a beneficial response to checkpoint inhibitor therapy. In some embodiments, each of the transformed TMB expression, normalized PD-1 expression, and normalized PD-L2 expression are positively correlated with the likelihood that the patient has a beneficial response to checkpoint inhibitor therapy and normalized CD4 expression is negatively correlated with the likelihood that the patient has a beneficial response to checkpoint inhibitor therapy. In some embodiments, the IRS is calculated as follows: IRS=4.10*exp(0.281*[transformed TMB measurement]+0.139*[PD-1 normalized level]+0.112*[PD-L2 normalized level]+−0.128*[CD4 normalized level]).

In some embodiments, the expression levels of RNA transcripts for PD-1, PD-L2, CD4, ADAM12, and PD-L1 are measured. In some embodiments, each of the transformed TMB expression, PD-1 expression, PD-L1, and PD-L2 expression are positively correlated with the likelihood that the patient has a beneficial response to checkpoint inhibitor therapy and CD4 and ADAM12 expression are negatively correlated with the likelihood that the patient has a beneficial response to checkpoint inhibitor therapy. In some embodiments, each of the transformed TMB expression, normalized PD-1 expression, normalized PD-L1, and normalized PD-L2 expression are positively correlated with the likelihood that the patient has a beneficial response to checkpoint inhibitor therapy and normalized CD4 expression and normalized ADAM12 expression are negatively correlated with the likelihood that the patient has a beneficial response to checkpoint inhibitor therapy. In some embodiments, the expression levels of RNA transcripts for PD-1, PD-L2, CD4, ADAM12, and PD-L1 are measured, and wherein the IRS is calculated as follows: IRS=3.95*exp(0.280*[transformed TMB measurement]+0.134*[PD-1 normalized level]+0.122*[PD-L2 normalized level]+−0.070*[ADAM12 normalized level]+−0.154*[CD4 normalized level]+0.052*[PD-L1 normalized level]).

In some embodiments, the expression levels of RNA transcripts for PD-1, PD-L2, CD4, and ADAM12 are measured. In some embodiments, each of the transformed TMB expression, PD-1 expression, and PD-L2 expression are positively correlated with the likelihood that the patient has a beneficial response to checkpoint inhibitor therapy and CD4 and ADAM12 expression are negatively correlated with the likelihood that the patient has a beneficial response to checkpoint inhibitor therapy. In some embodiments, each of the transformed TMB expression, normalized PD-1 expression, and normalized PD-L2 expression are positively correlated with the likelihood that the patient has a beneficial response to checkpoint inhibitor therapy and normalized CD4 expression and normalized ADAM12 expression are negatively correlated with the likelihood that the patient has a beneficial response to checkpoint inhibitor therapy. In some embodiments, the expression levels of RNA transcripts for PD-1, PD-L2, CD4, and ADAM12 are measured, and wherein the IRS is calculated as follows: IRS=4.03*exp(0.287*[transformed TMB measurement]+0.147*[PD-1 normalized level]+0.143*[PD-L2 normalized level]−0.138*[CD4 normalized level]−0.073*[ADAM12 normalized level]).

In some embodiments, step a. further comprises measuring the expression level of RNA transcripts for VTCN1. In some embodiments, each of the transformed TMB expression, PD-1 expression, VTCN1 expression, and PD-L2 expression are positively correlated with the likelihood that the patient has a beneficial response to checkpoint inhibitor therapy. In some embodiments, each of the transformed TMB expression, normalized PD-1 expression, normalized VTCN1 expression and normalized PD-L2 expression are positively correlated with the likelihood that the patient has a beneficial response to checkpoint inhibitor therapy. In some embodiments, the IRS is calculated as follows: IRS=3.90*exp(0.309*[transformed TMB measurement]+0.104*[PD-1 normalized level]+0.087*[PD-L2 normalized level]+0.021*[VTCN1 normalized level]).

In some embodiments, the expression levels of RNA transcripts for PD-1, PD-L2, CD4, ADAM12, and VTCN1 are measured In some embodiments, each of the transformed TMB expression, PD-1 expression, and PD-L2 expression are positively correlated with the likelihood that the patient has a beneficial response to checkpoint inhibitor therapy and CD4 expression is negatively correlated with the likelihood that the patient has a beneficial response to checkpoint inhibitor therapy. In some embodiments, each of the transformed TMB expression, normalized PD-1 expression, and normalized PD-L2 expression are positively correlated with the likelihood that the patient has a beneficial response to checkpoint inhibitor therapy and normalized CD4 expression is negatively correlated with the likelihood that the patient has a beneficial response to checkpoint inhibitor therapy. In some embodiments, the expression levels of RNA transcripts for PD-1, PD-L2, CD4, ADAM12, and VTCN1 are measured, and wherein the IRS is calculated as follows: IRS=3.95*exp(0.295*[transformed TMB measurement]+0.142*[PD-1 normalized level]+0.150*[PD-L2 normalized level]+0.020*[VTCN1 normalized level]+−0.070*[ADAM12 normalized level]+−0.139*[CD4 normalized level]).

Some aspects of the present disclosure are directed to a method of treatment of a subject in need thereof with a checkpoint inhibitor therapy, comprising administering to said subject the checkpoint inhibitor therapy, wherein said subject in need thereof was previously identified by a method comprising: a. measuring expression levels of RNA transcripts for PD-1, PD-L2 and, optionally, expression levels of RNA transcripts for one or more of CD4, ADAM12, PD-L1, and VTCN1, and at least one reference gene in a biological sample obtained from a tumor specimen from the subject; b. log 2 transforming, median centering, and normalizing the measured expression levels of the RNA transcripts against the level of the RNA transcripts of at the least one reference gene to provide normalized levels of the RNA transcripts; c. measuring tumor mutation burden (TMB) in the biological sample and log 2 transforming the TMB measurement to provide a transformed TMB measurement; d. calculating a Immunotherapy Response Score (IRS) from the normalized levels of the RNA transcripts of PD-1, PD-L2 and, optionally, one or more of CD4, ADAM12, PD-L1, and VTCN1, and the transformed TMB measurement, wherein the IRS is positively correlated with the likelihood that the patient has a beneficial response to checkpoint inhibitor therapy, and obtaining an IRS having a value indicating beneficial response to checkpoint inhibitor therapy.

In some embodiments, the IRS is calculated using the Cox model as 10 times the inverse of the patient hazard ratio as compared to the median hazard rate.

In some embodiments, each of the transformed TMB expression, PD-1 expression and PD-L2 expression are positively correlated with the likelihood that the patient has a beneficial response to checkpoint inhibitor therapy. In some embodiments, each of the transformed TMB expression, normalized PD-1 expression and normalized PD-L2 expression are positively correlated with the likelihood that the patient has a beneficial response to checkpoint inhibitor therapy. In some embodiments, the IRS is calculated as follows: IRS=3.97*exp(0.301*[transformed TMB measurement]+0.110*[PD-1 normalized level]+0.078*[PD-L2 normalized level]).

In some embodiments, step a. further comprises measuring the expression level of RNA transcripts for ADAM12. In some embodiments, each of the transformed TMB expression, PD-1 expression, and PD-L2 expression are positively correlated with the likelihood that the patient has a beneficial response to checkpoint inhibitor therapy and ADAM12 expression is negatively correlated with the likelihood that the patient has a beneficial response to checkpoint inhibitor therapy. In some embodiments, each of the transformed TMB expression, normalized PD-1 expression, and normalized PD-L2 expression are positively correlated with the likelihood that the patient has a beneficial response to checkpoint inhibitor therapy and normalized ADAM12 expression is negatively correlated with the likelihood that the patient has a beneficial response to checkpoint inhibitor therapy. In some embodiments, the IRS is calculated as follows: IRS=3.90*exp(0.307*[transformed TMB measurement]+0.115*[PD-1 normalized level]+0.106*[PD-L2 normalized level]+−0.070*[ADAM12 normalized level]).

In some embodiments, step a. further comprises measuring the expression level of RNA transcripts for PD-L1. In some embodiments, each of the transformed TMB expression, PD-1 expression, PD-L1 expression, and PD-L2 expression are positively correlated with the likelihood that the patient has a beneficial response to checkpoint inhibitor therapy. In some embodiments, each of the transformed TMB expression, normalized PD-1 expression, normalized PD-L1 expression and normalized PD-L2 expression are positively correlated with the likelihood that the patient has a beneficial response to checkpoint inhibitor therapy. In some embodiments, the IRS is calculated as follows: IRS=3.91*exp(0.296*[transformed TMB measurement]+0.097*[PD-1 normalized level]+0.056*[PD-L2 normalized level]+0.0043*[PD-L1 normalized level]).

In some embodiments, step a. further comprises measuring the expression level of RNA transcripts for CD4. In some embodiments, each of the transformed TMB expression, PD-1 expression, and PD-L2 expression are positively correlated with the likelihood that the patient has a beneficial response to checkpoint inhibitor therapy and CD4 expression is negatively correlated with the likelihood that the patient has a beneficial response to checkpoint inhibitor therapy. In some embodiments, each of the transformed TMB expression, normalized PD-1 expression, and normalized PD-L2 expression are positively correlated with the likelihood that the patient has a beneficial response to checkpoint inhibitor therapy and normalized CD4 expression is negatively correlated with the likelihood that the patient has a beneficial response to checkpoint inhibitor therapy. In some embodiments, the IRS is calculated as follows: IRS=4.10*exp(0.281*[transformed TMB measurement]+0.139*[PD-1 normalized level]+0.112*[PD-L2 normalized level]+−0.128*[CD4 normalized level]).

In some embodiments, the expression levels of RNA transcripts for PD-1, PD-L2, CD4, ADAM12, and PD-L1 are measured. In some embodiments, each of the transformed TMB expression, PD-1 expression, PD-L1, and PD-L2 expression are positively correlated with the likelihood that the patient has a beneficial response to checkpoint inhibitor therapy and CD4 and ADAM12 expression are negatively correlated with the likelihood that the patient has a beneficial response to checkpoint inhibitor therapy. In some embodiments, each of the transformed TMB expression, normalized PD-1 expression, normalized PD-L1, and normalized PD-L2 expression are positively correlated with the likelihood that the patient has a beneficial response to checkpoint inhibitor therapy and normalized CD4 expression and normalized ADAM12 expression are negatively correlated with the likelihood that the patient has a beneficial response to checkpoint inhibitor therapy. In some embodiments, the expression levels of RNA transcripts for PD-1, PD-L2, CD4, ADAM12, and PD-L1 are measured, and wherein the IRS is calculated as follows: IRS=3.95*exp(0.280*[transformed TMB measurement]+0.134*[PD-1 normalized level]+0.122*[PD-L2 normalized level]+−0.070*[ADAM12 normalized level]+−0.154*[CD4 normalized level]+0.052*[PD-L1 normalized level]).

In some embodiments, the expression levels of RNA transcripts for PD-1, PD-L2, CD4, and ADAM12 are measured. In some embodiments, each of the transformed TMB expression, PD-1 expression, and PD-L2 expression are positively correlated with the likelihood that the patient has a beneficial response to checkpoint inhibitor therapy and CD4 and ADAM12 expression are negatively correlated with the likelihood that the patient has a beneficial response to checkpoint inhibitor therapy. In some embodiments, each of the transformed TMB expression, normalized PD-1 expression, and normalized PD-L2 expression are positively correlated with the likelihood that the patient has a beneficial response to checkpoint inhibitor therapy and normalized CD4 expression and normalized ADAM12 expression are negatively correlated with the likelihood that the patient has a beneficial response to checkpoint inhibitor therapy. In some embodiments, the expression levels of RNA transcripts for PD-1, PD-L2, CD4, and ADAM12 are measured, and wherein the IRS is calculated as follows: IRS=4.03*exp(0.287*[transformed TMB measurement]+0.147*[PD-1 normalized level]+0.143*[PD-L2 normalized level]−0.138*[CD4 normalized level]−0.073*[ADAM12 normalized level]).

In some embodiments, step a. further comprises measuring the expression level of RNA transcripts for VTCN1. In some embodiments, each of the transformed TMB expression, PD-1 expression, VTCN1 expression, and PD-L2 expression are positively correlated with the likelihood that the patient has a beneficial response to checkpoint inhibitor therapy. In some embodiments, each of the transformed TMB expression, normalized PD-1 expression, normalized VTCN1 expression and normalized PD-L2 expression are positively correlated with the likelihood that the patient has a beneficial response to checkpoint inhibitor therapy. In some embodiments, the IRS is calculated as follows: IRS=3.90*exp(0.309*[transformed TMB measurement]+0.104*[PD-1 normalized level]+0.087*[PD-L2 normalized level]+0.021*[VTCN1 normalized level]).

In some embodiments, the expression levels of RNA transcripts for PD-1, PD-L2, CD4, ADAM12, and VTCN1 are measured. In some embodiments, each of the transformed TMB expression, PD-1 expression, and PD-L2 expression are positively correlated with the likelihood that the patient has a beneficial response to checkpoint inhibitor therapy and CD4 expression is negatively correlated with the likelihood that the patient has a beneficial response to checkpoint inhibitor therapy. In some embodiments, each of the transformed TMB expression, normalized PD-1 expression, and normalized PD-L2 expression are positively correlated with the likelihood that the patient has a beneficial response to checkpoint inhibitor therapy and normalized CD4 expression is negatively correlated with the likelihood that the patient has a beneficial response to checkpoint inhibitor therapy. In some embodiments, the expression levels of RNA transcripts for PD-1, PD-L2, CD4, ADAM12, and VTCN1 are measured, and wherein the IRS is calculated as follows: IRS=3.95*exp(0.295*[transformed TMB measurement]+0.142*[PD-1 normalized level]+0.150*[PD-L2 normalized level]+0.020*[VTCN1 normalized level]+−0.070*[ADAM12 normalized level]+−0.139*[CD4 normalized level]).

In some embodiments, the IRS value indicating beneficial response to checkpoint inhibitor therapy is 10 or more.

Some aspects of the present disclosure are directed to a method of treatment, comprising: a. measuring expression levels of RNA transcripts for PD-1, PD-L2, CD4, ADAM12, and at least one reference gene in a biological sample obtained from a tumor specimen from a subject; b. log 2 transforming, median centering, and normalizing the measured expression levels of the RNA transcripts of PD-1, PD-L2, CD4, and ADAM12 against the level of the RNA transcripts of at the least one reference gene to provide normalized levels of the RNA transcripts of PD-1, PD-L2, CD4, and ADAM12; c. measuring tumor mutation burden (TMB) in the biological sample and log 2 transforming the TMB measurement to provide a transformed TMB measurement; d. calculating an Immunotherapy Response Score (IRS) from the normalized levels of the RNA transcripts of PD-1, PD-L2, CD4, and ADAM12 and the transformed TMB measurement, wherein the IRS is positively correlated with the likelihood that the patient has a beneficial response to checkpoint inhibitor therapy, and obtaining an IRS having a value indicating a beneficial response to checkpoint inhibitor therapy; and e. administering the checkpoint inhibitor therapy to the subject.

In some embodiments, each of the transformed TMB expression, PD-1 expression and PD-L2 expression are positively correlated with the likelihood that the patient has a beneficial response to checkpoint inhibitor therapy. In some embodiments, each of the transformed TMB expression, normalized PD-1 expression and normalized PD-L2 expression are positively correlated with the likelihood that the patient has a beneficial response to checkpoint inhibitor therapy. In some embodiments, the IRS is calculated as follows: IRS=3.97*exp(0.301*[transformed TMB measurement]+0.110*[PD-1 normalized level]+0.078*[PD-L2 normalized level]).

In some embodiments, step a. further comprises measuring the expression level of RNA transcripts for ADAM12. In some embodiments, each of the transformed TMB expression, PD-1 expression, and PD-L2 expression are positively correlated with the likelihood that the patient has a beneficial response to checkpoint inhibitor therapy and ADAM12 expression is negatively correlated with the likelihood that the patient has a beneficial response to checkpoint inhibitor therapy. In some embodiments, each of the transformed TMB expression, normalized PD-1 expression, and normalized PD-L2 expression are positively correlated with the likelihood that the patient has a beneficial response to checkpoint inhibitor therapy and normalized ADAM12 expression is negatively correlated with the likelihood that the patient has a beneficial response to checkpoint inhibitor therapy. In some embodiments, the IRS is calculated as follows: IRS=3.90*exp(0.307*[transformed TMB measurement]+0.115*[PD-1 normalized level]+0.106*[PD-L2 normalized level]+−0.070*[ADAM12 normalized level]).

In some embodiments, step a. further comprises measuring the expression level of RNA transcripts for PD-L1. In some embodiments, each of the transformed TMB expression, PD-1 expression, PD-L1 expression, and PD-L2 expression are positively correlated with the likelihood that the patient has a beneficial response to checkpoint inhibitor therapy. In some embodiments, each of the transformed TMB expression, normalized PD-1 expression, normalized PD-L1 expression and normalized PD-L2 expression are positively correlated with the likelihood that the patient has a beneficial response to checkpoint inhibitor therapy. In some embodiments, the IRS is calculated as follows: IRS=3.91*exp(0.296*[transformed TMB measurement]+0.097*[PD-1 normalized level]+0.056*[PD-L2 normalized level]+0.0043*[PD-L1 normalized level]).

In some embodiments, step a. further comprises measuring the expression level of RNA transcripts for CD4. In some embodiments, each of the transformed TMB expression, PD-1 expression, and PD-L2 expression are positively correlated with the likelihood that the patient has a beneficial response to checkpoint inhibitor therapy and CD4 expression is negatively correlated with the likelihood that the patient has a beneficial response to checkpoint inhibitor therapy. In some embodiments, each of the transformed TMB expression, normalized PD-1 expression, and normalized PD-L2 expression are positively correlated with the likelihood that the patient has a beneficial response to checkpoint inhibitor therapy and normalized CD4 expression is negatively correlated with the likelihood that the patient has a beneficial response to checkpoint inhibitor therapy. In some embodiments, the IRS is calculated as follows: IRS=4.10*exp(0.281*[transformed TMB measurement]+0.139*[PD-1 normalized level]+0.112*[PD-L2 normalized level]+−0.128*[CD4 normalized level]).

In some embodiments, the expression levels of RNA transcripts for PD-1, PD-L2, CD4, ADAM12, and PD-L1 are measured. In some embodiments, each of the transformed TMB expression, PD-1 expression, PD-L1, and PD-L2 expression are positively correlated with the likelihood that the patient has a beneficial response to checkpoint inhibitor therapy and CD4 and ADAM12 expression are negatively correlated with the likelihood that the patient has a beneficial response to checkpoint inhibitor therapy. In some embodiments, each of the transformed TMB expression, normalized PD-1 expression, normalized PD-L1, and normalized PD-L2 expression are positively correlated with the likelihood that the patient has a beneficial response to checkpoint inhibitor therapy and normalized CD4 expression and normalized ADAM12 expression are negatively correlated with the likelihood that the patient has a beneficial response to checkpoint inhibitor therapy. In some embodiments, the expression levels of RNA transcripts for PD-1, PD-L2, CD4, ADAM12, and PD-L1 are measured, and wherein the IRS is calculated as follows: IRS=3.95*exp(0.280*[transformed TMB measurement]+0.134*[PD-1 normalized level]+0.122*[PD-L2 normalized level]+−0.070*[ADAM12 normalized level]+−0.154*[CD4 normalized level]+0.052*[PD-L1 normalized level]).

In some embodiments, the expression levels of RNA transcripts for PD-1, PD-L2, CD4, and ADAM12 are measured. In some embodiments, each of the transformed TMB expression, PD-1 expression, and PD-L2 expression are positively correlated with the likelihood that the patient has a beneficial response to checkpoint inhibitor therapy and CD4 and ADAM12 expression are negatively correlated with the likelihood that the patient has a beneficial response to checkpoint inhibitor therapy. In some embodiments, each of the transformed TMB expression, normalized PD-1 expression, and normalized PD-L2 expression are positively correlated with the likelihood that the patient has a beneficial response to checkpoint inhibitor therapy and normalized CD4 expression and normalized ADAM12 expression are negatively correlated with the likelihood that the patient has a beneficial response to checkpoint inhibitor therapy. In some embodiments, the expression levels of RNA transcripts for PD-1, PD-L2, CD4, and ADAM12 are measured, and wherein the IRS is calculated as follows: IRS=4.03*exp(0.287*[transformed TMB measurement]+0.147*[PD-1 normalized level]+0.143*[PD-L2 normalized level]−0.138*[CD4 normalized level]−0.073*[ADAM12 normalized level]).

In some embodiments, step a. further comprises measuring the expression level of RNA transcripts for VTCN1. In some embodiments, each of the transformed TMB expression, PD-1 expression, VTCN1 expression, and PD-L2 expression are positively correlated with the likelihood that the patient has a beneficial response to checkpoint inhibitor therapy. In some embodiments, each of the transformed TMB expression, normalized PD-1 expression, normalized VTCN1 expression and normalized PD-L2 expression are positively correlated with the likelihood that the patient has a beneficial response to checkpoint inhibitor therapy. In some embodiments, the IRS is calculated as follows: IRS=3.90*exp(0.309*[transformed TMB measurement]+0.104*[PD-1 normalized level]+0.087*[PD-L2 normalized level]+0.021*[VTCN1 normalized level]).

In some embodiments, the expression levels of RNA transcripts for PD-1, PD-L2, CD4, ADAM12, and VTCN1 are measured. In some embodiments, each of the transformed TMB expression, PD-1 expression, and PD-L2 expression are positively correlated with the likelihood that the patient has a beneficial response to checkpoint inhibitor therapy and CD4 expression is negatively correlated with the likelihood that the patient has a beneficial response to checkpoint inhibitor therapy. In some embodiments, each of the transformed TMB expression, normalized PD-1 expression, and normalized PD-L2 expression are positively correlated with the likelihood that the patient has a beneficial response to checkpoint inhibitor therapy and normalized CD4 expression is negatively correlated with the likelihood that the patient has a beneficial response to checkpoint inhibitor therapy. In some embodiments, the expression levels of RNA transcripts for PD-1, PD-L2, CD4, ADAM12, and VTCN1 are measured, and wherein the IRS is calculated as follows: IRS=3.95*exp(0.295*[transformed TMB measurement]+0.142*[PD-1 normalized level]+0.150*[PD-L2 normalized level]+0.020*[VTCN1 normalized level]+−0.070*[ADAM12 normalized level]+−0.139*[CD4 normalized level]).

In some embodiments, the IRS value indicating beneficial response to checkpoint inhibitor therapy is 10 or more. In some embodiments, the one or more reference genes comprise three genes selected from LRP1, MRPL13, TBP, HMBS, ITGB7, MYC, CIAO1, CTCF, EIF2B1, GGNBP2, SLC4A1AP. In some embodiments, the tumor specimen is a formalin-fixed paraffin-embedded (FFPE) tumor specimen. In some embodiments, the tumor specimen is adrenal cancer, biliary cancer, bladder cancer, brain cancer, breast cancer, cervical cancer, colon cancer, rectum cancer, endometrial cancer, esophageal cancer, head or neck cancer, kidney cancer, liver cancer, non-small cell lung cancer, lung cancer, lymphoma, melanoma, meniges cancer, non-melanoma skin cancer, ovarian cancer, pancreatic cancer, prostate cancer, sarcoma, small intestine cancer, or stomach cancer. In some embodiments, the expression levels of RNA transcripts are measured using PCR and next-generation sequencing. In some embodiments, the checkpoint inhibitor is an anti-PD-1 antibody, an anti-CTLA-4 antibody, an anti-PD-L1 antibody, or an anti-PD-L2. In some embodiments, the checkpoint inhibitor is nivolumab, pembrolizumab, atezolizumab, durvalumab, pidilizumab, PDR001, BMS-936559, avelumab, or SHR-1210.

Some aspects of the present disclosure are directed to a method of identifying a subject that would benefit from checkpoint inhibitor therapy, comprising: a. measuring expression levels of RNA transcripts for PD-1, PD-L2, CD4, ADAM12, and at least one reference gene in a biological sample obtained from a tumor specimen from the subject; b. log 2 transforming, median centering, and normalizing the measured expression levels of the RNA transcripts of PD-1, PD-L2, CD4, and ADAM12 against the level of the RNA transcripts of at the least one reference gene to provide normalized levels of the RNA transcripts of PD-1, PD-L2, CD4, and ADAM12; c. measuring tumor mutation burden (TMB) in the biological sample and log 2 transforming the TMB measurement to provide a transformed TMB measurement; d. calculating a Immunotherapy Response Score (IRS) from the normalized levels of the RNA transcripts of PD-1, PD-L2, CD4, and ADAM12 and the transformed TMB measurement, wherein the IRS is positively correlated with the likelihood that the patient has a beneficial response to checkpoint inhibitor therapy, and obtaining an IRS having a value indicating beneficial response to checkpoint inhibitor therapy; and e. identifying the subject as benefiting from the checkpoint inhibitor therapy.

In some embodiments, the tumor specimen is from a cancer not approved for labeled use of the checkpoint inhibitor therapy. In some embodiments, the calculated IRS value indicates that the median time-to-next-treatment (TNTT) is 24 months or greater.

In some embodiments, each of the transformed TMB expression, PD-1 expression and PD-L2 expression are positively correlated with the likelihood that the patient has a beneficial response to checkpoint inhibitor therapy. In some embodiments, each of the transformed TMB expression, normalized PD-1 expression and normalized PD-L2 expression are positively correlated with the likelihood that the patient has a beneficial response to checkpoint inhibitor therapy. In some embodiments, the IRS is calculated as follows: IRS=3.97*exp(0.301*[transformed TMB measurement]+0.110*[PD-1 normalized level]+0.078*[PD-L2 normalized level]).

In some embodiments, step a. further comprises measuring the expression level of RNA transcripts for ADAM12. In some embodiments, each of the transformed TMB expression, PD-1 expression, and PD-L2 expression are positively correlated with the likelihood that the patient has a beneficial response to checkpoint inhibitor therapy and ADAM12 expression is negatively correlated with the likelihood that the patient has a beneficial response to checkpoint inhibitor therapy. In some embodiments, each of the transformed TMB expression, normalized PD-1 expression, and normalized PD-L2 expression are positively correlated with the likelihood that the patient has a beneficial response to checkpoint inhibitor therapy and normalized ADAM12 expression is negatively correlated with the likelihood that the patient has a beneficial response to checkpoint inhibitor therapy. In some embodiments, the IRS is calculated as follows: IRS=3.90*exp(0.307*[transformed TMB measurement]+0.115*[PD-1 normalized level]+0.106*[PD-L2 normalized level]+−0.070*[ADAM12 normalized level]).

In some embodiments, step a. further comprises measuring the expression level of RNA transcripts for PD-L1. In some embodiments, each of the transformed TMB expression, PD-1 expression, PD-L1 expression, and PD-L2 expression are positively correlated with the likelihood that the patient has a beneficial response to checkpoint inhibitor therapy. In some embodiments, each of the transformed TMB expression, normalized PD-1 expression, normalized PD-L1 expression and normalized PD-L2 expression are positively correlated with the likelihood that the patient has a beneficial response to checkpoint inhibitor therapy. In some embodiments, the IRS is calculated as follows: IRS=3.91*exp(0.296*[transformed TMB measurement]+0.097*[PD-1 normalized level]+0.056*[PD-L2 normalized level]+0.0043*[PD-L1 normalized level]).

In some embodiments, step a. further comprises measuring the expression level of RNA transcripts for CD4. In some embodiments, each of the transformed TMB expression, PD-1 expression, and PD-L2 expression are positively correlated with the likelihood that the patient has a beneficial response to checkpoint inhibitor therapy and CD4 expression is negatively correlated with the likelihood that the patient has a beneficial response to checkpoint inhibitor therapy. In some embodiments, each of the transformed TMB expression, normalized PD-1 expression, and normalized PD-L2 expression are positively correlated with the likelihood that the patient has a beneficial response to checkpoint inhibitor therapy and normalized CD4 expression is negatively correlated with the likelihood that the patient has a beneficial response to checkpoint inhibitor therapy. In some embodiments, the IRS is calculated as follows: IRS=4.10*exp(0.281*[transformed TMB measurement]+0.139*[PD-1 normalized level]+0.112*[PD-L2 normalized level]+−0.128*[CD4 normalized level]).

In some embodiments, the expression levels of RNA transcripts for PD-1, PD-L2, CD4, ADAM12, and PD-L1 are measured. In some embodiments, each of the transformed TMB expression, PD-1 expression, PD-L1, and PD-L2 expression are positively correlated with the likelihood that the patient has a beneficial response to checkpoint inhibitor therapy and CD4 and ADAM12 expression are negatively correlated with the likelihood that the patient has a beneficial response to checkpoint inhibitor therapy. In some embodiments, each of the transformed TMB expression, normalized PD-1 expression, normalized PD-L1, and normalized PD-L2 expression are positively correlated with the likelihood that the patient has a beneficial response to checkpoint inhibitor therapy and normalized CD4 expression and normalized ADAM12 expression are negatively correlated with the likelihood that the patient has a beneficial response to checkpoint inhibitor therapy. In some embodiments, the expression levels of RNA transcripts for PD-1, PD-L2, CD4, ADAM12, and PD-L1 are measured, and wherein the IRS is calculated as follows: IRS=3.95*exp(0.280*[transformed TMB measurement]+0.134*[PD-1 normalized level]+0.122*[PD-L2 normalized level]+−0.070*[ADAM12 normalized level]+−0.154*[CD4 normalized level]+0.052*[PD-L1 normalized level]).

In some embodiments, the expression levels of RNA transcripts for PD-1, PD-L2, CD4, and ADAM12 are measured. In some embodiments, each of the transformed TMB expression, PD-1 expression, and PD-L2 expression are positively correlated with the likelihood that the patient has a beneficial response to checkpoint inhibitor therapy and CD4 and ADAM12 expression are negatively correlated with the likelihood that the patient has a beneficial response to checkpoint inhibitor therapy. In some embodiments, each of the transformed TMB expression, normalized PD-1 expression, and normalized PD-L2 expression are positively correlated with the likelihood that the patient has a beneficial response to checkpoint inhibitor therapy and normalized CD4 expression and normalized ADAM12 expression are negatively correlated with the likelihood that the patient has a beneficial response to checkpoint inhibitor therapy. In some embodiments, the expression levels of RNA transcripts for PD-1, PD-L2, CD4, and ADAM12 are measured, and wherein the IRS is calculated as follows: IRS=4.03*exp(0.287*[transformed TMB measurement]+0.147*[PD-1 normalized level]+0.143*[PD-L2 normalized level]−0.138*[CD4 normalized level]−0.073*[ADAM12 normalized level]).

In some embodiments, step a. further comprises measuring the expression level of RNA transcripts for VTCN1. In some embodiments, each of the transformed TMB expression, PD-1 expression, VTCN1 expression, and PD-L2 expression are positively correlated with the likelihood that the patient has a beneficial response to checkpoint inhibitor therapy. In some embodiments, each of the transformed TMB expression, normalized PD-1 expression, normalized VTCN1 expression and normalized PD-L2 expression are positively correlated with the likelihood that the patient has a beneficial response to checkpoint inhibitor therapy. In some embodiments, the IRS is calculated as follows: IRS=3.90*exp(0.309*[transformed TMB measurement]+0.104*[PD-1 normalized level]+0.087*[PD-L2 normalized level]+0.021*[VTCN1 normalized level]).

In some embodiments, the expression levels of RNA transcripts for PD-1, PD-L2, CD4, ADAM12, and VTCN1 are measured In some embodiments, each of the transformed TMB expression, PD-1 expression, and PD-L2 expression are positively correlated with the likelihood that the patient has a beneficial response to checkpoint inhibitor therapy and CD4 expression is negatively correlated with the likelihood that the patient has a beneficial response to checkpoint inhibitor therapy. In some embodiments, each of the transformed TMB expression, normalized PD-1 expression, and normalized PD-L2 expression are positively correlated with the likelihood that the patient has a beneficial response to checkpoint inhibitor therapy and normalized CD4 expression is negatively correlated with the likelihood that the patient has a beneficial response to checkpoint inhibitor therapy. In some embodiments, the expression levels of RNA transcripts for PD-1, PD-L2, CD4, ADAM12, and VTCN1 are measured, and wherein the IRS is calculated as follows: IRS=3.95*exp(0.295*[transformed TMB measurement]+0.142*[PD-1 normalized level]+0.150*[PD-L2 normalized level]+0.020*[VTCN1 normalized level]+−0.070*[ADAM12 normalized level]+−0.139*[CD4 normalized level]).

Some aspects of the present disclosure are directed to a method of identifying a subject that would benefit from checkpoint inhibitor therapy, comprising: a. receiving, by a processor, measured expression levels of RNA transcripts for PD-1, PD-L2, CD4, ADAM12, and at least one reference gene in a biological sample obtained from a tumor specimen from the subject; b. log 2 transforming, median centering, and normalizing, by a processor, the measured expression levels of the RNA transcripts of PD-1, PD-L2, CD4, and ADAM12 against the level of the RNA transcripts of at the least one reference gene to provide normalized levels of the RNA transcripts of PD-1, PD-L2, CD4, and ADAM12; c. receiving, by a processor, measured tumor mutation burden (TMB) in the biological sample; d. log 2 transforming, by a processor, the TMB measurement to provide a transformed TMB measurement; e. calculating, by a processor, a Immunotherapy Response Score (IRS) from the normalized levels of the RNA transcripts of PD-1, PD-L2, CD4, and ADAM12 and the transformed TMB measurement that is positively correlated with the likelihood that the patient has a beneficial response to checkpoint inhibitor therapy; and f. providing a determination if the subject has a checkpoint inhibitor responsive cancer.

In some embodiments, each of the transformed TMB expression, PD-1 expression and PD-L2 expression are positively correlated with the likelihood that the patient has a beneficial response to checkpoint inhibitor therapy. In some embodiments, each of the transformed TMB expression, normalized PD-1 expression and normalized PD-L2 expression are positively correlated with the likelihood that the patient has a beneficial response to checkpoint inhibitor therapy. In some embodiments, the IRS is calculated as follows: IRS=3.97*exp(0.301*[transformed TMB measurement]+0.110*[PD-1 normalized level]+0.078*[PD-L2 normalized level]).

In some embodiments, step a. further comprises measuring the expression level of RNA transcripts for ADAM12. In some embodiments, each of the transformed TMB expression, PD-1 expression, and PD-L2 expression are positively correlated with the likelihood that the patient has a beneficial response to checkpoint inhibitor therapy and ADAM12 expression is negatively correlated with the likelihood that the patient has a beneficial response to checkpoint inhibitor therapy. In some embodiments, each of the transformed TMB expression, normalized PD-1 expression, and normalized PD-L2 expression are positively correlated with the likelihood that the patient has a beneficial response to checkpoint inhibitor therapy and normalized ADAM12 expression is negatively correlated with the likelihood that the patient has a beneficial response to checkpoint inhibitor therapy. In some embodiments, the IRS is calculated as follows: IRS=3.90*exp(0.307*[transformed TMB measurement]+0.115*[PD-1 normalized level]+0.106*[PD-L2 normalized level]+−0.070*[ADAM12 normalized level]).

In some embodiments, step a. further comprises measuring the expression level of RNA transcripts for PD-L1. In some embodiments, each of the transformed TMB expression, PD-1 expression, PD-L1 expression, and PD-L2 expression are positively correlated with the likelihood that the patient has a beneficial response to checkpoint inhibitor therapy. In some embodiments, each of the transformed TMB expression, normalized PD-1 expression, normalized PD-L1 expression and normalized PD-L2 expression are positively correlated with the likelihood that the patient has a beneficial response to checkpoint inhibitor therapy. In some embodiments, the IRS is calculated as follows: IRS=3.91*exp(0.296*[transformed TMB measurement]+0.097*[PD-1 normalized level]+0.056*[PD-L2 normalized level]+0.0043*[PD-L1 normalized level]).

In some embodiments, step a. further comprises measuring the expression level of RNA transcripts for CD4. In some embodiments, each of the transformed TMB expression, PD-1 expression, and PD-L2 expression are positively correlated with the likelihood that the patient has a beneficial response to checkpoint inhibitor therapy and CD4 expression is negatively correlated with the likelihood that the patient has a beneficial response to checkpoint inhibitor therapy. In some embodiments, each of the transformed TMB expression, normalized PD-1 expression, and normalized PD-L2 expression are positively correlated with the likelihood that the patient has a beneficial response to checkpoint inhibitor therapy and normalized CD4 expression is negatively correlated with the likelihood that the patient has a beneficial response to checkpoint inhibitor therapy. In some embodiments, the IRS is calculated as follows: IRS=4.10*exp(0.281*[transformed TMB measurement]+0.139*[PD-1 normalized level]+0.112*[PD-L2 normalized level]+−0.128*[CD4 normalized level]).

In some embodiments, the expression levels of RNA transcripts for PD-1, PD-L2, CD4, ADAM12, and PD-L1 are measured. In some embodiments, each of the transformed TMB expression, PD-1 expression, PD-L1, and PD-L2 expression are positively correlated with the likelihood that the patient has a beneficial response to checkpoint inhibitor therapy and CD4 and ADAM12 expression are negatively correlated with the likelihood that the patient has a beneficial response to checkpoint inhibitor therapy. In some embodiments, each of the transformed TMB expression, normalized PD-1 expression, normalized PD-L1, and normalized PD-L2 expression are positively correlated with the likelihood that the patient has a beneficial response to checkpoint inhibitor therapy and normalized CD4 expression and normalized ADAM12 expression are negatively correlated with the likelihood that the patient has a beneficial response to checkpoint inhibitor therapy. In some embodiments, the expression levels of RNA transcripts for PD-1, PD-L2, CD4, ADAM12, and PD-L1 are measured, and wherein the IRS is calculated as follows: IRS=3.95*exp(0.280*[transformed TMB measurement]+0.134*[PD-1 normalized level]+0.122*[PD-L2 normalized level]+−0.070*[ADAM12 normalized level]+−0.154*[CD4 normalized level]+0.052*[PD-L1 normalized level]).

In some embodiments, the expression levels of RNA transcripts for PD-1, PD-L2, CD4, and ADAM12 are measured. In some embodiments, each of the transformed TMB expression, PD-1 expression, and PD-L2 expression are positively correlated with the likelihood that the patient has a beneficial response to checkpoint inhibitor therapy and CD4 and ADAM12 expression are negatively correlated with the likelihood that the patient has a beneficial response to checkpoint inhibitor therapy. In some embodiments, each of the transformed TMB expression, normalized PD-1 expression, and normalized PD-L2 expression are positively correlated with the likelihood that the patient has a beneficial response to checkpoint inhibitor therapy and normalized CD4 expression and normalized ADAM12 expression are negatively correlated with the likelihood that the patient has a beneficial response to checkpoint inhibitor therapy. In some embodiments, the expression levels of RNA transcripts for PD-1, PD-L2, CD4, and ADAM12 are measured, and wherein the IRS is calculated as follows: IRS=4.03*exp(0.287*[transformed TMB measurement]+0.147*[PD-1 normalized level]+0.143*[PD-L2 normalized level]−0.138*[CD4 normalized level]−0.073*[ADAM12 normalized level]).

In some embodiments, step a. further comprises measuring the expression level of RNA transcripts for VTCN1. In some embodiments, each of the transformed TMB expression, PD-1 expression, VTCN1 expression, and PD-L2 expression are positively correlated with the likelihood that the patient has a beneficial response to checkpoint inhibitor therapy. In some embodiments, each of the transformed TMB expression, normalized PD-1 expression, normalized VTCN1 expression and normalized PD-L2 expression are positively correlated with the likelihood that the patient has a beneficial response to checkpoint inhibitor therapy. In some embodiments, the IRS is calculated as follows: IRS=3.90*exp(0.309*[transformed TMB measurement]+0.104*[PD-1 normalized level]+0.087*[PD-L2 normalized level]+0.021*[VTCN1 normalized level]).

In some embodiments, the expression levels of RNA transcripts for PD-1, PD-L2, CD4, ADAM12, and VTCN1 are measured. In some embodiments, each of the transformed TMB expression, PD-1 expression, and PD-L2 expression are positively correlated with the likelihood that the patient has a beneficial response to checkpoint inhibitor therapy and CD4 expression is negatively correlated with the likelihood that the patient has a beneficial response to checkpoint inhibitor therapy. In some embodiments, each of the transformed TMB expression, normalized PD-1 expression, and normalized PD-L2 expression are positively correlated with the likelihood that the patient has a beneficial response to checkpoint inhibitor therapy and normalized CD4 expression is negatively correlated with the likelihood that the patient has a beneficial response to checkpoint inhibitor therapy. In some embodiments, the expression levels of RNA transcripts for PD-1, PD-L2, CD4, ADAM12, and VTCN1 are measured, and wherein the IRS is calculated as follows: IRS=3.95*exp(0.295*[transformed TMB measurement]+0.142*[PD-1 normalized level]+0.150*[PD-L2 normalized level]+0.020*[VTCN1 normalized level]+−0.070*[ADAM12 normalized level]+−0.139*[CD4 normalized level]).

Some aspects of the present disclosure are directed to a method of treatment of a subject in need thereof with a checkpoint inhibitor therapy, comprising administering to said subject the checkpoint inhibitor therapy, wherein said subject in need thereof was previously identified by a method comprising: a. measuring expression levels of RNA transcripts for PD-1, PD-L2, CD4, ADAM12, and at least one reference gene in a biological sample obtained from a tumor specimen from the subject; b. log 2 transforming, median centering, and normalizing the measured expression levels of the RNA transcripts of PD-1, PD-L2, CD4, and ADAM12 against the level of the RNA transcripts of at the least one reference gene to provide normalized levels of the RNA transcripts of PD-1, PD-L2, CD4, and ADAM12; c. measuring tumor mutation burden (TMB) in the biological sample and log 2 transforming the TMB measurement to provide a transformed TMB measurement; and d. calculating a Immunotherapy Response Score (IRS) from the normalized levels of the RNA transcripts of PD-1, PD-L2, CD4, and ADAM12 and the transformed TMB measurement that is positively correlated with the likelihood that the patient has a beneficial response to checkpoint inhibitor therapy and obtaining an IRS having a value indicating beneficial response to checkpoint inhibitor therapy.

In some embodiments, the IRS is calculated using the Cox model as 10 times the inverse of the patient hazard ratio as compared to the median hazard rate. In some embodiments, the IRS is calculated as follows: IRS=4.03*exp(0.287*[transformed TMB measurement]+0.147*[PD-1 normalized level]+0.143*[PD-L2 normalized level]−0.138*[CD4 normalized level]−0.073*[ADAM12 normalized level]). In some embodiments, the IRS value indicating beneficial response to checkpoint inhibitor therapy is 10 or more.

In some embodiments, each of the transformed TMB expression, PD-1 expression and PD-L2 expression are positively correlated with the likelihood that the patient has a beneficial response to checkpoint inhibitor therapy. In some embodiments, each of the transformed TMB expression, normalized PD-1 expression and normalized PD-L2 expression are positively correlated with the likelihood that the patient has a beneficial response to checkpoint inhibitor therapy. In some embodiments, the IRS is calculated as follows: IRS=3.97*exp(0.301*[transformed TMB measurement]+0.110*[PD-1 normalized level]+0.078*[PD-L2 normalized level]).

In some embodiments, step a. further comprises measuring the expression level of RNA transcripts for ADAM12. In some embodiments, each of the transformed TMB expression, PD-1 expression, and PD-L2 expression are positively correlated with the likelihood that the patient has a beneficial response to checkpoint inhibitor therapy and ADAM12 expression is negatively correlated with the likelihood that the patient has a beneficial response to checkpoint inhibitor therapy. In some embodiments, each of the transformed TMB expression, normalized PD-1 expression, and normalized PD-L2 expression are positively correlated with the likelihood that the patient has a beneficial response to checkpoint inhibitor therapy and normalized ADAM12 expression is negatively correlated with the likelihood that the patient has a beneficial response to checkpoint inhibitor therapy. In some embodiments, the IRS is calculated as follows: IRS=3.90*exp(0.307*[transformed TMB measurement]+0.115*[PD-1 normalized level]+0.106*[PD-L2 normalized level]+−0.070*[ADAM12 normalized level]).

In some embodiments, step a. further comprises measuring the expression level of RNA transcripts for PD-L1. In some embodiments, each of the transformed TMB expression, PD-1 expression, PD-L1 expression, and PD-L2 expression are positively correlated with the likelihood that the patient has a beneficial response to checkpoint inhibitor therapy. In some embodiments, each of the transformed TMB expression, normalized PD-1 expression, normalized PD-L1 expression and normalized PD-L2 expression are positively correlated with the likelihood that the patient has a beneficial response to checkpoint inhibitor therapy. In some embodiments, the IRS is calculated as follows: IRS=3.91*exp(0.296*[transformed TMB measurement]+0.097*[PD-1 normalized level]+0.056*[PD-L2 normalized level]+0.0043*[PD-L1 normalized level]).

In some embodiments, step a. further comprises measuring the expression level of RNA transcripts for CD4. In some embodiments, each of the transformed TMB expression, PD-1 expression, and PD-L2 expression are positively correlated with the likelihood that the patient has a beneficial response to checkpoint inhibitor therapy and CD4 expression is negatively correlated with the likelihood that the patient has a beneficial response to checkpoint inhibitor therapy. In some embodiments, each of the transformed TMB expression, normalized PD-1 expression, and normalized PD-L2 expression are positively correlated with the likelihood that the patient has a beneficial response to checkpoint inhibitor therapy and normalized CD4 expression is negatively correlated with the likelihood that the patient has a beneficial response to checkpoint inhibitor therapy. In some embodiments, the IRS is calculated as follows: IRS=4.10*exp(0.281*[transformed TMB measurement]+0.139*[PD-1 normalized level]+0.112*[PD-L2 normalized level]+−0.128*[CD4 normalized level]).

In some embodiments, the expression levels of RNA transcripts for PD-1, PD-L2, CD4, ADAM12, and PD-L1 are measured. In some embodiments, each of the transformed TMB expression, PD-1 expression, PD-L1, and PD-L2 expression are positively correlated with the likelihood that the patient has a beneficial response to checkpoint inhibitor therapy and CD4 and ADAM12 expression are negatively correlated with the likelihood that the patient has a beneficial response to checkpoint inhibitor therapy. In some embodiments, each of the transformed TMB expression, normalized PD-1 expression, normalized PD-L1, and normalized PD-L2 expression are positively correlated with the likelihood that the patient has a beneficial response to checkpoint inhibitor therapy and normalized CD4 expression and normalized ADAM12 expression are negatively correlated with the likelihood that the patient has a beneficial response to checkpoint inhibitor therapy. In some embodiments, the expression levels of RNA transcripts for PD-1, PD-L2, CD4, ADAM12, and PD-L1 are measured, and wherein the IRS is calculated as follows: IRS=3.95*exp(0.280*[transformed TMB measurement]+0.134*[PD-1 normalized level]+0.122*[PD-L2 normalized level]+−0.070*[ADAM12 normalized level]+−0.154*[CD4 normalized level]+0.052*[PD-L1 normalized level]).

In some embodiments, the expression levels of RNA transcripts for PD-1, PD-L2, CD4, and ADAM12 are measured. In some embodiments, each of the transformed TMB expression, PD-1 expression, and PD-L2 expression are positively correlated with the likelihood that the patient has a beneficial response to checkpoint inhibitor therapy and CD4 and ADAM12 expression are negatively correlated with the likelihood that the patient has a beneficial response to checkpoint inhibitor therapy. In some embodiments, each of the transformed TMB expression, normalized PD-1 expression, and normalized PD-L2 expression are positively correlated with the likelihood that the patient has a beneficial response to checkpoint inhibitor therapy and normalized CD4 expression and normalized ADAM12 expression are negatively correlated with the likelihood that the patient has a beneficial response to checkpoint inhibitor therapy. In some embodiments, the expression levels of RNA transcripts for PD-1, PD-L2, CD4, and ADAM12 are measured, and wherein the IRS is calculated as follows: IRS=4.03*exp(0.287*[transformed TMB measurement]+0.147*[PD-1 normalized level]+0.143*[PD-L2 normalized level]−0.138*[CD4 normalized level]−0.073*[ADAM12 normalized level]).

In some embodiments, step a. further comprises measuring the expression level of RNA transcripts for VTCN1. In some embodiments, each of the transformed TMB expression, PD-1 expression, VTCN1 expression, and PD-L2 expression are positively correlated with the likelihood that the patient has a beneficial response to checkpoint inhibitor therapy. In some embodiments, each of the transformed TMB expression, normalized PD-1 expression, normalized VTCN1 expression and normalized PD-L2 expression are positively correlated with the likelihood that the patient has a beneficial response to checkpoint inhibitor therapy. In some embodiments, the IRS is calculated as follows: IRS=3.90*exp(0.309*[transformed TMB measurement]+0.104*[PD-1 normalized level]+0.087*[PD-L2 normalized level]+0.021*[VTCN1 normalized level]).

In some embodiments, the expression levels of RNA transcripts for PD-1, PD-L2, CD4, ADAM12, and VTCN1 are measured. In some embodiments, each of the transformed TMB expression, PD-1 expression, and PD-L2 expression are positively correlated with the likelihood that the patient has a beneficial response to checkpoint inhibitor therapy and CD4 expression is negatively correlated with the likelihood that the patient has a beneficial response to checkpoint inhibitor therapy. In some embodiments, each of the transformed TMB expression, normalized PD-1 expression, and normalized PD-L2 expression are positively correlated with the likelihood that the patient has a beneficial response to checkpoint inhibitor therapy and normalized CD4 expression is negatively correlated with the likelihood that the patient has a beneficial response to checkpoint inhibitor therapy. In some embodiments, the expression levels of RNA transcripts for PD-1, PD-L2, CD4, ADAM12, and VTCN1 are measured, and wherein the IRS is calculated as follows: IRS=3.95*exp(0.295*[transformed TMB measurement]+0.142*[PD-1 normalized level]+0.150*[PD-L2 normalized level]+0.020*[VTCN1 normalized level]+−0.070*[ADAM12 normalized level]+−0.139*[CD4 normalized level]).

In some embodiments of each of the methods disclosed herein a subject is identified as benefiting from checkpoint inhibitor therapy (e.g., pembrolizumab) or treated with checkpoint inhibitor therapy (e.g., pembrolizumab) when the tumor type is not approved for use with the checkpoint inhibitor (off-label) and the IRS value is 10 or greater. In some embodiments, a subject is identified as benefiting from checkpoint inhibitor therapy (e.g., pembrolizumab) or treated with checkpoint inhibitor therapy (e.g., pembrolizumab) when the tumor type is not approved for use with the checkpoint inhibitor (off-label) and the IRS value is 12 or greater. In some embodiments, a subject is identified as benefiting from checkpoint inhibitor therapy (e.g., pembrolizumab) or treated with checkpoint inhibitor therapy (e.g., pembrolizumab) when the tumor type is not approved for use with the checkpoint inhibitor (off-label), the TMB is 10 mutations per megabase (MPM) or more and the IRS value is 10 or greater. In some embodiments, a subject is identified as benefiting from checkpoint inhibitor therapy (e.g., pembrolizumab) or treated with checkpoint inhibitor therapy (e.g., pembrolizumab) when the tumor type is not approved for use with the checkpoint inhibitor (off-label), the TMB is 10 MPM or more and the IRS value is 12 or greater. In some embodiments, a subject is identified as benefiting from checkpoint inhibitor therapy (e.g., pembrolizumab) or treated with checkpoint inhibitor therapy (e.g., pembrolizumab) when the TMB is 10 MPM or more and the IRS value is 10 or greater. In some embodiments, a subject is identified as benefiting from checkpoint inhibitor therapy (e.g., pembrolizumab) or treated with checkpoint inhibitor therapy (e.g., pembrolizumab) when the TMB is 10 MPM or more and the IRS value is 12 or greater.

In some embodiments of each of the methods disclosed herein, determining if the tumor will be or is more likely to be responsive to immune checkpoint therapy comprises collecting or providing a tumor specimen from a subject. In some embodiments, the tumor specimen is a fresh tumor specimen or a formalin-fixed paraffin-embedded (FFPE) tumor specimen. However, the specimen preparation is not limited and may be any suitable preparation known in the art. In some embodiments, the methods do not include collecting or providing a tumor. Instead, data (e.g., IRS value) or a qualitative assessment (e.g., a determination that the tumor has a suitable IRS value) is provided. In some embodiments, the data or qualitative assessment is provided to a physician or other health professional and such person uses such data or assessment to determine whether or not to administer the immune checkpoint therapy. The provided data or qualitative assessment can be calculated or determined by any of the methods disclosed herein.

In some embodiments of each of the methods disclosed herein, the tumor may be from any cancer is not limited. As used herein, the term “cancer” refers to a malignant neoplasm (Stedman's Medical Dictionary, 25th ed.; Hensyl ed.; Williams & Wilkins: Philadelphia, 1990). Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarinoma); Ewing's sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva). In some embodiments, the cancer is a solid cancer.

In some embodiments of each of the methods disclosed herein, the cancer is not a blood-borne or hematopoietic cancer. In some embodiments, the cancer is not an MSI-H cancer. In some embodiments, the cancer is not 1, 2, 3, 4, 5, 6 or all 7 of melanoma, lung cancer, kidney cancer, bladder cancer, head and neck cancer, and Hodgkin's lymphoma. In some embodiments, the cancer is adrenal cancer, biliary cancer, bladder cancer, brain cancer, breast cancer, cervical cancer, colon cancer, rectum cancer, endometrial cancer, esophageal cancer, head or neck cancer, kidney cancer, liver cancer, non-small cell lung cancer, lung cancer, lymphoma, melanoma, meninges cancer, non-melanoma skin cancer, ovarian cancer, pancreatic cancer, prostate cancer, sarcoma, small intestine cancer, or stomach cancer.. In some embodiments, the cancer is not a TMB-H cancer. In some embodiments, the cancer is not 1, 2, 3, 4, 5, 6, 7, 8, 9, or all 10 of melanoma, lung cancer, kidney cancer, bladder cancer, head and neck cancer, cervical cancer, esophagogastric cancer, hepatobiliary cancer, nonmelanoma skin cancer, and Hodgkin's lymphoma. In some embodiments, the cancer is adrenal cancer, biliary cancer, bladder cancer, brain cancer, breast cancer, cervical cancer, colon cancer, rectum cancer, endometrial cancer, esophageal cancer, head or neck cancer, kidney cancer, liver cancer, non-small cell lung cancer, lung cancer, lymphoma, melanoma, meninges cancer, non-melanoma skin cancer, ovarian cancer, pancreatic cancer, prostate cancer, sarcoma, small intestine cancer, or stomach cancer.

In some embodiments of each of the methods disclosed herein, determining or calculating if the tumor will be or is more likely to be responsive to immune checkpoint therapy comprises calculating, collecting or determining immune-response associated data derived from the tumor (e.g., the IRS value). In some embodiments, the methods disclosed herein comprise obtaining immune-response associated data (quantitative or qualitative) derived from the tumor from another party and determining if the tumor will be or is more likely to be responsive to immune checkpoint therapy. In some embodiments, immune-response associated data is collected or determined via NGS and/or multiplexed PCR. In some embodiments, immune-response associated data is obtained from NGS and/or multiplexed PCR performed by another party.

In some embodiments of each of the methods disclosed herein, PD-1 expression is determined or calculated via NGS of gene expression transcripts using multiplex PCR (amplicon). In some embodiments, PD-1 expression is obtained from NGS of gene expression transcripts using multiplex PCR (amplicon) data. In some embodiments, PD-1 expression is validated, confirmed, or combined using multiplex PCR and a second amplicon. In some embodiments, validation or confirmation of PD-1 requires that the second amplicon's percentile value is 70%, 75%, 80%, 85% or more of the calculated PD-1 percentile value. In some embodiments, validation or confirmation of PD-1 requires that the second amplicon's percentile value is 80% or more of the calculated PD-1 percentile value.

In some embodiments of each of the methods disclosed herein, PD-L2 expression is determined or calculated via NGS of gene expression transcripts using multiplex PCR (amplicon). In some embodiments, PD-L2 expression is obtained from NGS of gene expression transcripts using multiplex PCR (amplicon) data. In some embodiments, PD-L2 expression is validated, confirmed, or combined using multiplex PCR and a second amplicon. In some embodiments, validation or confirmation of PD-L2 requires that the second amplicon's percentile value is 70%, 75%, 80%, 85% or more of the calculated PD-L2 percentile value. In some embodiments, validation or confirmation of PD-L2 requires that the second amplicon's percentile value is 80% or more of the calculated PD-L2 percentile value.

In some embodiments of each of the methods disclosed herein, CD4 expression is determined or calculated via NGS of gene expression transcripts using multiplex PCR (amplicon). In some embodiments, CD4 expression is obtained from NGS of gene expression transcripts using multiplex PCR (amplicon) data. In some embodiments, CD4 expression is validated, confirmed, or combined using multiplex PCR (amplicon) to measure GZMA, GZMB, GZMK, PRF1, IFNG or CD8B expression. In some embodiments, CD4 expression is validated, confirmed, or combined using multiplex PCR (amplicon) to measure GZMA expression. CD4 and GZMA are both part of the interferon-y gene signature. In some embodiments, validation, confirmation or combination of CD4 requires that the second amplicon measurement's percentile value is 80% or more of the calculated CD4 percentile value.

In some embodiments of each of the methods disclosed herein, ADAM12 expression is determined or calculated via NGS of gene expression transcripts using multiplex PCR (amplicon). In some embodiments, ADAM12 expression is obtained from NGS of gene expression transcripts using multiplex PCR (amplicon) data. In some embodiments, ADAM12 expression is validated, confirmed, or combined using multiplex PCR and a second amplicon. In some embodiments, validation or confirmation of ADAM12 requires that the second amplicon's percentile value is 70%, 75%, 80%, 85% or more of the calculated ADAM12 percentile value. In some embodiments, validation or confirmation of ADAM12 requires that the second amplicon's percentile value is 80% or more of the calculated ADAM12 percentile value.

In some embodiments of each of the methods disclosed herein, PD-L1 expression is determined or calculated via NGS of gene expression transcripts using multiplex PCR (amplicon). In some embodiments, PD-L1 expression is obtained from NGS of gene expression transcripts using multiplex PCR (amplicon) data. In some embodiments, PD-L1 expression is validated, confirmed, or combined using multiplex PCR and a second amplicon. In some embodiments, validation or confirmation of PD-L1 requires that the second amplicon's percentile value is 70%, 75%, 80%, 85% or more of the calculated PD-L1 percentile value. In some embodiments, validation or confirmation of PD-L1 requires that the second amplicon's percentile value is 80% or more of the calculated PD-L1 percentile value.

In some embodiments of each of the methods disclosed herein, VTCN1 expression is determined or calculated via NGS of gene expression transcripts using multiplex PCR (amplicon). In some embodiments, VTCN1 expression is obtained from NGS of gene expression transcripts using multiplex PCR (amplicon) data. In some embodiments, VTCN1 expression is validated, confirmed, or combined using multiplex PCR and a second amplicon. In some embodiments, validation or confirmation of VTCN1 requires that the second amplicon's percentile value is 70%, 75%, 80%, 85% or more of the calculated VTCN1 percentile value. In some embodiments, validation or confirmation of VTCN1 requires that the second amplicon's percentile value is 80% or more of the calculated VTCN1 percentile value.

In some embodiments of each of the methods disclosed herein, TOP2A expression is determined or calculated via NGS of gene expression transcripts using multiplex PCR (amplicon). In some embodiments, TOP2A expression is obtained from NGS of gene expression transcripts using multiplex PCR (amplicon) data. In some embodiments, TOP2A expression is validated, confirmed, or combined using multiplex PCR and a second amplicon. In some embodiments, validation or confirmation of TOP2A requires that the second amplicon's percentile value is 70%, 75%, 80%, 85% or more of the calculated PD-L1 percentile value. In some embodiments, validation or confirmation of TOP2A requires that the second amplicon's percentile value is 80% or more of the calculated TOP2A percentile value.

In some embodiments of each of the methods disclosed herein, TMB is determined or calculated by NGS of tumor DNA. In some embodiments, TMB is obtained from another party. Methods of detecting mutations (e.g., TMB) are not limited. In some embodiments, mutations are detected, calculated or obtained via NGS. In some embodiments, TMB includes non-coding (at highly characterized genomic loci) and coding, synonymous and non-synonymous, single and multi-nucleotide (two bases) variants present at >10% variant allele frequency (VAF). In some embodiments, mutations per megabase (Mb) estimates and associated 90% confidence interval are calculated via the total number of positions with sufficient depth of coverage necessary for definitive assessment (maximum possible 1.7 Mb).

In some embodiments of each of the methods disclosed herein, the checkpoint inhibitor administered is an antibody against at least one checkpoint protein, e.g., PD-1, CTLA-4, PD-L1 or PD-L2. In some embodiments, the checkpoint inhibitor administered is an antibody that is effective against two or more of the checkpoint proteins selected from the group of PD-1, CTLA-4, PD-L1 and PD-L2. In some embodiments, the checkpoint inhibitor administered is a small molecule, non-protein compound that inhibits at least one checkpoint protein. In one embodiment, the checkpoint inhibitor is a small molecule, non-protein compound that inhibits a checkpoint protein selected from the group consisting of PD-1, CTLA-4, PD-L1 and PD-L2. In some embodiments, the checkpoint inhibitor administered is nivolumab (Opdivo®, BMS-936558, MDX1106, commercially available from BristolMyers Squibb, Princeton NJ), pembrolizumab (Keytruda® MK-3475, lambrolizumab, commercially available from Merck and Company, Kenilworth NJ), atezolizumab (Tecentriq®, Genentech/Roche, South San Francisco CA), durvalumab (MEDI4736, Medimmune/AstraZeneca), pidilizumab (CT-011, CureTech), PDR001 (Novartis), BMS-936559 (MDX1105, BristolMyers Squibb), avelumab (MSB0010718C, Merck Serono/Pfizer), or SHR-1210 (Incyte). Additional antibody PD1 pathway inhibitors for use in the methods described herein include those described in U.S. Pat. No. 8,217,149 (Genentech, Inc) issued Jul. 10, 2012; U.S. Pat. No. 8,168,757 (Merck Sharp and Dohme Corp.) issued May 1, 2012, U.S. Pat. No. 8,008,449 (Medarex) issued Aug. 30, 2011, and U.S. Pat. No. 7,943,743 (Medarex, Inc) issued May 17, 2011.

In some embodiments each of the methods disclosed herein, the disclosed methods include performing one or more normalization processes, such as for enabling sequencing outputs (e.g., associated with any suitable biomarkers described herein, etc.) to be comparable to thresholds and/or across different sequencing runs. In examples, determining IRS values can include background-subtracting sequence read counts; and normalizing the background-subtracted sequence read counts into normalized reads per million (nRPM). In a specific example, a fold-change ratio can be determined for a given gene (and/or suitable biomarker), according to: Ratio=Background Subtracted Read Count/Reads Per Million (RPM) profile. In a specific example, the RPM profile can be determined based on an average RPM (and/or other suitable aggregate RPM metric) of a plurality of replicates of biological samples across different validation sequencing runs. In a specific example, median values of determined ratios can be used for a Normalization Ratio for a given biological sample, where the nRPM can be calculated according to: nRPM=Background Subtracted Read Count/Normalization Ratio. Housekeeping genes usable for normalization processes (e.g., described herein) can include any one or more of: LRP1, MRPL13, TBP, HMBS, ITGB7, MYC, CIAO1, CTCF, EIF2B1, GGNBP2, SLC4A1AP and/or other suitable housekeeping genes (and/or any suitable genes). In some embodiments, two, three, four, five, six, seven, or eight of LRP1, MRPL13, TBP, HMBS, ITGB7, MYC, CIAO1, CTCF, EIF2B1, GGNBP2, SLC4A1AP are used for the normalization process. In some embodiments, three of LRP1, MRPL13, TBP, HMBS, ITGB7, MYC, CIAO1, CTCF, EIF2B1, GGNBP2, SLC4A1AP are used for the normalization process. In some embodiments, EIF2B1, HMBS, and CIAO1 are used for the normalization process. In a another specific example, median values of determined ratios can be used for a Normalization Ratio for a given biological sample, where the nRPM can be calculated according to: nRPM=Background Subtracted Read Count/Normalization Ratio. Housekeeping genes usable for normalization processes (e.g., described herein) can include any one or more of: CIAO1, EIF2B1, HMBS, CTCF, GGNBP2, ITGB7, MYC and SLC4A1AP. In some embodiments, three or more of CIAO1, EIF2B1, HMBS, CTCF, GGNBP2, ITGB7, MYC and SLC4A1AP are used for the normalization process. In some embodiments, the one or more reference genes comprise the combination of CIAO1, EIF2B1 with HMBS, CTCF, GGNBP2, ITGB7, MYC, SLC4A1AP and/or other suitable housekeeping genes (and/or any suitable genes). In some embodiments, two, three, four, five, six, seven, or all eight of CIAO1, EIF2B1, HMBS, CTCF, GGNBP2, ITGB7, MYC and SLC4A1AP are used for the normalization process. In some embodiments, three of CIAO1, EIF2B1, HMBS, CTCF, GGNBP2, ITGB7, MYC and SLC4A1AP are used for the normalization process. In some embodiments, EIF2B1, HMBS, and CIAO1 are used for the normalization process. Additionally or alternatively, any suitable backgrounding and/or normalizing processes can be performed (e.g., for comparison of values to thresholds; for comparison of values across sequencing runs; etc.).

In some embodiments of each of the methods disclosed herein, measurement of the housekeeping genes is omitted, and an internal standard is used for normalization. For example, when using real time quantitative PCR, which measures PCR product accumulation through a dual-labeled fluorigenic probe (i.e., TaqMan® probe). Real time PCR is compatible both with quantitative competitive PCR, where internal competitor for each target sequence is used for normalization, and with quantitative comparative PCR using a normalization gene contained within the sample, or a housekeeping gene for RT-PCR. For further details see, e.g. Held et al., Genome Research 6:986-994 (1996). Alternatively, in some embodiments of the methods disclosed herein, normalization is based on the mean or median signal (CT) of all of the assayed genes or a large subset thereof (global normalization approach).

In a specific example, the methods of the claimed invention can include one or more of: collecting a set of biological samples (e.g., FFPE tumor specimens) from a set of patients (e.g., cancer patients; etc.); generating one or more sequencing libraries (e.g., suitable for generating sequencing outputs indicative of biomarkers associated with patient responsiveness to one or more therapies; etc.) based on processing of the biological samples; determining sets of sequencing reads (e.g., for cDNA sequences derived from cDNA converted from mRNA indicating expression levels for the biomarkers provide herein, and, optionally, at least one reference gene) for the set of patients based on the one or more sequencing libraries; processing the sequencing reads for determining immune response-associated data (e.g., PD-L2 gene expression levels; PD-1 gene expression levels; one or more of CD4, ADAM12, TOP2A, PD-L1, and VTCN1 gene expression levels; cDNA sequence data, such as from cDNA converted from mRNA; DNA sequence data; TMB-associated data; MSI-associated data; etc.); determining treatment response characterizations (e.g., IRS) for the set of patients based on the immune response-associated data (e.g., based on independent and/or combined analyses of the different types of immune response-associated data; etc.); and facilitating treatment provision for one or more patients of the set of patients based on the treatment response characterizations (e.g., identifying a subset of patients with indications of positive responsiveness to therapies for clinical trials, such as for clinical trial enrollment; providing the treatment response characterizations to one or more care providers, such as for guiding care decisions by the one or more care providers; etc.).

In specific examples, IRS values can be used for clinical trials (e.g., clinical trial enrollment and patient selection; stratification of patient populations, such as based on different combinations of biomarkers; therapy characterization; results analysis; and/or other suitable purposes related to clinical trials; etc.), care provision (e.g., providing treatment response characterizations to care providers for guiding care decisions regarding patients; therapy determination for patients; etc.), and/or other suitable applications. Additionally or alternatively, embodiments of the methods and systems disclosed herein can function to conserve valuable biological samples, such as lung cancer tissue biopsies, tumor specimens, and/or suitable types of biological samples. In specific examples, immune response-associated data collection can be performed based on RNA sequencing e.g., PD-L1 gene expression levels; PD-1 gene expression levels; one or more of CD4, ADAM12, TOP2A, PD-L2, and VTCN1 gene expression levels; cDNA sequence data, such as from cDNA converted from mRNA; DNA sequence data; TMB-associated data; MSI-associated data; etc.) and/or other suitable processing approaches as an alternative to sample processing approaches that can require a relatively larger usage of biological sample (e.g., immunohistochemistry; etc.). However, embodiments of the methods and systems disclosed herein can include any suitable functionality.

Embodiments of the methods disclosed herein preferably apply, include, and/or are otherwise associated with next-generation sequencing (NGS) (e.g., processing biological samples to generate sequence libraries for sequencing with next-generation sequencing systems; etc.). Embodiments of the methods disclosed herein can include, apply, and/or otherwise be associated with semiconductor-based sequencing technologies. Additionally or alternatively, embodiments of the methods disclosed herein can include, apply, and/or otherwise be associated with any suitable sequencing technologies (e.g., sequencing library preparation technologies; sequencing systems; sequencing output analysis technologies; etc.). Sequencing technologies preferably include next-generation sequencing technologies. Next-generation sequencing technologies can include any one or more of high-throughput sequencing (e.g., facilitated through high-throughput sequencing technologies; massively parallel signature sequencing, Polony sequencing, 454 pyrosequencing, Illumina sequencing, SOLiD sequencing, Ion Torrent semiconductor sequencing and/or other suitable semiconductor-based sequencing technologies, DNA nanoball sequencing, Heliscope single molecule sequencing, Single molecule real time (SMRT) sequencing, Nanopore DNA sequencing, etc.), any generation number of sequencing technologies (e.g., second-generation sequencing technologies, third-generation sequencing technologies, fourth-generation sequencing technologies, etc.), sequencing-by-synthesis, tunneling currents sequencing, sequencing by hybridization, mass spectrometry sequencing, microscopy-based techniques, and/or any suitable next-generation sequencing technologies. In specific examples, embodiments of the methods disclosed herein can include applying next-generation sequencing technologies to sequence libraries prepared for facilitating generation of sequence reads associated with a plurality of biomarkers for responsiveness to one or more immune checkpoint therapies (e.g., PD-1/PD-L1 inhibitors; etc.).

Additionally or alternatively, sequencing technologies can include any one or more of: capillary sequencing, Sanger sequencing (e.g., microfluidic Sanger sequencing, etc.), pyrosequencing, nanopore sequencing (Oxford nanopore sequencing, etc.), and/or any other suitable types of sequencing facilitated by any suitable sequencing technologies.

Embodiments of the methods disclosed herein can include, apply, perform, and/or otherwise be associated with any one or more of: sequencing operations, alignment operation (e.g., sequencing read alignment; etc.), lysing operations, cutting operations, tagging operations (e.g., with barcodes; etc.), ligation operations, fragmentation operations, amplification operations (e.g., helicase-dependent amplification (HDA), loop mediated isothermal amplification (LAMP), self-sustained sequence replication (3SR), nucleic acid sequence based amplification (NASBA), strand displacement amplification (SDA), rolling circle amplification (RCA), ligase chain reaction (LCR), etc.), purification operations, cleaning operations, suitable operations for sequencing library preparation, suitable operations for facilitating sequencing and/or downstream analysis, suitable sample processing operations, and/or any suitable sample- and/or sequence-related operations. In specific examples, sample processing operations can be performed for processing biological samples to generate sequencing libraries for facilitating characterization of a plurality of biomarkers associated with responsiveness to one or more immune checkpoint therapies.

Additionally or alternatively, data described herein (e.g., immune response-associated data, thresholds, models, parameters, normalized data, IRS values, treatment determinations, sample data, sequencing data, etc.) can be associated with any suitable temporal indicators (e.g., seconds, minutes, hours, days, weeks, time periods, time points, timestamps, etc.) including one or more: temporal indicators indicating when the data was collected, determined, transmitted, received, and/or otherwise processed; temporal indicators providing context to content described by the data; changes in temporal indicators (e.g., data over time; change in data; data patterns; data trends; data extrapolation and/or other prediction; etc.); and/or any other suitable indicators related to time. In specific examples, treatment response characterizations can be performed over time for one or more patients, to facilitate patient monitoring, therapy effectiveness evaluation, additional treatment provision facilitation, and/or other suitable purposes.

Additionally or alternatively, parameters, metrics, inputs, outputs, and/or other suitable data can be associated with value types including any one or more of: binary values (e.g., binary status determinations of presence or absence of one or more biomarkers associated with positive responsiveness to immune checkpoint therapies and/or other suitable therapies, etc.), scores (e.g., aggregate scores indicative of a probability and/or degree of responsiveness to therapies described herein; etc.), values indicative of presence of, absence of, degree of responsiveness to one or more therapies described herein, classifications (e.g., patient classifications for sensitivity to therapies described herein; patent classifications based on absence or presence of different biomarkers of a set of biomarkers associated with responsiveness to therapies described herein, etc.), identifiers (e.g., sample identifiers; sample labels indicating association with different cancer conditions; patient identifiers; biomarker identifiers; etc.), values along a spectrum, and/or any other suitable types of values. Any suitable types of data described herein can be used as inputs (e.g., for different models; for comparison against thresholds), generated as outputs (e.g., of different models; for use in treatment response characterizations; etc.), and/or manipulated in any suitable manner for any suitable components associated with embodiments of the methods disclosed herein.

One or more instances and/or portions of embodiments of the methods disclosed herein can be performed asynchronously (e.g., sequentially), concurrently (e.g., in parallel; concurrently on different threads for parallel computing to improve system processing ability for immune response-associated data processing and/or treatment response characterization generation; multiplex sample processing; multiplex sequencing such as for a plurality of biomarkers in combination, such as in a minimized number of sequencing runs; etc.), in temporal relation to a trigger event (e.g., performance of a portion of a method disclosed herein), and/or in any other suitable order at any suitable time and frequency by and/or using one or more instances of embodiments of inventions described herein.

Embodiments of a system to perform the methods described herein can include one or more: sample handling systems (e.g., for processing samples; for sequencing library generation; etc.); sequencing systems (e.g., for sequencing one or more sequencing libraries; etc.); computing systems (e.g., for sequencing output analysis; for immune response-associated data collection and/or processing; for treatment response characterization generation; for any suitable computational processes; etc.); treatment systems (e.g., for providing treatment recommendations; for facilitating patient selection for clinical trials; for therapy provision; etc.); and/or any other suitable components.

Embodiments of the system and/or portions of embodiments of the system described herein can entirely or partially be executed by, hosted on, communicate with, and/or otherwise include one or more: remote computing systems (e.g., a server, at least one networked computing system, stateless, stateful; etc.), local computing systems, user devices (e.g., mobile phone device, other mobile device, personal computing device, tablet, wearable, head-mounted wearable computing device, wrist-mounted wearable computing device, etc.), databases (e.g., including sample data and/or analyses, sequencing data, user data, data described herein, etc.), application programming interfaces (APIs) (e.g., for accessing data described herein, etc.) and/or any suitable components. Communication by and/or between any components of the system and/or other suitable components can include wireless communication (e.g., WiFi, Bluetooth, radiofrequency, Zigbee, Z-wave, etc.), wired communication, and/or any other suitable types of communication.

Components of embodiments of methods and systems described herein can be physically and/or logically integrated in any manner (e.g., with any suitable distributions of functionality across the components). Portions of embodiments of methods and systems described herein are preferably performed by a first party but can additionally or alternatively be performed by one or more third parties, users, and/or any suitable entities. However, of methods and systems described herein can be configured in any suitable manner.

Embodiments of the methods disclosed herein can include collecting immune response-associated data derived from one or more biological samples, which can function to collect (e.g., generate, determine, receive, etc.) data associated with immune response functionality, for enabling characterization of one or more patients in relation to responsiveness to immune checkpoint therapy (e.g., calculating IRS values with one or more processors).

Immune response-associated data preferably includes data indicative of biological phenomena associated with (e.g., influencing, influenced by, related to, part of, including components of, etc.) the immune response and/or immune system; however, immune response-associated data can include any suitable data (e.g., derivable by sample processing techniques, bioinformatic techniques, statistical techniques, sensors, etc.) related to the immune response and/or immune system.

Any of the variants described herein (e.g., embodiments, variations, examples, specific examples, figures, etc.) and/or any portion of the variants described herein can be additionally or alternatively combined, aggregated, excluded, used, performed serially, performed in parallel, and/or otherwise applied.

Portions of embodiments of the methods and systems can be embodied and/or implemented at least in part as a machine (e.g., processor) configured to receive a computer-readable medium storing computer-readable instructions. The instructions can be executed by computer-executable components that can be integrated with embodiments of the systems and methods described herein. The computer-readable medium can be stored on any suitable computer-readable media such as RAMs, ROMs, flash memory, EEPROMs, optical devices (CD or DVD), hard drives, floppy drives, or any suitable device. The computer-executable component can be a general or application specific processor, but any suitable dedicated hardware or hardware/firmware combination device can alternatively or additionally execute the instructions.

As a person skilled in the art will recognize from the previous detailed description and from the figures and claims, modifications and changes can be made to embodiments of the methods and systems disclosed herein, and/or variants without departing from the scope defined in the claims. Variants described herein not meant to be restrictive. Certain features included in the drawings may be exaggerated in size, and other features may be omitted for clarity and should not be restrictive. The figures are not necessarily to scale. Section titles herein are used for organizational convenience and are not meant to be restrictive. The description of any variant is not necessarily limited to any section of this specification.

As used herein the term “comprising” or “comprises” is used in reference to compositions, methods, and respective component(s) thereof, that are essential to the method or composition, yet open to the inclusion of unspecified elements, whether essential or not.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of elements that do not materially affect the basic and novel or functional characteristic(s) of that embodiment.

The term “statistically significant” or “significantly” refers to statistical significance and generally means a “p” value greater than 0.05 (calculated by the relevant statistical test). Those skilled in the art will readily appreciate that the relevant statistical test for any particular experiment depends on the type of data being analyzed. Additional definitions are provided in the text of individual sections below.

Definitions of common terms in cell biology and molecular biology can be found in “The Merck Manual of Diagnosis and Therapy”, 19th Edition, published by Merck Research Laboratories, 2006 (ISBN 0-911910-19-0); RobertS. Porter et al. (eds.), The Encyclopedia of Molecular Biology, published by Blackwell Science Ltd., 1994 (ISBN 0-632-02182-9); The ELISA guidebook (Methods in molecular biology 149) by Crowther J. R. (2000); Immunology by Werner Luttmann, published by Elsevier, 2006. Definitions of common terms in molecular biology can also be found in Benjamin Lewin, Genes X, published by Jones & Bartlett Publishing, 2009 (ISBN-10: 0763766321); Kendrew et al. (eds.), Molecular Biology and Biotechnology: a Comprehensive Desk Reference, published by VCH Publishers, Inc., 1995 (ISBN 1-56081-569-8) and Cun-ent Protocols in Protein Sciences 2009, Wiley Intersciences, Coligan et al., eds.

Unless otherwise stated, the present invention was performed using standard procedures, as described, for example in Sambrook et al., Molecular Cloning: A Laboratory Manual (3 ed.), Cold Spring Harbor Laboratory Press, Cold Spring Harbor, N.Y., USA (2001) and Davis et al., Basic Methods in Molecular Biology, Elsevier Science Publishing, Inc., New York, USA (1995) which are both incorporated by reference herein in their entireties.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or prior publication, or for any other reason. All statements as to the date or representation as to the contents of these documents is based on the information available to the applicants and does not constitute any admission as to the correctness of the dates or contents of these documents.

One skilled in the art readily appreciates that the present invention is well adapted to carry out the objects and obtain the ends and advantages mentioned, as well as those inherent therein. The details of the description and the examples herein are representative of certain embodiments, are exemplary, and are not intended as limitations on the scope of the invention. Modifications therein and other uses will occur to those skilled in the art. These modifications are encompassed within the spirit of the invention. It will be readily apparent to a person skilled in the art that varying substitutions and modifications may be made to the invention disclosed herein without departing from the scope and spirit of the invention.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention provides all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. It is contemplated that all embodiments described herein are applicable to all different aspects of the invention where appropriate. It is also contemplated that any of the embodiments or aspects can be freely combined with one or more other such embodiments or aspects whenever appropriate. Where elements are presented as lists, e.g., in Markush group or similar format, it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. For example, any one or more active agents, additives, ingredients, optional agents, types of organism, disorders, subjects, or combinations thereof, can be excluded.

Where the claims or description relate to a composition of matter, it is to be understood that methods of making or using the composition of matter according to any of the methods disclosed herein, and methods of using the composition of matter for any of the purposes disclosed herein are aspects of the invention, unless otherwise indicated or unless it would be evident to one of ordinary skill in the art that a contradiction or inconsistency would arise. Where the claims or description relate to a method, e.g., it is to be understood that methods of making compositions useful for performing the method, and products produced according to the method, are aspects of the invention, unless otherwise indicated or unless it would be evident to one of ordinary skill in the art that a contradiction or inconsistency would arise.

Where ranges are given herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, the invention includes embodiments that relate analogously to any intervening value or range defined by any two values in the series, and that the lowest value may be taken as a minimum and the greatest value may be taken as a maximum. Numerical values, as used herein, include values expressed as percentages. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”.

“Approximately” or “about” generally includes numbers that fall within a range of 1% or in some embodiments within a range of 5% of a number or in some embodiments within a range of 10% of a number in either direction (greater than or less than the number) unless otherwise stated or otherwise evident from the context (except where such number would impermissibly exceed 100% of a possible value). It should be understood that, unless clearly indicated to the contrary, in any methods claimed herein that include more than one act, the order of the acts of the method is not necessarily limited to the order in which the acts of the method are recited, but the invention includes embodiments in which the order is so limited. It should also be understood that unless otherwise indicated or evident from the context, any product or composition described herein may be considered “isolated”.

### Examples

### Methods

### Biomarker Testing

PCR based comprehensive genomic profiling, including tumor mutation burden assessment was performed on formalin-fixed paraffin-embedded solid tumor tissue using StrataNGS (Strata Oncology, Ann Arbor, MI) as previously described (Tomlins et al, Journal of Precision Oncology, 2020). In parallel, immune gene expression levels were quantified by an analytically validated investigational assay (Strata Oncology, Ann Arbor, MI). Briefly, exon-spanning PCR amplicons were selected for each target gene and 3 housekeeping genes. After 20 or 30 cycles of PCR amplification, Ion Torrent-based next-generation sequencing was performed targeting ˜1,000,000 reads per sample. Target gene expression was normalized to housekeeping genes and reads per million (nRPM) as compared to a normal control sample.

### Patients

Subjects with advanced solid tumors who were treated with pembrolizumab and had molecular data were identified from the Strata Clinical Molecular Database. 708 solid tumor subjects with QC-passing tumor mutation burden data and immune gene expression data, tumor content>=20% and pembrolizumab started after to sample collection were identified. Patients treated with pembrolizumab monotherapy or combination pembrolizumab+chemotherapy were included.

### Endpoint Determination

Real-world time to next treatment (TTNT) was defined as the time in months from the therapy start date to the date of starting a new therapy after stopping the initial therapy or the date of death. To validate the endpoint as a surrogate for overall survival, TTNT was compared to time to death for patients having both events using Pearson's correlation.

### Algorithm Development

Standard Cox proportional hazards regression was performed to evaluate single biomarkers and combination biomarkers (Statistical Models and Methods for Lifetime Data, by J. F. Lawless. 1982, John Wiley & Sons, New York.) using software at: statpages.info/prophaz.html. For multivariate model building a backward stepwise regression was used, first including all variables in the model, then selectively removing the least significant variables so long as the overall model significance improved. TMB measurements were log 2-transformed and gene expression measurements were log 2-transformed and median-centered prior to analysis. The Chi-squared test was used to test statistical significance of models.

Patient immunotherapy response scores (IRS) were derived using the Cox model as 10 times the inverse of the patient hazard ratio as compared to the median hazard rate.

The Kaplan Meier (KM) method was used to visualize TTNT across patient groups and treatments. The log-rank test was used to test the difference of TTNT curves.

IRS groups were established by dividing the dataset into 8 equal IRS bins and then combining bins based on overlapping TTNT curves.

### Results

### Biomarker Analysis

708 pembrolizumab treated patients (from 24 cancer types 481 pembro+chemo; 227 pembro mono; 170 2nd line pembro mono with prior chemo) with StrataIO scores were analyzed. Aside from several outliers, pembrolizumab TTNT was correlated to OS (Spearman: n=43 r=0.74, or n=45, r=0.61 with outliers).

TABLE 1
Tumor types in the pembrolizumab treatment cohort.

| Cancer Type | Count | % |
|---|---|---|
| Lung - NSCLC | 293 | 41.4% |
| Unknown Primary | 63 | 8.9% |
| Head and Neck | 58 | 8.2% |
| Bladder | 54 | 7.6% |
| Melanoma | 43 | 6.1% |
| Endometrium | 30 | 4.2% |
| Kidney | 28 | 4.0% |
| Esophagus | 22 | 3.1% |
| Other | 117 | 16.5% |
| Total | 708 | 100.0% |

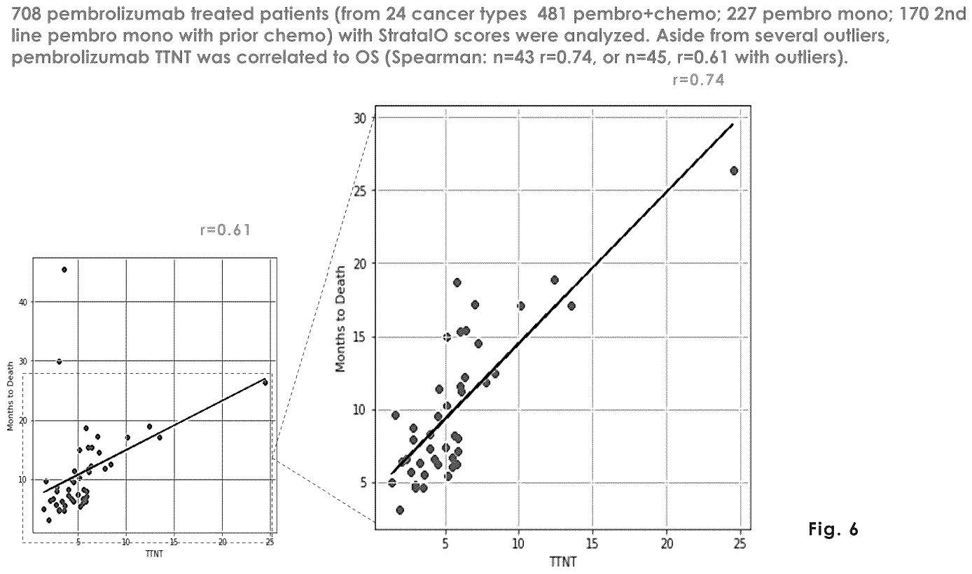

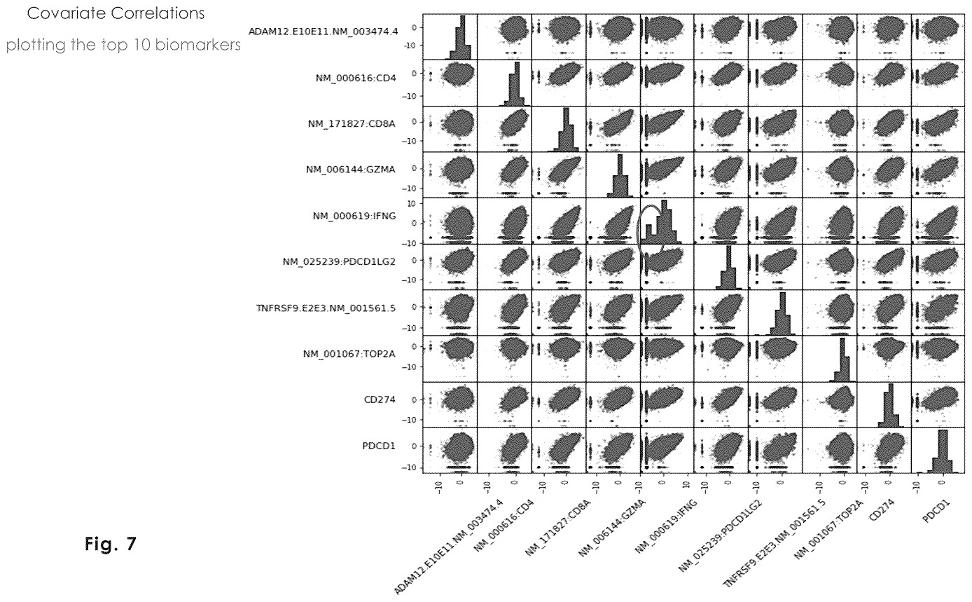

Real world time to next treatment (TTNT) was inferred for each subject as the time from starting pembrolizumab to the time of stopping pembrolizumab and starting a new therapy or death. To establish the appropriateness of TTNT for studying pembrolizumab treatment outcomes, TTNT was compared overall survival and found to be highly correlated (n=43 r=0.74, or n=45, r=0.61 with outliers, FIG. 6).

First, we evaluated the association of TMB and 10 gene expression biomarkers with pembrolizumab treatment outcome (Table 2).

TABLE 2
Univariate and multivariate biomarker analysis for predicting pebrolizumab
time to next treatment. The multivariate analysis includes only the
final variable set. P-value is from the Chi squared test.

| | Univariate | | | Multivariate | |
|---|---|---|---|---|---|
| Biomarker | HR (95% CI) | | p | HR | p |
| log2TMB | 0.77 | (0.70-28.02) | 3.7E−09 | 0.75 (0.69-0.82) | 5.8E−10 |
| CD274 (PD-L1) Average | 0.90 | (0.85-10.47) | 7.1E−04 | | |
| PDCDI (PD-1) Average | 0.89 | (0.84-9.83) | 1.1E−03 | 0.86 (0.79-0.94) | 5.0E−04 |
| PDCDILG2 (PD-L2) | 0.91 | (0.85-7.77) | 4.6E−03 | 0.87 (0.79-0.95) | 1.3E−03 |
| INFRSF9 | 0.92 | (0.87-7.07) | 7.4E−03 | | |
| IDO1 | 0.95 | (0.92-5.42) | 0.023 | | |
| UBE2C | 0.91 | (0.83-5.09) | 0.029 | | |
| TIGIT | 0.95 | (0.90-3.51) | 0.088 | | |
| LAG3 | 0.94 | (0.88-3.19) | 0.109 | | |
| CD8A | 0.95 | (0.90-3.17) | 0.111 | | |
| IFNG | 0.98 | (0.95-2.51) | 0.176 | | |
| TCF7 | 0.95 | (0.87-2.35) | 0.196 | | |
| CTLA4 | 0.97 | (0.92-2.10) | 0.233 | | |
| GZMA | 0.97 | (0.91-1.57) | 0.337 | | |
| TOP2A | 0.96 | (0.87-1.49) | 0.356 | | |
| VTCN1 | 0.99 | (0.96-1.28) | 0.413 | | |
| FOXP3 | 0.98 | (0.92-1.19) | 0.439 | | |
| ADAM12 | 1.02 | (0.95-0.69) | 0.620 | 1.08 (1.01-1.15) | 0.03 |
| CD4 | 1.02 | (0.93-0.63) | 0.646 | 1.15 (1.01-1.31) | 0.04 |
| AXL | 0.98 | (0.90-0.52) | 0.699 | | |
| HAVCR2 | 1.00 | (0.93-0.11) | 0.929 | | |
| TNFRSF4 | 1.00 | (0.93-0.08) | 0.946 | | |

Next, backward stepwise regression was performed to fit a multivariate Cox proportional hazards model. The final model included 5 of 10 input variables and was more significantly associated with pembrolizumab treatment outcome (p=1.2e-7) than any individual variable or other model. Notably, TMB, PD-L1, pd-1, and PD-L2 were all independent predictors of pembrolizumab treatment outcome (Table 2).

### Outcome Analysis

To evaluate the potential application of the Cox model to predict pembrolizumab treatment outcome in patients, patient Immunotherapy Response Scores (IRS) were derived as:

$$I R S = 4.03 * exp ( 0.287 * [ transformed TMB measurement ] + 0.147 * [ PD - 1 normalized level ] + 0. 1 \; 43 * [ PD - L 2 normalized level ] - 0.138 * [ CD 4 normalized level ] - 0.073 * [ ADAM 12 normalized level ] ) .$$

where an IRS of 10 is equal to the median hazard rate observed in the dataset, values greater than 10 represent decreased hazard (i.e. more benefit from pembrolizumab) and values less than 10 represent increased hazard (i.e. less benefit from pembrolizumab). Patients were assigned to one of three IRS groups to compare patient outcomes (Table 3).

TABLE 3
Pembrolizumab versus chemotherapy treatment outcomes
by Immunotherapy Response Group and Strata IO.

| | pembrolizumab | | | chemotherapy | | | | |
|---|---|---|---|---|---|---|---|---|
| IRS | | | Median | | | Median | | |
| Group | n | % | Survival | n | % | Survival | HR | p-value |
| Low | 266 | 0.38 | 7 | 174 | 0.41 | 7 | 0.90 (0.72-1.12) | 0.34 |
| Medium | 176 | 0.25 | 17.5 | 115 | 0.27 | 8.2 | 0.53 (0.39-0.73) | 7.77E−05 |
| High | 266 | 0.38 | 24.7 | 137 | 0.32 | 7.3 | 0.32 (0.23-0.44) | 7.38E−13 |
| | 708 | 100% | | 426 | 100% | | | |

TTNT—time to next treatment. HR—hazard ratio. P-value by the log-rank test.

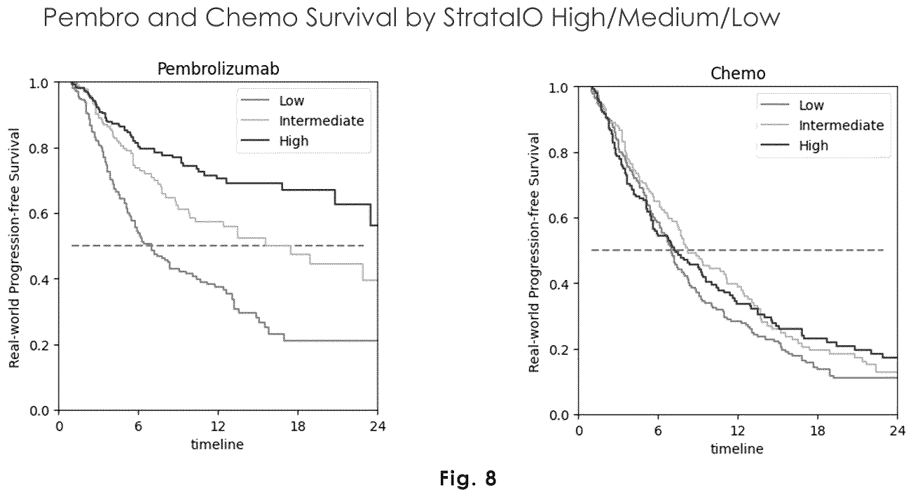

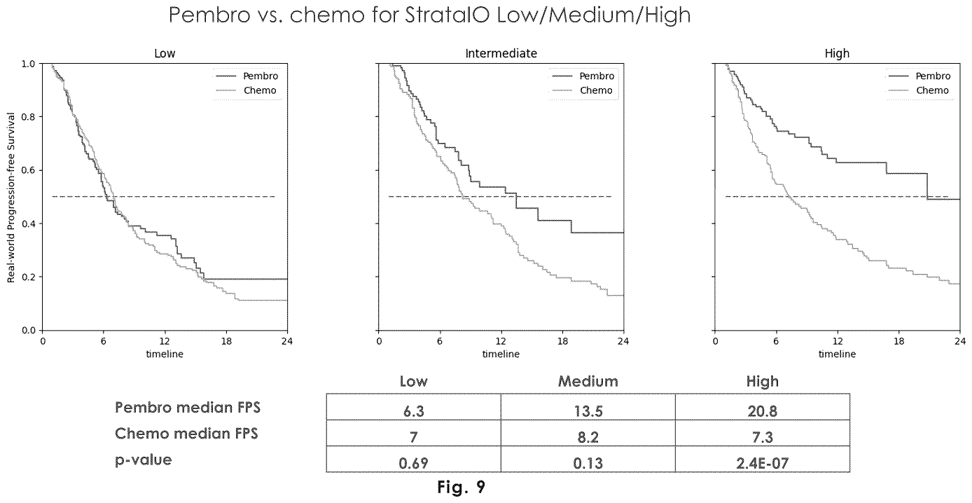

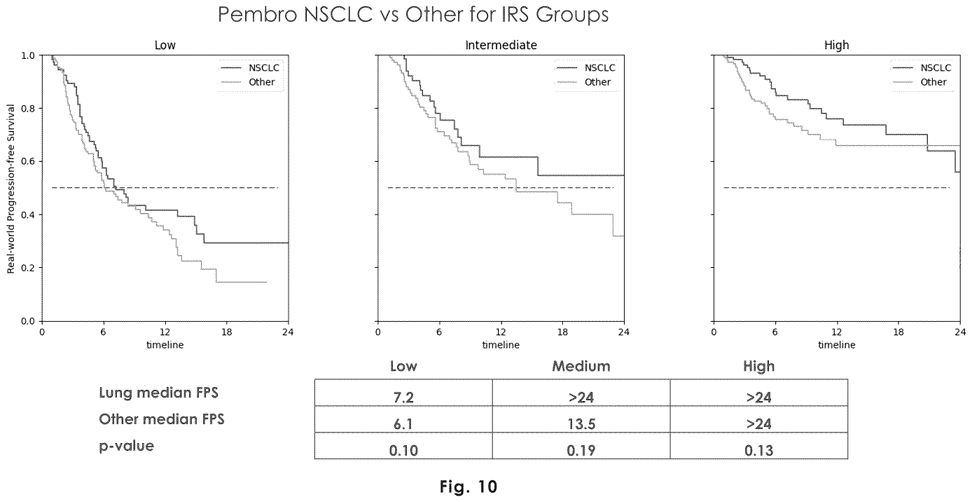

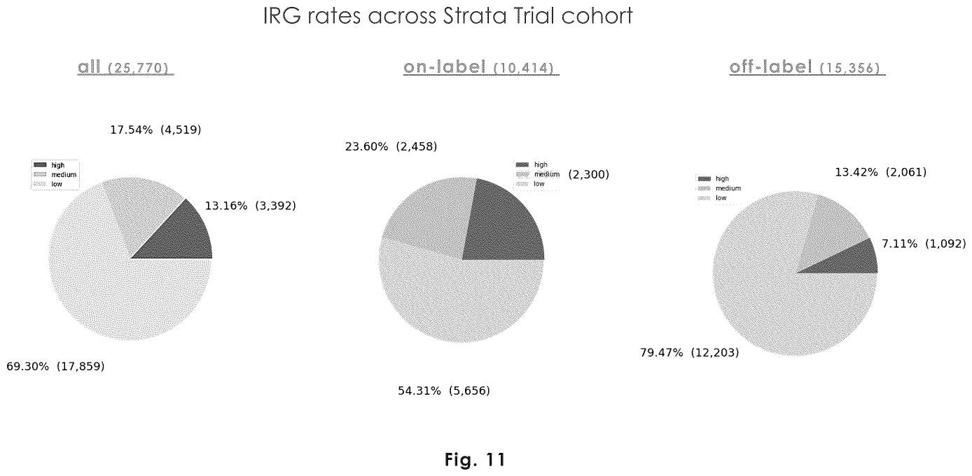

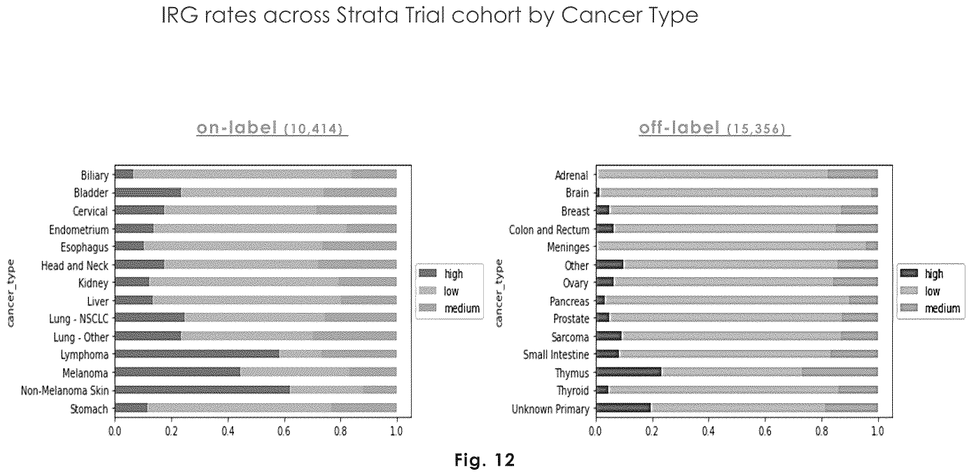

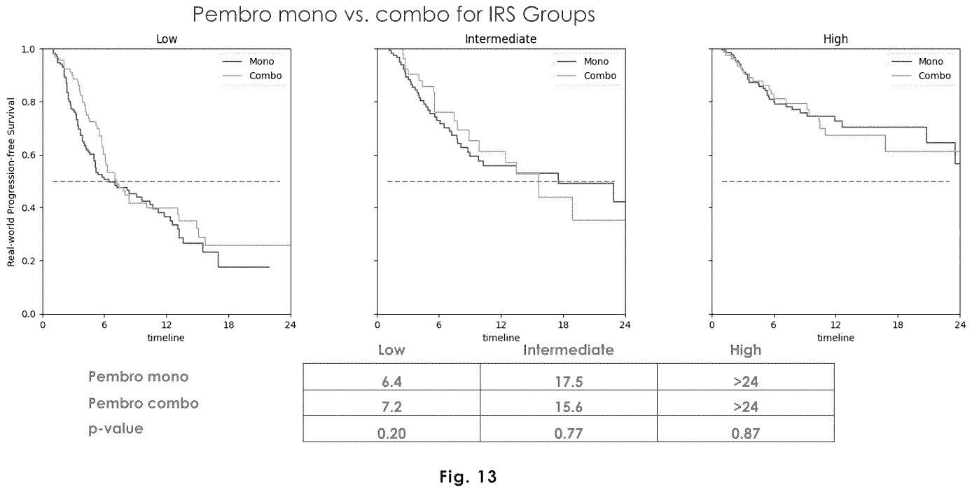

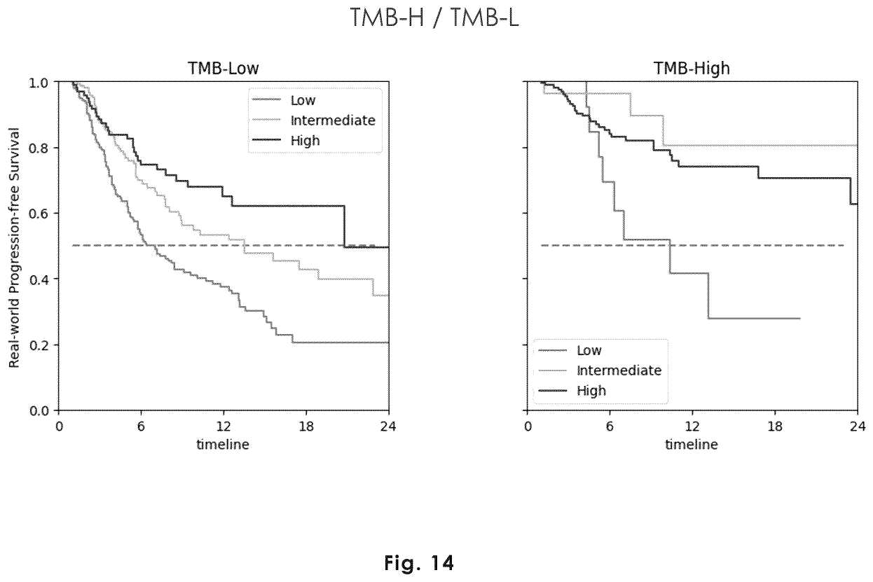

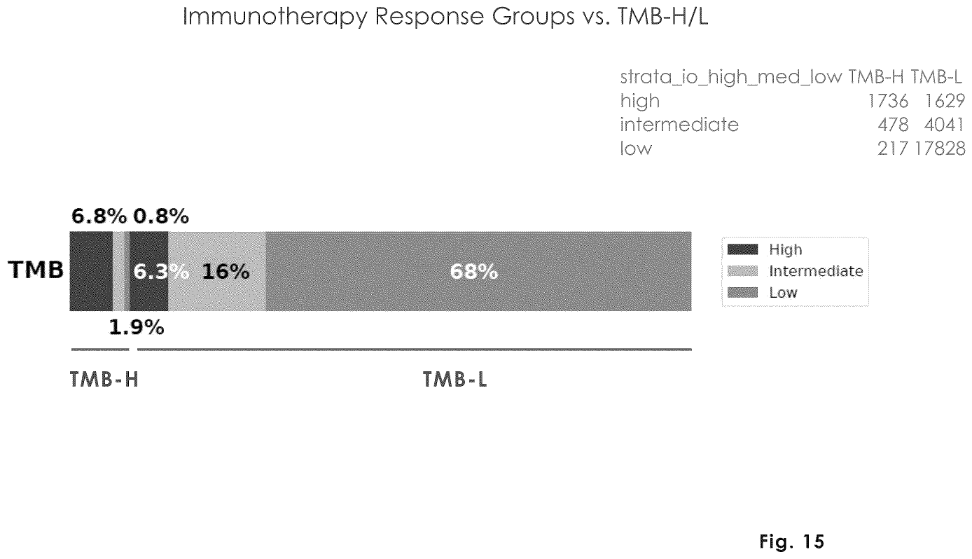

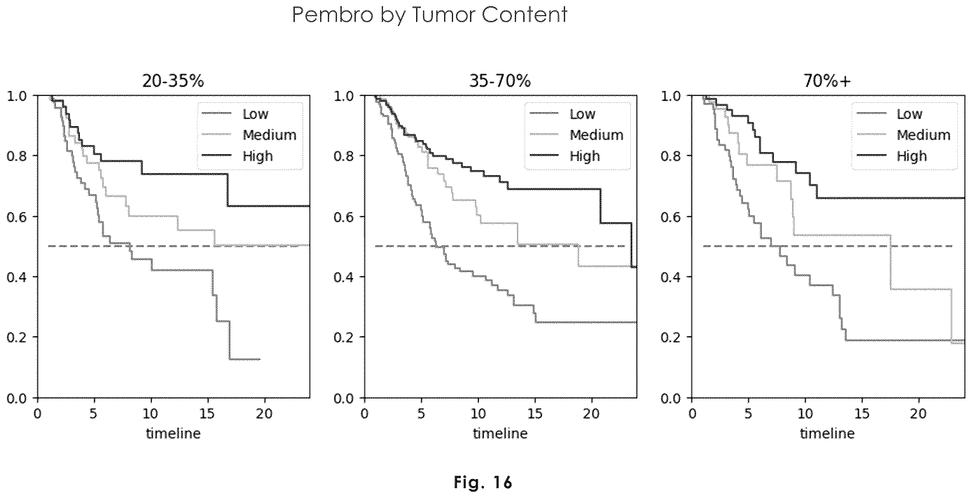

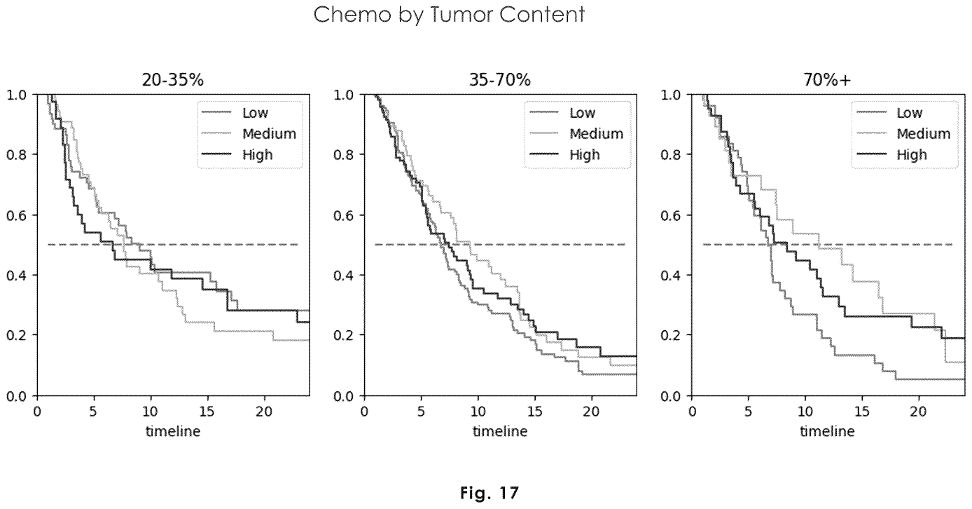

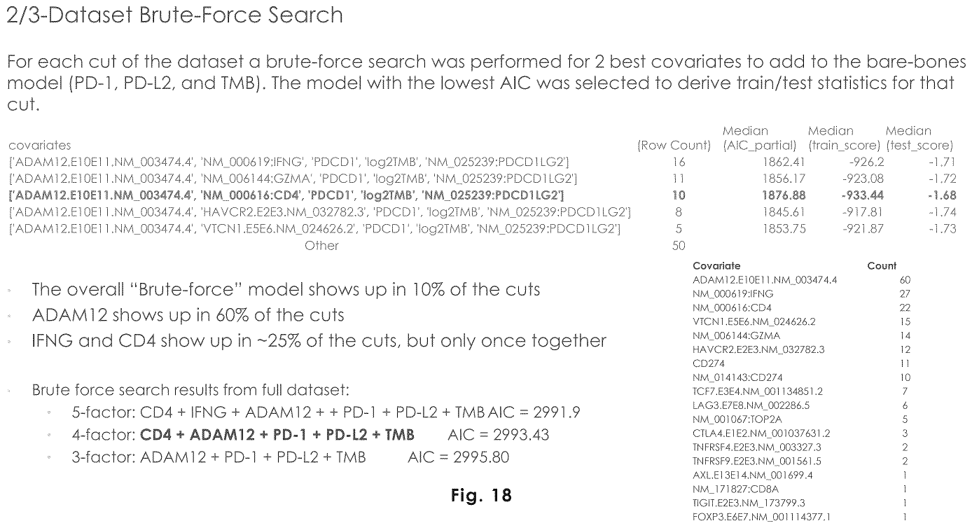

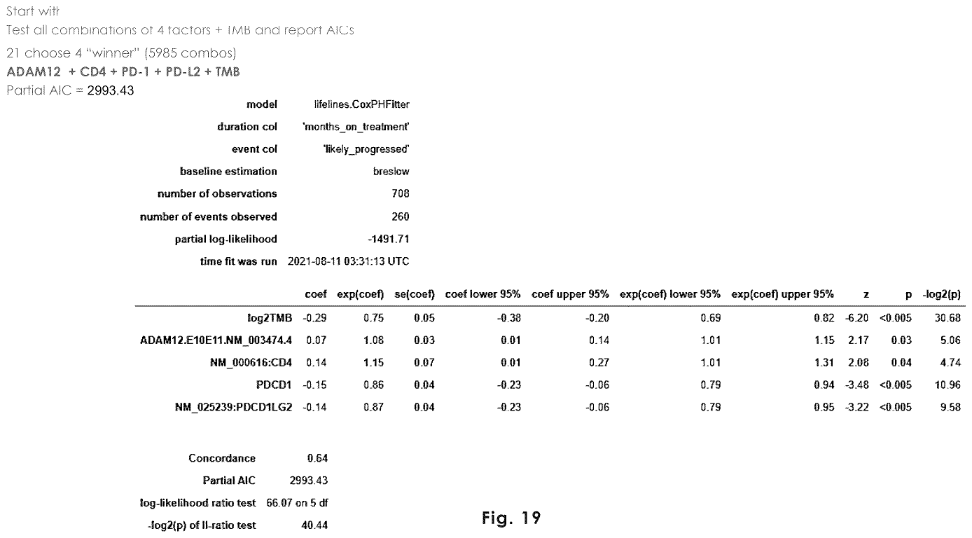

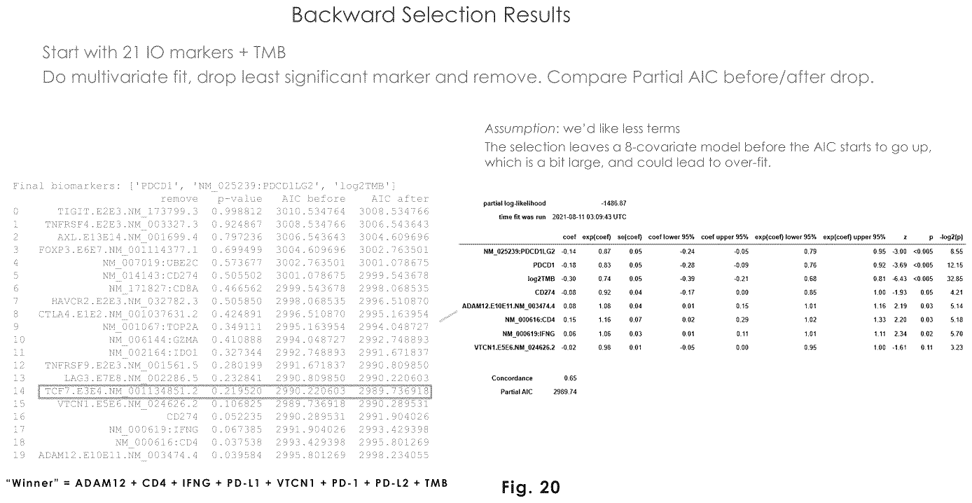

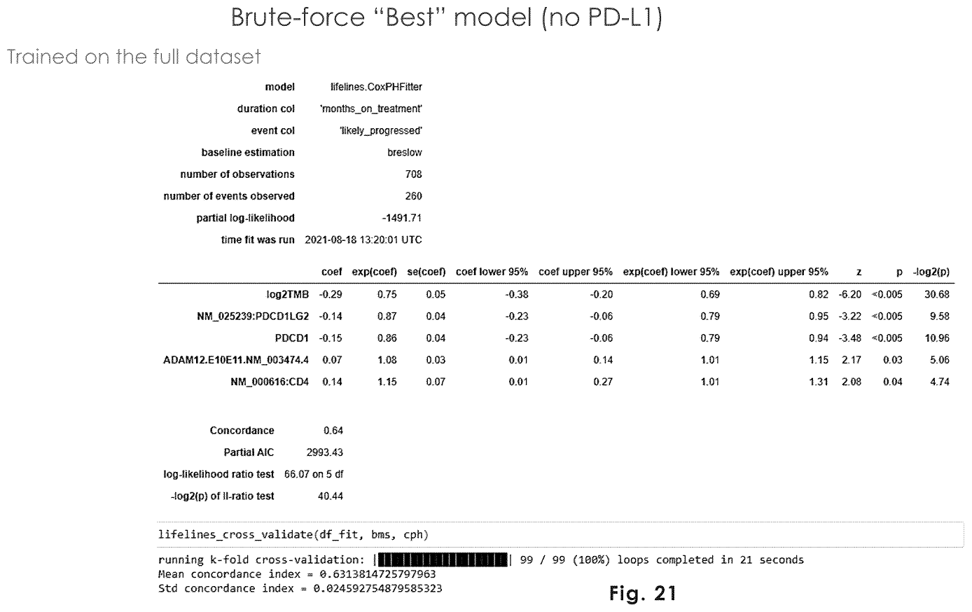

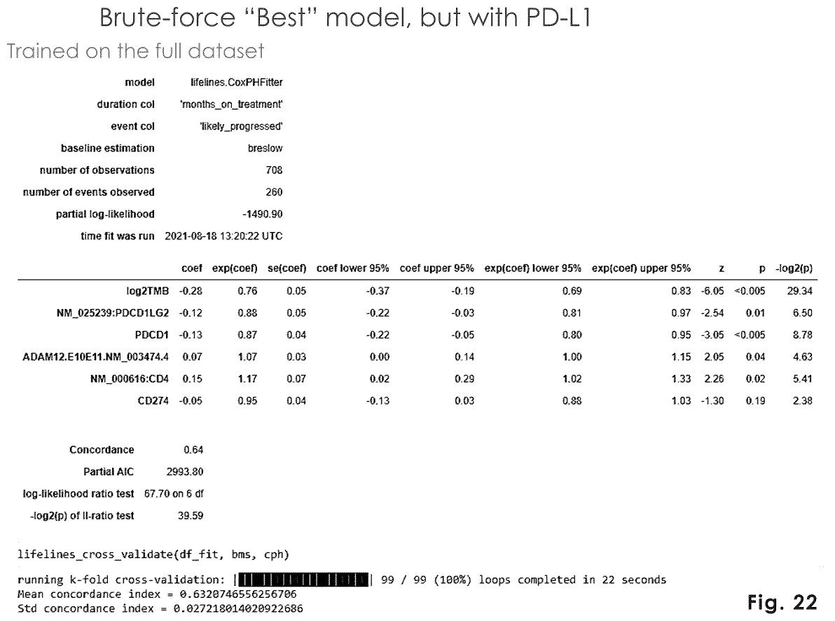

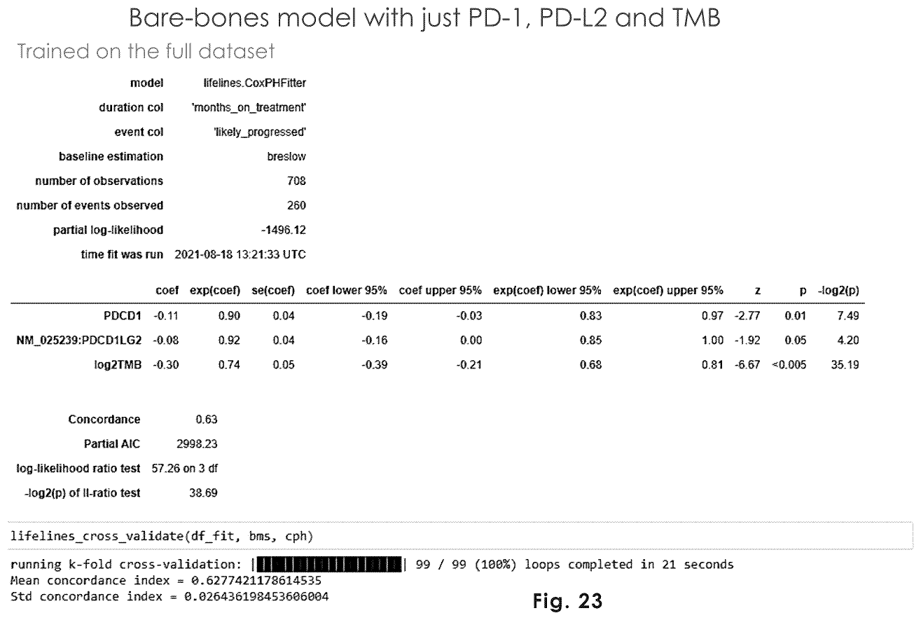

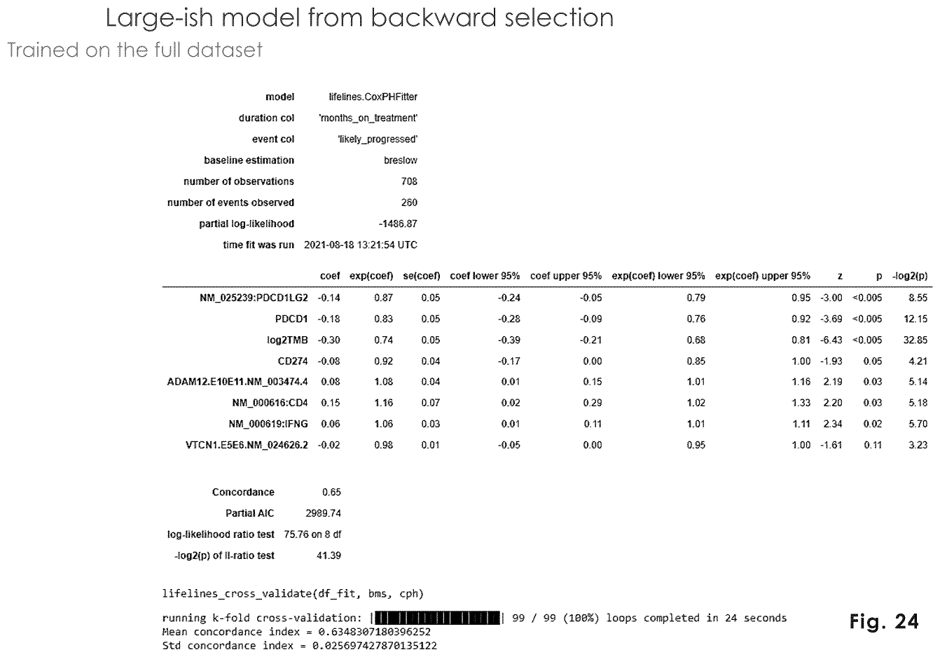

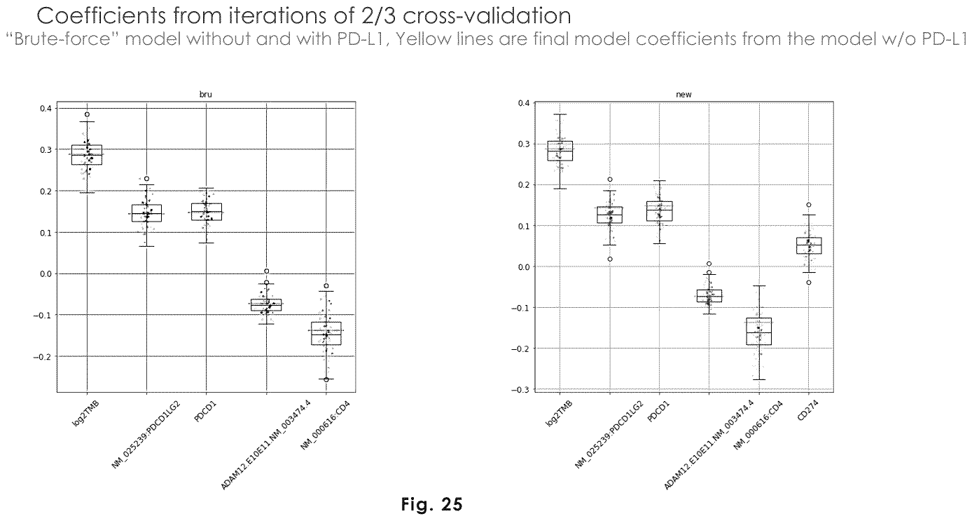

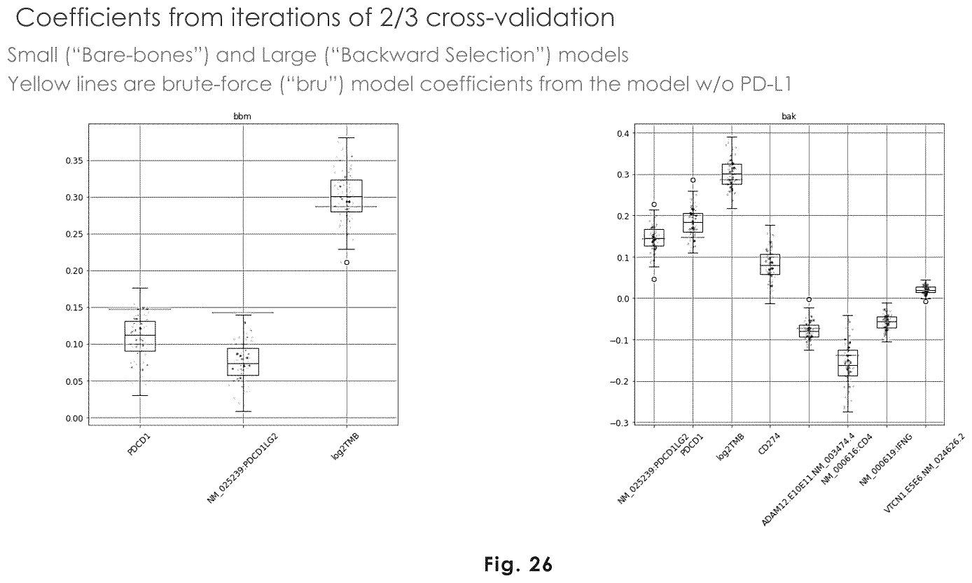

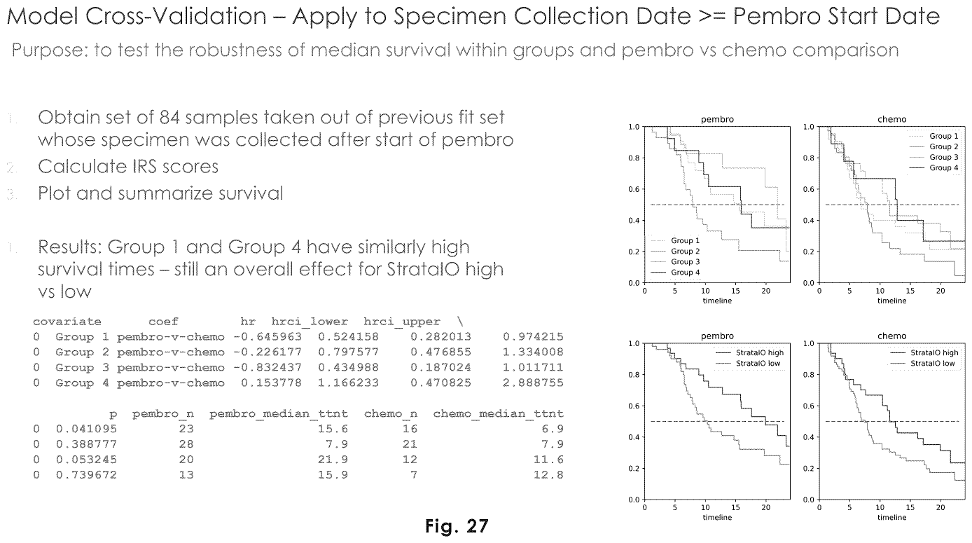

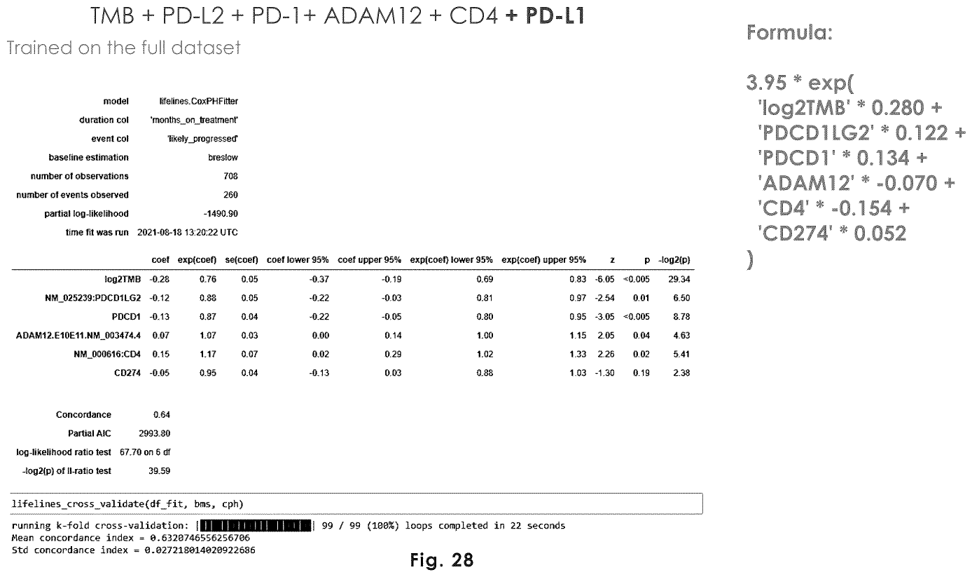

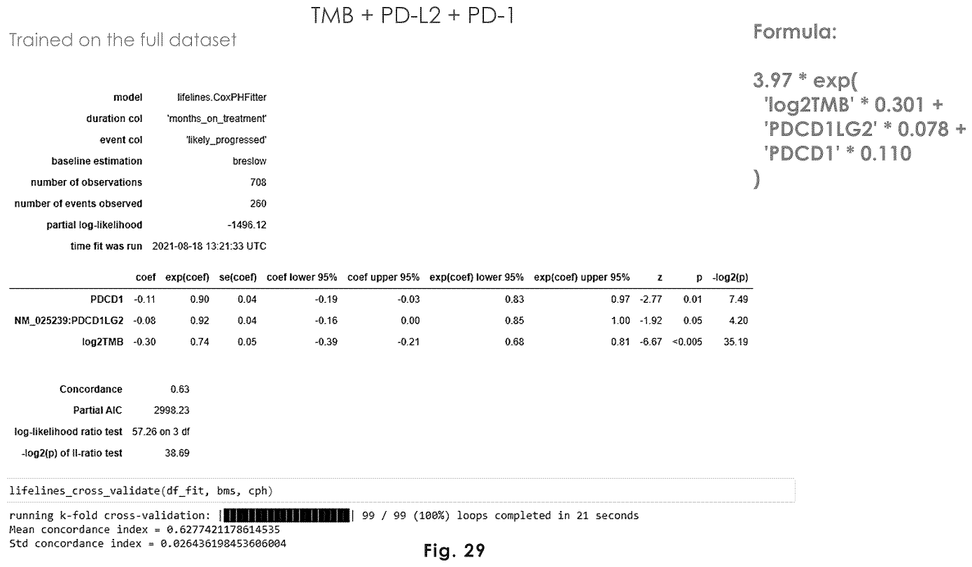

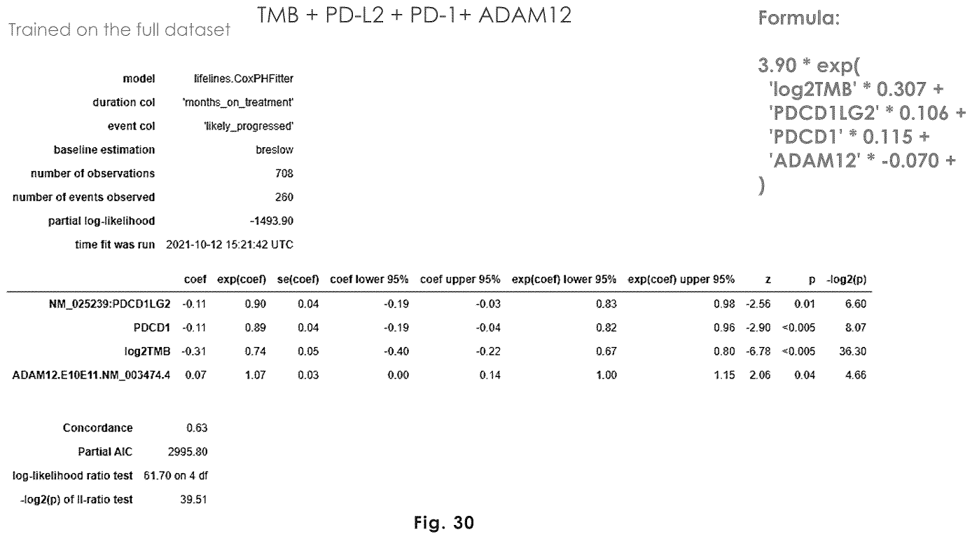

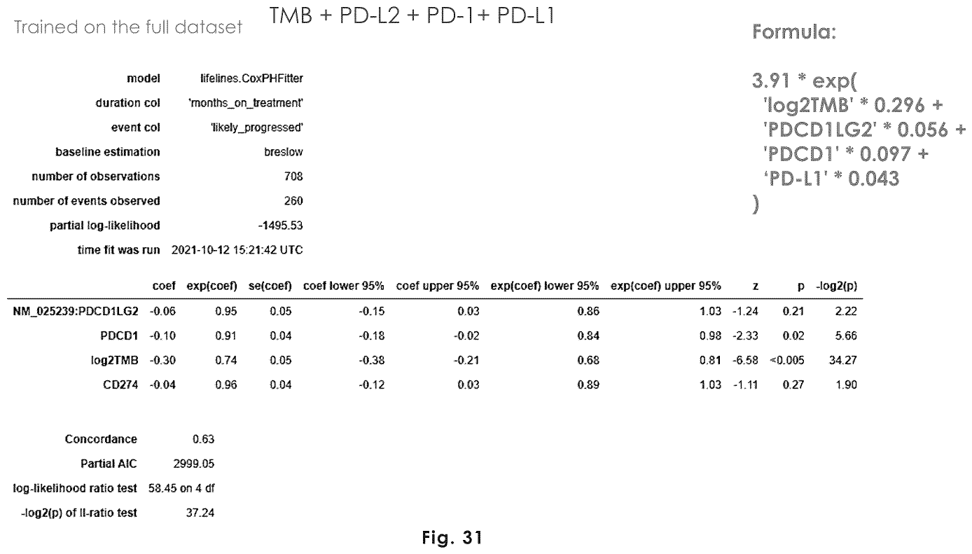

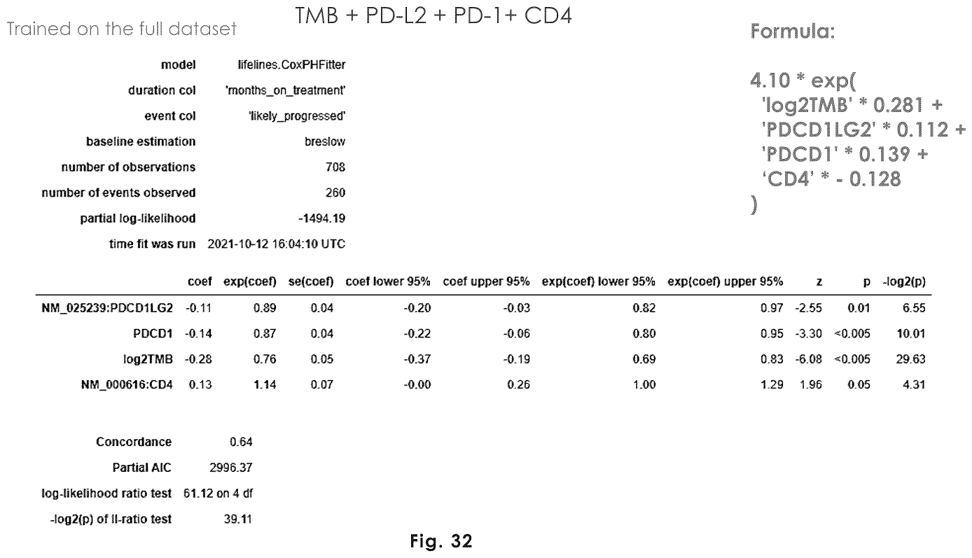

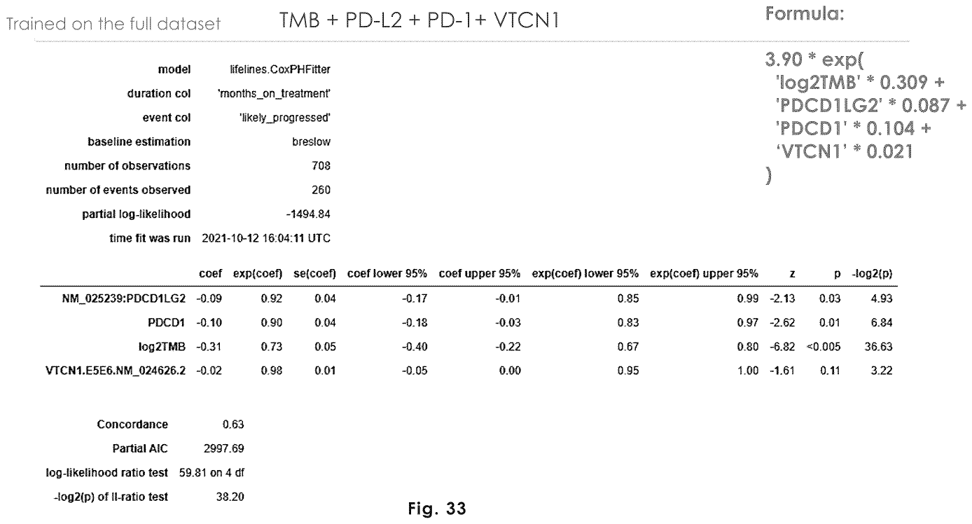

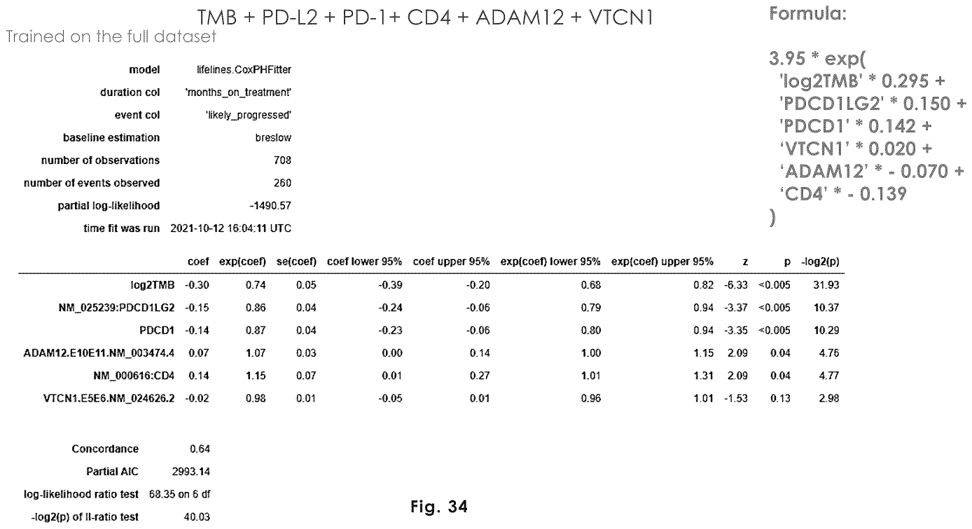

Kaplan Meier analysis showed that pembrolizumab treatment outcome varied widely across groups with median TTNT ranging from longer than 24 months in group HIGH to just 7 months in group LOW (FIG. 8).

TABLE 4
Pembro vs Chemo in TMB-Low Subset chemotherapy

| | pembrolizumab | | | chemotherapy | | | | |
|---|---|---|---|---|---|---|---|---|
| IRS | | | Median | | | Median | | |
| Group | n | % | Survival | n | % | Survival | HR | p-value |
| Low | 253 | 0.51 | 7 | 165 | 0.52 | 7.2 | 0.94 (0.74-1.18) | 0.58 |
| Medium | 148 | 0.30 | 13.5 | 92 | 0.29 | 9.1 | 0.62 (0.45-0.87) | 5.20E−03 |
| High | 99 | 0.20 | 20.8 | 62 | 0.19 | 5.9 | 0.38 (0.24-0.60) | 3.00E−05 |
| | 500 | 100% | | 319 | 100% | | | |

TABLE 5
Pembro vs Chemo by Tumor Content

| | | pembrolizumab | | | chemotherapy | | | | |
|---|---|---|---|---|---|---|---|---|---|
| | IRS | | | Median | | | Median | | |
| | Group | n | % | Survival | n | % | Survival | HR | p-value |
| <35% | Low | 73 | 0.41 | 8.2 | 45 | 0.41 | 9.1 | 1.13 (0.68-1.80) | 0.69 |
| | Medium | 54 | 0.30 | >24 | 36 | 0.33 | 7.7 | 0.52 (0.30-0.90) | 0.02 |
| | High | 51 | 0.29 | >24 | 29 | 0.26 | 6.7 | 0.31 (0.16-0.61) | 7.06E−04 |
| | | 178 | 100% | | 110 | 100% | | | |
| 35-70% | Low | 129 | 0.35 | 6.3 | 91 | 0.41 | 6.8 | 0.84 (0.62-1.13) | 0.25 |
| | Medium | 81 | 0.23 | 18.9 | 56 | 0.25 | 9.3 | 0.49 (0.31-0.79) | 3.08E−03 |
| | High | 150 | 0.42 | 23.5 | 77 | 0.34 | 7.5 | 0.31 (0.20-0.47) | 4.06E−08 |
| | | 360 | 100% | | 224 | 100% | | | |
| >70% | Low | 64 | 0.38 | 7 | 38 | 0.41 | 6.7 | 0.80 (0.51-1.27) | 0.35 |
| | Medium | 41 | 0.24 | 17.5 | 23 | 0.25 | 11.2 | 0.65 (0.32-1.32) | 0.23 |
| | High | 65 | 0.38 | >24 | 31 | 0.34 | 8.4 | 0.35 (0.18-0.66) | 1.22E−03 |
| | | 170 | 100% | | 92 | 100% | | | |

Continuous StrataIO score predicted pembrolizumab TTNT across the cohort (p=3.67e-12) and within all tested subsets, including NSCLC pembro mono (p=9.37e-05),NSCLC pembro+chemo (p=3.02e-03), non-NSCLC pembro mono (p=6.57e-06) and non-NSCLC pembro+chemo (p=2.88e-02), but did not predict chemotherapy TTNT (p=4.10e-01). *StrataIO “High” vs “Low” based on the Low/Medium/High grouping.

### Discussion

Herein, a highly significant multivariate model that combined TMB and immune gene expression to predict real-world pembrolizumab treatment outcomes in 700+ patients with diverse solid tumor types was developed. The model inputs were generated simultaneously from a clinically validated next-generation sequencing platform, requiring a single, small formalin-fixed paraffin-embedded biopsy specimen for testing. An Immunotherapy Response Score (IRS) was derived to predict individual patient's likelihood of benefit from pembrolizumab and demonstrated that patients in high IRS groups had far superior treatment outcomes as compared to chemotherapy, whereas patients in low IRS groups did not. The association of IRS groups with treatment outcomes was stable after separating NSCLC from other tumors, monotherapy from combination therapy and TMB high from TMB low tumors, suggesting that the model captures universal biological features of pembrolizumab benefit.

Notably, when applied to more than 25,000 advanced solid tumors, high IRS groups were more common in tumor types known to derived benefit from immunotherapy, but also occurred in subsets of nearly every tumor type. The proportion of tumors falling into high IRS groups was predictive of observed objective response rates in independent studies, indicating that IRS groups likely identify or enrich for patients likely to derive benefit from ICIs.

The model holds several potentially interesting biological insights. First, TMB, PD-L1 and PD-L2 were all independent predictors of benefit, indicating a multiplicative predictive effect across the three biomarkers, with increased antigenicity (TMB) and increased immune checkpoint activity (PD-L1 and/or PD-L2) driving benefit from immune checkpoint blockade. While many past studies have established TMB and PD-L1 as predictive biomarkers, and recent studies have established the PD-L2 is also predictive, this is the first to combine and optimize these three variables into a single model, likely providing a more comprehensive predictor of immunotherapy benefit.

IRS has potential application for both refining the use of pembrolizumab in tumor types for which immunotherapy is indicated and in selecting patients for immunotherapy outside of indicated tumor types (e.g., off-label). It is shown that in low IRS groups, among tumor types such as NSCLC for which pembrolizumab is approved, pembrolizumab has little to no benefit relative to chemotherapy. Given that immunotherapy is expensive and can pose serious toxicity, its use should be considered more cautiously in low IRS groups. Among tumor types for which immunotherapy is not indicated, patients that fall into high IRS groups should be considered for treatment given the potential for significant benefit relative to chemotherapy.

Pembrolizumab was recently approved for TMB high tumors (>10 mutations per megabase), independent of tumor type, eliciting a 25% objective response rate. The disclosed data suggests that an integrated model combining TMB with immune gene expression provides better prediction, stratifying treatment outcomes within TMB high and TMB low patients.

In summary, disclosed is a biologically rational predictive model of immunotherapy response integrating DNA mutation and RNA expression-based expression biomarkers. A single clinically validated NGS platform capable of simultaneously reading out mutations and quantifying gene expression was utilized, providing a clear diagnostic pathway for clinical application. Upon further validation in independent tumor cohorts, this work has the potential to both expand the benefit of ICIs to additional patients and reduce unnecessary toxicity and financial burden in patients unlikely to benefit from ICI treatment.

### Additional Examples

### Cohort

The Strata Trial (NCT03061305), is an observational clinical trial evaluating the impact of molecular profiling on patients with advanced solid tumors. It has been reviewed and approved by Advarra Institutional Review Board (IRB; IRB Pro00019183) prior to study start. At enrolling health care systems, all adult patients with locally advanced (stage III), unresectable or metastatic (stage IV) solid tumors and available FFPE tumor tissue were eligible; the protocol also allowed enrollment of patients with rare early-stage tumors.

The Strata Clinical Molecular Database (SCMD) contains deidentified subject, molecular profiling, treatment, and survival data for all enrolled NCT03061305 participants. Prior antineoplastic therapy, including start and stop dates, were collected for trial participants at the time of study entry. Antineoplastic therapy data and survival status were prospectively collected for up to 3 years from the time of enrollment and/or informed consent. First, a case series analysis was performed herein focusing on the development of an integrative CGP+qTP based PD-(L)1 benefit predictor, an exploratory aim of the trial. Post-hoc power analysis was not performed to determine the sample size of this discovery cohort. A power analysis was then performed to determine the cohort size needed for an independent validation cohort as described below. Patients in the SCMD tested by a version of StrataNGS assessing TMB (see Biomarker Data below) with parallel gene expression testing data completed between 25 Jan. 2017 to 12 Jul. 2022 were eligible for analysis with a data cutoff of 12 Jul. 2022; for the discovery cohort, only patients tested through 4 May 2021 were eligible and the data cutoff date was the same as the overall cohort. General validity analyses of the SCMD are described in the Supplementary Methods.

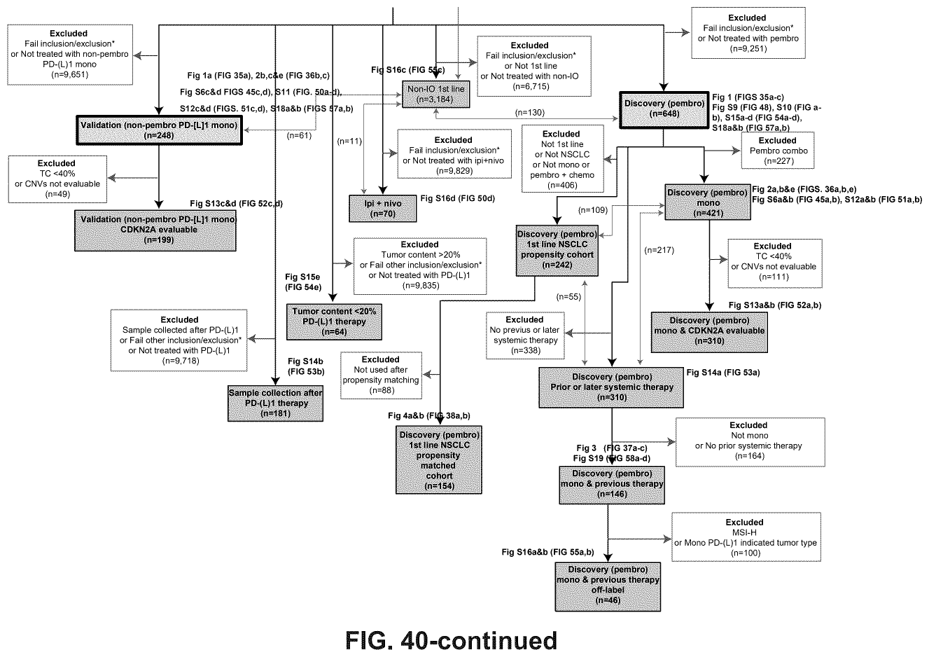

For both the discovery and validation cohorts, common inclusion criteria were: valid TMB measurements from StrataNGS testing (including meeting the overall 20% tumor content requirement), valid immune gene expression quantification from an investigative multiplex PCR based transcriptomic profiling test, and documented treatment with at least one antineoplastic agent. For the discovery cohort, additional inclusion and exclusion criteria included: 1) treatment with a pembrolizumab containing systemic line of therapy, 2) the tested tissue specimen was collected prior to the systemic pembrolizumab line start date, and 3) the patient had no prior anti-PD-(L)1 or CTLA4 blockade therapy prior to the pembrolizumab line start date. For the validation cohort, additional inclusion and exclusion criteria included: 1) treatment with systemic non-pembrolizumab anti-PD-(L)1 monotherapy, 2) the tested tissue specimen was collected prior to the PD-(L)1 therapy start date, 3) had no prior anti-PD-(L)1 or CTLA4 blockade therapy prior to the non-pembrolizumab PD-(L)1 line start date, and 4) patients were not in the discovery cohort. Additional inclusion/exclusion criteria for other analyses are described below and in the overall study diagram (FIG. 40). Except in the analysis specifically assessing IRS performance in samples collected after PD-(L)1 therapy, patients with samples collected after the start date of the analyzed therapy line were excluded from all analyses.

Source data verification in the Strata Trial was performed for high-risk data fields such as demographics and treatment history per an approved Trial Monitoring Plan. Data completeness, consistency, and quality assurance checks were performed across the Strata electronic data capture (EDC) system per an approved Data Management Plan; 100% source data verification was performed for the discovery cohort. Additional details on the Strata Trial experience and Strata molecular profiling have been described⁵⁴⁻⁵⁶.

### Real-World Treatment Data

Patient treatment history from electronic health records (EHRs) or manual updating was standardized to enable derivation of real-world progression free survival (rwPFS) by time to next therapy (TTNT) and overall survival (OS). All medications were classified into anti-neoplastic or non-antineoplastic treatments, and all anti-neoplastic treatments were further subclassified (e.g. chemotherapy, immune-oncology [IO; PD-(L)1 or CTLA4], oncogene TKI, hormonal, etc); non-antineoplastic treatments were excluded from further consideration. Line of therapy assignment was performed in two stages: first, single-dose treatments with consecutive doses administered within 90 days were combined into a course of treatment with a single start and end date; next, non-overlapping lines of treatment were inferred by considering each course of medication sequentially by start date. Subsequent treatment courses that began more than 30 days after the start of a given line of treatment, or whose duration of overlap with the line was less than 50%, were considered to establish a new line of treatment. Any treatment line with more than one anti-neoplastic therapy administered during the line was considered combination therapy. First line chemo and/or hormonal therapies which concluded 180 days or more prior to the start of subsequent therapy were considered as adjuvant.

To determine rwPFS, an effective end date was defined for each course of treatment as either a) date of last record if treatment is ongoing (censored), b) date of death (event), c) the start date of the subsequent therapy line (event), or d) the latest available end date (censored if no subsequent line of therapy or death). rwPFS was calculated as the difference, in months, between the start date and effective end date of the treatment line. OS was calculated as the difference, in months, between the start date of the treatment line and date of death (or censoring).

### Biomarker Data

Multiplex PCR-based comprehensive genomic profiling (PCR-CGP), including TMB assessment, was performed on FFPE solid tumor tissue using StrataNGS (Strata Oncology, Ann Arbor, MI). The current version of StrataNGS is a 437 gene laboratory-developed test (LDT) for FFPE tumor tissue samples performed on co-isolated DNA and RNA, which has been validated on over 1,900 FFPE tumor samples, and is covered for Medicare beneficiaries⁵⁵. While earlier StrataNGS versions were also used during the study period, all had similar performance for the TMB assessment (and MSI) used herein⁵⁶. In parallel, immune gene expression was determined by analytically and clinically validated multiplex PCR-based qTP via an investigational/supplementary test performed on the same co-isolated RNA as described⁵⁴; different versions of this quantitative transcriptomic profiling test have been run in parallel with StrataNGS (assessing 26, 46 and currently 103 expression targets), with panel specific scaling validated by concordance analyses performed as needed. One or more exon-spanning PCR amplicons were selected for each target gene and multiple housekeeping genes (see Supplementary Methods) were included, with 3 pan-cancer stable housekeeping genes used for clinical testing. qTP was performed using Ampliseq after reverse transcription followed by Ion Torrent-based next-generation sequencing. Expression target transcripts were measured in normalized reads per million (nRPM), whereby raw expression target read counts were normalized by a factor that results in the median housekeeping gene expression value matching the same gene's standard reads per million in a reference FFPE normal cell line sample (GM24149) run in parallel with all clinically tested samples.⁵⁴ Relevant components of the analytical and clinical validation of the current version of the integrated CGP+qTP LDT that includes the IRS model are described in the Supplementary Methods.

### Statistical Analysis

Unadjusted rwPFS and overall-survival (OS) across groups and treatments were visualized using the Kaplan Meier method. Adjusted rwPFS and OS analyses were performed to compare group outcomes (by adjusted hazard ratios and two-sided p-values) using Cox proportional hazard models unless otherwise specified. Covariate adjustments shared between all models include age and gender. Repeated measures were accounted for in settings where participants had multiple records (e.g., prior treatment then pembrolizumab monotherapy). Analysis dependent covariates, as appropriate, included IRS group, tumor type (most prevalent in cohort vs. all other types), systemic therapy line number, TMB status (High vs. Low), therapy type (monotherapy or combination), PD-(L)1 therapy type (PD-1 or PD-L1 therapy), CDKN2A status (wild type or deep deletion) and tumor content (continuous). The indicated analysis used MSKCC definition of TMB sensitive tumor types (MSI-H, POLE^{mutant}, NSCLC, head and neck cancer, or melanoma as TMB sensitive; all other samples as TMB insensitive)⁵⁷ instead of most prevalent tumor type vs. all others. Performance status (or surrogates) were not available from data collected as part of the Strata Trial. Proportional hazard assumptions were checked for each model and cohort of interest using Schoenfeld residuals. Unstratified analysis results are presented throughout, as stratifying analyses to preserve proportional hazards produced similar covariate effect sizes where the assumption was not met (discovery cohort); all monotherapy discovery cohort analyses and validation cohort analyses met proportional hazard assumptions. Where specified, the two-sided log-rank test was used to test rwPFS and OS curve differences (Benjamini Hochberg adjusted as appropriate).

For the predictive analysis using the internal comparator cohort considering rwPFS on the immediately preceding systemic therapy vs. subsequent pembrolizumab monotherapy, adjusted Cox proportional hazards models were utilized to examine the interaction between pembrolizumab vs. prior chemotherapy rwPFS within the same patient and IRS status (IRS-High vs. Low). The likelihood ratio test for interaction compared the reduced model, which excluded the IRS by treatment interaction, with the competing full model, which included the IRS by treatment interaction.

To determine the performance of IRS in a setting where both PD-(L)1 monotherapy and combination therapy are used in the same line, we limited the discovery cohort to the subset of patients with NSCLC treated with first line pembrolizumab monotherapy or pembrolizumab+chemotherapy combination therapy. As PD-L1 IHC status and performance status were not available and known confounders driving this treatment decision, we performed nearest neighbor propensity score matching (with a 0.25 standard deviation caliper applied)⁵⁸ using age, gender, TMB, IRS, and the normalized PD-L1 expression component of the IRS biomarker (see Supplementary Results for validation of this biomarker vs. IHC in a separate cohort). All patients in the combination therapy cohort who could not be matched to within 0.25*standard deviation a monotherapy patient's propensity score were dropped. Confirmation that the final matched monotherapy and combination therapy did not significantly differ (two tailed t-tests for continuous variables and two-tailed Fisher's exact test for categorical variables; both at p<0.05 as significant) was performed. Kaplan Meier analysis was used to visualize monotherapy vs. combination therapy rwPFS in the separate IRS-H and IRS-L populations, using a two-sided log-rank test to compare therapy group outcomes.

The correlation between rwPFS and overall survival (OS) was calculated using Spearman's p among patients with both a documented death event and at least two lines of therapy. Throughout this study, TMB-H was defined as ≥10 Muts/Mb by StrataNGS, given the previous validation of TMB by StrataNGS and high concordance with TMB estimates from FoundationOne tissue testing (see Supplementary Methods)⁵⁵. All statistical analyses were performed in R (v. 4), and SAS (v. 9.4). For all cohort analyses, two-sided p-values<0.05 were considered statistically significant.

### Immunotherapy Response Score (IRS) Model Development and Validation

The association of TMB and 23 candidate immune and proliferation gene expression biomarkers with pembrolizumab rwPFS was determined using Cox proportional hazards regression in the 648-patient pembrolizumab (both monotherapy and combination therapy) discovery cohort. TMB measurements were log₂-transformed and gene expression measurements were log₂-transformed and median-centered per laboratory workflow prior to analysis. Feature selection was performed via Lasso-penalized Cox proportional hazards regression in this 648-patient discovery cohort, with the Lasso penalty term chosen as the value which maximized the concordance index via 5-fold cross validation. Model coefficients for the five features with non-0 coefficients in the Lasso model were finalized via standard Cox regression. Individual patient IRS were derived from the Cox model as:

$$I R S = 0.2 \; 73758 * TMB + 0.1 \; 12641 * PD - 1 + 0.0 \; 61904 * PD - L 1 - 0.077011 * TOP 2 A - 0.057991 * ADAM 12$$

We assigned patients to one of two IRS groups to compare patient outcomes (i.e., Low (L)<0.873569 and High (H)≥0.873569; more likely to benefit) based on balancing minimization of the hazard ratio for IRS-H vs. IRS-L with maximization of the IRS-H monotherapy population.

After locking the IRS model (and -H vs. -L threshold), a power analysis was performed to determine the size of an appropriate independent validation cohort. In the overall discovery cohort, 46% patients were IRS-H, and we observed an adjusted hazard ratio for IRS-H vs. IRS-L rwPFS of 0.49 (47% event rate); therefore, assuming an IRS-H to IRS-L ratio of 1:1 and a 50% event rate, a validation cohort of 180 patients would have 90% power to detect a similar (0.5) hazard ratio. We then identified all (n=248) patients in the SCMD meeting the above-described validation cohort inclusion/exclusion criteria (the same as the discovery cohort except only including any non-pembrolizumab PD-(L)1 monotherapy treatment); the locked IRS model (and -H vs. -L threshold) was then applied to these subjects.

### Results

### Clinical Molecular Data

The Strata Trial (NCT03061305) is an observational clinical trial evaluating the impact of tumor molecular profiling for patients with advanced solid tumors. De-identified demographic, clinical and molecular data from patients in the Strata Trial is maintained in the Strata Clinical Molecular Database (SCMD). With a data-cutoff of 12 Jul. 2022, the SCMD contains clinical and molecular data from a total of 57,648 unique patients with advanced solid tumors (from 47 tumor types) from 59 United States health care systems who had routine FFPE tumor tissue molecularly profiled by the StrataNGS CGP test^{55,56}, with 9,899 Strata Trial patients from 30 United States health care systems (from 43 tumor types) having treatment data from at least one systemic antineoplastic agent (FIG. 40, and Tables S1 & S2).

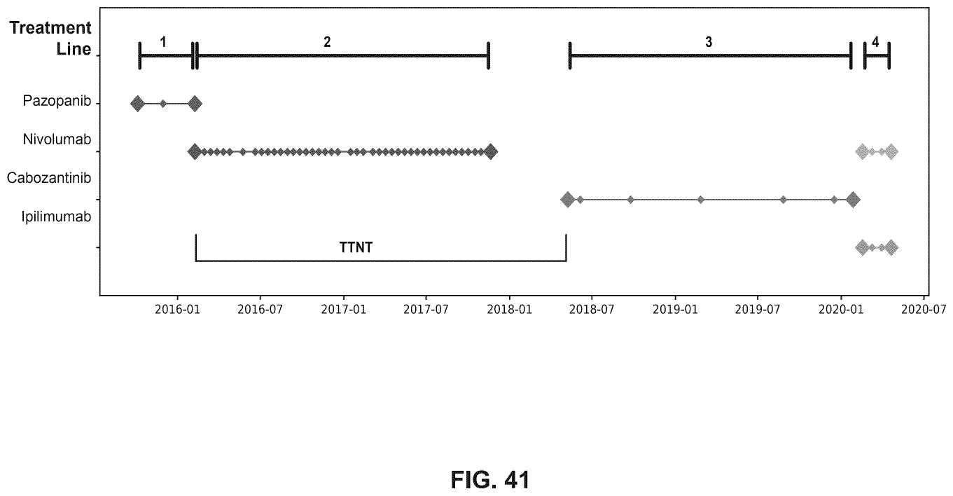

For all Strata Trial patients with treatment data in the SCMD, antineoplastic treatment start and stop dates (for all prior therapies and up to 3 years after Strata trial enrollment) were obtained from automated electronic health record queries or manual entry; data was updated regularly by submitting institutions, and date of death was obtained similarly. Time to next therapy (TTNT) as a measure of real-world progression free survival (rwPFS) was determined directly from treatment start and stop dates for each line of therapy accounting for adjuvant/systemic therapy, monotherapy/combination therapy, potential overlap of treatment start/stop dates, repeating lines of therapy (whether monotherapy in combination) given the variance in real world treatment patterns (FIG. 41).

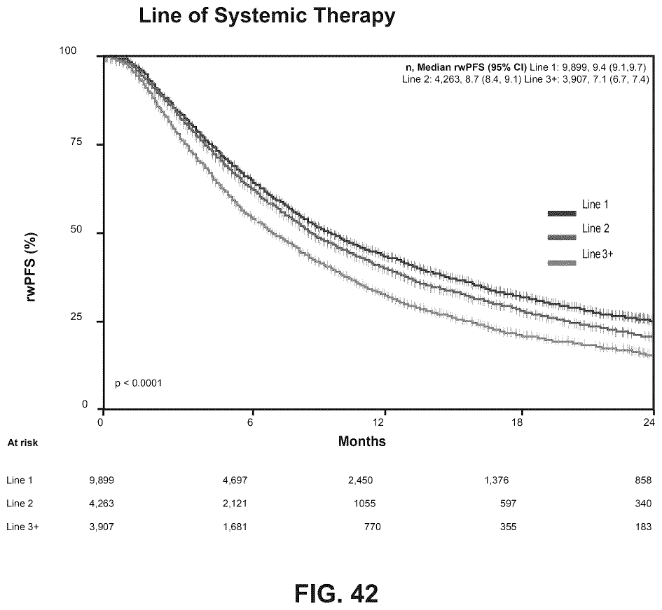

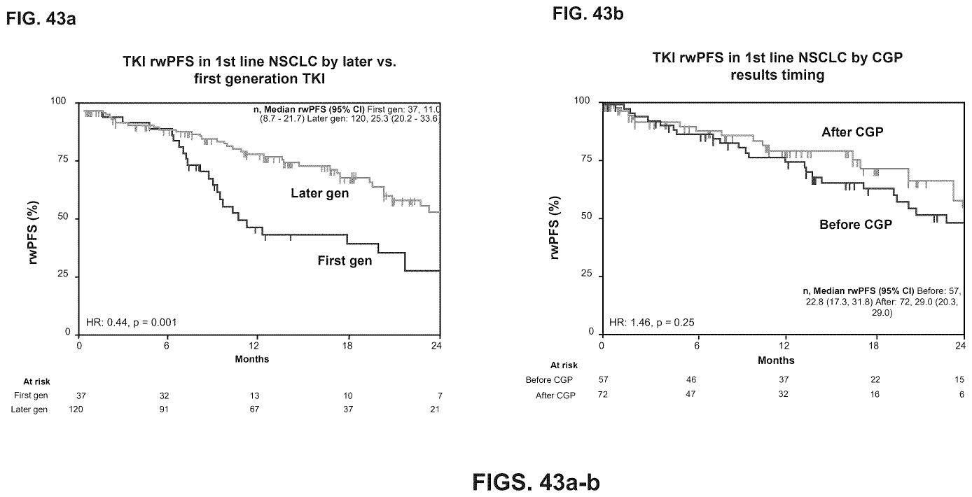

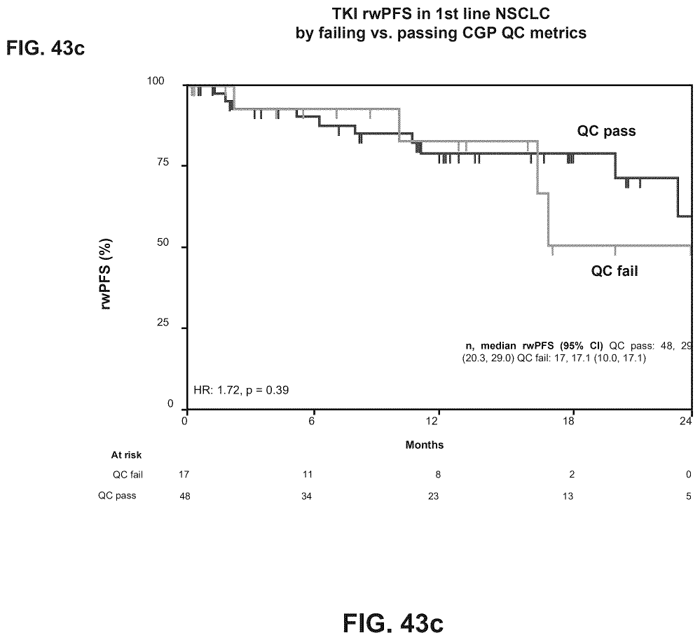

Among the 9,899 patients, the median follow-up from start of first systemic treatment was 15.4 months [interquartile range (IQR) 6.9-29.4 months]. The median number of total systemic lines of therapy per patient was 1 (IQR 1-2), with 49.2% of systemic lines being monotherapy, and the median number of systemic therapies per line was 2 (IQR 1-2). The median number of total systemic lines of therapy per patient after Strata trial enrollment was 1 (IQR 0-1), with 47.2% of systemic lines being monotherapy, and the median number of systemic therapies per line was 2 (IQR 1-2). As expected, median rwPFS was shorter in subsequent therapy lines (median rwPFS in 1^{st}, 2^{nd} and 3^{rd+} lines of 9.4 [95% CI 9.1-9.7], 8.7 [95% CI 8.4-9.1] and 7.1 [95% CI 6.7-7.4] months, respectively, unadjusted log rank p<0.0001; FIG. 42). We have previously demonstrated that molecular alteration frequency in the first -30,000 consecutive patients enrolled in the Strata Trial⁵⁶ was similar to that observed in the Memorial Sloan Kettering single institution pan-cancer profiling effort, MSK-IMPACT⁵⁹, supporting the generalizability of the SCMD and additional clinical and molecular analyses supporting the validity of the SCMD are described in the Supplementary Results and FIGS. 43a-c.

### Biomarkers of Anti-PD-1/PD-L1 Blockade Benefit Analysis

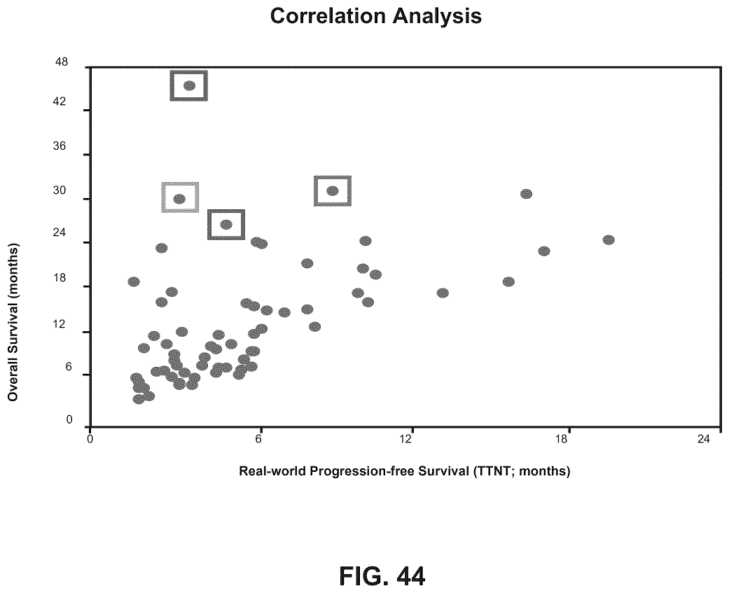

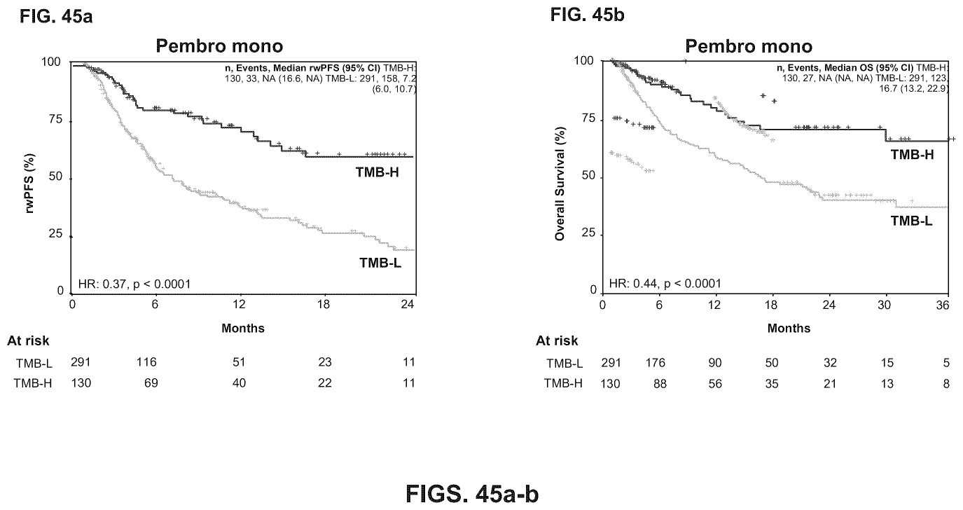

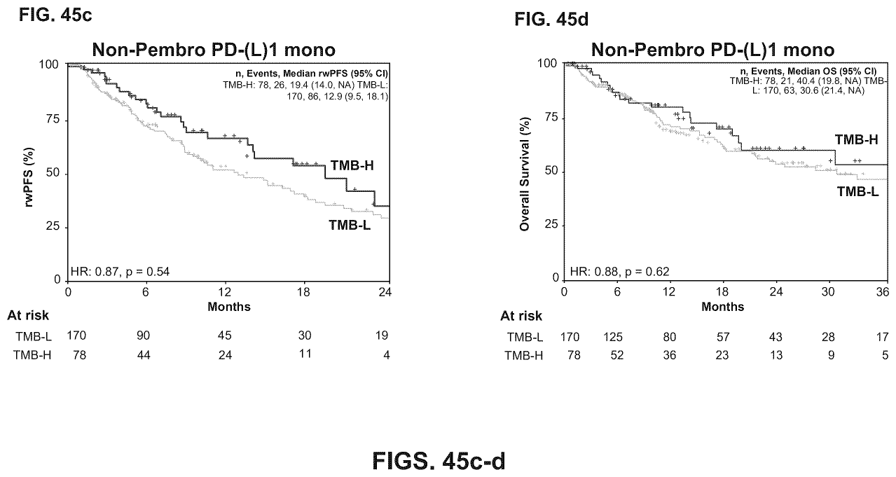

To develop an integrative, CGP+qTP based tumor-agnostic PD-(L)1 blockade predictive biomarker, we first limited results to the 648 of 9,899 (6.5%) patients in the SCMD who met all of the inclusion/exclusion criteria (see Methods) including: valid TMB measurements from StrataNGS testing (including meeting the overall 20% tumor content requirement), valid immune gene expression quantification from an investigative multiplex PCR based qTP test, and with a pembrolizumab containing systemic line of therapy (FIG. 40). As shown in FIG. 45a, this discovery cohort was comprised of patients with 26 tumor types, with NSCLC accounting for 265 (40.9%); tumor types and demographics are provided in Tables S1 & S2. rwPFS was inferred for each patient as the time from starting the pembrolizumab containing therapy line to the time of stopping that line and starting a new therapy line or death; both rwPFS and OS were used for studying treatment outcome based on comparisons of these endpoints (Supplementary Results and FIG. 44). Importantly, to confirm the validity of ≥10 Muts/Mb from StrataNGS testing to define TMB-H, we demonstrated that TMB-H patients (n=130) had significantly longer pembrolizumab monotherapy rwPFS vs. TMB-L patients (n=291; median rwPFS Not reached [95% CI 16.6−NA] vs. 7.2 [95% CI 6.0-10.7] months, adjusted hazard ratio 0.37 [95% CI 0.25-0.54], p<0.0001 when adjusted for age, gender, most common tumor type [NSCLC] vs. others, and line of systemic therapy FIG. 45a), as well as significantly longer OS (median OS Not reached [95% CI NA-NA] vs. 16.7 [95% CI 13.2−22.9] months, adjusted hazard ratio 0.44 [95% CI 0.29-0.67], p=0.0001; FIG. 45b).

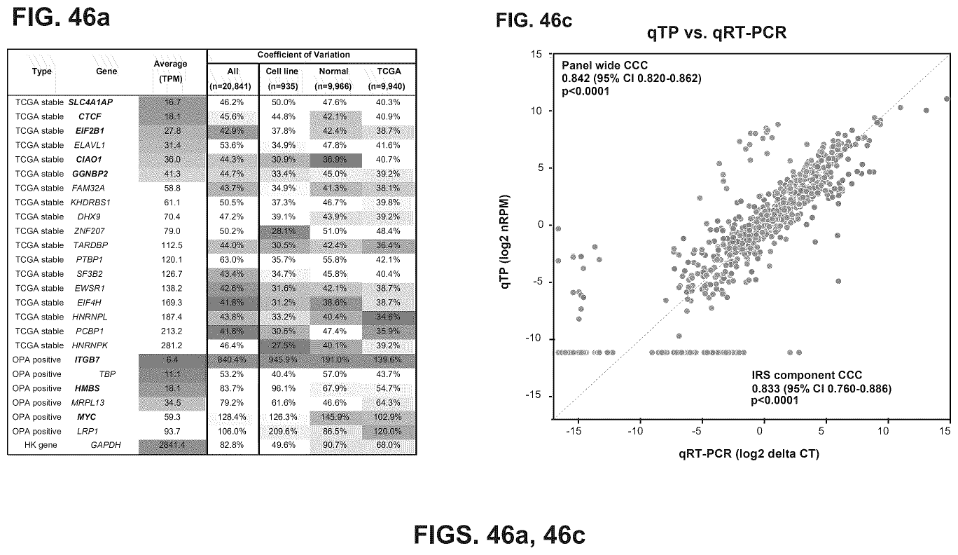

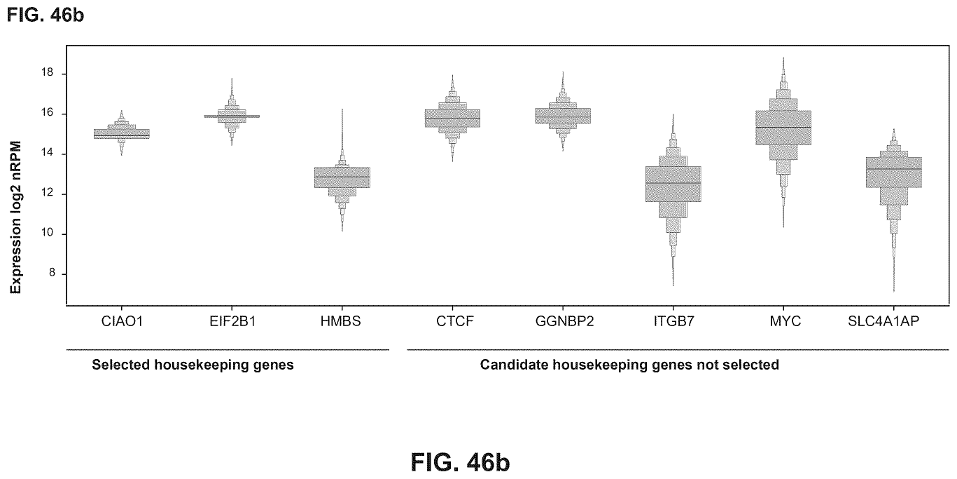

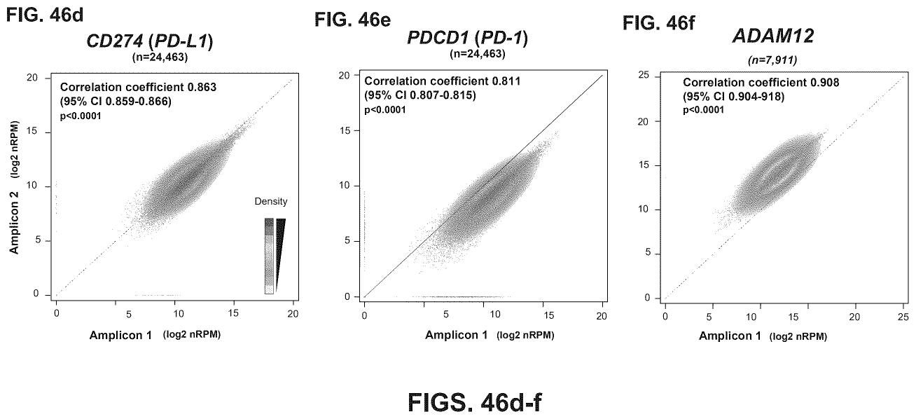

To identify potential expression-based biomarkers of PD-(L) therapy benefit, we first considered 23 candidate immune and proliferation gene expression biomarkers (from 21 genes; two amplicons targeting separate exon-exon junctions of PDCD1 [PD-1] and CD274 [PD-L1] were included) assessed across clinical RNA tests run in parallel with the StrataNGS CGP test (which generates TMB). Data on housekeeping gene selection, correlation of independent PD-1 and PD-L1 amplicons, correlation of tumor-type expression profiles for candidate gene expression biomarkers between SCMD and The Cancer Genome Atlas (TCGA) profiled tumors, and analytical and clinical validation of the qTP component of the CGP+qTP test (including qRT-PCR and clinical IHC data from >1,000 total FFPE tumors) is described in the Supplementary Methods, Supplementary Results, Table S3, and FIGS. 46a-f & 47a-d. We therefore assessed the association of pembrolizumab rwPFS with StrataNGS derived TMB and the 23-candidate immune/proliferation gene expression biomarkers in the 648-patient discovery cohort. As shown in Table S4, significant (p<0.01) univariate predictors included TMB (HR=0.79; p<0.0001), PD-1 expression (HR=0.91; p=0.001) and PD-L1 expression by both amplicons (both HR=0.92; both p=0.005).

### Integrative Immunotherapy Response Score (IRS) to Predict PD-(L)1 Blockade Benefit

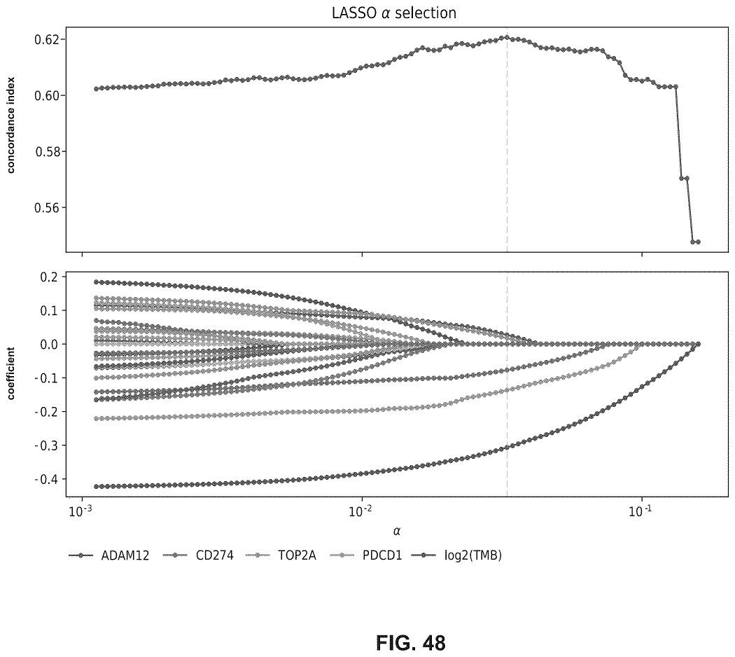

To develop an integrative model predictive of PD-(L)1 therapy benefit, we performed Lasso-penalized Cox proportional hazards regression with five-fold cross-validation in this 648 patient discovery cohort, with the highest concordance index obtained using a five-term model that included TMB, PD-1, PD-L1, ADAM12, and TOP2A (FIG. 48), with increasing TMB, PD-1 and PD-L1 associated with longer pembrolizumab rwPFS, while increasing ADAM12 and TOP2A were associated with shorter pembrolizumab rwPFS. As the same feature set was also obtained via exhaustive combinatorial search of all five-term models by standard Cox proportional hazards regression, the five term Cox proportional hazards model was used to generate the final integrative model (multivariate analysis on the final five variable set is shown in Table S4). As shown in Table S5, across 24,463 Strata Trial samples in the SCMD with informative TMB and gene expression (regardless of treatment data availability), TMB was minimally correlated with all final model gene expression biomarkers (Spearman p=0.032 [ADAM12] to 0.211 [TOP2A]), while correlation of individual gene expression biomarkers ranged from p=0.033 (PD-1 vs. TOP2A) to p=0.571 (PD-1 vs. PD-L1).

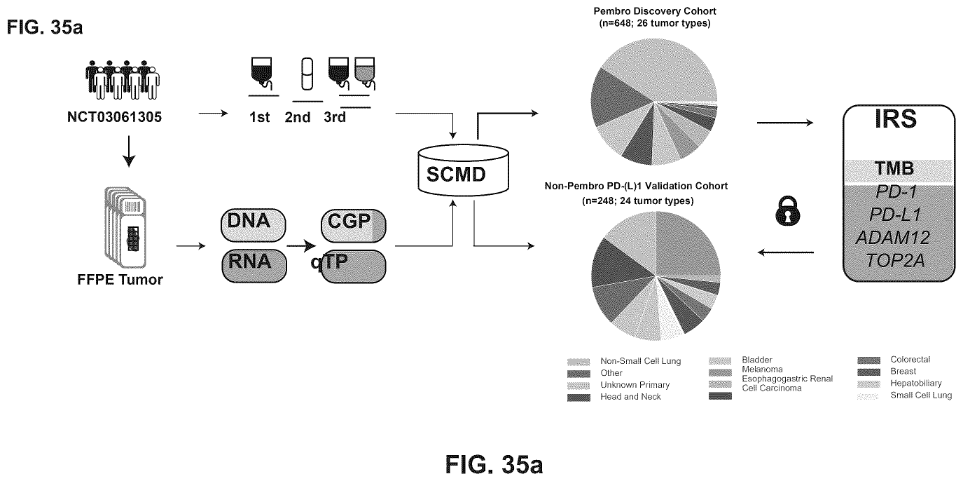

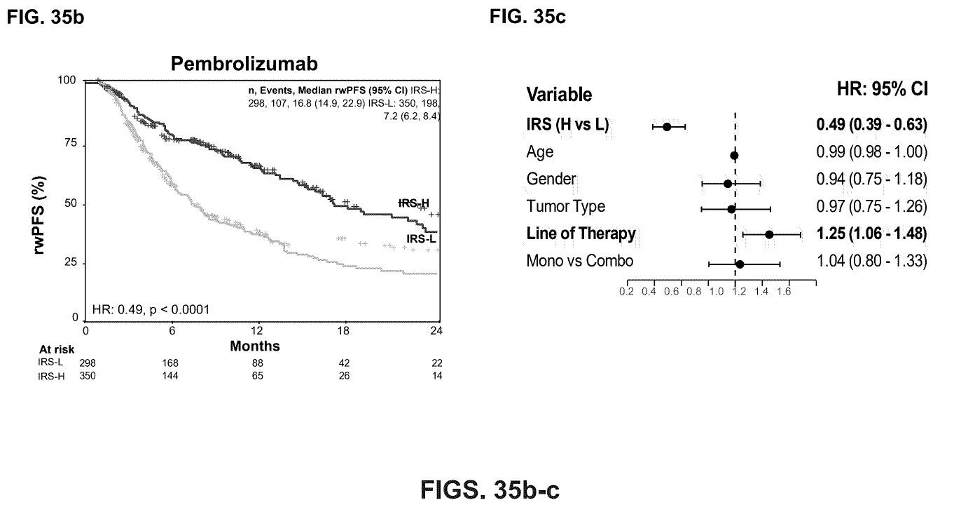

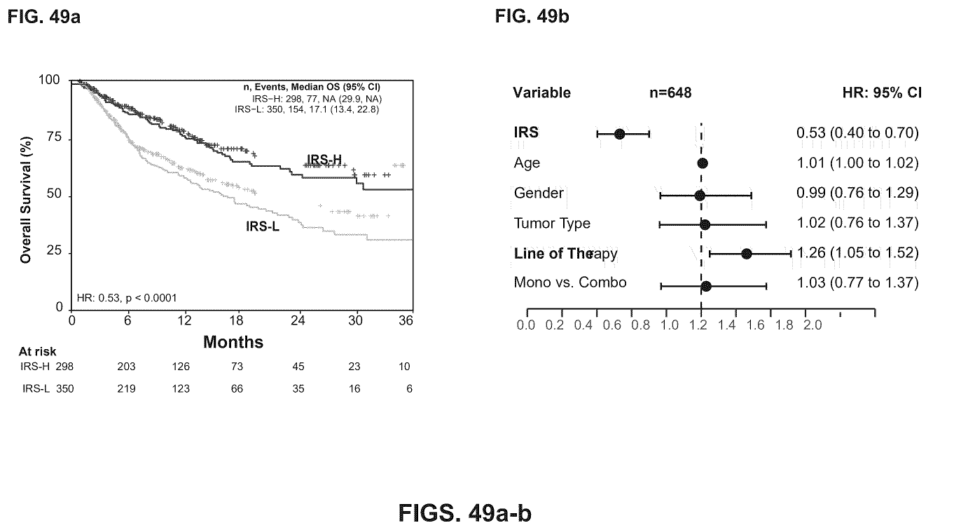

To evaluate the potential of the multivariate model to predict PD-1/PD-L1 blockade treatment outcome, we derived individual Immunotherapy Response Scores (IRS) from the final five variable model, assigned the 648 patients to either IRS-High [-H; n=298 (46.0%); associated with greater benefit of PD-1/PD-L1 blockade] or IRS-Low groups (threshold set by balancing maximization of IRS-H group size vs. minimization of the unadjusted rwPFS IRS hazard ratio), and compared group outcomes by Kaplan Meier analysis and Cox proportional hazards modeling after adjusting for age, gender, most frequent tumor type (NSCLC) vs. others, line type (monotherapy/combination therapy) and line of systemic therapy. As shown in FIGS. 35b&c, IRS-H patients had significantly longer pembrolizumab rwPFS (IRS-H vs. IRS-L median rwPFS 16.8 [95% CI 14.9-22.9] vs. 7.2 [95% CI 6.2-8.4] months, adjusted hazard ratio 0.49 [95% CI 0.39-0.63], p<0.0001) and OS (IRS-H vs. IRS-L median OS Not Reached [95% CI 29.9-NA] vs. 17.1 [95% CI 13.4-22.8] months, adjusted hazard ratio 0.53 [95% CI 0.40-0.70], p<0.0001; FIGS. 49a&b). IRS-H also showed significant rwPFS and OS benefit when using restricted mean survival time analysis (prespecified periods of 24 months and 36 months, respectively), both in an unadjusted analysis (IRS-H vs. IRS-L average event free rwPFS 15.70 [95% CI 14.53-16.88] vs. 10.63 [95% CI 9.61-11.65]; OS 25.50 [95% CI 23.61-27.39] vs. 19.24 [95% CI 17.48-21.00] and when adjusting for the same CPH model covariates above (rwPFS IRS-H vs. IRS-L 4.80 [95% CI 3.20-6.41], p<0.0001; OS IRS-H vs. IRS-L 6.00 [95% CI 3.37-8.63], p<0.0001). Table S6).

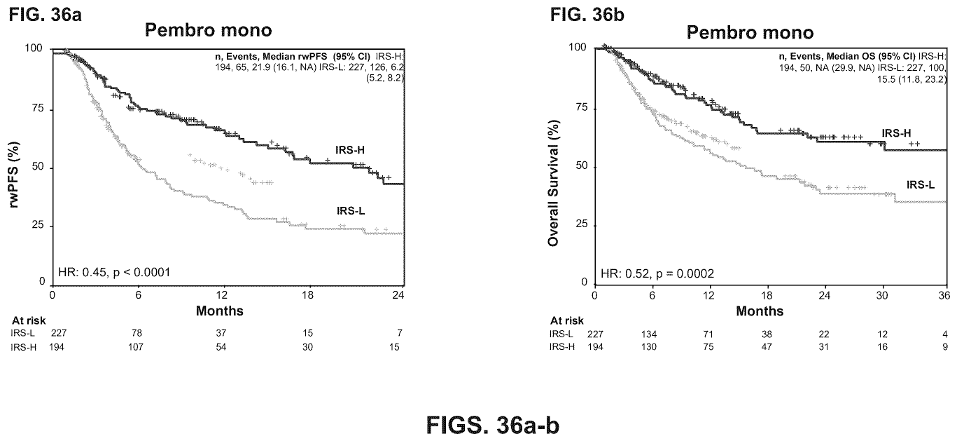

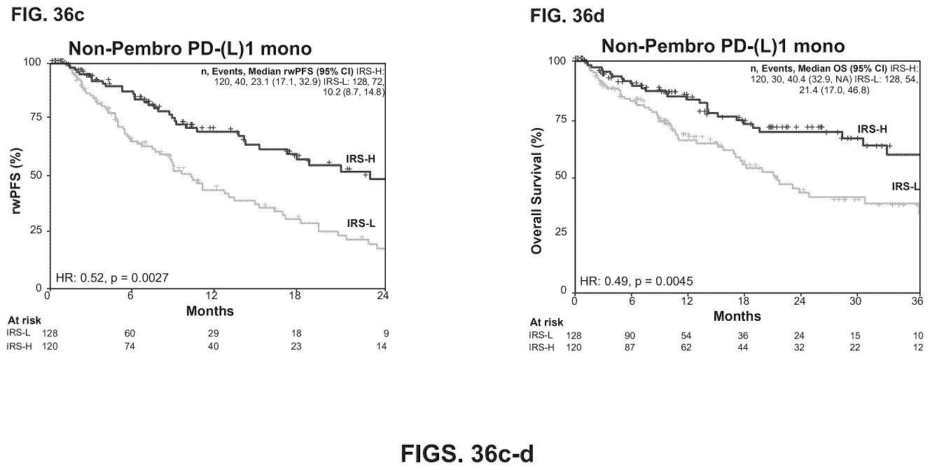

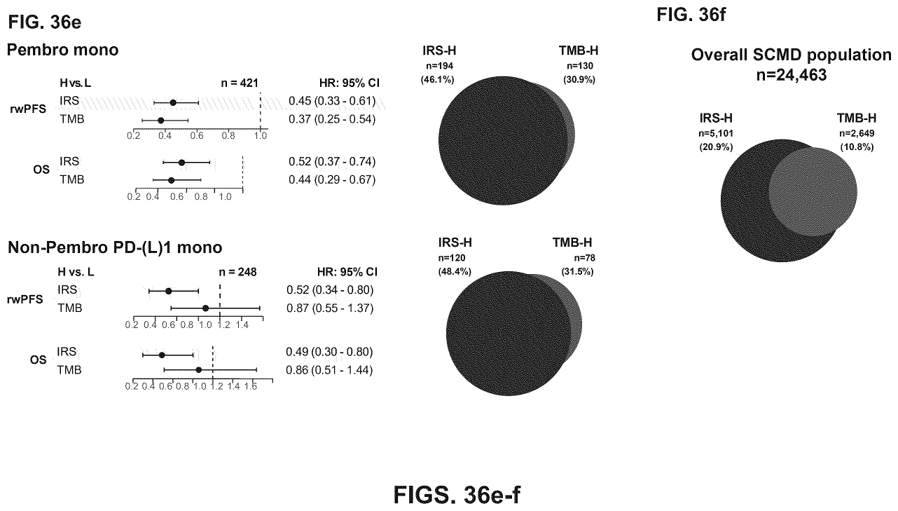

As PD-(L)1 combination therapy regimens vary across tumor types and there is little evidence of even additive benefit from currently approved PD-(L)1 combination regimens³³, while TMB has shown to be broadly predictive of monotherapy PD-(L)1 benefit²⁵⁻³², we also restricted results in the discovery cohort to just those patients treated with pembrolizumab monotherapy (n=421; 46.1% IRS-H). As shown in FIGS. 36a&b, IRS-H patients had significantly longer pembrolizumab rwPFS (IRS-H vs. IRS-L median rwPFS 21.9 [95% CI 16.1-NA] vs. 6.2 [95% CI 5.2-8.2] months, adjusted [as for the entire cohort except for line type] hazard ratio 0.45 [95% CI 0.33-0.61], p<0.0001) and OS (IRS-H vs. IRS-L median OS Not Reached [95% CI 29.9-NA] vs. 15.5 [95% CI 11.8-23.2] months, adjusted hazard ratio 0.52 [95% CI 0.37-0.74], p=0.0002).

### Validation of the Integrative IRS Model to Predict PD-1/PD-L1 Blockade Benefit

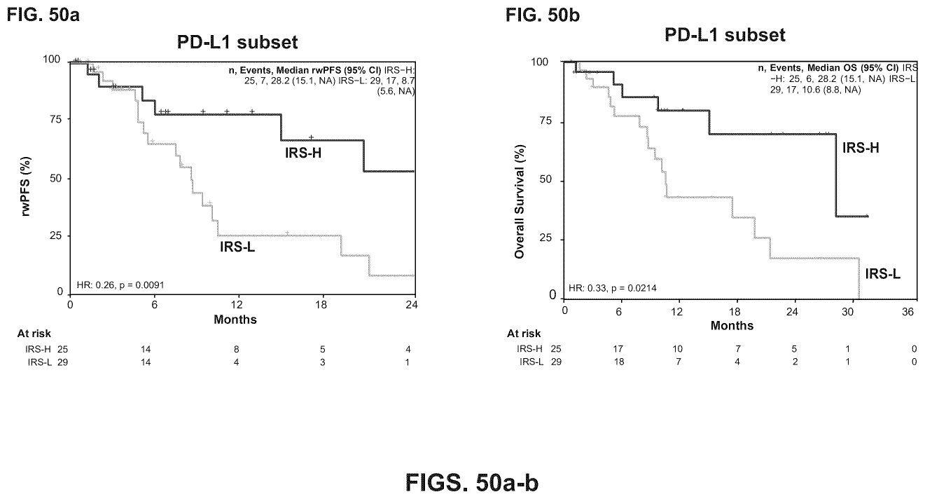

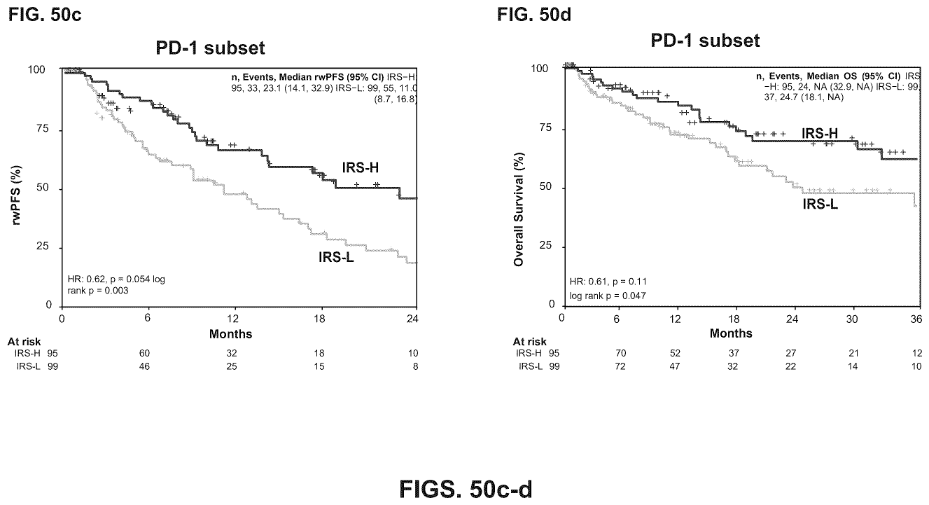

We next sought to validate the ability of IRS to predict PD-(L)1 monotherapy treatment outcome by both rwPFS and OS in an independent cohort. Based on a power analysis (see Methods) we identified a sufficient cohort of all 248 of the 9,899 (2.5%) eligible patients in the SCMD (valid TMB and gene expression with documented anti-neoplastic agent treatment) who met the same inclusion/exclusion criteria as the discovery cohort, except they were treated with systemic non-pembrolizumab anti-PD-(L)1 monotherapy (and were not in the discovery cohort). As shown in FIG. 35a, the PD-(L)1 monotherapy validation cohort (n=248; PD-1 n=194 [78%] and n=54 [22%] PD-L1) was comprised of patients with 24 tumor types (25% melanoma [most frequent tumor type]); tumor types and demographics are provided in Table S1&S2. All patients in the validation cohort were assigned to IRS-H or IRS-L groups using the locked IRS model (48.4% IRS-H), and group outcomes were compared after adjusting as for the discovery monotherapy analysis (except adding therapy type [PD-1 vs. PD-L1] as a covariate). As shown in FIGS. 36c&d, by Kaplan Meier analysis, IRS-H patients had significantly longer PD-(L)1 monotherapy rwPFS (IRS-H vs. IRS-L median rwPFS 23.1 [95% CT 17.1-32.9] vs. 10.2 [95% CT 8.7-14.8] months, adjusted hazard ratio=0.52 [95% CI 0.34-0.80], p=0.003) and OS (IRS-H vs. IRS-L median OS 40.4 [95% CI 32.9-NA] vs. 21.4 [95% CI 17.0-46.8] months, adjusted hazard ratio=0.49 [95% CI 0.30-0.80], p=0.005) compared to IRS-L patients. As described in the Supplementary Results and shown in FIGS. 50a-d, results were similar when stratifying patients by PD-1 vs. PD-L1 therapy. Taken together, these results demonstrate the development and validation of an integrative, DNA and RNA based predictor of PD-(L)1 blockade benefit, with IRS-H patients showing significantly longer rwPFS and OS in an independent validation cohort.

### Comparison of IRS to TMB and Emerging Single Gene Biomarkers for Predicting PD-1/PD-L1 Blockade Benefit

As described above, TMB has been shown to predict both monotherapy PD-1 (pembrolizumab and nivolumab) and PD-L1 (atezolizumab) benefit through both retrospective and prospective studies, although ORRs at the same TMB cutoff vary across agents and TMB cutoffs. Hence, although quantitative TMB is a component of the IRS model, both TMB and IRS are reported as binary predictors (given the near requirement of categorical biomarkers for clinical implementation), therefore, to have clinical utility, the IRS model should identify a population of patients at least as large as the TMB-H population with similar PD-(L)1 benefit. As shown in FIG. 36e, in the 421-patient monotherapy treated subset of the discovery pembrolizumab cohort, 194 (46.1%) and 130 (30.9%) patients were identified as IRS-H and TMB-H, respectively, while in the 248-patient validation cohort, 120 (48.4%) and 78 (31.5%) patients were identified as IRS-H and TMB-H, respectively. In the pembrolizumab cohort, by Cox proportional hazards analysis, both categorical TMB (TMB-H vs. TMB-L) and IRS (IRS-H vs. IRS-L) were significant predictors of pembrolizumab monotherapy rwPFS (TMB-H vs. TMB-L adjusted hazard ratio 0.37 [95% CI 0.25-0.54], p<0.0001; IRS-H vs. IRS-L adjusted hazard ratio 0.45 [95% CI 0.33-0.61], p<0.0001) and OS (TMB-H vs. TMB-L adjusted hazard ratio 0.44 [95% CI 0.29-0.67]; IRS-H vs. IRS-L adjusted hazard ratio 0.52 [95% CI 0.37-0.74]; FIG. 36e) in models separately adjusted for IRS and TMB. However, in the validation cohort, IRS, but not TMB, was an independent predictor of PD-(L)1 rwPFS (TMB-H vs. TMB-L adjusted hazard ratio 0.87 [95% CI 0.55-1.37], p=0.54; IRS-H vs. IRS-L adjusted hazard ratio 0.52 [95% CI 0.34-0.80], p=0.003) and OS (TMB-H vs. TMB-L adjusted hazard ratio 0.86 [95% CI 0.51-1.44], p=0.56; IRS-H vs. IRS-L adjusted hazard ratio 0.49 [95% CI 0.30-0.80], p=0.005, FIG. 36e) in models separately adjusted for IRS and TMB (Kaplan-Meier plots of rwPFS and OS stratified by TMB status are shown in FIGS. 45c&d). As shown in FIG. 36f, across 24,463 Strata Trial samples in the SCMD with informative TMB and gene expression (regardless of treatment data availability), the overall IRS-H population was nearly twice as large as the TMB-H population (20.9% vs. 10.8%).

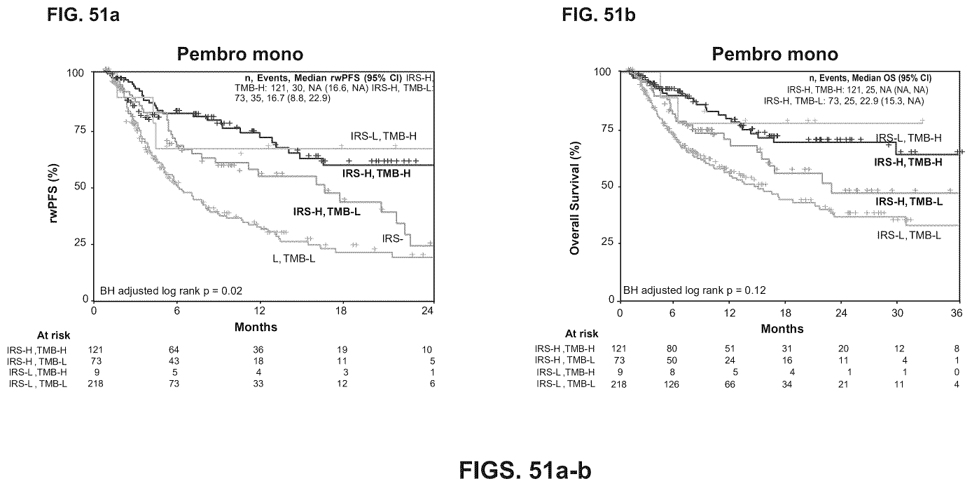

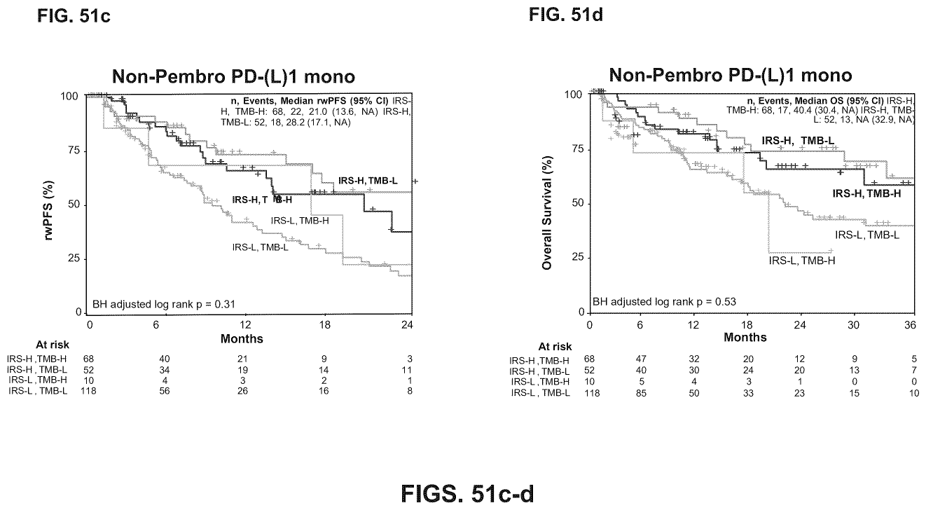

As shown in FIGS. 51a&b, by Kaplan-Meier analysis of the pembrolizumab monotherapy cohort stratified by IRS and TMB status, while median rwPFS was significantly longer in IRS-H/TMB-H vs. IRS-H/TMB-L (median rwPFS Not reached [95% CI 16.6-NA] vs. 16.7 [95% CI 8.8-22.9] months, pairwise log-rank with Benjamini Hochberg adjusted p=0.02), median OS was not significantly different between IRS-H/TMB-H vs. IRS-H/TMB-L patients (median rwPFS Not reached [95% CI NA-NA] vs. 22.9 [95% CI 15.3-NA] months, pairwise log-rank with Benjamini Hochberg adjusted p=0.12). As shown in FIGS. 51c&d, in the validation cohort, neither median PFS nor OS significantly differed between IRS-H/TMB-H vs. IRS-H/TMB-L patients (IRS-H/TMB-H vs. IRS-H/TMB-L median rwPFS 21.0 [95% CI 13.6-NA] vs. 28.2 [95% CI 17.1-NA] months, pairwise log-rank with Benjamini Hochberg adjusted p=0.31; median OS 40.4 [95% CI 30.4-NA] vs. Not Reached [95% CI 32.9-NA] months, pairwise log-rank with Benjamini Hochberg adjusted p=0.53). Of interest, in both the discovery and validation cohort (and overall SCMD population as described below), only a small minority of patients were IRS-L/TMB-H (2.1% and 4.0% of the discovery [monotherapy] and validation cohorts, respectively), and hence their benefit from PD-(L)1 monotherapy is unclear.

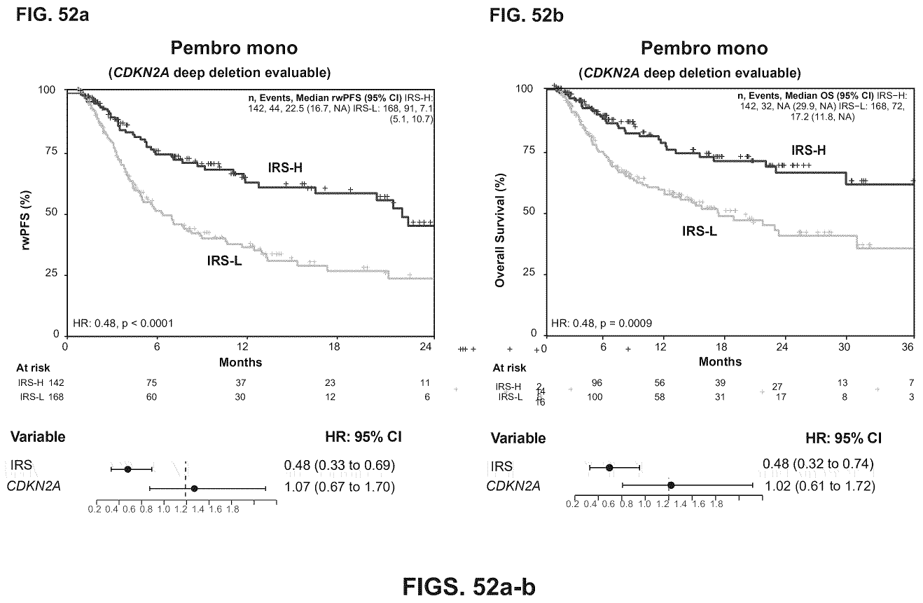

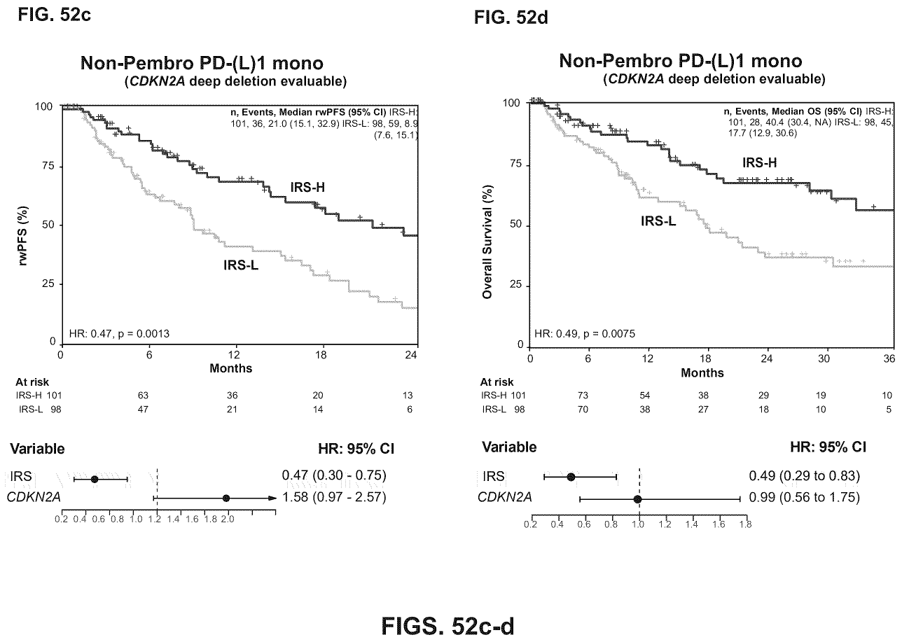

Despite the fundamental limitations of all genomic biomarkers (including TMB) for predicting PD-(L)1 therapy response, multiple single genes have also been identified from translational research studies that may add to the ability of TMB and/or PD-L1 IHC to predict PD-(L)1 benefit⁶⁰⁻⁶⁸. Two recent reports have suggested that CDKN2A deep deletion (homozygous loss) status may improve upon TMB alone for predicting monotherapy PD-(L)1 benefit^{60,68}. Hence, we assessed whether the inclusion of CDKN2A deep deletion status was an independent predictor of monotherapy PD-(L)1 benefit by adjusted Cox proportional hazards modeling in the subset of patients with valid CDKN2A deep deletion status (the StrataNGS limit of detection for deep deletions is 40% tumor content) from the discovery (n=310 [47.8%] of 648 after also excluding those treated with combination therapy) and validation (n=199 [79.9%] of 249) cohorts. As shown in FIGS. 52a-d, IRS status, but not CDKN2A deep deletion status, was a significant predictor of rwPFS and OS in both the discovery (rwPFS IRS-H vs. -L adjusted hazard ratio 0.48 [95% CI 0.33-0.69], p<0.0001; rwPFS CDKN2A deep deletion vs. CDKN2A wt adjusted hazard ratio 1.07 [95% CI 0.67-1.70], p=0.78; OS IRS-H vs. -L adjusted hazard ratio 0.48 [95% CI 0.32-0.74], p=0.0009; OS CDKN2A deep deletion vs. CDKN2A wt adjusted hazard ratio 1.02 [95% CI 0.61-1.72], p=0.94) and validation cohorts (rwPFS IRS-H vs. -L adjusted hazard ratio 0.47 [95% CI 0.30-0.75], p=0.001; rwPFS CDKN2A deep deletion vs. CDKN2A wt adjusted hazard ratio 1.58 [95% CI 0.97-2.57], p=0.07; OS IRS-H vs. -L adjusted hazard ratio 0.49 [95% CI 0.29-0.83], p=0.008; OS CDKN2A deep deletion vs. CDKN2A wt adjusted hazard ratio 0.99 [95% CI 0.56-1.75], p=0.96) cohorts when CDKN2A deep deletion status was added to the appropriate adjusted Cox proportional hazards model, confirming the limitations of genomic markers alone for predicting PD-(L)1 therapy response.

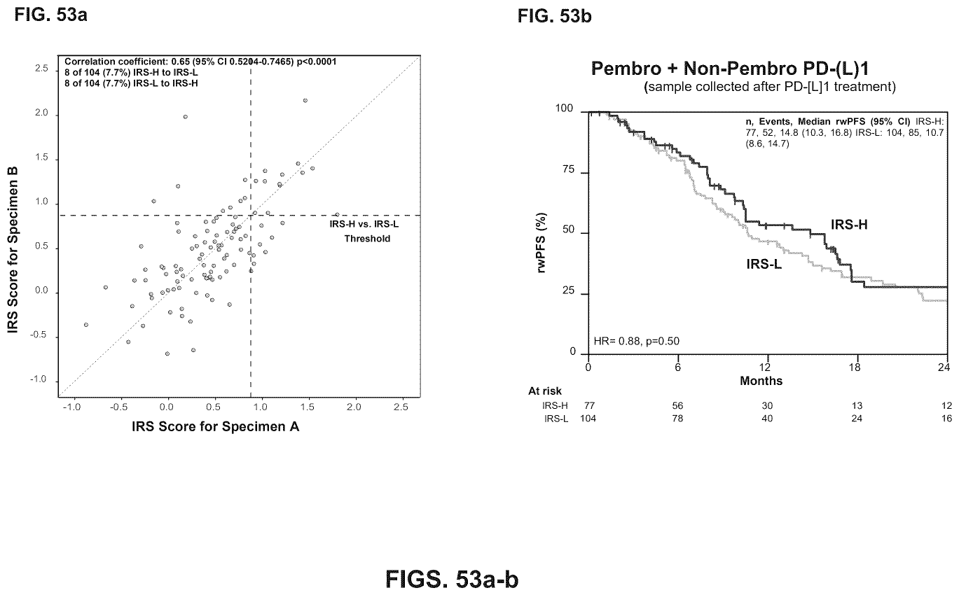

Taken together, these results demonstrate that in both the discovery and independent validation cohorts, IRS identifies a larger proportion of patients than TMB alone with similar benefit from PD-(L1) therapy, establishing clinical utility of the IRS biomarker and demonstrating the value of integrating quantitative gene expression with TMB for predicting PD-1/PD-L1 monotherapy treatment benefit. Additional analyses supporting the robustness of the IRS model to temporal sample collection (prior to CPI treatment) and variable tumor content are described in the Supplementary Results and FIGS. 53a-b & 54a-e.

### Confirmation of the Predictive Nature of IRS

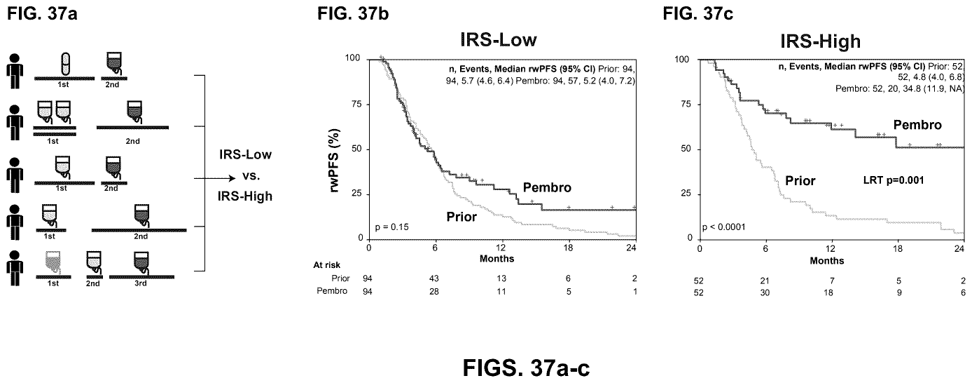

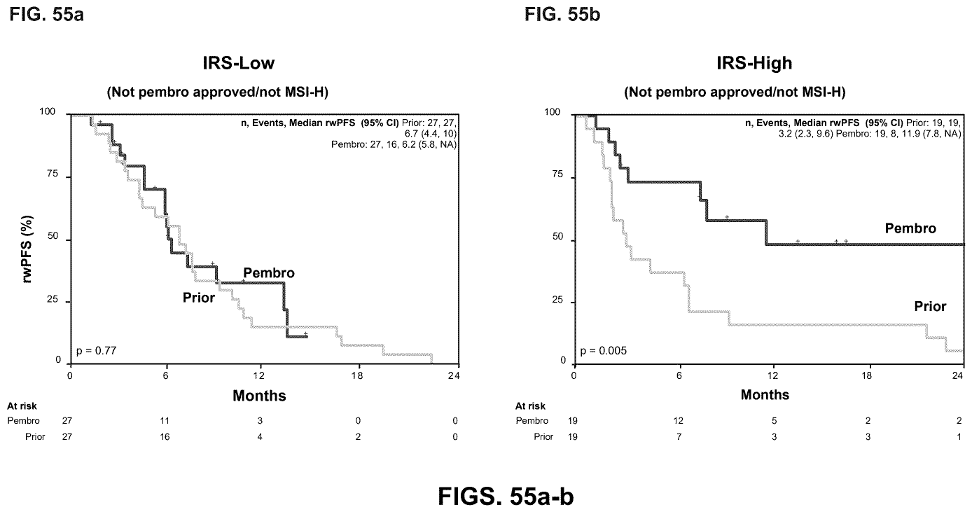

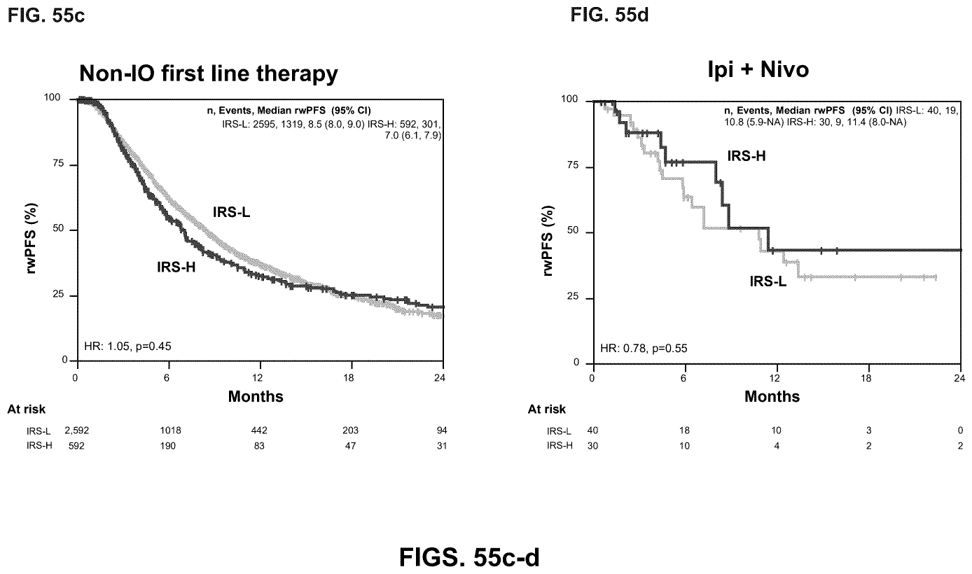

To establish the IRS model as predictive and not prognostic, we first assessed an internal comparator cohort for the pembrolizumab monotherapy cohort, consisting of the 146 of 648 (22.5%) of patients who had received a previous line of systemic therapy prior to pembrolizumab monotherapy (demographics and therapy types are shown in Table S7). For each patient, rwPFS was determined for the line of systemic therapy immediately preceding pembrolizumab and the pembrolizumab monotherapy line, with rwPFS stratified by IRS status assessed by Kaplan Meier analysis (FIG. 37a). While pembrolizumab monotherapy compared to the immediately preceding therapy line rwPFS did not significantly differ in IRS-L patients (IRS-L pembrolizumab vs. immediately preceding therapy median rwPFS 5.2 [95% CI 4.0-7.2] vs. 5.7 [95% CI 4.6-6.4] months, log-rank p=0.15; FIG. 37b), pembrolizumab rwPFS was significantly longer than the immediately preceding therapy line in IRS-H patients (IRS-H pembrolizumab vs. immediately preceding therapy median rwPFS 34.8 [95% CI 11.9-NA] vs. 4.8 [95% CI 4.0-6.8] months, log-rank p<0.0001; FIG. 37c). The test for interaction (models shown in Table S8) between pembrolizumab vs. immediately preceding treatment line and IRS status (IRS-H vs. IRS-L) was significant (likelihood ratio test for interaction p=0.001). Notably, when this analysis was restricted to the 46 patients with non-MSI-H (StrataNGS clinical testing) tumors in non-PD(L)1 monotherapy approved tumor types, only IRS-H patients still had significantly longer pembrolizumab monotherapy rwPFS than the immediately preceding line of therapy (IRS-H pembrolizumab vs. immediately preceding therapy median rwPFS 11.9 [95% 7.8-NA] vs. 3.2 [95% CI 2.3-9.6] months, log rank, p=0.005; FIGS. 55a&b). Taken together, these results confirm the predictive nature of the IRS biomarker across tumor types.

As only 76 subjects in the validation cohort received PD-(L)1 blockade therapy in at least the second line, we instead leveraged the compendium of treatment data across SCMD patients not included in the discovery or validation cohorts to further confirm the predictive nature of the IRS biomarker. Across all 3,184 patients in the SCMD (n=592 IRS-H) with a non-PD-(L)1 or CTLA4 systemic therapy first line who otherwise met criteria for the discovery and validation cohorts, IRS status was not a significant predictor of non-PD-(L)1 (or CTLA4 systemic therapy rwPFS by Cox proportional hazards modeling when adjusting for age, gender, most common tumor type (colorectal cancer) vs. others, and monotherapy vs. combination therapy (IRS-H vs. -L median rwPFS 7.0 [95% CI 6.1-7.9] vs. 8.5 [95% CI 8.0-9.0] months, adjusted hazard ratio 1.05 [95% CI 0.92-1.19], p=0.45), confirming the predictive nature of the IRS model (FIG. 55c). Next, given the mechanistic differences between CTLA4 and PD-(L)1 blockade, and the lack of additive or synergistic treatment effect between these agents in melanoma³³, we assessed the ability of IRS to stratify combined ipilimumab+nivolumab (CTLA4+PD-1) benefit in a cohort of 70 patients (n=30 IRS-H) who otherwise would have been eligible for the validation cohort but received combined ipilimumab+nivolumab treatment (8 tumor types; 47% melanoma). As shown in FIG. 55d, after adjusting for age, gender, most common tumor type (melanoma) vs. others, and line of therapy, combined ipilimumab+nivolumab rwPFS (IRS-H vs. IRS-L median rwPFS 11.4 [95% CI 8.4-NA] vs. 10.8 [95% CI 5.9-NA] months, adjusted hazard ratio 0.78 [95% CI 0.34-1.76], p=0.55) did not significantly differ by IRS status.

### Exploratory Analysis of IRS In First-Line NSCLC Patients

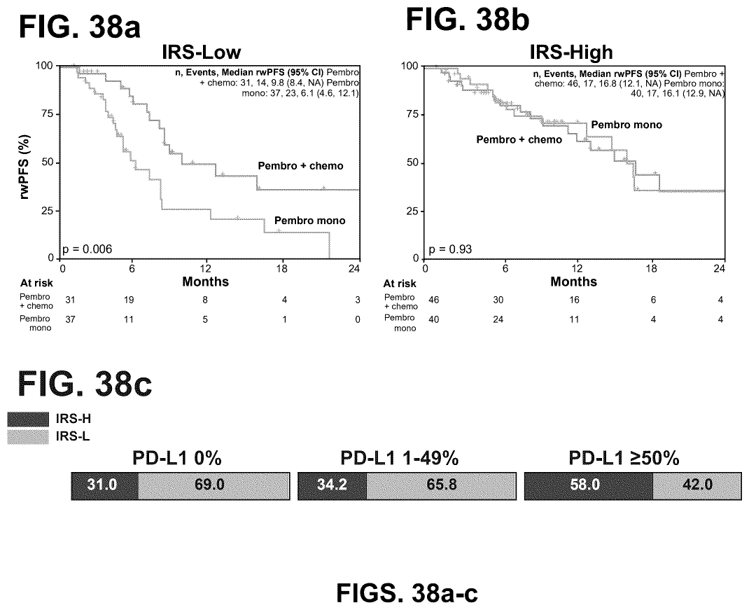

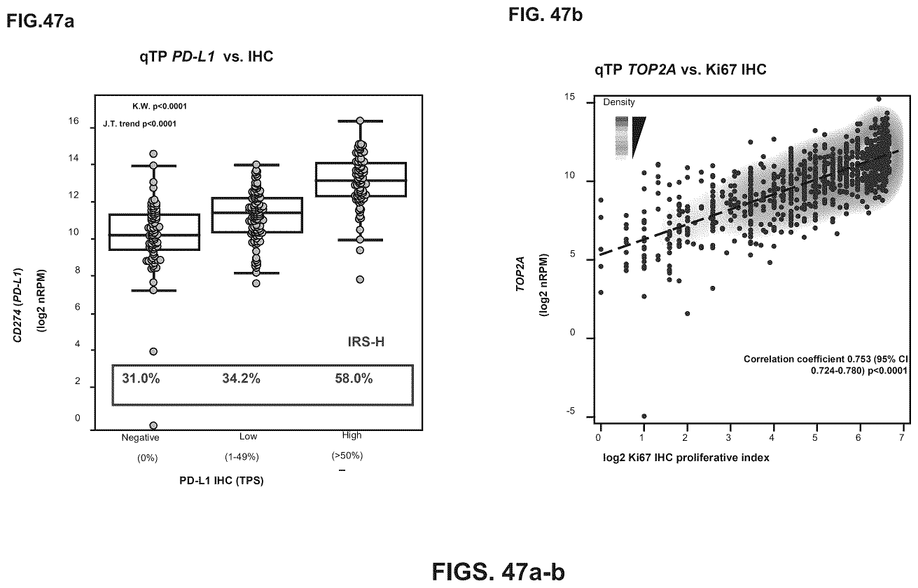

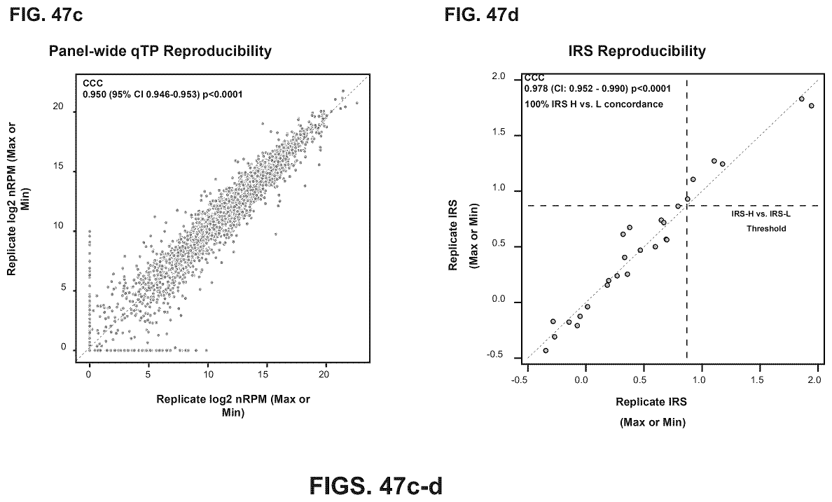

As described above, despite little, if any, evidence for additive or synergistic benefit of PD-(L)1 and other agents in approved combination regimens, PD(L)-1 combination regimens are rapidly being developed and moved to earlier lines of therapy, highlighting the need for improved biomarkers that can predict PD-(L)1 monotherapy benefit. For example, in 1^{st} line advanced NSCLC, both monotherapy pembrolizumab and pembrolizumab+chemotherapy are approved for patients with PD-L1 IHC (TPS)≥1% and ≥50%, however prospective data is not available to guide monotherapy vs. combination therapy decision making. Hence, in the pembrolizumab cohort, we identified 242 patients with NSCLC who were treated with 1^{st} line systemic pembrolizumab monotherapy (n=109) or pembrolizumab+chemotherapy (n=133; FIG. 40). Although this cohort is limited by a lack of PD-L1 TPS data, IRS includes qTP expression of PD-L1, and we have validated the accuracy of this individual transcript vs. TPS in NSCLC FFPE tumor samples (FIGS. 47a-d). Consistent with both TPS and performance status largely driving the monotherapy vs. combination therapy treatment decision, we confirmed that monotherapy treated patients were significantly older and had higher PD-L1 qTP expression compared to combination therapy treated patients (Table S9). Hence, we performed propensity score matching (see Methods) between the monotherapy and combination therapy groups using patient age, PD-L1 qTP expression, TMB, gender and IRS, which after excluding 88 unmatchable patients resulted in a final cohort of 154 patients (77 patients in each group) without significant differences in any of these variables (Table S9). As shown by Kaplan Meier analysis of the matched cohorts, in IRS-L patients, rwPFS was significantly shorter in those treated with monotherapy vs. combination therapy (median rwPFS 6.1 [95% CI 4.6-12.1] vs. 9.8 [95% CI 8.4-NA] months, log rank p=0.006; FIG. 38a). In contrast, in IRS-H patients, rwPFS was not significantly different in those treated with monotherapy vs. combination therapy (median rwPFS 16.1 [95% CI 12.9-NA] vs. 16.8 [95% CI 12.1-NA] months, log rank p=0.93; FIG. 38b). Taken together, these results support pembrolizumab monotherapy as a potentially reasonable treatment option for the 34% of patients with TPS scores 1-49% who are IRS-H (FIG. 38c & FIGS. 47a-d), consistent with a recent report assessing TMB across PD-L1 IHC strata in patients with 1^{st} line NSCLC treated with PD-(L)1 monotherapy²⁷, and more broadly suggests potential utility in identifying patients who may benefit from monotherapy PD-(L)1 vs. combination therapy in current indications.

### Pan Solid Tumor Distribution of IRS Groups

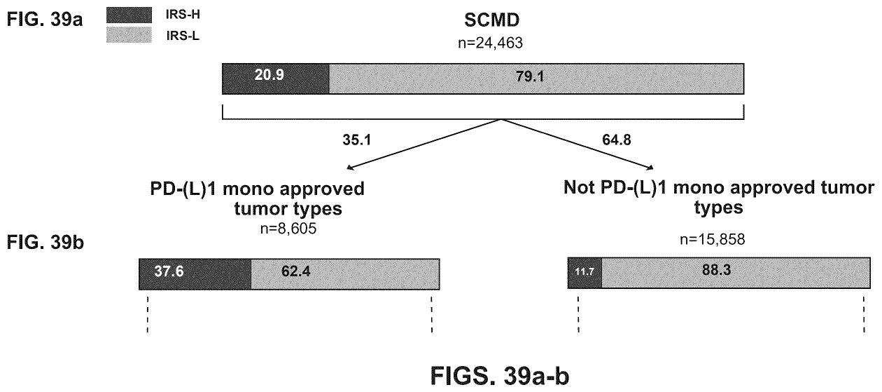

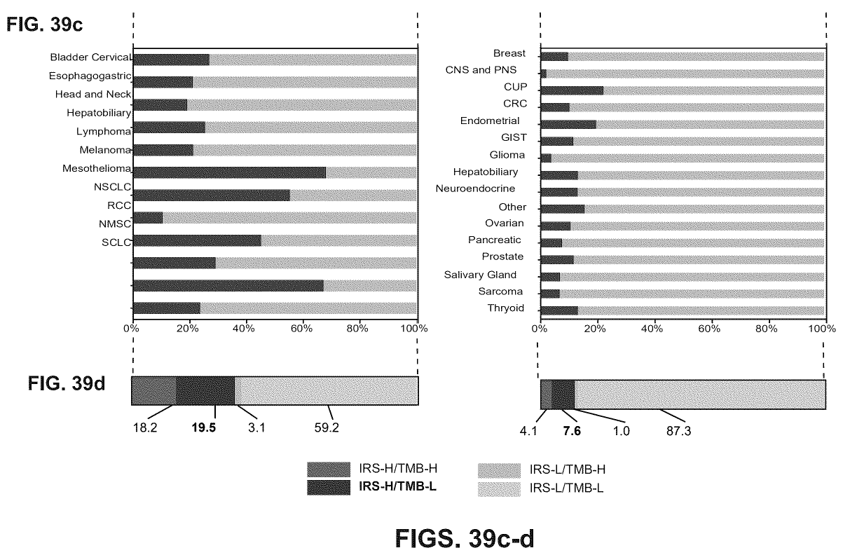

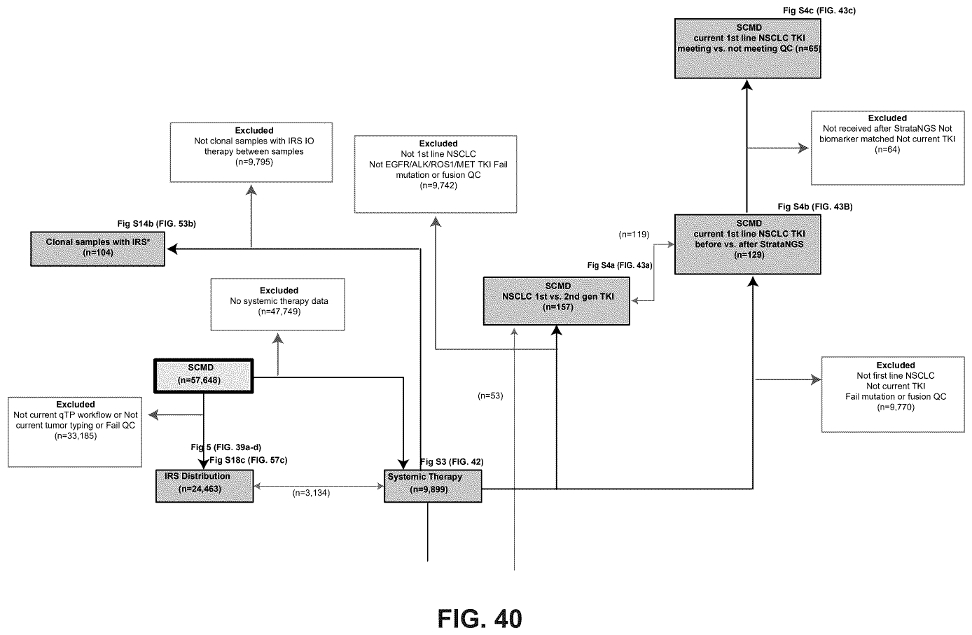

In both the discovery and separate validation cohorts, we demonstrated that IRS identifies a larger population of patients than TMB but with similar PD-(L)1 monotherapy benefit, however this analysis is limited by the requirement that patients received PD-1/PD-L1 treatment. Hence, we sought to leverage IRS distributions across tumor types (and pan-cancer biomarkers) in the entire SCMD to understand the potential impact of IRS both within and outside of currently approved PD-(L)1 monotherapy indications. Thus, we determined IRS for the 24,463 patients in the SCMD (NCT03061305) with informative TMB and gene expression data, with 20.9% and 79.1% of all patients classified as IRS-H and -L, respectively (FIG. 39a). PD-(L)1 monotherapy approved tumor types⁶⁹ (without consideration of PD-L1 IHC status) had a substantially higher proportion of IRS-H patients (37.6%) than non-PD-(L)1 monotherapy approved tumor types (11.7%) (FIG. 39b). Tumor types with the highest proportion of IRS-H group patients include several known to be highly responsive to PD-(L)1 therapy, including lymphoma, non-melanoma skin cancer, melanoma, NSCLC, and renal cell carcinoma (which nearly invariably has low TMB) (FIG. 39c).

We lastly examined the pan-solid tumor distribution of IRS groups by TMB status, given the pan-tumor approval of pembrolizumab in TMB-H tumors and prospective trials showing efficacy of other PD-(L)1 monotherapies patients with TMB-H. In both PD-(L)1 monotherapy approved and non-approved tumor types, the vast majority of TMB-H patients were also IRS-H (only 1.8% of overall patients were IRS-L/TMB-H [3.1% and 1.0% in approved and non-approved tumor types, respectively), however the overall IRS-H population was nearly twice as large as the TMB-H population (20.9% IRS-H vs. 10.8% TMB-H overall (FIG. 36f); 37.7% IRS-H vs. 22.6% TMB-H in approved tumor types and 11.7% IRS-H vs. 5.1% TMB-H in non-approved tumor types, respectively; FIG. 39d) with similar PD-(L)1 monotherapy benefit as established herein. Critically, this analysis demonstrates that 7.6% of patients in non-approved tumor types are IRS-H/TMB-L, representing a sizable population predicted to have benefit from PD-(L)1 monotherapy.

### Discussion

Leveraging a robust clinical molecular database from the Strata Trial (NCT03061305), herein we developed an integrative Immunotherapy Response Score (IRS) algorithm combining TMB and quantitative gene expression from simultaneously performed, clinically validated, multiplex PCR based DNA and RNA NGS (StrataNGS CGP and a separate RNA panel for quantitative transcriptomic profiling)⁵⁴⁻⁵⁶ to predict pembrolizumab (anti-PD-1) rwPFS (by time to next therapy) in 648 patients from 26 solid tumor types. We then validated the locked IRS model—which incorporates TMB and quantitative gene expression of PD-1, PD-L1, ADAM12 and TOP2A in a Cox proportional hazards model—and IRS-H vs. -L threshold (IRS-H as more likely to benefit) in an independent cohort of 248 patients from 24 solid tumor types treated with other PD-(L)1 monotherapies. In this validation cohort, IRS-H status was associated with significantly longer PD-(L)1 rwPFS (IRS-H vs. IRS-L median 23.1 vs. 10.2 months; adjusted hazard ratio 0.52, p=0.003) and OS (median OS 40.4 vs. 21.4 months, adjusted hazard ratio 0.49, p=0.005) when adjusted for age, gender, line of therapy, PD-1 vs. PD-L1 therapy, and tumor type. Notably, TMB alone was not a significant predictor of PD-(L)1 rwPFS, nor OS, in this cohort. When applied to all 24,463 patients in the SCMD where IRS could be generated, the IRS-H population was nearly twice the size of the TMB-H population (20.9 vs. 10.8%). While IRS-H was more frequent in tumor types known to derive benefit from PD-(L)1 therapy, IRS-H occurred in subsets of nearly every tumor type. Most importantly, among TMB-L patients in tumor types without approved PD-(L)1 monotherapy, 7.6% were IRS-H (a potentially conservative estimate as many approved indications have PD-L1 IHC requirements), representing a substantial population of patients with advanced solid tumor who could immediately benefit from PD-(L)1 monotherapy treatment.

We confirmed the predictive nature of the IRS biomarker through multiple approaches. Most importantly, in the subset of pembrolizumab monotherapy treated patients who had at least one prior line of systemic therapy, we confirmed the predictive nature of the IRS model, as IRS-H patients had significantly longer rwPFS on pembrolizumab vs. their immediately preceding systemic therapy, with a significant test for interaction between IRS and pembrolizumab vs. prior therapy. Likewise, IRS status was not significantly associated with first line rwPFS in >3,000 patients treated with non-immunotherapy. Although the association of IRS with PD-(L)1 rwPFS and OS were similar in the discovery (pembrolizumab) and validation (non-pembrolizumab PD-(L)1 monotherapy) cohorts and was largely insensitive to tumor type, TMB status, and pre- vs. post-non CPI therapy sample collection—suggesting that the model captures universal biological features of PD-(L)1 monotherapy benefit—the model was less predictive in patients treated with pembrolizumab combination therapy (combination and monotherapy pembrolizumab rwPFS adjusted hazard ratio 0.60 [95% CI 0.41-0.89], p=0.01 and 0.45 [95% CI 0.33-0.61], p<0.0001, respectively) and did not significantly predict combination PD-1+CTLA4 (nivolumab+ipilumumab) benefit. These results suggest that while different approaches are likely needed to best predict combination therapy (or monotherapy of the non-IO component) responses, particularly in light of a recent meta-analysis demonstrating that there is little evidence for synergy between CPIs and other agents in approved combination regimens³³, the IRS model can likely identify patients expected to benefit from PD-(L)1 monotherapy in settings where only combination (or both monotherapy and combination) PD-(L)1 therapy is indicated. In support of this, in an exploratory analysis on a propensity score matched cohort of patients with NSCLC treated with first line systemic pembrolizumab monotherapy vs. combination chemotherapy, while rwPFS was significantly worse in IRS-L patients treated with monotherapy vs. combination chemotherapy, no significant difference in rwPFS was present in IRS-H patients treated with monotherapy vs. combination chemotherapy. In a separate cohort of patients with NSCLC, we demonstrate that approximately one third of those with PD-L1 IHC TPS 1-49% (where the monotherapy vs. combination chemotherapy decision is most relevant) are IRS-H, suggesting potential utility in identifying those patients most likely to benefit from pembrolizumab monotherapy alone.

Current FDA-approved PD-(L)1 biomarkers include PD-L1 IHC, TMB and MSI-H (the latter indication was initially approved without a companion diagnostic biomarker), however these biomarkers have several practical challenges for clinical use including variations in assay parameters, platforms, and predictive thresholds^{4,70-73}. For example, although there are multiple tissue TMB assays commercially available (LDTs, FDA cleared devices, and a single FDA approved companion diagnostic device), TMB testing typically has a large tissue requirement, which is frequently not feasible in patients with advanced cancers, and such approaches do not allow for parallel clinical assessment of gene expression biomarkers. Likewise, liquid biopsy based TMB is not directly translatable to tissue TMB, even when both tissue and liquid biopsies are performed using FDA approved CGP devices, as in a recent study of both single agent nivolumab and nivolumab+ipilimumab combination therapy, where blood TMB's predictive ability was conditional on tissue TMB status, but not vice versa³². Hence, it is notable that our study herein used assays performed as part of routine clinical testing on co-isolated DNA and RNA, and are now integrated in a combined analytically and validated clinical CGP+qTP test with key sample input requirements defined from over 30,000 consecutively received FFPE tumor samples for CGP testing: ≥20% tumor content and 2 mm² tumor surface area (from 10×5 um FFPE sections)^{55,56}. Of note, only 37.5% and 43.5% of the discovery and validation cohort, respectively, and 35.5% of the 24,463 total patients in the SCMD used to assess IRS distribution, met the minimum tumor surface area requirements (≥25 mm²) of FoundationOne CDx⁷⁴, the FDA approved companion diagnostic device to identify TMB-H tumors for pembrolizumab treatment. Although outside the scope of the current manuscript, integration of clinically validated qTP also has clinical utility outside of immunotherapy treatment decision making for patients with advanced solid tumors (FIGS. 46a-f), however detailed discussion is outside the scope of the current manuscript.

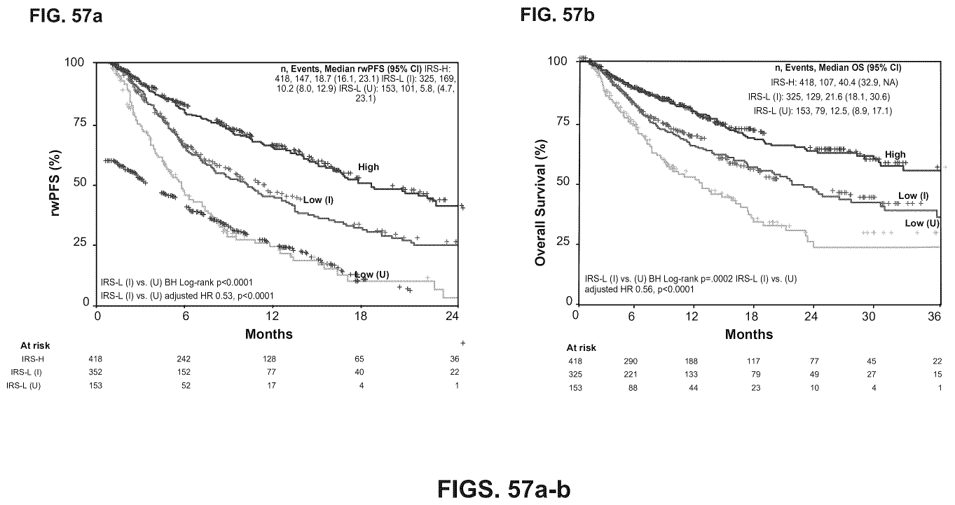

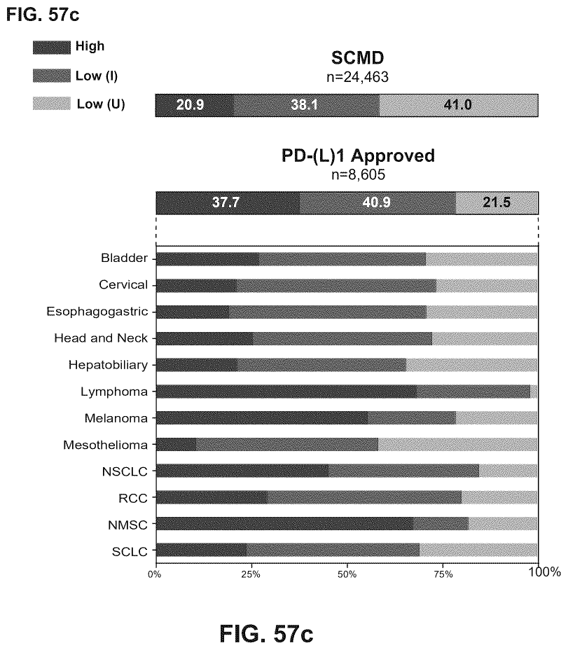

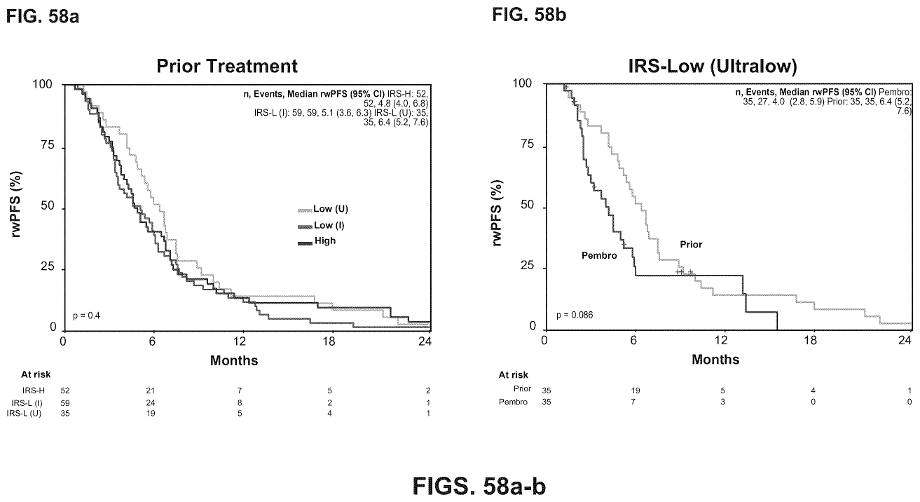

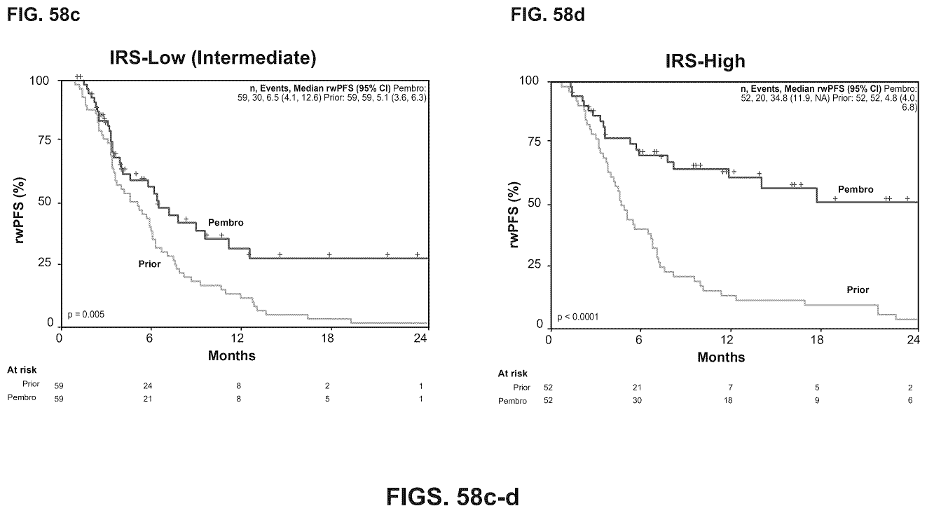

Our analysis has several potential limitations. First, our real-world Strata Trial treatment dataset was biased toward tumor types for which PD-(L)1 therapy is indicated, and thus, as expected, was enriched for patients benefiting from PD-(L)1 therapy. Indeed, the proportion of IRS-H patients was greater in the discovery and validation cohorts (combined 46.7%) than the broader Strata Trial profiling dataset (20.9%). However, patients with more than 20 tumor types were included in both the discovery and validation cohorts, and these cohorts consisted of both pembrolizumab and other non-pembrolizumab PD-(L)1 monotherapy-treated patients respectively. Additionally, we confirmed the predictive nature IRS in the >1^{st} line, off label (non-MSI-H, non-approved tumor types) population, similar to the pivotal study of pembrolizumab in the TMB-H population.²⁶ Second, the rwPFS endpoint includes some patients who stopped treatment due to treatment toxicity (not assessable herein) or switching therapy to a more appropriate regimen based on molecular results (as described in the Supplementary Results) and not disease progression, although this likely represents a minority of events, and both rwPFS and OS results were highly similar in both the discovery and validation cohorts. Additionally, although we developed and validated the IRS model across patients treated with multiple PD-(L)1 monotherapies and tumor types, not all solid tumor types were represented in these analyses and prospective studies could determine if more optimized thresholds (or further stratification beyond two IRS groups) may improve performance in specific tumor types or better predict PD-1 vs. PD-L1 therapy benefit. Of note, IRS had essentially similar predictive ability in both the training and validation cohorts (Table S10) when the tumor type term in our adjusted models (most common tumor type vs. others) was replaced with a term using MSKCC defined TMB sensitive vs. insensitive tumor types (MSI-H, POLE^{mutant}, NSCLC, head and neck cancer or melanoma as sensitive; all others as insensitive⁵⁷), supporting the more pan-solid tumor nature of IRS vs. TMB alone. Likewise, although we showed that the inclusion of CDKN2A copy loss, which has been identified in two studies as improving upon TMB status for predicting PD-(L)1 response^{60,68}, was not a significant predictor of PD-(L)1 rwPFS or OS in either the discovery or validation cohorts, future studies will be required to determine whether inclusion of other single gene-based DNA biomarkers identified as potentially predictive in one or more tumor types (e.g. STK11, PBRM1, ARID1A)⁶⁰⁻⁶⁸ or additional immune related genes assessed on the current expanded qTP panel can improve the performance of the IRS model; given the clearly established clinical utility for MSI-H status, this biomarker was not included in IRS model development. Limited PD-L1 IHC data was available for subjects in the SCMD with PD-(L)1 treatment outcomes, and hence we are not able to directly compare performance of IRS and PD-L1 IHC (or other immunotherapy response biomarkers beyond TMB), which is particularly relevant for our exploratory analysis of pembrolizumab monotherapy vs. combination therapy in first line NSCLC, however we used propensity score matching by PD-L1 qTP expression to mitigate this limitation. Notably, we chose to use standard multivariate regression with a minimum number of variables versus other approaches that have included a larger number of immune related genes^{34,41,46} or used more advanced machine learning approaches⁷⁵ to leverage the highly quantitative nature of CGP+qTP and minimize the risk of overfitting and additional biological insights derived from the IRS model are described in the Supplementary Discussion. Importantly, although demonstration that IRS predicts PD-(L)1 monotherapy rwPFS and OS at least as well as TMB in both the discovery and independent validation cohorts establishes clinical utility in the 7.6% of IRS-H/TMB-L patients outside of currently approved PD-(L)1 monotherapy indications, additional studies will be required to establish the clinical utility of IRS-H in patients with conflicting biomarker results (e.g. IRS-H/TMB-L) or where both monotherapy and combination therapy are indicated (e.g. IRS-H in PD-L1 IHC 1-49%). Likewise, in a post-hoc, exploratory analysis in the combined discovery and validation cohorts, we identified an ultra-low subset of the IRS-L population that shows particularly poor PD-(L)1 rwPFS and OS (FIGS. 57a-c & FIGS. 59a-d), suggesting that it may be possible to identify patients more likely to benefit from other therapies in PD-(L)1 approved tumor types when therapeutic choice is present.

In summary, using treatment data and molecular profiling from nearly 900 patients in the Strata Trial, a large observational trial of patients with advanced cancer, we report the development and validation of IRS, a biologically rational, integrative predictor of pan-solid tumor PD-(L)1 monotherapy benefit (by both rwPFS and OS) across solid tumors that identifies a population that is nearly twice as large as TMB-H alone with similar PD-(L)1 monotherapy benefit. Importantly, IRS was developed and validated using a single, clinically validated NGS platform capable of simultaneously performing CGP (required for TMB but also for assessing non-immunotherapy treatment biomarkers) and simultaneous, precise quantification of tumor- and tumor microenvironment (TME-relevant gene expression from minute FFPE tumor specimens. In addition to potential utility of IRS for refining treatment decisions in patients with approved PD-(L)1 indications, we show that across the >20,000 patient Strata Trial population with evaluable IRS status, 7.6% of patients with tumor types not approved for PD-(L)1 monotherapy were IRS-H/TMB-L-a population shown herein to have similar or better PD-(L)1 benefit as TMB-H-markedly expanding the benefit of immunotherapy across solid tumors by addressing one of the most important challenges in precision oncology.

### REFERENCES

1 Sharma, P. et al. The Next Decade of Immune Checkpoint Therapy. Cancer Discov 11, 838-857, doi:10.1158/2159-8290.CD-20-1680 (2021).
2 Chamoto, K., Hatae, R. & Honjo, T. Current issues and perspectives in PD-1 blockade cancer immunotherapy. Int J Clin Oncol 25, 790-800, doi:10.1007/s10147-019-01588-7 (2020).
3 Ribas, A. & Wolchok, J. D. Cancer immunotherapy using checkpoint blockade. Science 359, 1350-1355, doi:10.1126/science.aar4060 (2018).
4 Doroshow, D. B. et al. PD-L1 as a biomarker of response to immune-checkpoint inhibitors. Nat Rev Clin Oncol 18, 345-362, doi:10.1038/s41571-021-00473-5 (2021).
5 Gavrielatou, N., Shafi, S., Gaule, P. & Rimm, D. L. PD-L1 Expression Scoring: Non-Interchangeable, Non-Interpretable, Neither, or Both. J Natl Cancer Inst, doi:10.1093/jnci/djab109 (2021).
6 Rimm, D. L. et al. A Prospective, Multi-institutional, Pathologist-Based Assessment of 4 Immunohistochemistry Assays for PD-L1 Expression in Non-Small Cell Lung Cancer. JAMA Oncol 3, 1051-1058, doi:10.1001/jamaoncol.2017.0013 (2017).
7 Salgado, R. et al. How current assay approval policies are leading to unintended imprecision medicine. Lancet Oncol 21, 1399-1401, doi:10.1016/S1470-2045(20)30592-1 (2020).
8 Herbst, R. S. et al. Atezolizumab for First-Line Treatment of PD-L1-Selected Patients with NSCLC. N Engl J Med 383, 1328-1339, doi:10.1056/NEJMoa1917346 (2020).
9 Abdul Karim, L., Wang, P., Chahine, J. & Kallakury, B. Harmonization of PD-L1 Immunohistochemistry Assays for Lung Cancer: A Working Progress. J Thorac Oncol 12, e45, doi:10.1016/j.jtho.2016.12.022 (2017).
10 Dolled-Filhart, M. et al. Development of a Prototype Immunohistochemistry Assay to Measure Programmed Death Ligand-1 Expression in Tumor Tissue. Arch Pathol Lab Med 140, 1259-1266, doi:10.5858/arpa.2015-0544-OA (2016).
11 Dolled-Filhart, M. et al. Development of a Companion Diagnostic for Pembrolizumab in Non-Small Cell Lung Cancer Using Immunohistochemistry for Programmed Death Ligand-1. Arch Pathol Lab Med 140, 1243-1249, doi:10.5858/arpa.2015-0542-OA (2016).
12 Hirsch, F. R. et al. PD-L1 Immunohistochemistry Assays for Lung Cancer: Results from Phase 1 of the Blueprint PD-L1 IHC Assay Comparison Project. J Thorac Oncol 12, 208-222, doi:10.1016/j.jtho.2016.11.2228 (2017).
13 Velcheti, V. et al. Real-world PD-L1 testing and distribution of PD-L1 tumor expression by immunohistochemistry assay type among patients with metastatic non-small cell lung cancer in the United States. PLoS One 13, e0206370, doi:10.1371/journal.pone.0206370 (2018).
14 Grant, M. J., Herbst, R. S. & Goldberg, S. B. Selecting the optimal immunotherapy regimen in driver-negative metastatic NSCLC. Nat Rev Clin Oncol 18, 625-644, doi:10.1038/s41571-021-00520-1 (2021).
15 Litchfield, K. et al. Meta-analysis of tumor- and T cell-intrinsic mechanisms of sensitization to checkpoint inhibition. Cell 184, 596-614 e514, doi:10.1016/j.cell.2021.01.002 (2021).
16 Rizvi, H. et al. Molecular Determinants of Response to Anti-Programmed Cell Death (PD)-1 and Anti-Programmed Death-Ligand 1 (PD-L1) Blockade in Patients With Non-Small-Cell Lung Cancer Profiled With Targeted Next-Generation Sequencing. J Clin Oncol 36, 633-641, doi:10.1200/JCO.2017.75.3384 (2018).
17 Singal, G. et al. Association of Patient Characteristics and Tumor Genomics With Clinical Outcomes Among Patients With Non-Small Cell Lung Cancer Using a Clinicogenomic Database. JAMA 321, 1391-1399, doi:10.1001/jama.2019.3241 (2019).
18 Rizvi, N. A. et al. Cancer immunology. Mutational landscape determines sensitivity to PD-1 blockade in non-small cell lung cancer. Science 348, 124-128, doi:10.1126/science.aaa1348 (2015).
19 McGranahan, N. et al. Clonal neoantigens elicit T cell immunoreactivity and sensitivity to immune checkpoint blockade. Science 351, 1463-1469, doi:10.1126/science.aafl490 (2016).
20 Van Allen, E. M. et al. Genomic correlates of response to CTLA-4 blockade in metastatic melanoma. Science 350, 207-211, doi:10.1126/science.aad0095 (2015).
21 Samstein, R. M. et al. Tumor mutational load predicts survival after immunotherapy across multiple cancer types. Nat Genet 51, 202-206, doi:10.1038/s41588-018-0312-8 (2019).
22 Vega, D. M. et al. Aligning tumor mutational burden (TMB) quantification across diagnostic platforms: phase II of the Friends of Cancer Research TMB Harmonization Project. Ann Oncol, doi:10.1016/j.annonc.2021.09.016 (2021).
23 Steuer, C. E. & Ramalingam, S. S. Tumor Mutation Burden: Leading Immunotherapy to the Era of Precision Medicine? J Clin Oncol 36, 631-632, doi:10.1200/JCO.2017.76.8770 (2018).
24 Anagnostou, V. et al. Multimodal genomic features predict outcome of immune checkpoint blockade in non-small-cell lung cancer. Nat Cancer 1, 99-111, doi:10.1038/s43018-019-0008-8 (2020).
25 Marcus, L. et al. FDA Approval Summary: Pembrolizumab for the Treatment of Tumor Mutational Burden-High Solid Tumors. Clin Cancer Res 27, 4685-4689, doi:10.1158/1078-0432.CCR-21-0327 (2021).
26 Marabelle, A. et al. Association of tumour mutational burden with outcomes in patients with advanced solid tumours treated with pembrolizumab: prospective biomarker analysis of the multicohort, open-label, phase 2 KEYNOTE-158 study. Lancet Oncol 21, 1353-1365, doi:10.1016/S1470-2045(20)30445-9 (2020).
27 Ricciuti, B. et al. Association of High Tumor Mutation Burden in Non-Small Cell Lung Cancers With Increased Immune Infiltration and Improved Clinical Outcomes of PD-L1 Blockade Across PD-L1 Expression Levels. JAMA Oncol, doi:10.1001/jamaoncol.2022.1981 (2022).
28 Hellmann, M. D. et al. Tumor Mutational Burden and Efficacy of Nivolumab Monotherapy and in Combination with Ipilimumab in Small-Cell Lung Cancer. Cancer Cell 33, 853-861 e854, doi:10.1016/j.ccell.2018.04.001 (2018).
29 Hanna, G. J. et al. Real-world outcomes treating patients with advanced cutaneous squamous cell carcinoma with immune checkpoint inhibitors (CPI). Br J Cancer 123, 1535-1542, doi:10.1038/s41416-020-01044-8 (2020).
Hellmann, M. D. et al. Nivolumab plus Ipilimumab in Lung Cancer with a High Tumor Mutational Burden. N Engl J Med 378, 2093-2104, doi:10.1056/NEJMoa1801946 (2018).
31 Friedman, C. F. et al. Atezolizumab Treatment of Tumors with High Tumor Mutational Burden from MyPathway, a Multicenter, Open-Label, Phase IIa Multiple Basket Study. Cancer Discov 12, 654-669, doi:10.1158/2159-8290.CD-21-0450 (2022).
32 Schenker, M. et al. Abstract CT022: CheckMate 848: A randomized, open-label, phase 2 study of nivolumab in combination with ipilimumab or nivolumab monotherapy in patients with advanced or metastatic solid tumors of high tumor mutational burden. Cancer Research 82, CT022-CT022, doi:10.1158/1538-7445.Am2022-ct022 (2022).
33 Palmer, A. C., Izar, B., Hwangbo, H. & Sorger, P. K. Predictable Clinical Benefits without Evidence of Synergy in Trials of Combination Therapies with Immune-Checkpoint Inhibitors. Clin Cancer Res 28, 368-377, doi:10.1158/1078-0432.CCR-21-2275 (2022).
34 Cristescu, R. et al. Pan-tumor genomic biomarkers for PD-1 checkpoint blockade-based immunotherapy. Science 362, doi:10.1126/science.aar3593 (2018).
35 Lee, J. S. & Ruppin, E. Multiomics Prediction of Response Rates to Therapies to Inhibit Programmed Cell Death 1 and Programmed Cell Death 1 Ligand 1. JAMA Oncol 5, 1614-1618, doi:10.1001/jamaoncol.2019.2311 (2019).
36 Rolfo, C. et al. Liquid Biopsy for Advanced NSCLC: A Consensus Statement From the International Association for the Study of Lung Cancer. J Thorac Oncol 16, 1647-1662, doi:10.1016/j.jtho.2021.06.017 (2021).
37 Herbst, R. S. et al. Predictive correlates of response to the anti-PD-L1 antibody MPDL3280A in cancer patients. Nature 515, 563-567, doi:10.1038/nature14011 (2014).
38 Taube, J. M. et al. Colocalization of inflammatory response with B7-h1 expression in human melanocytic lesions supports an adaptive resistance mechanism of immune escape. Sci Transl Med 4, 127ra137, doi:10.1126/scitranslmed.3003689 (2012).
39 Chen, D. S. & Mellman, I. Elements of cancer immunity and the cancer-immune set point. Nature 541, 321-330, doi:10.1038/nature21349 (2017).
40 Sanmamed, M. F. & Chen, L. A Paradigm Shift in Cancer Immunotherapy: From Enhancement to Normalization. Cell 175, 313-326, doi:10.1016/j.cell.2018.09.035 (2018).
41 Ayers, M. et al. IFN-gamma-related mRNA profile predicts clinical response to PD-1 blockade. J Clin Invest 127, 2930-2940, doi:10.1172/JC191190 (2017).
42 Fountzilas, E., Kurzrock, R., Hiep Vo, H. & Tsimberidou, A. M. Wedding of Molecular Alterations and Immune Checkpoint Blockade: Genomics as a Matchmaker. J Natl Cancer Inst, doi:10.1093/jnci/djab067 (2021).
43 Ott, P. A. et al. T-Cell-Inflamed Gene-Expression Profile, Programmed Death Ligand 1 Expression, and Tumor Mutational Burden Predict Efficacy in Patients Treated With Pembrolizumab Across 20 Cancers: KEYNOTE-028. J Clin Oncol 37, 318-327, doi:10.1200/JCO.2018.78.2276 (2019).
44 Lu, S. et al. Comparison of Biomarker Modalities for Predicting Response to PD-1/PD-L1 Checkpoint Blockade: A Systematic Review and Meta-analysis. JAMA Oncol 5, 1195-1204, doi:10.1001/jamaoncol.2019.1549 (2019).
Zhang, Z. et al. RNF2 ablation reprograms the tumor-immune microenvironment and stimulates durable NK and CD4+ T-cell-dependent antitumor immunity. Nature Cancer 2, 1018-1038, doi:10.1038/s43018-021-00263-z (2021).
46 Cristescu, R. et al. Transcriptomic Determinants of Response to Pembrolizumab Monotherapy Across Solid Tumor Types. Clin Cancer Res, doi:10.1158/1078-0432.CCR-21-3329 (2021).
47 Zaitsev, A. et al. Precise reconstruction of the TME using bulk RNA-seq and a machine learning algorithm trained on artificial transcriptomes. Cancer Cell 40, 879-894 e816, doi:10.1016/j.ccell.2022.07.006 (2022).
48 Necchi, A. et al. Comprehensive Assessment of Immuno-oncology Biomarkers in Adenocarcinoma, Urothelial Carcinoma, and Squamous-cell Carcinoma of the Bladder. Eur Urol 77, 548-556, doi:10.1016/j.eururo.2020.01.003 (2020).
49 Zahoor, H. & Grivas, P. The Cancer Immunogram: A Pledge for a Comprehensive Biomarker Approach for Personalized Immunotherapy in Urothelial Cancer. Eur Urol 75, 445-447, doi:10.1016/j.eururo.2018.12.005 (2019).
50 van Dijk, N. et al. The Cancer Immunogram as a Framework for Personalized Immunotherapy in Urothelial Cancer. Eur Urol 75, 435-444, doi:10.1016/j.eururo.2018.09.022 (2019).
51 Bellmunt, J. et al. Putative Biomarkers of Clinical Benefit With Pembrolizumab in Advanced Urothelial Cancer: Results from the KEYNOTE-045 and KEYNOTE-052 Landmark Trials. Clin Cancer Res 28, 2050-2060, doi:10.1158/1078-0432.CCR-21-3089 (2022).
52 Jun, T., Anker, J. & Galsky, M. D. Biomarkers for therapy selection in metastatic urothelial cancer. Journal of Cancer Metastasis and Treatment 8, 1, doi:10.20517/2394-4722.2021.171 (2022).
53 Powles, T. et al. Avelumab maintenance in advanced urothelial carcinoma: biomarker analysis of the phase 3 JAVELIN Bladder 100 trial. Nat Med 27, 2200-2211, doi:10.1038/s41591-021-01579-0 (2021).
54 Harms, K. L. et al. Virus-positive Merkel Cell Carcinoma Is an Independent Prognostic Group with Distinct Predictive Biomarkers. Clin Cancer Res 27, 2494-2504, doi:10.1158/1078-0432.CCR-20-0864 (2021).
55 Tomlins, S. A. et al. Development and Validation of StrataNGS, a Multiplex PCR, Semiconductor Sequencing-Based Comprehensive Genomic Profiling Test. J Mol Diagn 23, 1515-1533, doi:10.1016/j.jmoldx.2021.08.005 (2021).
56 Tomlins, S. A. et al. Real-World Performance of a Comprehensive Genomic Profiling Test Optimized for Small Tumor Samples. JCO Precis Oncol 5, doi:10.1200/PO.20.00472 (2021).
57 Rousseau, B. et al. The Spectrum of Benefit from Checkpoint Blockade in Hypermutated Tumors. N Engl J Med 384, 1168-1170, doi:10.1056/NEJMc2031965 (2021).
58 Ho, D., Imai, K., King, G. & Stuart, E. A. MatchIt: Nonparametric Preprocessing for Parametric Causal Inference. Journal of Statistical Software 42, 1-28, doi:10.18637/jss.v042.i08 (2011).
59 Zehir, A. et al. Mutational landscape of metastatic cancer revealed from prospective clinical sequencing of 10,000 patients. Nat Med 23, 703-713, doi:10.1038/nm.4333 (2017).
60 Han, G. et al. 9p21 loss confers a cold tumor immune microenvironment and primary resistance to immune checkpoint therapy. Nat Commun 12, 5606, doi:10.1038/s41467-021-25894-9 (2021).
61 Sholl, L. M. Biomarkers of response to checkpoint inhibitors beyond PD-L1 in lung cancer. Mod Pathol, doi:10.1038/s41379-021-00932-5 (2021).
62 Di Federico, A., De Giglio, A., Parisi, C. & Gelsomino, F. STK11/LKB1 and KEAP1 mutations in non-small cell lung cancer: Prognostic rather than predictive? Eur J Cancer 157, 108-113, doi:10.1016/j.ejca.2021.08.011 (2021).
63 Skoulidis, F. et al. STK11/LKB1 Mutations and PD-1 Inhibitor Resistance in KRAS-Mutant Lung Adenocarcinoma. Cancer Discov 8, 822-835, doi:10.1158/2159-8290.CD-18-0099 (2018).
64 Shen, J. et al. ARID1A deficiency promotes mutability and potentiates therapeutic antitumor immunity unleashed by immune checkpoint blockade. Nat Med 24, 556-562, doi:10.1038/s41591-018-0012-z (2018).
65 Miao, D. et al. Genomic correlates of response to immune checkpoint therapies in clear cell renal cell carcinoma. Science 359, 801-806, doi:10.1126/science.aan5951 (2018).
66 Braun, D. A. et al. Clinical Validation of PBRM1 Alterations as a Marker of Immune Checkpoint Inhibitor Response in Renal Cell Carcinoma. JAMA Oncol 5, 1631-1633, doi:10.1001/jamaoncol.2019.3158 (2019).
67 Liu, X. D. et al. PBRM1 loss defines a nonimmunogenic tumor phenotype associated with checkpoint inhibitor resistance in renal carcinoma. Nat Commun 11, 2135, doi:10.1038/s41467-020-15959-6 (2020).
68 Ebot, E. M. et al. Deletions on 9p21 are associated with worse outcomes after anti-PD-1/PD-L1 monotherapy but not chemoimmunotherapy. NPJ Precis Oncol 6, 44, doi:10.1038/s41698-022-00286-4 (2022).
69 Institute, C. R. FDA Approval Timeline of Active Immunotherapies, <https://www.cancerresearch.org/en-us/scientists/immuno-oncology-landscape/fda-approval-timeline-of-active-immunotherapies> (2022).
70 Cottrell, T. R. & Taube, J. M. PD-L1 and Emerging Biomarkers in Immune Checkpoint Blockade Therapy. Cancer J 24, 41-46, doi:10.1097/PPO.0000000000000301 (2018).
71 Kerr, K. M. The PD-L1 Immunohistochemistry Biomarker: Two Steps Forward, One Step Back? J Thorac Oncol 13, 291-294, doi:10.1016/j.jtho.2018.01.020 (2018).
72 Sholl, L. M. et al. The Promises and Challenges of Tumor Mutation Burden as an Immunotherapy Biomarker: A Perspective from the International Association for the Study of Lung Cancer Pathology Committee. J Thorac Oncol 15, 1409-1424, doi:10.1016/j.jtho.2020.05.019 (2020).
73 Addeo, A., Friedlaender, A., Banna, G. L. & Weiss, G. J. TMB or not TMB as a biomarker: That is the question. Crit Rev Oncol Hematol 163, 103374, doi:10.1016/j.critrevonc.2021.103374 (2021).
74 Medicine, F. (2021).
75 Banchereau, R. et al. Molecular determinants of response to PD-L1 blockade across tumor types. Nat Commun 12, 3969, doi:10.1038/s41467-021-24112-w (2021).

### Supplementary Information

### Supplementary Methods

### Strata Clinical Molecular Database (SCMD) Validity Analysis

Analyses to assess the clinical validity of the SCMD included an analysis of real-world progression-free survival (rwPFS; by time to next therapy) in patients with non-small cell lung cancer (NSCLC) treated with systemic first-line, first-generation monotherapy tyrosine kinase inhibitor (TKI) against EGFR, ALK, ROS1 or MET (i.e., erlotinib, gefitinib or crizotinib) vs. those treated with later-generation monotherapy (i.e., afatinib, alectinib, brigatinib, capmatinib, ceritinib, entrectinib, lorlatinib, osimertinib or tepotinib). Next, we assessed rwPFS in patients with NSCLC treated with systemic first-line, current National Comprehensive Cancer Network (NCCN; v3.2022) preferred first-line targeted oncogene TKI (i.e., osimertinib, afatinib, alectinib, brigatinib, capmatinib, entrectinib, dabrafenib+trametinib, entrectinib, lorlatinib, selpercatinib or tepotinib), based on whether the line of therapy started before or after completion of StrataNGS testing. Lastly, we assessed rwPFS in patients with NSCLC treated with biomarker (by StrataNGS testing) matched, systemic first-line, current preferred first-line targeted oncogene TKI (with therapy line start date after completion of StrataNGS testing), stratified by whether the sample: 1) passed both StrataNGS sample input criteria (≥2 mm2 tumor surface area, ≥1 ng/ul isolated DNA or RNA, sample age<5 yrs) and relevant sequencing QC metrics (≥20% tumor content, mutation QC pass [for 1st line standard of care mutations], and fusion QC pass [for 1st line standard of care fusions]) or 2) did not meet sample input or sequencing QC metrics but reported a therapy matched biomarker.

Validation of Quantitative Transcriptomic Profiling (qTP) and IRS Integrated Comprehensive Genomic Profiling (CGP)+qTP Test Introduction

The integrative comprehensive genomic profiling and quantitative transcriptomic profiling (CGP+qTP) laboratory developed test (LDT) is performed in a Clinical Laboratory Improvement Amendments (CLIA) certified, College of American Pathologist (CAP) accredited laboratory. The test is performed on co-isolated DNA and RNA from FFPE tissue tumor specimens and simultaneously assesses mutations (SNVs, short [<40 bp] indels and SSVs), CNAs (amplifications or deep deletions [equivalent to homozygous loss if diploid]), MSI status, and TMB (in mutations/megabase [Muts/Mb]), along with qTP of individual target genes; the integrative IRS algorithm is also reported by combining TMB and target gene expression of four amplicons as described in the Methods. Three multiplexed PCR-based panels (two DNA and one RNA) are used, with all three libraries per sample combined prior to sequencing; all variant classes are analyzed and subjected to independent quality control (QC) metrics and bioinformatics pipelines for reporting as a single integrative test report. Analyses related to the analytical and clinical validation of the general qTP component (where relevant to IRS) as well as the integrative IRS model are described herein.

Specimen evaluation, processing, and molecularly informed tumor content determination, nucleic acid isolation, quantification, normalization, library preparation, sequencing, quality control and CGP reporting are essentially as described in the validation of the StrataNGS CGP test1. For tumor content determination, briefly, given the challenges of estimating tumor content in difficult samples, the final molecularly informed tumor content is set after completion of StrataNGS testing, initially incorporating variant allele frequencies (VAF) and copy state of mutations in tumor suppressors known to nearly always be homozygous2, with subsequent refinement integrating these results with the remainder of the mutational and copy number profiles along with b-allele frequencies from high confidence SNPs1. For all cases in the SCMD (including those used in IRS development and validation) the final MTC was set by a single pathologist (S.A.T.) with automatic updating to all applicable variants/variant classes.

The current CGP component of the CGP+qTP is an updated version of the validated StrataNGS CGP test, which is covered for Medicare beneficiaries with advanced solid tumors with performance characteristics characterized across >30,000 consecutively submitted FFPE tumor tissue samples1,3. Compared to the previous version, the current version targets 59 fusion driver genes (vs. 46 previously), resulting in a total of 437 unique genes across the CGP test. Additionally, the updated version leverages an independently trained and validated random forest classifier to detect gene fusions, and an independently trained and validated random forest classifier to detect mutations (with equivalent or better performance to the previous version for all variant classes). The TMB component of the CGP+qTP test has been extensively validated1 and has not been updated. Briefly, the TMB assessment for the CGP+qTP LDT (and IRS component) includes non-coding (at well characterized genomic loci) and coding, synonymous and non-synonymous, SNV and multi-nucleotide (two bases) variants from a panel with a maximum footprint of 1.7 Mb. Notably, only mutations with VAF>¼ of the final molecularly informed tumor content are included in the TMB estimate (inclusion of only clonal mutations and tumor content correction have both been shown to improve prediction of checkpoint inhibitor response 4,5). Additional custom filtering is employed to exclude high likelihood technical artifacts and germline variants and the TMB (Muts/Mb) estimate is calculated via the total number of positions with sufficient depth of coverage necessary for definitive assessment (maximum possible 1.7 Mb).

The qTP component of the CGP+qTP test is completed using the same aliquot of RNA and multiplex PCR panel used in StrataNGS (for gene fusions), with the current version of the panel containing 950 amplicons targeting individual gene fusion isoforms involving 59 driver genes [chimeric amplicons; reported as CGP variants as described above], and 106 non-chimeric amplicons (103 target genes and 3 housekeeping genes) used for quantitative expression profiling; pre-clinical and investigational versions of the quantitative expression profiling component have included 26, 46 and 103 target genes. Target gene expression is determined in scaled, log 2, median-centered units of normalized reads per million [nRPM]6, representing target gene expression normalized to housekeeping gene expression, then scaled to the distribution observed in a common control (normal FFPE genome in a bottle Ashkenazi father cell line [GIAB; GM24149 from Horizon Discovery]) per panel. For reporting, results are then scaled to a fixed pan-cancer median value of 10 after log 2 normalization. Hence, all individually reported targets have a median value of 10.0 across all pan-solid tumor samples tested, with each unit increase representing a doubling from the median.

### Validation of qTP and IRS Housekeeping Gene Selection

To evaluate the suitability of 6 positive “control” genes included in initial pre-clinical versions of the qTP panel and identify novel housekeeping genes, we downloaded uniformly realigned, gene expression quantified, quantile normalized, and batch effect removed TCGA expression data (in fragments per kilobase per million [FPKM]) from 7. Correlation of variation (CV) was determined for each gene per tumor type, and candidate housekeeping genes were ranked by the number of tumor types in which they ranked in the top 20 most stable genes (by lowest CV). Uniformly processed gene expression data (in transcripts per million [TPM]) from the highest-ranking housekeeping genes, the 6 pre-clinical “positive control genes”, as well as the commonly used housekeeping gene GAPDH were then downloaded for 20,841 total samples contained in the MiPanda database8 and CV for each gene was determined across all samples (cell line, normal and TCGA) in MiPanda and used to prioritize genes for inclusion in panels as potential housekeeping genes.

### Validation of qTP and IRS Quality Control

As the qTP component of the CGP+qTP test was run in parallel to StrataNGS CGP clinical testing, quality control metrics (and several validation analyses) which leveraged a consecutive 4-month period of StrataNGS clinical testing using the final version of the qTP panel for the integrated CGP+qTP test (October 2021 to January 2022; n=3,904 total FFPE tumor tissue samples). In addition to a minimum total mapped read count, previous clinical versions of the qTP component modeled the expected distribution of housekeeping gene expression (Mahalanobis distance of sample housekeeping target expression<4 standard deviations away from the population as passing QC) and confirmed that each housekeeping gene is within its LOQ (equivalent to reportable range given this includes both upper and lower bounds)9. Based on pre-validation experiments, we compared the correlation of panel-wide normalized target gene expression in replicate RNA aliquots from clinical FFPE tumor samples with reportable expression from the previous panel (using the previous total mapped read and housekeeping QC metrics) to those from the current qTP panel using a single metric of >150,000 total mapped reads as having reportable expression. Panel wide concordance correlation of >0.8 was considered acceptable. Additional QC is performed through inclusion of the FFPE GIAB sample in all clinical runs of the gene expression panel subjected to the same approach. The correlation of target gene expression from replicate PD-L1, PD-1 and ADAM12 amplicons was determined across the 24,463 Strata Trial samples with complete sample information (including current tumor type reporting), informative TMB, informative gene expression, and tumor content≥20% (regardless of treatment data availability).

### Validation of qTP and IRS: Limit of Quantification (LOQ), Linearity and Reportable Range

We determined LOQ and linearity for individual target genes by determining the lowest nRPM that can be precisely quantified in replicate RNA aliquots subjected to repeat multiplex PCR based library preparation (followed by templating and sequencing). Importantly, independent library preparations represent independent RNA aliquots, cDNA (from reverse transcription) and library preparation, so the nRPM level below which repeat samples show increased dispersion represents the lowest amount of RNA library that can be precisely quantified. We determined the LOQ for normalized expression of all individual target gene amplicons by evaluating the weighted root mean square error (WRMSE) using a 40 sample window (beginning with the highest expressing target gene expression in replicate #1) and evaluated the minimum RWSE, as well as additional windows (again beginning with the highest expressing target gene expression in replicate #1) where the WRMSE is first observed to be below the 50th, 25th, 15th and 10th quantile. Using this approach, the clinical implications of residual error distribution were weighed vs. the overall linearity and dynamic range of quantification in setting the most appropriate LOQ for each target gene amplicon. Linearity was thus determined by the concordance correlation coefficient for each target gene after setting all sub-LOQ values to LOQ, and the dynamic range was defined as the LOQ to the highest expressing value for that gene in replicate #1. No upper LOQ is established as there is essentially no chance of clinical misinterpretation of a value higher than that established in this approach given the observed linearity. Hence, the reportable range for each amplicon is floored at the LOQ but has no upper limit. Linearity LOQs have not been applied to any other validation analysis performed herein (unless specified) to present the full range of qTP generated data.

As a Cox proportional hazard-based algorithm, IRS produces a quantitative hazard that is proportional to the hazard rate observed in the dataset, so that higher values represent decreased hazard (i.e., more benefit from PD-(L)1 therapy benefit) and smaller values represent increased hazard (i.e., less benefit from PD-(L)1 therapy). The individual scaled hazard rate is reported for informational purposes however the IRS result is reported categorically as IRS-High or IRS-Low. Hence, for the IRS test result, measurand LOD/LOQ and linearity is not applicable, and the reportable range of the quantitative scaled hazard rate is reported as determined without upper or lower bounds. Performance of the IRS model with or without LOQs applied to the individual expression components was compared and concordance correlation of IRS scores and quantification of the % of patients changing IRS groups (IRS-H to IRS-L or vice versa) was determined.

### Validation of qTP and IRS Accuracy

The accuracy of the qTP component was validated through a multi-part accuracy study leveraging qRT-PCR, comparison to known target gene expression across tumor types, and clinical immunohistochemistry. For representational qRT-PCR validation, clinical FFPE tumor samples from StrataNGS testing and 3 control RNA samples were subjected to qTP and qRT-PCR on replicate RNA aliquots. For qRT-PCR, 2-20ng clinically isolated FFPE RNA per sample underwent reverse transcription using SuperScript IV VILO Master Mix (Invitrogen) and pre-amplification using TaqMan PreAmp Master Mix (Applied Biosystems) using a pooled Taqman primer/hydrolysis probe assays and 14 cycles. qPCR was then performed in duplicate on a Quantstudio 3 Real Time PCR system using a 1:20 dilution of amplified product per qPCR reaction and TaqMan Fast Universal PCR Master Mix (Applied Biosystems). Individual amplicon level thresholds and baselines were set during the exponential amplification phase to determine cycle crossing threshold (Ct) values. Samples with duplicate qPCR values>2 Ct difference were excluded unless both values were >30 or singlicate experiments were performed. All undefined Ct values were considered as having Ct of 40. qRT-PCR ΔCt values were determined as: target amplicon Ct—(median of housekeeping gene amplicon Ct) and were otherwise scaled as for qTP results using the same FFPE reference sample run in all clinical and validation runs. Panel-wide concordance correlation coefficient were determined across all included target genes and samples in the cohort. Acceptable concordance correlation coefficient of >0.7 was pre-specified given the expected range of transcript expression across the amplicons/samples.

To indirectly compare qTP generated target gene expression to that expected in a given tumor type, we compared target gene expression profiles for all 103 target genes from qTP (log 2) to the pan-cancer TCGA tumor set10 (RSEM batch normalized log [n+1]2, from Illumina HiSeq_RNASeqV2; downloaded from cBioPortal) consisting of 9,618 samples from 30 TCGA tumor types that could be mapped directly to 28 Strata defined primary and/or secondary tumor types across the 4-month period of clinical StrataNGS testing described above. Results were summarized across the entire 103 target gene set per tumor type, and correlation (Spearman rho) of per-target gene mean expression in each dataset was determined; no acceptance criteria were pre-specified. As IRS was trained on data from both the current qTP panel and the previous 46 gene version (with appropriate panel-specific scaling), we also compared TCGA and qTP results for the 20 immune and proliferation expression candidates included in IRS development (IFNG was excluded from this analysis as it could not be reliably quantified across all qTP panels) across the 24,463 Strata Trial samples with complete sample information used to assess IRS distribution (see FIG. 39a-d). Spearman correlation was determined between TCGA and qTP profiled tumors for all candidate biomarkers.

To validate qTP and IRS components vs IHC, we used optical character recognition and natural language processing to prioritize accompanying pathology reports received with StrataNGS test requests for abstraction of IHC biomarker results by trained reviewers according to a documented SOP into a clinical database. Where orthogonal IHC biomarkers and StrataNGS were performed on different specimens from the same case (pathology accession) that are presumed to come from the same tumor (e.g., single case with a primary colon cancer, lymph node metastasis, liver metastasis resection), testing was considered as performed on the same specimen and suitable for comparison (representing usual clinical practice). Where orthogonal biomarker and StrataNGS testing were performed on different specimen from the same case grossly or histologically distinct tumors were described and commented on in the specimen report, testing was considered as performed on distinct specimens and not suitable for comparison. For Ki67 and PD-L1, orthogonal results were excluded if a range of IHC expression >20% was provided (e.g., Ki67 staining reported as >50% were excluded); for orthogonal IHC results with <20% range, the average of the range was used (e.g., Ki67>80%=90%). All analyses used samples with reportable qTP and tumor content ≥20%.

As the CGP+qTP test reports a composite proliferation biomarker (TOP2A+UBE2C expression), and previous analyses using microarray expression profiling and/or research grade multiplex PCR RNAseq have shown that TOP2A and UBE2C are robust cell cycle/proliferation biomarkers11-14, the entire available cohort (n=956) was used to establish accuracy of this proliferation biomarker by determining the correlation coefficient with clinical IHC proliferation index by percent Ki67 positive (with acceptable correlation coefficient of ≥0.7 pre-specified). Hence no acceptable correlation coefficient was prespecified for the analysis of TOP2A only (the biomarker included in the IRS model) vs. IHC proliferation index).

Likewise, the CGP+qTP test reports PD-L1 individually through averaging target gene expression from the two PD-L1 amplicons. Hence, we determined the accuracy of our qTP PD-L1 quantification vs. clinical IHC using 256 NSCLC FFPE tumor samples with clinical PD-L1 IHC expression by the 22C3 clone (using tumor proportion score [TPS]) in accompanying pathology reports. As TPS does not include PD-L1 expression in non-tumor cells (as for CPS using 22C3 in other tumor types and routinely included in PD-L1 expression by other PD-L1 IHC clones [e.g., SP142], acceptable accuracy was pre-specified as statistically significant, ordinally increasing differences in median qTP PD-L1 expression between the three clinically relevant TPS groups (Kruskal Wallis test, p<0.05; Jonckheere-Terpstra trend test [increasing median from 0%, 1-49%, and ≥50%, p<0.05). As the PD-L1 component of IRS only uses target gene expression from a single PD-L1 amplicon (CD274.E4E5.NM_014143.3) based on the Lasso penalized CPH modeling, no acceptable correlation coefficient was prespecified for the analysis of target gene expression from only this PD-L1 amplicon vs. TPS IHC.

### Validation of qTP and IRS Tumor Content Limit of Detection (LOD)

Tumor content LOD was determined by binning the development and validation cohort samples by tumor content (20-35%, 40-70% and >70%) and visualizing TTNT by Kaplan Meier analysis, given that the established LOD for accurate TMB estimation was determined as 20% tumor content, and included a tumor content term (continuous tumor content) in the overall adjusted CPH model in the IRS development cohort (including age, gender, most common tumor type [NSCLC] vs. others, therapy type [monotherapy/combination], and line of therapy) and validation cohort (including age, gender, most common tumor type [melanoma] vs. others, therapy type [PD-1 vs. PD-L1], and line of therapy). Additionally, we identified subjects in the SCMD that otherwise would have been included in the discovery or validation cohorts, but the tumor content of the tested sample was <20%, and determined the performance of IRS by CPH modeling (adjusting for age, gender, therapy line, most common tumor type (NSCLC) vs. others, PD-(L)1 type (pembrolizumab vs. other PD-[L]1), and monotherapy vs. combination therapy (for pembrolizumab).

### Validation of qTP and IRS Reproducibility

Formal reproducibility of the gene expression panel used for the CGP+qTP test and the IRS algorithm was established separately from the previous validation of TMB reproducibility1, as the current version of the qTP panel was not performed in parallel with the initial TMB reproducibility experiments15. Hence, panel-wide qTP and IRS reproducibility between operators, lots, and instrumentation was established using separate replicate nucleic acid aliquots isolated from FFPE tumor samples. Twenty-seven unique samples were assessed by two operators on different days using different library preparation instrumentation, different library preparation reagent lots, and different templating and sequencing lots. Each operator performed templating and sequencing sequentially for each run on different Ion Chefs and on different S5XL or S5 Prime sequencing instruments. For each sample, the maximum and minimum nRPM for each target gene across all replicates was plotted. Overall panel-wide acceptable concordance correlation coefficient was set at 0.8. Concordance correlation of maximum and minimum IRS score for each sample was also determined. Qualitative agreement of IRS status (High vs. Low) from the maximum and minimum IRS score across all replicates was determined. Acceptable IRS score concordance correlation coefficient was pre-specified at 0.8 and acceptable qualitative IRS agreement accuracy was set at >90%.

Clinical Validity and Clinical Utility of qTP in Patients with Advanced Breast Cancer

For orthogonal validation of breast cancer biomarkers, identification of StrataNGS tested cases with estrogen receptor (ER; ESRI), progesterone receptor (PR; PGR), or ERBB2 (HER2) was performed as described above for Ki67 and PD-L1. Cases were excluded if they only referenced results from a previous sample, and only cases with quantitative results were included; ER and PR reported as <1% were considered negative (0% staining). HER2 IHC expression was binned into the four currently distinct clinical groups (0, 1+, 2+, and 3+). As ER, PR and ERBB2 expression by clinical IHC have established clinical utility in therapy selection for patients with advanced breast cancer, we performed a similar accuracy analysis vs. clinical IHC to that described above for the TOP2A and PD-L1 components of IRS, however cohorts were randomly split into training and validation cohorts (for clinical validity analysis after setting thresholds in the training cohort as described below) before performing the accuracy analyses. ER and PR have only been assessed on the current qTP panel, while HER2 has been assessed on previous versions.

Accuracy of ER by qTP was validated against clinical IHC using a cohort of 300 breast FFPE tumor samples with reportable qTP (including tumor content [TC]≥20%) and ER IHC expression (by % tumor cells positive) in accompanying pathology reports. The entire cohort was used for accuracy, however the cohort was randomly split into equivalent training (n=150) and validation (n=150) cohorts to establish clinical validity prior to performing the accuracy assessment. Accuracy of PR by qTP was performed similarly using a cohort of 291 breast cancer samples with reportable qTP and PR IHC (training cohort, n=145; validation cohort, n=146). Accuracy of HER2 by qTP was performed similarly using a cohort of 545 breast cancer samples with reportable qTP and HER2 IHC (training cohort, n=273; validation cohort, n=272). For ER and PR, acceptable Pearson correlation coefficient of >0.7 was pre-specified. Given current semi-quantitative clinical HER2 IHC reporting, acceptable concordance was pre-specified as statistically significant, ordinally increasing differences in median qTP ERBB2 expression between each of the four groups (Kruskal Wallis test, p<0.05; Jonckheere-Terpstra trend test [increasing median from 0 to 1+ to 2+ to 3+], p<0.05).

Clinical validity for ER status by qTP was established by setting thresholds for qTP ER Negative (<12.75) and Positive (>14.5) in the training cohort (n=150) of breast cancer FFPE tissue samples with clinical ER status (by % tumor cells positive) based on the clinical IHC defined categories of ER Negative (0%), Low (1-10%) and Positive (>10%). Expression between the Negative and Positive thresholds were defined as qTP ER inconclusive. Desired sensitivity (positive percent agreement [PPA]) and specificity (negative percent agreement [NPA]) for qTP ER Negative/Positive status (vs. IHC Negative and Positive) was pre-specified as >95% each. Locked thresholds were then applied to the validation cohort (n=150). Clinical validity for PR status by qTP was established by setting a threshold for qTP PR Negative (<12.3) in the training cohort (n=145) of breast cancer FFPE tissue samples with clinical PR status (by % tumor cells positive). Although PR does not have a “Low” clinical IHC reporting group, three clinical IHC defined categories of PR Negative (Neg.; 0%), Low (1-10%) and Positive (Pos; >10%) were used in the training cohort to facilitate appropriate balancing of PPA and NPA in the threshold setting. As the potential clinical implications of false positive PR status, namely inappropriately considering an ER negative/HER2 negative breast cancer as hormone receptor positive (vs. triple negative) are more impactful than false negative PR status (it is unclear if ER negative/PR positive breast cancer are biologically plausible), the threshold was set to favoring NPA and pre-specified acceptable NPA (versus PR 0% IHC) of greater than 95% was set. The locked threshold was then applied to the validation cohort (n=146). Clinical validity for HER2 status by qTP was established by setting thresholds in the training cohort (n=273) of breast cancer FFPE tissue samples with clinical HER2 status (0, 1+, 2+ or 3+ categories) in accompanying pathology reports. As with ER, given that the clinical utility of HER2 IHC 2+ is to reflex to FISH/ISH (and StrataNGS provides ERBB2 copy status), and the unclear validity of 0 vs. 1+ expression in retrospective samples clinically scored before the FDA approval of trastuzumab deruxtecan in HER2 1+ and 2+ (FISH/ISH negative) breast cancer, we set thresholds for qTP HER2 Low (<18.0) and High (>19.2), with expression in between those thresholds reported as qTP HER2 Inconclusive (light gray); the threshold was set by balancing desired maximum sensitivity vs. IHC 3+ with the observation that the majority of IHC 3+ tumors with the lowest qTP HER2 expression also lacked ERBB2 amplifications in the training cohort. Hence, desired NPA and PPA for qTP HER2 Low/High status (vs. IHC Negative and 3+) was pre-specified as NPA>95% and PPA>70%; no performance metrics for IHC 2+ samples were prespecified. Locked thresholds were then applied to the validation cohort (n=272 [including 51 IHC 2+ not formally evaluated]).

As ER/PR/HER2 IHC results may not be available in pathology reports submitted for CGP or may be inconclusive, we determined the impact of integrating qTP results with CGP results given that standard of care PIK3CA mutations are associated with FDA-approved alpelisib+fulvestrant therapy only in patients with hormone receptor positive/HER2 negative breast cancer. Hence, across the 4-month period of consecutively tested pan-solid FFPE tumor samples (n=3,904) submitted for clinical CGP testing described above, we identified those that were breast cancer and met qTP and CGP QC metrics (including the final ≥20% tumor content requirement) needed to evaluate HR status, PIK3CA mutations and ERBB2 copy number status. For all cases with standard of care PIK3CA mutations, we stratified results by hormone receptor (HR) status by qTP (qTP ER and PR Negative as HR Negative) and ERBB2 amplification status by StrataNGS CGP testing.

### Supplementary Results

### Clinical and Molecular Analyses Supporting the Validity of the Strata Clinical Molecular Database (SCMD)

Given the substantial proportion of patients in the SCMD with NSCLC and extensive previous characterization of molecular subtypes and associated therapies, we leveraged the NSCLC cohort to assess the clinical validity of using the SCMD to support this study. Of the 9,899 Strata Trial patients, 1,416 (14.3%) had NSCLC, with 157 patients with NSCLC receiving a first line systemic NSCLC targeted oncogene targeted monotherapy against EGFR, ALK, ROS1 or MET (regardless of whether currently preferred), those receiving a second generation or later inhibitor showed significantly longer TTNT vs. patients receiving first-generation (erlotinib, gefitinib or crizotinib) inhibitor (later [n=120] vs. first generation inhibitor [n=37], median TTNT 25.3 [20.3-33.6] vs. 11.0 [95% CI 8.7-21.7] months; adjusted hazards ratio for later vs. first-generation inhibitor 0.44 [95% CI 0.27-0.73], p=0.001, FIG. 43a. Importantly, in the 129 patients with NSCLC receiving a current first-line systemic targeted oncogene monotherapy, whether the treatment occurred before (treatment decision made from orthogonal testing, n=57) or after (n=72) receiving StrataNGS test results was not a significant predictor or TTNT (before vs. after StrataNGS results, median TTNT 22.8 [95% CI 17.3-31.8] months vs. 29.0 [95% CI 20.3-29.0] months; adjusted hazards ratio 1.46 [95% CI 0.77-2.79], p=0.25; FIG. 43b). Lastly, given the challenging nature of specimens received for CGP in our real-world experience, we routinely run samples not meeting our input sample characteristics and perform molecular pathologist review in all cases to “rescue” variants from samples not meeting usual CGP QC metrics from one or more variant classes. Hence, although limited in number, we compared TTNT in SCMD NSCLC patients treated with a biomarker matched, first line oncogene NCCN preferred targeted monotherapy TKI after receiving StrataNGS results based on whether the sample 1) passed both StrataNGS sample input criteria and relevant sequencing QC metrics (see Supplementary Methods) or 2) did not meet sample input or sequencing QC metrics but reported a therapy matched biomarker. Samples failing sample and/or sequencing QC metrics (n=17) vs. meeting all QC metrics (n=48) was not a significant predictor of TTNT (failing vs. meeting QC, median TTNT 17.1 [95% CI 10.0-17.1] vs 29.0 [95% CI 20.3-29.0] months; adjusted hazards ratio 1.72 [95% CI 0.49-5.99], p=0.39; FIG. 43c).

Appropriateness of Real-World Progression-Free Survival (rwPFS) for Studying Pembrolizumab Treatment Outcomes

To establish the appropriateness of real-world progression free survival (rwPFS; by time to next therapy) for studying PD-(L)1 treatment outcomes, rwPFS was compared to overall survival (OS) in the discovery cohort. As shown in FIG. 44, the overall correlation (Spearman p=0.58) was impacted by several outliers. Of the four patients with the greatest difference in months of OS vs. TTNT, the first was a patient with metastatic NSCLC harboring an EML4-ALK fusion by StrataNGS testing who was briefly treated with pembrolizumab and chemotherapy before prolonged treatment with crizotinib and lorlatinib (FIG. 44 red box), the second was a patient with metastatic melanoma who was briefly treated with pembrolizumab, then ipilimumab+nivolumab, prior to an extended course with imatinib (the patient harbored two VUS in KIT; FIG. 44 blue box), the third was a patient with metastatic melanoma who was treated with pembrolizumab prior to prolonged treatment with ipilumumab monotherapy (FIG. 44 green box), and the fourth was a patient with metastatic NSCLC harboring EGFR p.G719C and p.S768I mutations who was briefly treated with pembrolizumab and chemotherapy before prolonged treatment with osimertinb followed by pembrolizumab and chemotherapy (FIG. 44 purple box). Taken together, although correlated, these results support evaluation of both TTNT and OS herein, as well as the real-world importance of identifying patients with oncogenic alterations who may be more appropriately treated with targeted therapy vs. PD-(L)1 therapy.

### Validation of qTP and IRS: Introduction

The qTP component of the CGP+qTP test used to report IRS is performed from the same aliquot of RNA and multiplex PCR panel used in StrataNGS (for gene fusions), with the current version targeting 106 non-chimeric amplicons (103 target genes and 3 housekeeping genes) used for quantitative expression profiling. Target gene expression is determined in scaled, log 2, median-centered units of normalized reads per million [nRPM]16,17, representing target gene expression normalized to housekeeping gene expression (from HMBS, CIAO1 and EIF2B1; see below), then scaled to the distribution observed in a common FFPE cell line control per panel. All 106 amplicons (median insert length 94.5, range 61-120) use primers (median primer length 23, range 10-31) that span exon-exon boundaries and only full length reads (<2 base-level mismatches) are counted, ensuring specificity of all normalized target gene values from each amplicon. Beyond IRS, the qTP panel includes amplicons for quality control (candidate housekeeping genes, separate amplicons targeting different regions the same transcript) and multiple classes of target gene expression biomarkers, including those potentially useful for determining hormone receptor status (see below), those measuring proliferation index, and individual biomarkers that may have utility in predicting response (or clinical trial suitability/enrollment,) to investigational or approved expression based therapies (e.g., antibodies, antibody drug conjugates [ADCs], bispecific antibodies, radiopharmaceuticals, CAR-T cells, TCRs, etc.).

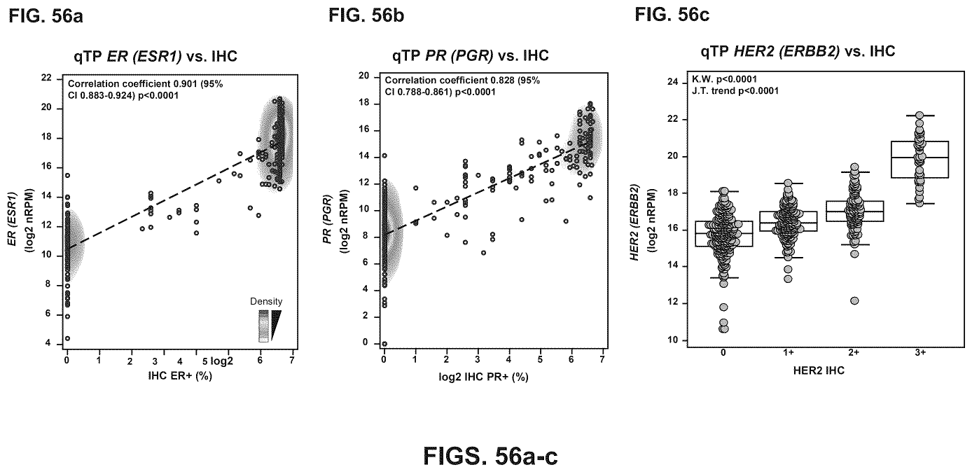

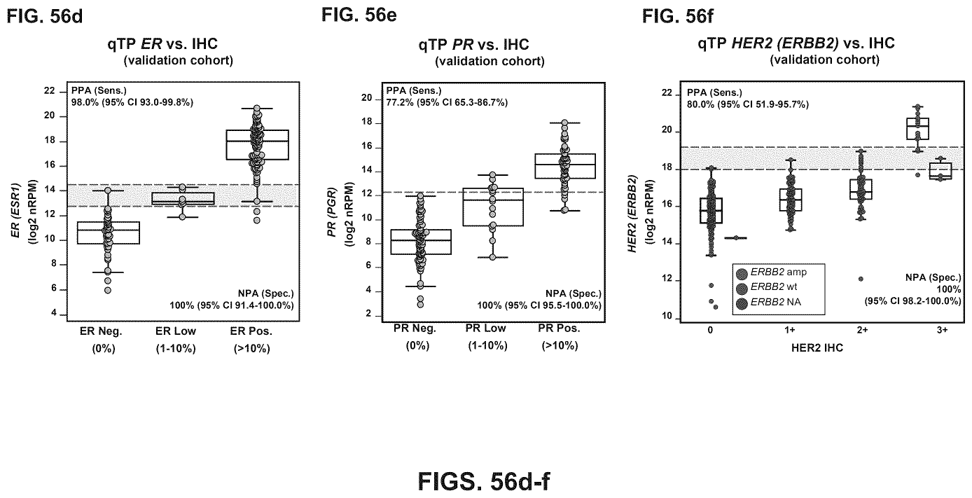

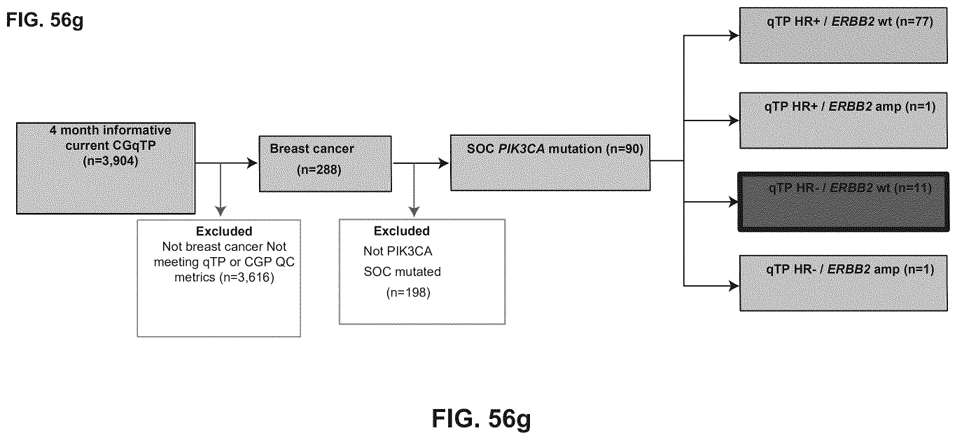

To be clinically useful, qTP (and derived integrated biomarkers such as IRS), must provide precise, quantitative results that can be used to predict therapeutic efficacy.^{16,17} As described in the validation of Omniseq—a New York state approved multiplex RT-PCR NGS assay—“standard measures that are routinely used to describe the analytical performance of variant detection assays (such as sensitivity and specificity) are not equally applicable to GEX [gene expression] by RNAseq, and well characterized reference standards for quantitative measurement of transcript levels are currently lacking”.^{16,17} Likewise, approaches used in validating assay linearity and quantitative bias in multi-gene expression assays not performed in multiplex (e.g., proportionality of threshold cycle [Ct] values vs. input RNA concentration^{16,17}), cannot be used directly in the validation of a multiplex RNAseq based-test. Hence, qTP panel-wide validation analyses applicable to IRS, as well as validation of the IRS biomarker, are described herein (see FIGS. 56a-g for validation of breast cancer related biomarkers).

### Validation of qTP and IRS: Housekeeping Gene Validation

Accurate quantitative gene expression by multiplex PCR based RNAseq requires appropriate housekeeping genes for target gene normalization; importantly, unlike approaches only applicable to a single tumor type (e.g., predictive gene expression assays for breast cancer) or only assessing a single class of biomarkers (e.g. immune cell and tumor microenvironment characterization), our approach required pan-tumor and pan-normal stable housekeeping genes given the inclusion of both tumor (e.g., ERBB2, ES1R, PGR) and non-tumor (e.g., PDCD1) components and the need to be robust to variable tumor content across tissues. Initial pre-clinical versions of the qTP panel contained 6 “positive control” genes across two RNA primer pools previously used in the RNA fusion component of the Oncomine Focus/Precision Assay. To evaluate the suitability of these markers as pan-cancer housekeeping genes for quantitative expression profiling and/or identify other candidates, we performed a multi-part evaluation of transcriptome profiles of pan-cancer, pan-normal tissue stability >20,000 tumor, normal and cancer cell line samples as shown in FIG. 46a. First, uniformly realigned, gene expression quantified, quantile normalized, and batch effect removed TCGA expression data (in fragments per kilobase per million [FPKM]) was downloaded for 6,875 tumor samples (from 18 tumor types) from 7. Correlation of variation (CV) was determined for each gene per tumor type, and candidate housekeeping genes were ranked by the number of tumor types in which they ranked in the top 20 most stable genes (by lowest CV). Uniformly processed gene expression data (in transcripts per million [TPM]) from the 18 highest ranking housekeeping genes, the 6 OPA “positive control genes”, as well as the commonly used housekeeping gene GAPDH were then downloaded for 20,841 total samples contained in the MiPanda database8, which includes 935 Cancer Cell Line Encyclopedia cell lines (from 20 tumor types), 9,966 normal tissue samples (730 TCGA samples from 20 tissue types and 9,236 GTEX samples from 30 tissue types), and 9,940 TCGA cancer tissue samples (9,496 primary samples from 25 tumor types and 444 metastases from 16 tumor types). Given that most target genes are expressed at much lower levels than typical control genes (such as GAPDH), we prioritized inclusion of pan-cancer, pan-normal tissue stable genes with the lowest average expression (in TPM) as candidate housekeeping genes for the qTP component of the CGP+qTP test thus selected five genes for inclusion: SLC4A1AP, CTCF, EIF2B1, CIAO1 and GGNBP2.

Three candidate housekeeping genes, EIF2B1, CIAO1 and HMBS, had the most stable expression patterns and widest LOQ range across clinical FFPE tumor samples in previous panel versions, and these three housekeeping genes are used in the validated version of the qTP panel defined herein, with target gene expression normalized to the expression of the median housekeeping gene. Normalized expression for these three housekeeping genes and the other candidates on the current version of the qTP panel are shown from the consecutive 4-month period of StrataNGS clinical testing described above (n=3,417 samples with reportable qTP, regardless of tumor content) in FIG. 46b, supporting the pan-tumor stability of the housekeeping genes used in the quantitative expression component of the qTP panel and IRS.

### Validation of qTP and IRS: Expression QC Metric Validation

Previous clinical versions of the expression component modeled the expected distribution of housekeeping gene expression and confirmed that each housekeeping gene is within its LOQ9; however, by comparing qTP panel wide, normalized target gene expression from 394 FFPE clinical tumor samples processed on the current version of the qTP panel validated herein, we observed highly correlated expression in all samples with >150,000 total mapped reads in the current version regardless of housekeeping gene distribution or LOQ status in the previous version (correlation coefficient=0.942 [95% CI 0.941-0.944], p-value<0.0001); in 129 samples with <150,000 mapped reads in the current qTP version, correlation coefficient vs. the previous version was only 0.649 (95% CI 0.639-0.659), supporting the use of this QC metric to determine reportability of individual qTP biomarkers and IRS. Importantly, through this series of analyses, we have demonstrated that each sample can serve as its own internal control to confirm suitability for quantitative gene expression.

Unlike traditional RNAseq, where overall gene quantification values (e.g., in FPKM) are reported and are dependent on alignment approaches, our multiplex PCR based RNAseq approach enables unambiguous read assignment to each target gene amplicon. As described above, two separate amplicons targeting different exon-exon junctions of the same gene are present in the qTP panel for several clinically relevant genes, including PD-L1, PD-1 and ADAM12 in the current qTP panel (only one of two ADAM12 amplicons was also present on all previous qTP panels used in IRS validation). Hence, we determined the correlation of replicate PD-L1, PD-1 and ADAM12 amplicons across the 24,463 Strata Trial clinical samples with complete sample information used to assess IRS distribution (see FIGS. 39a-d). As shown in FIGS. 46d-f, across the 24,463 samples (n=7,911 samples on panels with both ADAM12 amplicons), observed correlation coefficients for PD-L1, PD-1 and ADAM12 were 0.863 (95% CI 0.859-0.866; p<0.0001), 0.811 (95% CI 0.807-0.815; p<0.0001) and 0.908 (95% CT 0.904-0.912; p<0.0001), respectively.

### Validation of qTP and IRS: Limit of Quantification (LOQ), Linearity and Reportable Range

Limit of quantification (LOQ) and linearity experiments are complicated in multiplex RNA sequencing due to the lack of absolute standards at the extremes of expression that can be assessed in a complex RNA mixture with variable RNA composition and amplification efficiency. Hence, we determined LOQ and linearity for individual qTP target gene expression by determining the lowest nRPM that can be precisely quantified in replicate RNA aliquots from 619 clinical FFPE tumor samples subjected to repeat qTP, as the nRPM level below which repeat samples show increased dispersion represents the lowest amount of RNA library that can be precisely quantified. Across the 103 target gene amplicons, median linearity by concordance correlation coefficient of normalized target gene expression was 0.88 (range 0.50 to 0.99) across a median dynamic range (lower LOQ to highest replicate sample) of 252 fold (range 13-92,682 fold). For IRS, the individual scaled hazard rate is reported for informational purposes, however the IRS result is categorical as IRS-High or IRS-Low. As a Cox proportional hazard-based algorithm, IRS measurand LOD/LOQ and linearity is not applicable, and the reportable range of the quantitative scaled hazard rate is reported as determined without upper or lower bounds. Performance of the IRS model with or without LOQs applied to the individual expression components was compared and concordance correlation of IRS scores and quantification of the % of patients changing IRS groups (IRS-H to IRS-L or vice versa) was determined.

### Validation of qTP and IRS Accuracy Introduction

As described above, sensitivity/specificity/accuracy and linearity/LOQ are complicated in multiplex RNA sequencing due to the lack of absolute standards that can be assessed in a complex RNA mixture with variable RNA amplifiability, individual amplicon efficiency, and the difficulty in appropriately choosing a diluent (water to reduce input RNA amount, normal DNA for genomic alterations, or “normal” RNA to reduce relative amount of highly expressed tumor-specific transcripts are all inappropriate for a multiplex RNAseq pan-tumor approach targeting tumor and tumor microenvironment targets). Hence, the accuracy of the qTP panel and IRS expression components was validated through a multi-part accuracy study leveraging representational qRT-PCR validation, comparison to known target gene expression across tumor types (via comparison to TCGA expression data), and clinical IHC.

Validation of qTP and IRS Accuracy by qRT-PCR

The accuracy of the qTP component of the integrate CGP+qTP test was first determined by representational validation through evaluating target gene expression concordance with hydrolysis probe based qRT-PCR, the gold standard for gene-expression measurement.18 Clinical FFPE tumor samples from StrataNGS testing and 3 control RNA samples were subjected to qTP and qRT-PCR on replicate RNA aliquots. Comparison of target gene expression by qTP and qRT-PCR was performed for 32 target genes in 3 control samples and 24 clinical FFPE tumor samples (analytical validation) and the 24 FFPE tumor samples only (clinical validation). In the analytical validation, the observed concordance correlation coefficient was 0.837 (95% CI 0.816-856; p<0.0001). As shown in FIG. 46c, in the clinical validation, the observed concordance correlation coefficient was 0.842 (95% CI 0.820-862; p<0.0001); Likewise, when limited to just the four target genes comprising the expression component of IRS (PD-1, PD-L1, TOP2A, and ADAM12), the concordance correlation coefficient in the clinical validation was 0.833 (95% CI 0.760-0.886). Taken together, these results demonstrate the highly quantitative nature of the qTP panel and the gene expression component of IRS.

Validation of qTP and IRS Accuracy Vs. The Cancer Genome Atlas (TCGA) Profiled Tumors

To further evaluate the accuracy of the entire qTP panel, we compared qTP results for all 103 target genes to established gene expression profiles from the pan-cancer TCGA tumor set. Although TCGA data is largely from localized tumors, StrataNGS and the qTP panel is performed on either localized (from patients who later developed advanced/metastatic disease) or metastatic tumor samples. Hence, although an indirect measure of overall accuracy, expression profiles would be expected to be robust both across tumor types and transcriptional programs. As described in the methods, we compared Illumina HiSeq generated target gene expression profiles from the pan-cancer TCGA RNAseq tumor set, consisting of 9,618 samples from 30 TCGA tumor types that could be mapped directly to 28 Strata defined primary and/or secondary tumor types, to the 4-month period of clinical StrataNGS testing described above (3,222 FFPE tumor samples with reportable qTP and tumor content≥20%). Target gene expression profiles were highly concordant across TCGA and qTP when stratified by tumor type (median Spearman correlation across 19 tumor types with at least 10 samples in each group=0.897, range 0.838 to 0.925).

As the IRS model was trained using expression biomarkers present on both the current and 46 target gene qTP panels, we performed an expanded comparison of TCGA and qTP results for the immune and proliferation candidates included in IRS development across the 24,463 with complete sample information used to assess IRS distribution (see FIGS. 39a-d). As shown in Table S3, after limiting to 27 directly comparable tumor types, target gene expression per tumor type was compared for the 20 candidate expression biomarkers included in IRS development across 9,223 TCGA tumors and 18,305 qTP. Across all 20 candidates (IFNG was excluded from this analysis as it could not be reliably quantified across all versions of the quantitative expression profiling panel), the median Spearman correlation between TCGA and qTP tumors was 0.831 (range 0.624-0.938), while the median correlation of the four IRS components was 0.823, as TOP2A, PD-L1, PD-1 and ADAM12 showed correlations of 0.896, 0.831, 0.815, 0.707 (all p<0.0001), respectively.

Validation of qTP and IRS Accuracy Vs. Clinical Immunohistochemistry (IHC)

Although hydrolysis probe based qRT-PCR is the gold standard for RNA transcript quantification, IHC is the clinical gold standard for clinically relevant target expression evaluation. Therefore, we used optical character recognition and natural language processing to prioritize accompanying pathology reports received with StrataNGS test requests for abstraction of IHC biomarker results as described in the Supplementary Methods. Accuracy results relevant to IRS are described here, with results relevant to breast cancer biomarkers shown in FIGS. 56a-g.

Accuracy of the PD-L1 qTP component of IRS was validated against clinical IHC using a cohort of 276 NSCLC FFPE tumor samples with reportable qTP and PD-L1 IHC expression by the 22C3 clone (using TPS) in accompanying pathology reports. As shown in FIG. 47a, PD-L1 expression by qTP showed ordinally increasing median expression across the clinically relevant TPS bins (0%, 1-49%, and ≥50%; Kruskal Wallis p<0.0001, Jonckheere-Terpstra trend test p<0.0001). Only 24 of these samples came from the 154 patients in the propensity matched first line NSCLC treatment analysis (see FIG. 43a-c), precluding direct assessment of IRS vs. PD-L1 IHC for predicting pembrolizumab benefit. However, IRS status could be generated for all 276 NSCLC samples in the PD-L1 IHC cohort, with 31.0%, 34.2% and 58.0% of TPS 0%, 1-49%, and ≥50% samples being IRS-H, respectively. Accuracy of the TOP2A qTP component of IRS was validated against clinical IHC using a cohort of 956 FFPE tumor tissue samples (36 tumor types) with reportable qTP with proliferation index (percentage of Ki67 positive tumor cells) in accompanying pathology reports. As shown in FIG. 47b, TOP2A by qTP was strongly correlated to Ki67 proliferative index (correlation coefficient 0.753 [95% CI 0.724-0.780], p<0.0001). These results further support the accuracy of the gene expression component of IRS.

### Validation of qTP and IRS Reproducibility

Panel-wide qTP and IRS reproducibility between operators, lots, and instrumentation was established using separate replicate nucleic acid aliquots isolated from FFPE tumor samples. Twenty-seven unique samples were assessed by two operators on different days using different library preparation instrumentation, different library preparation reagent lots, and different templating and sequencing lots. As shown in FIG. 47c, concordance correlation coefficient of panel-wide (n=103 target genes) maximum vs. minimum nRPM for each target gene across all replicates for the 27 samples was 0.950 (95% CI 0.946-0.953; p<0.0001). Similarly, concordance correlation of maximum vs. minimum IRS score for each sample was 0.978 (95% CI 0.952-0.990; p<0.0001) as shown in FIG. 47d, with 100% concordance for IRS-H vs. -L status. Taken together, these results demonstrate the highly reproducible nature of qTP and the integrative IRS biomarker.

### Validation of qTP and IRS Summary

All qTP and IRS validation analyses met pre-specified acceptance criteria. Additional data supporting accuracy vs. other IHC biomarkers, analyses supporting the 20% overall qTP tumor content requirement beyond IRS, analyses supporting the utility of the current QC metrics, and clinical utility and validity of biomarkers beyond IRS and breast cancer biomarkers will be reported separately. IRS robustness to sample collection timing (e.g. immediately prior to PD-(L)1 therapy vs. prior to a preceding systemic therapy) and tumor content (supporting the overall 20% tumor content requirement) are described below.

Stratification of the Validation Cohort by PD-1 Vs. PD-L1 Therapy

In the overall 248 patient non-pembrolizumab PD-(L)1 monotherapy validation cohort (FIGS. 36c&d), by Kaplan Meier analysis, IRS-H patients had significantly longer PD-(L)1 monotherapy rwPFS (IRS-H vs. IRS-L median TTNT 23.1 [95% CI 17.1-32.9] vs. 10.2 [95% CI 8.7-14.8] months, adjusted hazard ratio=0.52 [95% CI 0.34-0.80], p=0.003) and OS (IRS-H vs. IRS-L median OS 40.4 [95% CI 32.9-NA] vs. 21.4 [95% CI 17.0-46.8] months, adjusted hazard ratio=0.49 [95% CI 0.30-0.80], p=0.005) compared to IRS-L patients. In the rwPFS analysis, PD-1 vs. PD-L1 inhibitor was not a significant term in the adjusted model (rwPFS adjusted hazard ratio 0.89 [95% CI 0.70-1.81], p=0.64), while in the OS analysis, PD-L1 inhibitors (vs. PD-1 inhibitors) were associated with significantly shorter OS (adjusted hazard ratio 1.93 [95% CI 1.14-3.25], p=0.014).

In the PD-L1 treated subset of the validation cohort (n=54 patients), by Kaplan Meier analysis, IRS-H patients had significantly longer rwPFS (IRS-H vs. IRS-L median rwPFS 28.2 [95% CI 15.1-NA] vs. 8.7 [95% CI 5.6-NA] months, adjusted hazard ratio=0.26 [95% CI 0.09-0.72], p=0.009; FIG. 50a) and OS (IRS-H vs. IRS-L median OS 28.2 [95% CI 15.1-NA] vs. 10.6 [95% CI 8.8-NA] months, adjusted hazard ratio=0.33 [95% CI 0.13-0.85], p=0.02; FIG. 50b) compared to IRS-L patients when adjusted for age, gender, most common tumor type (bladder cancer) vs. others, and line of therapy. In the PD-1 treated subset of the validation cohort (n=194 patients), by Kaplan Meier analysis, IRS-H patients had significantly longer TTNT by log-rank testing (IRS-H vs. IRS-L median TTNT 23.1 [95% CI 14.1-32.9] vs. 11.0 [95% CI 8.7-16.8] months, log-rank p=0.003), however when adjusted for age, gender, most common tumor type (melanoma) vs. others, and line of therapy, IRS status was not a significant predictor of TTNT (adjusted hazard ratio 0.62 [95% CI 0.38-1.01], p=0.054; FIG. 50c). Results were similar for OS analysis of the PD-1 subset, where IRS-H patients had significantly longer OS by log-rank testing (IRS-H vs. IRS-L median O Not reached [95% CI 32.9-NA] vs. 24.7 [95% CI 18.1-NA] months, log-rank p=0.047), however when adjusted IRS status was not a significant predictor of OS (adjusted hazard ratio 0.61 [95% CI 0.34-1.11], p=0.11 FIG. 50d). Taken together, despite the validation cohort including both PD-L1 and PD-1 therapies and having differing tumor type distributions compared to the discovery cohort, these results support the pan-solid tumor applicability of IRS to predict benefit of PD-(L)1 monotherapy.

### Validation of qTP and IRS: Stability of IRS Across Temporal Sample Collection Variability Prior to CPI Treatment

Tissue based TMB has recently been shown to be stable for nearly all patients with advanced cancer through whole genome sequencing of sequential tissue samples,¹⁹ however less is known about the stability of an integrative CGP+qTP model predicting pembrolizumab benefit. Hence, we assessed the impact of sample collection timing on IRS performance in the 310 patients in the discovery cohort that were also treated with a separate systemic line of therapy by stratifying patients by whether their samples were pre-systemic therapy and pembrolizumab vs. post-systemic therapy but pre-pembrolizumab. In the adjusted Cox proportional hazard model, IRS status (IRS-H [n=113] vs. IRS-L [n=197], adjusted hazard ratio 0.51 [95% CI 0.38-0.70], p<0.0001), but not pre-/post-systemic therapy collection timing (pre-systemic therapy [n=229] vs. post [n=81], adjusted hazard ratio 1.39 [95% CI 0.97-1.99], p=0.07), was significantly associated with pembrolizumab rwPFS, demonstrating that sample collection timing does not significantly impact IRS performance.

Next, we directly assessed IRS stability across patients in the SCMD with sequentially tested tissue samples. As analyses presented thus far were limited to the most recently tested sample per patient (if testing had been performed more than once), we therefore identified 104 total patients in the SCMD who 1) had valid IRS scores from two specimens with different collection dates, 2) were confirmed to be of clonal origin as part of routine StrataNGS clinical testing, and 3) did not have PD(L)-1 or anti-CTLA4 therapy starting between the collection dates of the samples. As shown in FIG. 53a, the integrative IRS model scores were highly correlated (correlation coefficient=0.65) in paired specimens, and only 16 (15.3%) patients moved from the IRS-H to -L (n=6) or IRS-L to -H (n=6) (or vice versa), supporting the stability of the IRS across temporal sampling in the absence of immunotherapy.

Lastly, we assessed the performance of IRS in 181 patients (from 17 tumor types) who otherwise would have been included in the discovery or validation cohorts, but had their sample collected after starting PD-(L)1 therapy (any pembrolizumab containing line [n=92 patients] or other PD-(L)1 monotherapy [n=89 patients]). Hypothesizing that CGP testing in this clinical scenario would usually be performed as the patient was progressing on (or had already progressed on) PD-(L)1 therapy, we predicted that IRS would be minimally predictive of PD-(L)1 TTNT. In these 181 patients, IRS status was not predictive of PD-(L)1 TTNT by Kaplan Meier analysis (IRS-H [n=77] vs. IRS-L [n=104], median 14.8 [95% CI 10.8-16.8] vs. 10.7 [95% CI 8.6-14.7] months, adjusted hazard ratio IRS-H vs. IRS-L 0.88 [95% CI 0.62-1.27], p=0.50) when adjusted for age, gender, therapy line, most common tumor type (NSCLC) vs. others, PD-(L)1 type (pembrolizumab vs. other PD-[L]1), and monotherapy vs. combination therapy (for pembrolizumab); FIG. 53b). Together, these results support the stability and validity of IRS in tumor tissue samples collected prior to CPI treatment.

### Validation of qTP and IRS Robustness of IRS to Tumor Content

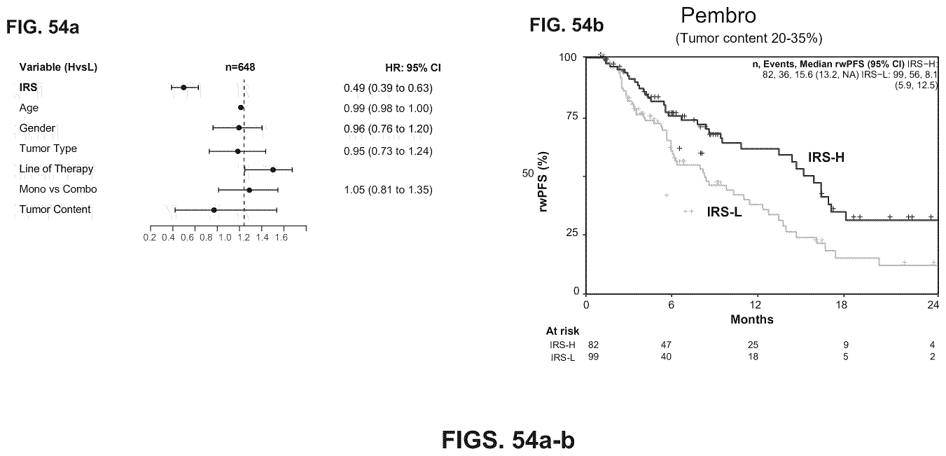

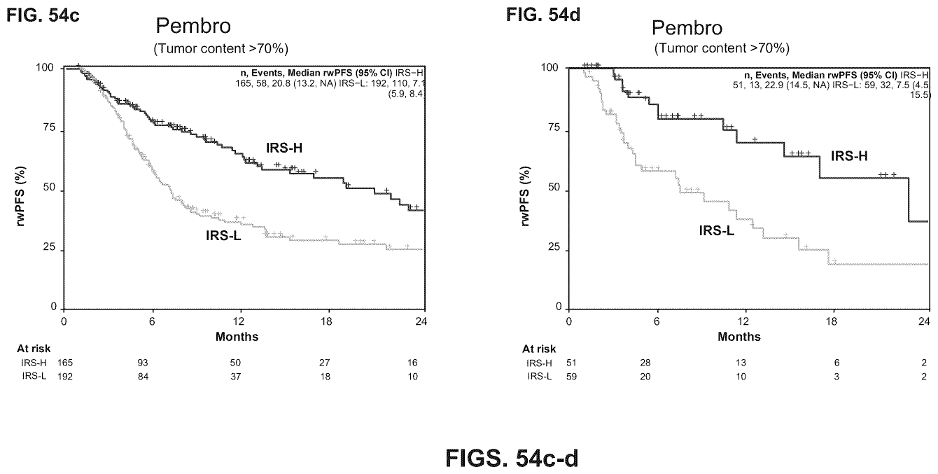

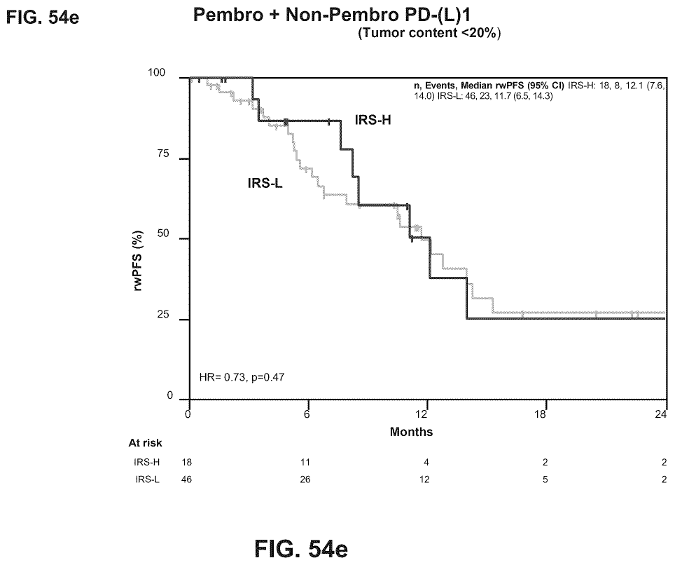

For the integrative IRS model, the actual tumor content of a given sample is impacted both by normal cells unrelated to the gene expression component of the IRS model (such as benign epithelial cells), as well as tumor infiltrating lymphocytes (such as those that express PDCD1 and/or PD-L1) and cancer associated fibroblasts that express ADAM1220-25, with these components (and the actual tumor content) directly relevant to the predictive ability of the IRS algorithm. Hence, an approach using “normal” RNA in a simple tumor content-based dilution series would be inappropriate to determine the clinical tumor content LOD for the IRS algorithm, even if not precluded by the difficulties in such an approach in multiplex RNA sequencing. Therefore to determine the tumor content limit of detection (LOD) for the IRS algorithm, we assessed the impact of tumor content on the predictive nature of the IRS algorithm. Thus, we included a continuous tumor content term (range: 20-100% [as the established LOD for accurate TMB estimation was determined as 20% tumor content]) and included this tumor content term in the overall adjusted CPH model for the IRS discovery cohort (including age, gender, most common tumor type [NSCLC] vs. others, therapy type [monotherapy/combination], and line of therapy). Importantly, while IRS remained a significant predictor of pembrolizumab TTNT (adjusted hazard ratio 0.49 [95% CI 0.39-0.63], p<0.0001), tumor content was not a significant predictor (adjusted hazard ratio 0.75 [95% CI 0.42-1.34], p=0.33; FIG. 54a). Kaplan Meier plots of pembrolizumab rwPFS stratified by IRS status is shown for relevant tumor content bins (20-35%, 40-70%, 75-100%) from analysis are shown in FIGS. 54b-d. Similar results were observed in the validation cohort, as adding the same tumor content term to the overall adjusted CPH model in this cohort demonstrated that while IRS remained as a significant predictor of PD-(L)1 rwPFS (adjusted hazard ratio 0.55 [95% CI 0.36-0.84], p=0.006), tumor content was not a significant predictor (adjusted hazard ratio 2.5 [95% CI 0.94-6.56], p=0.07).

Lastly, we identified 64 subjects in the SCMD that otherwise would have been included in the discovery or validation cohorts, but the tumor content of the tested sample was <20%. As shown in FIG. 54e, the IRS model was not predictive of pembrolizumab TTNT (IRS-H [n=18] vs. IRS-L [n=46], median TTNT 12.1 [95% CI 7.6-14.0] vs. 11.7 [95% CI 6.5-14.3] months; adjusted hazards ratio 0.73 [95% 0.32-1.70], p=0.47) when adjusted for age, gender, therapy line, most common tumor type (NSCLC) vs. others, PD-(L)1 type (pembrolizumab vs. other PD-[L]1), and monotherapy vs. combination therapy (for pembrolizumab). Taken together, these results further support the overall 20% tumor content LOD for the integrative IRS algorithm.

### Supplementary Discussion

As the IRS was developed from a single integrative clinical platform using co-isolated DNA and RNA to generate TMB and highly quantitative gene expression assessment of the tumor and TME from 648 patients across 24 tumor types, the IRS model holds several potentially interesting biological insights. First, TMB, PD-1 expression, and PD-L1 expression were each independent predictors of pembrolizumab benefit, indicating a multiplicative predictive effect across these biomarkers representing increased antigenicity (TMB) and the direct targets of both PD-1 and PD-L1 monoclonal antibodies. While PD-L1 evaluation by IHC is the current FDA-approved biomarker to predict PD-(L)1 benefit either individually or in models²⁶⁻²⁸, expression varies by antibody clone and nearly all studies show at least some responsive PD-L1-IHC low/negative patients²⁹⁻³³. In addition, PD-1 expression in both CD8+ and all lymphocytes has also been shown to be predictive of PD-(L)1 therapy benefit, most notably in Merkel cell carcinoma, where PD-1+ and PD-L1+ cell density, as well as close proximity of PD-1 and PD-L1+ cells, were associated with treatment response, while CD8+ cell density (nor CD8+ and PD-L1+ cell proximity) was not²⁸. Our results are also consistent with the numerous translational research studies showing that while both high TMB and immune gene expression (or PD-L1 IHC) are predictive of PD-(L)1 benefit, these biomarkers are largely uncorrelated^{18,31,34-45}.

Increasing TOP2A and ADAM12 expression were associated with decreased benefit from PD-(L)1 therapy in the IRS model. Although we have validated TOP2A as a marker of proliferating tumor cells (FIGS. 47a-d), its significance in the IRS model is unclear. Although less is known about the direct role of ADAM12 in CPI response, it is highly expressed by cancer associated fibroblasts CAFs—as shown through single cell sequencing studies and bulk tumor profiling—as a driver of feed forward TGF-β signaling, has been shown to act as a T cell co-stimulatory molecule expressed on some regulatory T cells, and has been identified in a signature of negative response to ICI in melanoma²⁰⁻²⁵. Of note, in colorectal cancer, where single cell sequencing demonstrated high ADAM12 expression in CAFs⁴⁶, as well as urothelial carcinoma, TGF-β signaling from CAFs has been shown to drive T cell exclusion, a hallmark of low response to ICI⁴⁷⁻⁵¹. Taken together, these results support additional investigation into a potential mechanistic role for ADAM12 in ICI resistance, as well as demonstrate the complementary nature of the integrative biomarkers in the IRS model, which integrates measurement of tumor neo-antigenicity (TMB), with quantification of key tumor and TME biomarkers.

### SUPPLEMENTARY REFERENCES

1 Tomlins, S. A. et al. Development and Validation of StrataNGS, a Multiplex PCR, Semiconductor Sequencing-Based Comprehensive Genomic Profiling Test. J Mol Diagn, doi:10.1016/j.jmoldx.2021.08.005 (2021).
2 Priestley, P. et al. Pan-cancer whole-genome analyses of metastatic solid tumours. Nature 575, 210-216, doi:10.1038/s41586-019-1689-y (2019).
3 Tomlins, S. A. et al. Real-World Performance of a Comprehensive Genomic Profiling Test Optimized for Small Tumor Samples. JCO Precis Oncol 5, doi:10.1200/PO.20.00472 (2021).
4 McGranahan, N. et al. Clonal neoantigens elicit T cell immunoreactivity and sensitivity to immune checkpoint blockade. Science 351, 1463-1469, doi:10.1126/science.aafl490 (2016).
5 Anagnostou, V. et al. Multimodal genomic features predict outcome of immune checkpoint blockade in non-small-cell lung cancer. Nature Cancer 1, 99-111, doi:10.1038/s43018-019-0008-8 (2020).
6 Conroy, J. M. et al. Analytical Validation of a Next-Generation Sequencing Assay to Monitor Immune Responses in Solid Tumors. J Mol Diagn 20, 95-109, doi:10.1016/j.jmoldx.2017.10.001 (2018).
7 Wang, Q. et al. Unifying cancer and normal RNA sequencing data from different sources. Sci Data 5, 180061, doi:10.1038/sdata.2018.61 (2018).
8 Niknafs, Y. S. et al. MiPanda: A Resource for Analyzing and Visualizing Next-Generation Sequencing Transcriptomics Data. Neoplasia 20, 1144-1149, doi:10.1016/j.neo.2018.09.001 (2018).
9 Harms, K. L. et al. Virus-positive Merkel Cell Carcinoma Is an Independent Prognostic Group with Distinct Predictive Biomarkers. Clin Cancer Res 27, 2494-2504, doi:10.1158/1078-0432.CCR-20-0864 (2021).
10 Cerami, E. et al. The cBio cancer genomics portal: an open platform for exploring multidimensional cancer genomics data. Cancer Discov 2, 401-404, doi:10.1158/2159-8290.CD-12-0095 (2012).
11 Hovelson, D. H. et al. Targeted DNA and RNA Sequencing of Paired Urothelial and Squamous Bladder Cancers Reveals Discordant Genomic and Transcriptomic Events and Unique Therapeutic Implications. Eur Urol 74, 741-753, doi:10.1016/j.eururo.2018.06.047 (2018).
12 Plaska, S. W. et al. Targeted RNAseq of Formalin-Fixed Paraffin-Embedded Tissue to Differentiate Among Benign and Malignant Adrenal Cortical Tumors. Horm Metab Res 52, 607-613, doi:10.1055/a-1212-8803 (2020).
13 Salami, S. S. et al. Transcriptomic heterogeneity in multifocal prostate cancer. JCI Insight 3, doi:10.1172/jci.insight.123468 (2018).
14 Tomlins, S. A. et al. Integrative molecular concept modeling of prostate cancer progression. Nat Genet 39, 41-51, doi:10.1038/ng1935 (2007).
15 Tomlins, S. A. et al. Development and Validation of StrataNGS, a Multiplex PCR, Semiconductor Sequencing-Based Comprehensive Genomic Profiling Test. J Mol Diagn 23, 1515-1533, doi:10.1016/j.jmoldx.2021.08.005 (2021).
16 Cronin, M. et al. Analytical validation of the Oncotype DX genomic diagnostic test for recurrence prognosis and therapeutic response prediction in node-negative, estrogen receptor-positive breast cancer. Clin Chem 53, 1084-1091, doi:10.1373/clinchem.2006.076497 (2007).
17 Simon, R. Roadmap for developing and validating therapeutically relevant genomic classifiers. J Clin Oncol 23, 7332-7341, doi:10.1200/JCO.2005.02.8712 (2005).
18 Litchfield, K. et al. Meta-analysis of tumor- and T cell-intrinsic mechanisms of sensitization to checkpoint inhibition. Cell 184, 596-614 e514, doi:10.1016/j.cell.2021.01.002 (2021).
19 van de Haar, J. et al. Limited evolution of the actionable metastatic cancer genome under therapeutic pressure. Nat Med 27, 1553-1563, doi:10.1038/s41591-021-01448-w (2021).
20 Yu, C. et al. A five-gene signature is a prognostic biomarker in pan-cancer and related with immunologically associated extracellular matrix. Cancer Med 10, 4629-4643, doi:10.1002/cam4.3986 (2021).
21 Liu, Y. et al. ADAM12 is a costimulatory molecule that determines Th1 cell fate and mediates tissue inflammation. Cell Mol Immunol 18, 1904-1919, doi:10.1038/s41423-020-0486-8 (2021).
22 Cheon, D. J. et al. ADAM12 is a prognostic factor associated with an aggressive molecular subtype of high-grade serous ovarian carcinoma. Carcinogenesis 36, 739-747, doi:10.1093/carcin/bgv059 (2015).
23 Cui, C. et al. Ratio of the interferon-gamma signature to the immunosuppression signature predicts anti-PD-1 therapy response in melanoma. NPJ Genom Med 6, 7, doi:10.1038/s41525-021-00169-w (2021).
24 Ruff, M. et al. The Disintegrin and Metalloprotease ADAM12 Is Associated with TGF-beta-Induced Epithelial to Mesenchymal Transition. PLoS One 10, e0139179, doi:10.1371/journal.pone.0139179 (2015).
25 Atfi, A. et al. The disintegrin and metalloproteinase ADAM12 contributes to TGF-beta signaling through interaction with the type II receptor. J Cell Biol 178, 201-208, doi:10.1083/jcb.200612046 (2007).
26 Medicine, F. (2021).
27 Yearley, J. H. et al. PD-L2 Expression in Human Tumors: Relevance to Anti-PD-1 Therapy in Cancer. Clin Cancer Res 23, 3158-3167, doi:10.1158/1078-0432.CCR-16-1761 (2017).
28 Giraldo, N. A. et al. Multidimensional, quantitative assessment of PD-1/PD-L1 expression in patients with Merkel cell carcinoma and association with response to pembrolizumab. J Immunother Cancer 6, 99, doi:10.1186/s40425-018-0404-0 (2018).
29 Garon, E. B. et al. Pembrolizumab for the treatment of non-small-cell lung cancer. N Engl J Med 372, 2018-2028, doi:10.1056/NEJMoa1501824 (2015).
30 Robert, C. et al. Pembrolizumab versus Ipilimumab in Advanced Melanoma. N Engl J Med 372, 2521-2532, doi:10.1056/NEJMoa1503093 (2015).
31 Herbst, R. S. et al. Predictive correlates of response to the anti-PD-L1 antibody MPDL3280A in cancer patients. Nature 515, 563-567, doi:10.1038/nature14011 (2014).
32 Tumeh, P. C. et al. PD-1 blockade induces responses by inhibiting adaptive immune resistance. Nature 515, 568-571, doi:10.1038/nature13954 (2014).
33 Mahoney, K. M. & Atkins, M. B. Prognostic and predictive markers for the new immunotherapies. Oncology (Williston Park) 28 Suppi 3, 39-48 (2014).
34 Cristescu, R. et al. Pan-tumor genomic biomarkers for PD-1 checkpoint blockade-based immunotherapy. Science 362, doi:10.1126/science.aar3593 (2018).
35 Lee, J. S. & Ruppin, E. Multiomics Prediction of Response Rates to Therapies to Inhibit Programmed Cell Death 1 and Programmed Cell Death 1 Ligand 1. JAMA Oncol 5, 1614-1618, doi:10.1001/jamaoncol.2019.2311 (2019).
36 Rolfo, C. et al. Liquid Biopsy for Advanced NSCLC: A Consensus Statement From the International Association for the Study of Lung Cancer. J Thorac Oncol 16, 1647-1662, doi:10.1016/j.jtho.2021.06.017 (2021).
37 Taube, J. M. et al. Colocalization of inflammatory response with B7-h1 expression in human melanocytic lesions supports an adaptive resistance mechanism of immune escape. Sci Transl Med 4, 127rai37, doi:10.1126/scitranslmed.3003689 (2012).
38 Chen, D. S. & Mellman, I. Elements of cancer immunity and the cancer-immune set point. Nature 541, 321-330, doi:10.1038/nature21349 (2017).
39 Sanmamed, M. F. & Chen, L. A Paradigm Shift in Cancer Immunotherapy: From Enhancement to Normalization. Cell 175, 313-326, doi:10.1016/j.cell.2018.09.035 (2018).
40 Ayers, M. et al. IFN-gamma-related mRNA profile predicts clinical response to PD-1 blockade. J Clin Invest 127, 2930-2940, doi:10.1172/JCI91190 (2017).
41 Fountzilas, E., Kurzrock, R., Hiep Vo, H. & Tsimberidou, A. M. Wedding of Molecular Alterations and Immune Checkpoint Blockade: Genomics as a Matchmaker. J Natl Cancer Inst, doi:10.1093/jnci/djab067 (2021).
42 Ott, P. A. et al. T-Cell-Inflamed Gene-Expression Profile, Programmed Death Ligand 1 Expression, and Tumor Mutational Burden Predict Efficacy in Patients Treated With Pembrolizumab Across 20 Cancers: KEYNOTE-028. J Clin Oncol 37, 318-327, doi:10.1200/JCO.2018.78.2276 (2019).
43 Lu, S. et al. Comparison of Biomarker Modalities for Predicting Response to PD-1/PD-L1 Checkpoint Blockade: A Systematic Review and Meta-analysis. JAMA Oncol 5, 1195-1204, doi:10.1001/jamaoncol.2019.1549 (2019).
44 Zhang, Z. et al. RNF2 ablation reprograms the tumor-immune microenvironment and stimulates durable NK and CD4+ T-cell-dependent antitumor immunity. Nature Cancer 2, 1018-1038, doi:10.1038/s43018-021-00263-z (2021).
45 Cristescu, R. et al. Transcriptomic Determinants of Response to Pembrolizumab Monotherapy Across Solid Tumor Types. Clin Cancer Res, doi:10.1158/1078-0432.CCR-21-3329 (2021).
46 Pelka, K. et al. Spatially organized multicellular immune hubs in human colorectal cancer. Cell 184, 4734-4752 e4720, doi:10.1016/j.cell.2021.08.003 (2021).
47 Mariathasan, S. et al. TGFbeta attenuates tumour response to PD-L1 blockade by contributing to exclusion of T cells. Nature 554, 544-548, doi:10.1038/nature25501 (2018).
48 Tauriello, D. V. F. et al. TGFbeta drives immune evasion in genetically reconstituted colon cancer metastasis. Nature 554, 538-543, doi:10.1038/nature25492 (2018).
49 Jiang, P. et al. Signatures of T cell dysfunction and exclusion predict cancer immunotherapy response. Nat Med 24, 1550-1558, doi:10.1038/s41591-018-0136-1 (2018).
50 Bonaventura, P. et al. Cold Tumors: A Therapeutic Challenge for Immunotherapy. Front Immunol 10, 168, doi:10.3389/fimmu.2019.00168 (2019).
51 Gajewski, T. F., Schreiber, H. & Fu, Y. X. Innate and adaptive immune cells in the tumor microenvironment. Nat Immunol 14, 1014-1022, doi:10.1038/ni.2703 (2013).
52 Rousseau, B. et al. The Spectrum of Benefit from Checkpoint Blockade in Hypermutated Tumors. N Engl J Med 384, 1168-1170, doi:10.1056/NEJMc2031965 (2021).

### Supplementary Tables

TABLE S1
Tumor types of study cohorts

| | | | | | | | Validation | | Additional | | | |
|---|---|---|---|---|---|---|---|---|---|---|---|---|
| | IRS | | Systemic | | Discovery | | Cohort | | Cohorts | | | |
| | Distribution | | Therapy | | Cohort | | PD-(L)1 | | Nivolumab + | | | |
| Primary Tumor | Cohort | | Cohort | | Pembrolizumab | | monotherapy | | ipilimumab | | Non-IO | |
| Type | n (%) | | n (%) | | n (%) | | n (%) | | n (%) | | n (%) | |
| Cohort total | 24,463 | (100%) | 9,899 | (100%) | 648 | (100%) | 248 | (100%) | 70 | (100%) | 3,184 | (100%) |
| n (%) | | | | | | | | | | | | |
| Total number | 45 | | 43 | | 26 | | 24 | | 8 | | 35 | |
| of tumor types | | | | | | | | | | | | |
| Adrenocortical | 31 | (0.1%) | 13 | (0.1%) | 0 | (0%) | 0 | (0%) | 0 | (0%) | 5 | (0.2%) |
| Carcinoma | | | | | | | | | | | | |
| Ampullary | 55 | (0.2%) | 18 | (0.2%) | 0 | (0%) | 0 | (0%) | 0 | (0%) | 7 | (0.2%) |
| Carcinoma | | | | | | | | | | | | |
| Anal Cancer | 79 | (0.3%) | 24 | (0.3%) | 1 | (0.2%) | 2 | (0.8%) | 1 | (1.4%) | 10 | (0.3%) |
| Appendiceal | 81 | (0.3%) | 22 | (0.3%) | 0 | (0%) | 0 | (0%) | 0 | (0%) | 6 | (0.2%) |
| Cancer | | | | | | | | | | | | |
| Bladder | 741 | (3.0%) | 239 | (3.0%) | 48 | (7.4%) | 17 | (6.9%) | 0 | (0%) | 63 | (2.0%) |
| Cancer | | | | | | | | | | | | |
| Breast Cancer | 2438 | (10%) | 1329 | (10%) | 7 | (1.1%) | 8 | (3.2%) | 0 | (0%) | 413 | (13.0%) |
| Cancer of | 1767 | (7.2) | 514 | (5.2%) | 62 | (9.6%) | 16 | (6.5%) | 4 | (5.7%) | 178 | (5.6%) |
| Unknown | | | | | | | | | | | | |
| Primary | | | | | | | | | | | | |
| Cervical Cancer | 157 | (0.6%) | 73 | (0.7%) | 12 | (1.9%) | 1 | (0.4%) | 0 | (0%) | 35 | (1.1%) |
| CNS and PNS | 101 | (0.4%) | 8 | (0.1%) | 0 | (0%) | 0 | (0%) | 0 | (0%) | 0 | (0%) |
| Cancer | | | | | | | | | | | | |
| Colorectal | 3147 | (12.9%) | 1398 | (14.1%) | 13 | (2.0%) | 9 | (3.6%) | 3 | (4.3%) | 606 | (19.0%) |
| Cancer | | | | | | | | | | | | |
| Embryonal | 8 | (0%) | 3 | (0%) | 0 | (0%) | 0 | (0%) | 0 | (0%) | 0 | (0%) |
| Tumor | | | | | | | | | | | | |
| Endometrial | 978 | (4%) | 280 | (2.8%) | 29 | (4.5%) | 1 | (0.4%) | 0 | (0%) | 144 | (4.5%) |
| Cancer | | | | | | | | | | | | |
| Esophagogastric | 1097 | (4.5%) | 405 | (4.1%) | 31 | (4.8%) | 4 | (1.6%) | 0 | (0%) | 148 | (4.6%) |
| Cancer | | | | | | | | | | | | |
| Gastrointestinal | 182 | (0.7%) | 32 | (0.3%) | 0 | (0%) | 1 | (0.4%) | 0 | (0%) | 10 | (0.3%) |
| Stromal Tumor | | | | | | | | | | | | |
| Germ Cell Tumor | 54 | (0.2%) | 6 | (0.1%) | 0 | (0%) | 0 | (0%) | 0 | (0%) | 4 | (0.1%) |
| Glioma | 971 | (4.0%) | 492 | (5.0%) | 5 | (0.8%) | 2 | (0.8%) | 0 | (0%) | 230 | (7.2%) |
| Head and Neck | 576 | (2.4%) | 265 | (2.7%) | 53 | (8.2%) | 14 | (5.6%) | 0 | (0%) | 101 | (3.2%) |
| Cancer | | | | | | | | | | | | |
| Hepatobiliary | 567 | (2.3%) | 195 | (2.0%) | 4 | (0.6%) | 9 | (3.6%) | 0 | (0%) | 65 | (2.0%) |
| Cancer | | | | | | | | | | | | |
| Histiocytosis | 4 | (0%) | 2 | (0%) | 0 | (0%) | 0 | (0%) | 0 | (0%) | 0 | (0%) |
| Lymphoma | 94 | (0.4%) | 136 | (1.4%) | 0 | (0%) | 1 | (0.4%) | 0 | (0%) | 63 | (2.0%) |
| Melanoma | 897 | (3.7%) | 312 | (3.2%) | 37 | (5.7%) | 62 | (25%) | 33 | (47.1%) | 22 | (0.7%) |
| Mesothelioma | 86 | (0.4%) | 15 | (0.2%) | 1 | (0.2%) | 1 | (0.4%) | 0 | (0%) | 4 | (0.1%) |
| Miscellaneous | 9 | (0%) | 0 | (0%) | 0 | (0%) | 0 | (0%) | 0 | (0%) | 0 | (0%) |
| Neuroepithelial | | | | | | | | | | | | |
| Tumor | | | | | | | | | | | | |
| Nerve Sheath | 21 | (0.1%) | 5 | (0.1%) | 0 | (0%) | 0 | (0%) | 0 | (0%) | 5 | (0.2%) |
| Tumor | | | | | | | | | | | | |
| Neuroendocrine | 278 | (1.1%) | 56 | (0.6%) | 4 | (0.6%) | 1 | (0.4%) | 1 | (1.4%) | 14 | (0.4%) |
| Tumor | | | | | | | | | | | | |
| Non-Small | 3895 | (15.9%) | 1416 | (14.3%) | 265 | (40.9%) | 37 | (14.9%) | 2 | (2.9%) | 244 | (7.7%) |
| Cell Lung | | | | | | | | | | | | |
| Cancer | | | | | | | | | | | | |
| NUT Midline | 4 | (0%) | 0 | (0%) | 0 | (0%) | 0 | (0%) | 0 | (0%) | 0 | (0%) |
| Carcinoma | | | | | | | | | | | | |
| Other | 8 | (0%) | 119 | (1.2%) | 10 | (1.5%) | 3 | (1.2%) | 0 | (0%) | 53 | (1.7%) |
| Cancer | | | | | | | | | | | | |
| Ovarian | 1329 | (5.4%) | 541 | (5.5%) | 7 | (1.1%) | 0 | (0%) | 0 | (0%) | 223 | (7.0%) |
| Cancer | | | | | | | | | | | | |
| Pancreatic | 991 | (4.1%) | 614 | (6.2%) | 7 | (1.1%) | 0 | (0%) | 0 | (0%) | 158 | (5.0%) |
| Cancer | | | | | | | | | | | | |
| Penile Cancer | 14 | (0.1%) | 3 | (0%) | 1 | (0.2%) | 0 | (0%) | 0 | (0%) | 0 | (0%) |
| Pheochromocytoma | 4 | (0%) | 3 | (0%) | 0 | (0%) | 0 | (0%) | 0 | (0%) | 1 | (0%) |
| Pineal Tumor | 2 | (0%) | 1 | (0%) | 0 | (0%) | 0 | (0%) | 0 | (0%) | 0 | (0%) |
| Prostate | 1427 | (5.8) | 548 | (5.5%) | 2 | (0.3%) | 1 | (0.4%) | 0 | (0%) | 168 | (5.4%) |
| Cancer | | | | | | | | | | | | |
| Renal Cell | 468 | (1.9%) | 272 | (2.7%) | 23 | (3.5%) | 32 | (12.9%) | 24 | (34.3%) | 33 | (1.0%) |
| Carcinoma | | | | | | | | | | | | |
| Sarcoma | 826 | (3.4%) | 197 | (2.0%) | 9 | (1.4%) | 3 | (1.2%) | 2 | (2.9%) | 79 | (2.5%) |
| Salivary | 117 | (0.5%) | 5 | (0.1%) | 0 | (0%) | 0 | (0%) | 0 | (0%) | 0 | (0%) |
| Gland Cancer | | | | | | | | | | | | |
| Sellar Tumor | 11 | (0%) | 1 | (0%) | 0 | (0%) | 0 | (0%) | 0 | (0%) | 0 | (0%) |
| Sex Cord | 30 | (0.1%) | 2 | (0%) | 0 | (0%) | 0 | (0%) | 0 | (0%) | 0 | (0%) |
| Stromal Tumor | | | | | | | | | | | | |
| Skin Cancer, | 131 | (0.5%) | 59 | (0.6%) | 9 | (1.4%) | 7 | (2.8%) | 0 | (0%) | 12 | (0.4%) |
| Non- | | | | | | | | | | | | |
| Melanoma | | | | | | | | | | | | |
| Small Bowel | 94 | (0.4%) | 42 | (0.4%) | 0 | (0%) | 0 | (0%) | 0 | (0%) | 18 | (0.6%) |
| Cancer | | | | | | | | | | | | |
| Small Cell | 283 | (1.2%) | 150 | (1.5%) | 3 | (0.5%) | 15 | (6.0%) | 0 | (0%) | 30 | (0.9%) |
| Lung Cancer | | | | | | | | | | | | |
| Thymic Tumor | 50 | (0.2%) | 19 | (0.2%) | 2 | (0.3%) | 0 | (0%) | 0 | (0%) | 6 | (0.2%) |
| Thyroid | 291 | (1.2%) | 55 | (0.6%) | 3 | (0.5%) | 0 | (0%) | 0 | (0%) | 23 | (0.7%) |
| Cancer | | | | | | | | | | | | |
| Vaginal | 69 | (0.3%) | 10 | (0.1%) | 0 | (0%) | 1 | (0.4%) | 0 | (0%) | 3 | (0.1%) |
| Cancer | | | | | | | | | | | | |

IRS = Immunotherapy Response Score, n = sample size, % = percentage, PD-(L)1 = PD-L1 or PD-1 therapy, IO = immunotherapy (PD-[L]1 or CTLA4 therapy), CNS = central nervous system, PNS = peripheral nervous system.
Breakdown by StrataNGS CGP assigned primary tumor type (total number and percentage of each cohort) for the cohorts used for discovery, validation, and additional analysis. The total number of tumor types for each cohort includes each primary tumor type for which there was at least one sample.

TABLE S2
Demographics by study cohorts

| | | | | | | | | Validation | | Additional | | | |
|---|---|---|---|---|---|---|---|---|---|---|---|---|---|
| | | IRS | | Systemic | | Discovery | | Cohort | | Cohorts | | | |
| | | Distribution | | Therapy | | Cohort | | PD- (L)1 | | Nivolumab + | | | |
| Variable | Statistic | Cohort | | Cohort | | Pembrolizumab | | monotherapy | | ipilimumab | | Non-IO | |
| Age | n | 24,463 | | 9,899 | | 648 | | 248 | | 70 | | 3,184 | |
| | mean (std) | 66.5 | (13.01) | 66.4 | (12.47) | 69.1 | (11.27) | 69.3 | (12.36) | 64.3 | (10.67) | 65.6 | (12.88) |
| | median | 68 | | 68 | | 70 | | 69.5 | | 65.5 | | 67 | |
| | min, max | 0, 89 | | 19, 89 | | 27, 89 | | 29, 89 | | 33, 83 | | 19, 89 | |
| Gender | n (%) | | | | | | | | | | | | |
| Female | | 12,948 | (52.9%) | 5,317 | (53.7%) | 295 | (45.5%) | 103 | (41.5%) | 19 | (27.1%) | 1,778 | (55.8%) |
| Male | | 11,513 | (47.1%) | 4,582 | (46.3%) | 353 | (54.5%) | 145 | (58.5%) | 51 | (72.9%) | 1,406 | (44.2%) |
| Unknown/ | | 2 | (0%) | 0 | (0%) | 0 | (0%) | 0 | (0%) | 0 | (0%) | 0 | (0%) |
| Missing | | | | | | | | | | | | | |
| Race | n (%) | | | | | | | | | | | | |
| American | | 59 | (0.2%) | 25 | (0.3%) | 2 | (0.3%) | 0 | | 1 | (1.4%) | 7 | (0.2%) |
| Indian or | | | | | | | | | | | | | |
| Alaskan | | | | | | | | | | | | | |
| Native | | | | | | | | | | | | | |
| Asian | | 1,421 | (5.8%) | 111 | (1.1%) | 6 | (0.9%) | 1 | (0.4%) | 1 | (1.4%) | 37 | (1.2%) |
| Black or | | 1,501 | (6.1%) | 788 | (8.0%) | 53 | (8.2%) | 6 | (2.4%) | 4 | (5.7%) | 233 | (7.3%) |
| African | | | | | | | | | | | | | |
| American | | | | | | | | | | | | | |
| Native | | 48 | (0.2%) | 11 | (0.1%) | 0 | | 0 | | 0 | | 5 | (0.2%) |
| Hawaiian or | | | | | | | | | | | | | |
| Other | | | | | | | | | | | | | |
| Pacific | | | | | | | | | | | | | |
| Islander | | | | | | | | | | | | | |
| White or | | 9,782 | (40.0%) | 6,142 | (62.0%) | 411 | (63.5%) | 66 | (67%) | 52 | (74.3%) | 2,001 | (62.8%) |
| Caucasian | | | | | | | | | | | | | |
| Other | | 1,232 | (5.0%) | 170 | (1.7%) | 14 | (2.2%) | 3 | (1.2%) | 1 | (1.4%) | 61 | (1.9%) |
| Unknown, | | 10,420 | (42.6%) | 2,652 | (26.8%) | 162 | (25%) | 72 | (29%) | 11 | (15.7%) | 840 | (26.4%) |
| Missing, or | | | | | | | | | | | | | |
| Not | | | | | | | | | | | | | |
| Reported | | | | | | | | | | | | | |
| Ethnicity | n (%) | | | | | | | | | | | | |
| Hispanic | | 766 | (3.1%) | 208 | (2.1%) | 16 | (2.5%) | 2 | (0.8%) | 2 | (2.9%) | 78 | (2.4%) |
| or Latino | | | | | | | | | | | | | |
| Not | | 12,420 | (50.8%) | 6,402 | (64.7%) | 435 | (67.1%) | 144 | (58.1%) | 53 | (75.7%) | 2,075 | (65.2%) |
| Hispanic or | | | | | | | | | | | | | |
| Latino | | | | | | | | | | | | | |
| Unknown, | | 11,277 | (46.1%) | 3,289 | (33.2%) | 197 | (30.4%) | 102 | (41.1%) | 15 | (21.4%) | 1,031 | (32.4%) |
| Missing, or | | | | | | | | | | | | | |
| Not | | | | | | | | | | | | | |
| Reported | | | | | | | | | | | | | |
| Basic Line | n (%) | | | | | | | | | | | | |
| 1 | | | | | | 467 | (72.1%) | 163 | (65.7%) | 55 | (78.6%) | 3,184 | (100%) |
| 2 | | | | | | 129 | (19.9%) | 60 | (24.2%) | 10 | (14.3%) | | |
| 3+ | | | | | | 52 | (8.0%) | 25 | (10.1%) | 5 | (7.1%) | | |
| Therapy | n (%) | | | | | | | | | | | | |
| Monotherapy | | | | | | 421 | (65.0%) | 248 | (100%) | 0 | (0%) | 1,256 | (39.4%) |
| Combination | | | | | | 227 | (35.0%) | 0 | (0%) | 70 | (100%) | 1,928 | (60.6%) |
| Combination w/ | | | | | | 192 | (84.6%) | NA | | 0 | (0%) | 1,731 | (89.8%) |
| chemotherapy | | | | | | | | | | | | | |
| Combination w/ | | | | | | 35 | (15.4%) | NA | | 70 | (100%) | 197 | (10.2%) |
| other therapy | | | | | | | | | | | | | |
| PD-(L)1 | n (%) | | | | | | | | | | | | |
| Therapy | | | | | | | | | | | | | |
| PD-1 | | | | | | 648 | (100%) | 194 | (78.2%) | | | | |
| Blockade | | | | | | | | | | | | | |
| Pembrolizumab | | | | | | 648 | (100%)* | 0 | (0%)* | | | | |
| Nivolumab | | | | | | 0 | (0%) | 190 | (97.9%)* | | | | |
| Cemiplimab | | | | | | 0 | (0%) | 4 | (2.1%)* | | | | |
| PD-L1 | | | | | | 0 | (0%) | 54 | (21.8%) | | | | |
| Blockade | | | | | | | | | | | | | |
| Atezolizumab | | | | | | 0 | (0%) | 49 | (90.7%)* | | | | |
| Avelumab | | | | | | 0 | (0%) | 5 | (9.3%)* | | | | |

IO = immunotherapy (PD-[L]1 or CTLA4 therapy), w/= with, n = sample size, std = standard deviation, min = minimum, max = maximum, % = percentage
*Percentages based on PD-1 or PD-L1 participant totals, respectively
Demographics considered for each cohort included gender, race, ethnicity, and line of therapy. For the discovery cohort, both pembrolizumab monotherapy and combination therapy containing lines were included. For the PD-(L)1 validation cohort, only PD-1 or PD-L1 monotherapy (excluding pembrolizumab) were included.

TABLE S3
Pan-cancer correlation of candidate IRS target gene expression
between Strata multiplex PCR-based NGS and TCGA

Target gene expression
Strata (n = 18,305) vs.
TCGA (n = 9,223)

| Gene | Type | ρ | p-value |
|---|---|---|---|
| TOP2A | IRS component | 0.896 | 9.71E−17 |
| | (Proliferation) | | |
| CD274 | IRS component | 0.831 | 1.62E−12 |
| (PD-L1) | (Immune | | |
| | candidate) | | |
| PDCD1 | IRS component | 0.815 | 9.22E−12 |
| (PD-1) | (Immune | | |
| | candidate) | | |
| ADAM12 | IRS component | 0.707 | 5.63E−08 |
| | (Immune | | |
| | candidate) | | |

Subset Median: 0.823

| | | | |
|---|---|---|---|
| VTCN1 | Immune candidate | 0.938 | 1.92E−21 |
| IDO1 | Immune candidate | 0.915 | 1.44E−18 |
| UBE2C | Proliferation | 0.887 | 4.67E−16 |
| TNFRSF9 | Immune candidate | 0.869 | 9.41E−15 |
| CTLA4 | Immune candidate | 0.869 | 1.02E−14 |
| HAVCR2 | Immune candidate | 0.845 | 2.79E−13 |
| LAG3 | Immune candidate | 0.833 | 1.28E−12 |
| GZMA | Immune candidate | 0.831 | 1.56E−12 |
| TIGIT | Immune candidate | 0.809 | 1.82E−11 |
| TCF7 | Immune candidate | 0.778 | 3.26E−10 |
| TNFRSF4 | Immune candidate | 0.761 | 1.28E−09 |
| FOXP3 | Immune candidate | 0.749 | 3.17E−09 |
| CD8A | Immune candidate | 0.749 | 3.20E−09 |
| CD4 | Immune candidate | 0.708 | 5.27E−08 |
| AXL | Immune candidate | 0.679 | 3.02E−07 |
| PDCD1LG2 | Immune candidate | 0.624 | 4.72E−06 |

Overall Median: 0.831
The correlation of expression profiles of 20 candidate genes between 9,223 TCGA tumors and 18,305 quantitative transcriptomic profiling (qTP) assessed tumors (of 24,463 samples, limited to 27 directly comparable tumor types) was determined. The number of samples used in comparison (n), the correlation (Spearman rho, P) and the significance with respect to no correlation (p-value, reported in scientific notation). TCGA data was obtained from cBioPortal. Components of the final Immunotherapy Response Score (IRS) model are bolded. For PD-L1 (CD274) and PD-1 (PDCD1), two independent target amplicons were assessed for each gene; normalized target gene expression was averaged from the independent amplicons (per gene) to yield a composite result. TCGA = the Cancer Genome Atlas. As limit of quantification (LOQ) could not be established for IFNG by Strata Multiplex PCR based-NGS profiling, it was excluded from this analysis.

TABLE S4
Univariate and multivariate associations of comprehensive genomic
and quantitative transcriptomic profiling derived biomarkers
and pembrolizumab real-world progression-free survival

| | Univariate | | | Multivariate (IRS model) | | |
|---|---|---|---|---|---|---|
| Biomarker | HR (95% CI) | | p-value | HR | | p-value |
| TMB | 0.79 | (0.73-0.86) | 1.6E−08 | 0.76 | (0.7-0.83) | 7.1E−11 |
| (PD-1) PDCD1 | 0.91 | (0.86-0.96) | 0.001 | 0.89 | (0.84-0.95) | 0.001 |
| Amplicon 1 | | | | | | |
| (PD-L1) CD274 | 0.92 | (0.87-0.98) | 0.005 | 0.94 | (0.88-1) | 0.048 |
| Amplicon 1 | | | | | | |
| (PD-L1) CD274 | 0.92 | (0.87-0.98) | 0.005 | | | |
| Amplicon 2 | | | | | | |
| IFNG** | 0.97 | (0.94-1) | 0.028 | | | |
| (PD-1) PDCD1 | 0.94 | (0.9-0.99) | 0.029 | | | |
| Amplicon 2 | | | | | | |
| CD8A | 0.95 | (0.9-1) | 0.038 | | | |
| TIGIT | 0.95 | (0.9-1) | 0.054 | | | |
| TNFRSF9 | 0.95 | (0.9-1.01) | 0.086 | | | |
| IDO1 | 0.97 | (0.93-1.01) | 0.096 | | | |
| PDCD1LG2 | 0.95 | (0.89-1.01) | 0.102 | | | |
| GZMA | 0.96 | (0.91-1.01) | 0.126 | | | |
| LAG3 | 0.95 | (0.89-1.02) | 0.155 | | | |
| UBE2C* | 0.95 | (0.88-1.03) | 0.225 | | | |
| ADAM12 | 1.03 | (0.97-1.1) | 0.276 | 1.06 | (1-1.13) | 0.055 |
| TCF7 | 0.97 | (0.9-1.05) | 0.486 | | | |
| CD4 | 1.02 | (0.94-1.12) | 0.618 | | | |
| VTCN1 | 0.99 | (0.97-1.02) | 0.639 | | | |
| CTLA4 | 0.99 | (0.94-1.04) | 0.648 | | | |
| HAVCR2 | 0.99 | (0.92-1.06) | 0.724 | | | |
| AXL | 1.01 | (0.94-1.1) | 0.725 | | | |
| TNFRSF4 | 1.00 | (0.94-1.07) | 0.930 | | | |
| TOP2A* | 1.00 | (0.92-1.09) | 0.972 | 1.08 | (0.99-1.18) | 0.080 |

| | | | |
|---|---|---|---|
| FOXP3 | 1.00 | (0.94-1.06) | 0.988 |

Derived candidate biomarkers and real-world progression-free survival in pembrolizumab treated patients (n = 648). For each biomarker amplicon, the hazard ratio (HR; with 95% confidence interval [CI]) and log-likelihood p-value are shown. TMB (log₂) was from StrataNGS CGP testing; the remaining biomarkers were target gene expression from in-parallel quantitative transcriptomic profiling (qTP). The multivariate analysis was performed using the final five component Immunotherapy Response Score (IRS) model.
*Candidate proliferation markers.
**Limit of quantification (LOQ) could not be established for this gene, therefore, it was not used for subsequent analysis.

TABLE S5
Correlation of individual components of the IRS model

| | PD-L1 | PD-1 | | | |
|---|---|---|---|---|---|
| | (CD274) | (PDCD1) | ADAM12 | TOP2A | TMB |
| PD-L1 | | 0.571 | 0.251 | 0.049 | 0.134 |
| (CD274) | | | | | |
| PD-1 | 0.571 | | 0.219 | 0.033 | 0.102 |
| (PDCD1) | | | | | |
| ADAM12 | 0.251 | 0.219 | | 0.06 | 0.032 |
| TOP2A | 0.049 | 0.033 | 0.06 | | 0.211 |
| TMB | 0.134 | 0.102 | 0.032 | 0.211 | |

The Spearman correlation (ρ) of individual components of IRS, including TMB (log2) and the four individual gene expression biomarkers (PD-L1, PD-1, ADAM12, and TOP2A) was determined across 24,463 samples with informative IRS within the cohort used to evaluated IRS distribution across tumors submitted for comprehensive genomic profiling. Increasing color intensity indicates increasing strength of the Spearman correlation. TMB = tumor mutation burden, IRS = Immunotherapy response score.

TABLE S6
Real-world PFS (rwPFS) and overall survival (OS) benefit after
unadjusted and adjusted restricted mean survival time analysis

| | | Restricted | | Estimate | |
|---|---|---|---|---|---|
| Analysis | Endpoint | Time (tau) | Variable | (95% CI) | p-value |
| Unadjusted | rwPFS | 24 Months | IRS-H | 15.70 | <0.0001* |
| RMST | | | | (14.53-16.88) | |
| | | | IRS-L | 10.63 | |
| | | | | (9.61-11.65) | |
| | OS | 36 Months | IRS-H | 25.50 | <0.0001* |
| | | | | (23.61-27.39) | |
| | | | IRS-L | 19.24 | |
| | | | | (17.48-21.00) | |
| Adjusted | rwPFS | 24 Months | Intercept | 10.63 | 0.0001 |
| RMST | | | | (5.27-15.99) | |
| | | | IRS | 4.80 | <0.0001 |
| | | | (H vs. L) | (3.20-6.41) | |
| | | | Age | 0.02 | 0.5984 |
| | | | | (−0.05-0.09) | |
| | | | Gender | 0.24 | 0.7662 |
| | | | (M vs. F) | (−1.31-1.79) | |
| | | | Tumor Type | 0.03 | 0.9748 |
| | | | (NSCLC vs. | (−1.74-1.80) | |
| | | | Other) | | |
| | | | Line of | −1.22 | 0.0298 |
| | | | Therapy | (−2.32-−0.12) | |
| | | | Type of | 0.48 | 0.5891 |
| | | | Therapy | (−1.26-2.22) | |
| | | | (Mono vs. | | |
| | | | Combo) | | |
| | OS | 36 Months | Intercept | 27.95 | <0.0001 |
| | | | | (18.80-37.10) | |
| | | | IRS | 6.00 | <0.0001 |
| | | | (H vs. L) | (3.37-8.63) | |
| | | | Age | −0.10 | 0.0920 |
| | | | | (−0.22-0.02) | |
| | | | Gender | −0.02 | 0.9897 |
| | | | (M vs. F) | (−2.62-2.59) | |
| | | | Tumor Type | 0.12 | 0.9382 |
| | | | (NSCLC vs. | (−2.92-3.17) | |
| | | | Other) | | |
| | | | Line of | −1.66 | 0.0825 |
| | | | Therapy | (−3.53-0.21) | |
| | | | Type of | 0.91 | 0.5524 |
| | | | Therapy | (−2.08-3.89) | |
| | | | (Mono vs. | | |
| | | | Combo) | | |

rwPFS = real-world progression-free survival, OS = overall survival, CI = confidence interval, IRS = immunotherapy response score, H = high, L = low, vs. = versus, RMST = restricted mean survival time, M = male, F = female, NSCLC = Non-Small Cell Lung Cancer, mono = monotherapy, combo = combination therapy.
*p-value is for a test of RMST equality between IRS-H and IRS-L

TABLE S7
Characteristics of the internal comparator cohort (n =
146) consisting of patients treated with pembrolizumab
monotherapy who had prior systemic therapy

| Variable | Statistic | Result | |
|---|---|---|---|
| | n | 146 | |
| Age | Median | 67 | (27, 89) |
| | (min, max) | | |
| Gender | n (%) | | |
| Female | | 65 | (44.5%) |
| Male | | 81 | (55.5%) |
| Unknown/Missing | | 0 | (0%) |
| Race | n (%) | | |
| American Indian or Alaskan | | 0 | (0%) |
| Native | | | |
| Asian | | 3 | (2.1%) |
| Black or African American | | 11 | (7.5%) |
| Native Hawaiian or Other | | 0 | (0%) |
| Pacific Islander | | | |
| White or Caucasian | | 94 | (64.4%) |
| Other | | 1 | (0.7%) |
| Unknown, Missing, or Not | | 37 | (25.3%) |
| Reported | | | |
| Ethnicity | n (%) | | |
| Hispanic or Latino | | 5 | (3.4%) |
| Not Hispanic or Latino | | 96 | (65.8%) |
| Unknown, Missing, or Not | | 45 | (30.8%) |
| Reported | | | |
| Prior Therapy Class | n (%) | | |
| Chemotherapy | | 128 | (87.7%) |
| Monoclonal antibodies | | 9 | (6.2%) |
| Antiangiogenesis | | 6 | (4.1%) |
| Oncogene targeted | | 2 | (1.4%) |
| Vaccine | | 1 | (0.7%) |
| Tumor Type | n (%) | | |
| Bladder Cancer | | 13 | (8.9%) |
| Breast Cancer | | 1 | (0.7%) |
| Cancer of Unknown Primary | | 16 | (11.0%) |
| Cervical Cancer | | 10 | (6.8%) |
| Colorectal Cancer | | 6 | (4.1%) |
| Endometrial Cancer | | 12 | (8.2%) |
| Esophagogastric Cancer | | 24 | (16.4%) |
| Head and Neck Cancer | | 24 | (16.4%) |
| Hepatobiliary Cancer | | 2 | (1.4%) |
| Melanoma | | 2 | (1.4%) |
| Neuroendocrine Tumor | | 1 | (0.7%) |
| Non-Small Cell Lung Cancer | | 12 | (8.2%) |
| Other Cancer | | 6 | (4.1%) |
| Ovarian Cancer | | 3 | (2.1%) |
| Pancreatic Cancer | | 5 | (3.4%) |
| Prostate Cancer | | 1 | (0.7%) |
| Sarcoma | | 3 | (2.1%) |
| Skin Cancer, Non-Melanoma | | 1 | (0.7%) |
| Small Cell Lung Cancer | | 2 | (1.4%) |
| Thymic Tumor | | 2 | (1.4%) |

n = sample size,
% = percentage

TABLE S8
Dependency of rwPFS from immediately prior therapy to

| | Reduced Model | | Full Model | |
|---|---|---|---|---|
| Covariate | HR (05% CI) | p-value | HR (95% CI) | p-value |
| IRS group | 0.69 | 0.015 | 0.98 | 0.92 |
| (IRS-high | (0.52-0.93) | | (0.70-1.38) | |
| vs. IRS-low) | | | | |
| Age | 1.00 | 0.7 | 1.00 | 0.81 |
| | (0.99-1.02) | | (0.99-1.02) | |
| Gender | 0.90 | 0.397 | 0.90 | 0.43 |
| (Male vs. | (0.70-1.16) | | (0.70-1.17) | |
| Female) | | | | |
| Tumor Type | 1.10 | 0.659 | 1.09 | 0.72 |
| (NSCLC vs. | (0.72-1.69) | | (0.69-1.70) | |
| Other) | | | | |
| Line of | 1.06 | 0.59 | 1.05 | 0.68 |
| Therapy | (0.85-1.33) | | (0.82-1.34) | |
| Type of | 0.53 | 0.001 | 0.74 | 0.14 |
| Therapy | (0.36-0.76) | | (0.50-1.10) | |
| (pembro | | | | |
| monotherapy | | | | |
| vs. prior | | | | |
| therapy) | | | | |
| Type of | NA | NA | 0.37 | 0.002 |
| Therapy × | | | (0.19-0.69) | |
| IRS Group | | | | |
| Interaction | | | | |
| test: p- | | | | |
| value = | | | | |
| 0.0011 | | | | |

rwPFS = real-world progression-free survival,
HR = hazard ratio,
CI = confidence interval,
IRS = immunotherapy response score,
NSCLC = Non-Small Cell Lung Cancer,
pembro = pembrolizumab
pembrolizumab monotherapy after adjustment for various covariates

TABLE S9
Pre-/post-propensity score matching covariates in first-
line NSCLC cohort treated with pembrolizumab monotherapy
or pembrolizumab + chemotherapy combination therapy

| Matching | | | Pembro + | Pembro | |
|---|---|---|---|---|---|
| Method | Variable | Statistic | Chemo | Mono | p-value * |
| None | | n | 133 | 109 | NA |
| | Age | Mean | 66.5 | 71.9 | <0.0001 |
| | TMB | Mean | 2.8 | 3 | 0.293 |
| | CD274 | Mean | 10.63 | 11.82 | <0.0001 |
| | Gender | n (%) | 60 (45.1%) | 54 (49.5%) | 0.5193 |
| | (Female) | | | | |
| | IRS | n (%) | 69 (51.9%) | 61 (56%) | 0.6044 |
| | (High) | | | | |
| NN - | | n | 77 | 77 | NA |
| 0.25*sd | Age | Mean | 68.94 | 69.97 | 0.5258 |
| caliper | TMB | Mean | 3.01 | 2.84 | 0.375 |
| | CD274 | Mean | 11.23 | 11.42 | 0.4596 |
| | Gender | n (%) | 41 (53.2%) | 35 (45.5%) | 0.4204 |
| | (Female) | | | | |
| | IRS | n (%) | 46 (59.7%) | 40 (51.9%) | 0.4172 |
| | (High) | | | | |

NN = nearest neighbor, sd = standard deviation, n = sample size, % = percent, NA = not applicable, IRS = immunotherapy response score, TMB = tumor mutation burden.
* p-value is based on a t-test for difference in means for continuous variables and a Fisher's exact test for categorical variables

TABLE S10
Impact of the tumor type term on the adjusted hazard ratio (HR) of IRS in
the Cox proportional hazards model in the discovery and validation cohorts

| | | | Most Common vs. | | MSKCC Sensitive vs. | |
|---|---|---|---|---|---|---|
| | | | Other Tumor Type | | Insensitive Tumors | |
| | | | IRS | | IRS | |
| | | | (-H vs. -L) | | (-H vs. -L) | |
| | | | Adjusted | | Adjusted | |
| Cohort | n | Analysis | HR (95% CI) | p-value | HR (95% CI) | p-value |
| Discovery | 648 | rwPFS | 0.49 | <0.0001 | 0.50 | <0.0001 |
| | | | (0.39-0.63) | | (0.39-0.64) | |
| Discovery | 648 | OS | 0.53 | <0.0001 | 0.54 | <0.0001 |
| | | | (0.40-0.70) | | (0.41-0.71) | |
| Validation | 248 | rwPFS | 0.52 | 0.003 | 0.46 | 0.0004 |
| | | | (0.34-0.80) | | (0.30-0.71) | |
| Validation | 248 | OS | 0.49 | 0.005 | 0.43 | 0.001 |
| | | | (0.30-0.80) | | (0.26-0.73) | |

The primary analyses presented herein used most common vs. other (e.g. NSCLC vs. all other tumor types in the discovery cohort) as the tumor type term in the Cox proportional hazards model used to evaluate the predictive ability of IRS for rwPFS and OS in the discovery (monotherapy and combination therapy) and validation cohorts. The impact on the adjusted HR (for IRS-High [H] vs. IRS-Low [L]) and p-value in the model was determined after replacing that tumor type term with the MSKCC definition of TMB sensitive tumor types (MSI-H, POLE^{mutant}, NSCLC, head and neck cancer, or melanoma as TMB sensitive; all other samples as TMB insensitive)⁵².

1. A method of treatment, comprising:
a. measuring expression levels of RNA transcripts for at least two of PD-1, TOP2A, PD-L1 and ADAM12 obtained from a tumor specimen from a subject;
b. measuring tumor mutation burden (TMB) in the biological sample and log 2 transforming the TMB measurement to provide a transformed TMB measurement;
c. calculating an Immunotherapy Response Score (IRS) from the expression levels of the RNA transcripts of the at least two of PD-1, TOP2A, PD-L1 and ADAM12 obtained in step a), and the transformed TMB measurement, wherein the IRS is positively correlated with the likelihood that the patient has a beneficial response to checkpoint inhibitor therapy, and obtaining an IRS having a value indicating a beneficial response to checkpoint inhibitor therapy; and
d. administering the checkpoint inhibitor therapy to the subject.

2.-5. (canceled)

6. The method of claim 1, wherein step a. further comprises ii) measuring expression levels of RNA transcripts for at least one reference gene in the biological sample, and iii) normalizing the measured expression levels of the measured RNA transcripts of the at least two, the at least three, or all of PD-1, TOP2A, PD-L1 and ADAM12 against the level of the RNA transcripts of the at least one reference gene to provide normalized expression levels of the PD-1, TOP2A, PD-L1 and ADAM12 RNA transcripts, and wherein the expression levels of RNA transcripts used to calculate the IRS comprises normalized expression levels of RNA transcripts.

7. (canceled)

8. The method of claim 6, wherein step a. further comprises iv) median centering the measured expression levels of RNA transcripts of the at least two of PD-1, TOP2A, PD-L1 and ADAM12, prior or after to normalizing the expression levels of the measured RNA transcripts.

9. The method of claim 8, wherein step a. further comprises v) log 2 transforming the measured expression levels, the median centered expression levels, the normalized expression levels or the median centered normalized expression levels of RNA transcripts of the at least two, the at least three, or all of PD-1, TOP2A, PD-L1 and ADAM12, and wherein the expression levels utilized to calculate the IRS in step c are transformed expression levels, transformed median centered expression levels, transformed normalized expression levels or transformed median centered normalized expression levels.

10.-14. (canceled)

15. The method of claim 6, wherein the one or more reference genes comprise three genes selected from the group consisting of CIAO1, EIF2B1, HMBS, CTCF, GGNBP2, ITGB7, MYC and SLC4A1AP.

16. (canceled)

17. The method of claim 1, wherein the tumor specimen is a formalin-fixed paraffin-embedded (FFPE) tumor specimen and the tumor specimen contains at least 20% tumor content.

18. (canceled)

19. The method of claim 1, wherein the tumor specimen is breast cancer, central or peripheral nervous system cancer, cancer of unknown primary, colorectal cancer, endometrial cancer, a gastrointestinal stromal tumor, glioma, hepatobiliary cancer, neuroendocrine cancer, ovarian cancer, pancreatic cancer, prostate cancer salivary gland cancer, sarcoma, or thyroid cancer.

20. The method of claim 1, wherein the tumor specimen is assessed as having either microsatellite instability-low or microsatellite stability and/or is assessed as having a low tumor mutational burden, wherein low tumor mutational burden is classified as having less than 10 mutations per megabase (muts/Mb).

21. (canceled)

22. The method of claim 1, wherein the expression levels of RNA transcripts are measured using PCR and next-generation sequencing.

23. The method of claim 1, wherein the checkpoint inhibitor is an anti-PD-1 antibody, an anti-CTLA-4 antibody, an anti-PD-L1 antibody, or an anti-PD-L2 antibody.

24. The method of claim 23, wherein the checkpoint inhibitor is nivolumab, pembrolizumab, atezolizumab, cemiplimab, durvalumab, pidilizumab, atezolimumab, PDR001, BMS-936559, avelumab, ipilimumab, or SHR-1210.

25.-29. (canceled)

30. The method of claim 1, wherein step a. comprises measuring expression levels of RNA transcripts for PD-1, TOP2A, PD-L1 and ADAM12, and one or more reference genes in a biological sample obtained from a tumor specimen from the subject, wherein the one or more reference gene comprises three genes selected from the group consisting of CIAO1, EIF2B1, HMBS, CTCF, GGNBP2, ITGB7, MYC and SLC4A1AP, and further log 2 transforming, median centering, and normalizing the measured expression levels of the RNA transcripts against the level of the RNA transcripts of at the least one reference gene to provide normalized levels of the RNA transcripts; and wherein step d. comprises calculating a Immunotherapy Response Score (IRS) from the transformed normalized levels of the RNA transcripts of PD-1, TOP2A, PD-L1 and ADAM12, and the transformed TMB measurement, wherein the IRS is positively correlated with the likelihood that the patient has a beneficial response to checkpoint inhibitor therapy.

31. The method of claim 1, wherein the tumor specimen is from a cancer not approved for labeled use of the checkpoint inhibitor therapy.

32.-33. (canceled)

34. The method of claim 1, wherein the tumor specimen shows a TPS score of 1-49%.

35. A method of identifying a subject that would benefit from checkpoint inhibitor therapy, comprising:
a. receiving, by a processor, measured expression levels of RNA transcripts for at least two of PD-1, TOP2A, PD-L1 and ADAM12, and at least one reference gene in a biological sample obtained from a tumor specimen from the subject;
b. log 2 transforming, median centering, and normalizing, by a processor, the measured expression levels of the RNA transcripts against the level of the RNA transcripts of at the least one reference gene to provide transformed normalized levels of the RNA transcripts;
c. receiving, by a processor, measured tumor mutation burden (TMB) in the biological sample;
d. log 2 transforming, by a processor, the TMB measurement to provide a transformed TMB measurement;
e. calculating, by a processor, an Immunotherapy Response Score (IRS) from the transformed normalized levels of the RNA transcripts of the at least two of PD-1, TOP2A, PD-L1 and ADAM12, and the transformed TMB measurement, that is positively correlated with the likelihood that the patient has a beneficial response to checkpoint inhibitor therapy; and
f. providing a determination if the subject has a checkpoint inhibitor responsive cancer.

36.-41. (canceled)

42. A method of treatment of a subject in need thereof with a checkpoint inhibitor therapy, comprising administering to said subject the checkpoint inhibitor therapy, wherein said subject in need thereof was previously identified by a method comprising:
a. measuring expression levels of RNA transcripts of PD-1, TOP2A, PD-L1 and ADAM12, and one or more reference genes in a biological sample obtained from a tumor specimen from the subject, wherein the one or more reference gene comprises three genes selected from the group consisting of CIAO1, EIF2B1, HMBS, CTCF, GGNBP2, ITGB7, MYC and SLC4A1AP;
b. log 2 transforming, median centering, and normalizing the measured expression levels of the RNA transcripts against the level of the RNA transcripts of the one or more reference genes to provide transformed normalized levels of the RNA transcripts;
c. measuring tumor mutation burden (TMB) in the biological sample and log 2 transforming the TMB measurement to provide a transformed TMB measurement; and
d. calculating an Immunotherapy Response Score (IRS) from the transformed normalized levels of the RNA transcripts of the at least two of PD-1, TOP2A, PD-L1 and ADAM12, and the transformed TMB measurement, wherein the IRS is positively correlated with the likelihood that the patient has a beneficial response to checkpoint inhibitor therapy, and obtaining an IRS having a value indicating beneficial response to checkpoint inhibitor therapy.

43.-58. (canceled)

59. A method of treatment, comprising:
a. measuring expression levels of RNA transcripts for PD-1 and PD-L2 obtained from a tumor specimen from a subject;
b. measuring tumor mutation burden (TMB) in the biological sample and log 2 transforming the TMB measurement to provide a transformed TMB measurement;
c. calculating an Immunotherapy Response Score (IRS) from the expression levels or normalized levels of the RNA transcripts of PD-1 and PD-L2, and the transformed TMB measurement, wherein the IRS is positively correlated with the likelihood that the patient has a beneficial response to checkpoint inhibitor therapy, and obtaining an IRS having a value indicating a beneficial response to checkpoint inhibitor therapy; and
d. administering the checkpoint inhibitor therapy to the subject.

60.-79. (canceled)

80. A method of identifying a subject that would benefit from checkpoint inhibitor therapy, comprising:
a. measuring expression levels of RNA transcripts for PD-1, PD-L2 and, optionally, expression levels of RNA transcripts for one or more of CD4, ADAM12, PD-L1, and VTCN1, and at least one reference gene in a biological sample obtained from a tumor specimen from the subject;
b. log 2 transforming, median centering, and normalizing the measured expression levels of the RNA transcripts against the level of the RNA transcripts of at the least one reference gene to provide normalized levels of the RNA transcripts;
c. measuring tumor mutation burden (TMB) in the biological sample and log 2 transforming the TMB measurement to provide a transformed TMB measurement;
d. calculating a Immunotherapy Response Score (IRS) from the normalized levels of the RNA transcripts of PD-1, PD-L2 and, optionally, one or more of CD4, ADAM12, PD-L1, and VTCN1, and the transformed TMB measurement, wherein the IRS is positively correlated with the likelihood that the patient has a beneficial response to checkpoint inhibitor therapy, and obtaining an IRS having a value indicating beneficial response to checkpoint inhibitor therapy; and
e. identifying the subject as benefiting from the checkpoint inhibitor therapy.

81.-82. (canceled)

83. A method of identifying a subject that would benefit from checkpoint inhibitor therapy, comprising:
a. receiving, by a processor, measured expression levels of RNA transcripts for PD-1, PD-L2 and, optionally, expression levels of RNA transcripts for one or more of CD4, ADAM12, PD-L1, and VTCN1, and at least one reference gene in a biological sample obtained from a tumor specimen from the subject;
b. log 2 transforming, median centering, and normalizing, by a processor, the measured expression levels of the RNA transcripts against the level of the RNA transcripts of the at least one reference gene to provide normalized levels of the RNA transcripts;
c. receiving, by a processor, measured tumor mutation burden (TMB) in the biological sample;
d. log 2 transforming, by a processor, the TMB measurement to provide a transformed TMB measurement;
e. calculating, by a processor, a Immunotherapy Response Score (IRS) from the normalized levels of the RNA transcripts of PD-1, PD-L2 and, optionally, one or more of CD4, ADAM12, PD-L1, and VTCN1, and the transformed TMB measurement, that is positively correlated with the likelihood that the patient has a beneficial response to checkpoint inhibitor therapy; and
f. providing a determination if the subject has a checkpoint inhibitor responsive cancer.

84.-91. (canceled)

92. A method of treatment of a subject in need thereof with a checkpoint inhibitor therapy, comprising administering to said subject the checkpoint inhibitor therapy, wherein said subject in need thereof was previously identified by a method, comprising:
a. measuring expression levels of RNA transcripts for PD-1, PD-L2, CD4, ADAM12, and one or more reference genes in a biological sample obtained from a tumor specimen from a subject, wherein the one or more reference genes comprise three genes selected from the group consisting of LRP1, MRPL13, TBP, HMBS, ITGB7, MYC, CIAO1, CTCF, EIF2B1, GGNBP2, and SLC4A1AP;
b. log 2 transforming, median centering, and normalizing the measured expression levels of the RNA transcripts of PD-1, PD-L2, CD4, and ADAM12 against the level of the RNA transcripts of at the least one reference gene to provide normalized levels of the RNA transcripts of PD-1, PD-L2, CD4, and ADAM12;
c. measuring tumor mutation burden (TMB) in the biological sample and log 2 transforming the TMB measurement to provide a transformed TMB measurement;
d. calculating a Immunotherapy Response Score (IRS) from the normalized levels of the RNA transcripts of PD-1, PD-L2, CD4, and ADAM12 and the transformed TMB measurement, wherein the IRS is positively correlated with the likelihood that the patient has a beneficial response to checkpoint inhibitor therapy, and obtaining an IRS having a value indicating a beneficial response to checkpoint inhibitor therapy; and
e. administering the checkpoint inhibitor therapy to the subject.

93.-99. (canceled)
